(19) United States
(12) Patent Application Publication
(10) Pub. No.: US 2025/0092032 A1
(43) Pub. Date: Mar. 20, 2025

(54) DEOXYCYTIDINE KINASE INHIBITORS

(71) Applicants: CENTRE NATIONAL DE LA RECHERCHE SCIENTIFIQUE, PARIS (FR); UNIVERSITE D'AIX-MARSEILLE, MARSEILLE (FR); INSTITUT NATIONAL DE LA SANTÉ ET DE LA RECHERCHE MÉDICALE, PARIS (FR); INSTITUT JEAN PAOLI & IRENE CALMETTES CENTRE REGIONAL DE LUTTE CONTRE LE CANCER, MARSEILLE (FR)

(72) Inventors: SÉBASTIEN ABEL, MARSEILLE (FR); STÉPHANE BETZI, MARSEILLE (FR); KHAOULA BEN YAALA-BEN MOUSSA, MARSEILLE (FR); PAUL BREMOND, SAINT-MAXIMIN-LA-SAINTE-BAUME (FR); SÉBASTIEN COMBES, FUVEAU (FR); PATRICE DUBREUIL, MARSEILLE (FR); LAURENT HOFFER, TORONTO (CA); XAVIER MORELLI, MARSEILLE (FR); ETIENNE REBUFFET, MARSEILLE (FR); PHILIPPE ROCHE, MARSEILLE (FR); MAGALI SAEZ-AYALA, MARSEILLE (FR); BENOIT SICARD, NANTES (FR)

(21) Appl. No.: 18/729,249

(22) PCT Filed: Jan. 17, 2023
(86) PCT No.: PCT/EP2023/050993

§ 371 Date: Jul. 16, 2024

(30) Foreign Application Priority Data
Jan. 17, 2022 (EP) 22305038.6

(51) Int. Cl. C07D 417/14; A61K 31/506; A61K 31/5377; A61K 31/55; A61P 35/02; C07D 417/04; C07D 487/10

(52) U.S. Cl. CPC C07D 417/14; A61K 31/506; A61K 31/5377; A61K 31/55; A61P 35/02; C07D 417/04; C07D 487/10

(57) ABSTRACT

The present invention relates to compounds of formula (I) as deoxycytidine kinase inhibitors and pharmaceutical compositions comprising the same. The present invention further relates to the use of such compounds of formula (I) for use for treating a cancer.

### FIELD OF THE INVENTION

The present invention relates to the field of medicine, in particular to Deoxycytidine kinase (dCK) inhibitors and their uses for treating a cancer.

### BACKGROUND OF THE INVENTION

Deoxycytidine kinase (dCK) is an enzyme having a crucial role in cellular division. This enzyme allows the phosphorylation of a large number of deoxyribonucleosides and their nucleoside analogs. More particularly, dCK catalyzes the 5′-phosphorylation of physiologic pyrimidines and purines, such as 2′-deoxycytosine (dC), 2′-deoxyadenosine (dA) and 2′-deoxyguanosine (dG). dCK has been observed to be predominantly expressed in a large panel of human cancer models in the Cancer Cell Line Encyclopedia (https://portals.broadinstitute.org/ccle) and was associated with certain forms of resistance to antiviral and anticancer chemotherapeutic agents. In this context, dCK has been validated as an interesting target in oncology. So far, there remains a need to develop further dCK inhibitors for the treatment of diseases and disorders for which dCK activity is implicated, such as cell proliferative diseases, particularly cancer.

### SUMMARY OF THE INVENTION

In this context, the inventors have provided new compounds having the properties to bind to dCK and inhibit its activity, demonstrating thereby the therapeutic interest of such compounds in medicine, more particularly in cancer therapies.

The present invention thus provides a compound having the following formula (I):

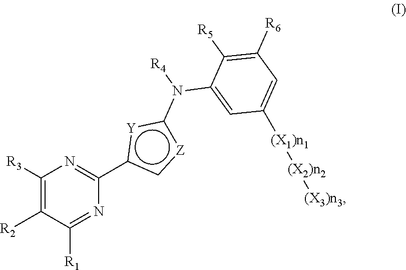

wherein:
Y and Z represent independently NH, N, O, or S;
R₁, R₂, and R₃ represent independently a radical selected in the group consisting of a hydrogen, a —NR₇R₈ group with R₇ and R₈ being independently a hydrogen or a (C₁-C₆)alkyl group, and a halogen;
R₄ and R₅ represent independently a hydrogen, a (C₁-C₆)alkyl group optionally substituted by a radical selected in the group consisting of a cycloalkyl, and a —NR₉R₁₀ group with R₉ and R₁₀ being independently a hydrogen or a (C₁-C₆)alkyl group, or R₄ and R₅ form together an azepanyl;
R₆ represents a hydrogen or a halogen;
X₁ represents a —NHCO— group, an oxygen atom, a halogen, a —C≡C— group, or a —O—CH₂— group;
X₂ represents a 5-12 membered ring optionally substituted by at least one radical selected in the group consisting of a (C₁-C₆)alkyl group optionally substituted by at least one halogen, a (C₁-C₆)alkoxy group optionally substituted by at least one halogen, a halogen, and a hydroxy;
X₃ represents a radical selected in the group consisting of:
   a (C₁-C₆)alkyl substituted by at least one radical A
   a (C₁-C₆)alkoxy substituted by at least one radical A,
   a —C(O)— substituted by at least one radical A,
   a —SO₂— substituted by at least one radical A,
   a —NH—SO₂— substituted by at least one radical A,
   a —NHCO— substituted by at least one radical A,
in which said at least one radical A is selected in the group consisting of:
   a piperazinyl optionally substituted by a (C₁-C₆)alkyl or by a (C₁-C₆)alkyl substituted by a —COOH,
   a —CO-piperazinyl substituted by a (C₁-C₆)alkyl,
   a morpholinyl,
   a piperidinyl optionally substituted by a —NR₇R₈ group with R₇ and R₈ being independently a hydrogen or a (C₁-C₆)alkyl group, or
   a 2,6-diazaspiro[3.3]heptanyl substituted by a (C₁-C₆)alkyl,
   a —NR₁₁R₁₂ group with R₁₁ and R₁₂ being independently a hydrogen, a (C₁-C₆)alkyl group, or a —SO₂—CH₃ group,
   a —NH—(C₁-C₆)alkyl-NR₁₃R₁₄ group with R₁₃ and R₁₄ being independently a hydrogen, or a (C₁-C₆)alkyl group,
   a (C₁-C₆)alkoxy, and
   a hydroxy,
   a hydroxy, and
   a —NR₁₅R₁₆ group with R₁₅ and R₁₆ being independently a hydrogen or a (C₁-C₆)alkyl group; and
n₁, n₂, and n₃ are independently 0 or 1;
   and the pharmaceutically acceptable salts, the tautomers, and the solvates thereof.

In a particular embodiment, the compound of formula (I) is such that:
Y and Z represent independently NH, N, O, or S;
R₁, R₂, and R₃ represent independently a radical selected in the group consisting of a hydrogen, a —NR₇R₈ group with R₇ and R₈ being independently a hydrogen or a (C₁-C₆)alkyl group, and a halogen;
R₄ and R₅ represent independently a hydrogen, a (C₁-C₆)alkyl group optionally substituted by a radical selected in the group consisting of a cycloalkyl, and a —NR₉R₁₀ group with R₉ and R₁₀ being independently a hydrogen or a (C₁-C₆)alkyl group, or R₄ and R₅ form together an azepanyl;
R₆ represents a hydrogen or a halogen;
X₁ represents a —NHCO— group, an oxygen atom, a —C≡C— group, or a —O—CH₂— group;
X₂ represents a 5-12 membered ring optionally substituted by at least one radical selected in the group consisting of a (C₁-C₆)alkyl group optionally substituted by at least one halogen, a (C₁-C₆)alkoxy group optionally substituted by at least one halogen, and a halogen;
X₃ represents a radical selected in the group consisting of:
   a (C₁-C₆)alkyl substituted by at least one radical A
   a (C₁-C₆)alkoxy substituted by at least one radical A,
   a —C(O)— substituted by at least one radical A,
   a —SO₂— substituted by at least one radical A,
   a —NH—SO₂— substituted by at least one radical A,
   a —NHCO— substituted by at least one radical A,
in which said at least one radical A is selected in the group consisting of:
   a piperazinyl optionally substituted by a (C₁-C₆)alkyl or by a (C₁-C₆)alkyl substituted by a —COOH,
   a —CO-piperazinyl substituted by a (C₁-C₆)alkyl,
   a morpholinyl,
   a piperidinyl optionally substituted by a —NR₇R₈ group with R₇ and R₈ being independently a hydrogen or a (C₁-C₆)alkyl group, or
   a 2,6-diazaspiro[3.3]heptanyl substituted by a (C₁-C₆)alkyl,
   a —NR₁₁R₁₂ group with R₁₁ and R₁₂ being independently a hydrogen, a (C₁-C₆)alkyl group, or a —SO₂—CH₃ group,
   a —NH—(C₁-C₆)alkyl-NR₁₃R₁₄ group with R₁₃ and R₁₄ being independently a hydrogen, or a (C₁-C₆)alkyl group,
   a (C₁-C₆)alkoxy, and
   a hydroxy,
   a hydroxy, and
   a —NR₁₅R₁₆ group with R₁₅ and R₁₆ being independently a hydrogen or a (C₁-C₆)alkyl group; and
n₁, n₂, and n₃ are independently 0 or 1;
   and the pharmaceutically acceptable salts, the tautomers, and the solvates thereof.

Preferably, said compound has the following formula (I′):

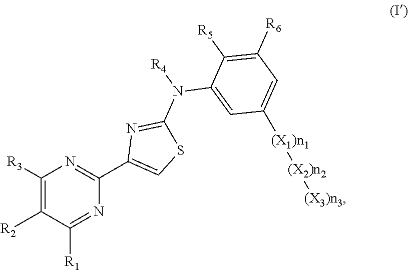

in which, R₁, R₂, R₃, R₄, R₅, R₆, X₁, X₂, X₃, n₁, n₂, and n₃ are such as defined herein.

In a particular embodiment, the compound of formula (I) or (I′) is such that at least one group chosen among R₁, R₂, and R₃ is not a hydrogen.

In a particular embodiment, the compound of formula (I) or (I′) is such that:
R₁ represents a —NR₇R₈ group with R₇ and R₈ being a hydrogen;
R₂ represents a hydrogen or a halogen, preferably a hydrogen; and
R₃ represents a hydrogen or a —NR₇R₈ group with R₇ and R₈ being a hydrogen, preferably a —NR₇R₈ group with R₇ and R₈ being a hydrogen.

In a particular embodiment, the compound of formula (I) or (I′) is such that:
R₄ represents a hydrogen, or a (C₁-C₆)alkyl group optionally substituted by a cycloalkyl or by a —NR₉R₁₀ group with R₉ and R₁₀ being a hydrogen, preferably R₄ represents a (C₁-C₆)alkyl group; and
R₅ represents a (C₁-C₆)alkyl group, preferably a methyl group.

In a particular embodiment, the compound of formula (I) or (I′) is such that R₆ represents a hydrogen.

In a particular embodiment, the compound of formula (I) or (I′) is such that n₁+n₂+n₃ is equal or superior to 1, preferably n₁+n₂+n₃ is 1, 2, or 3.

In a particular embodiment, the compound of formula (I) or (I′) is such that n₁ is 0.

In a further particular embodiment, the compound of formula (I) or (F) is such that n₁ is 1 and X₁ represents a —NHCO— group.

In a particular embodiment, the compound of formula (I) or (I′) is such that n₂ is 1 and X₂ represents a 5-12 membered ring selected in the group consisting of a phenyl, a pyrimidinyl, a thiophenyl, a pyridinyl, a triazolyl, and an indolyl, said 5-12 membered ring is optionally substituted by at least one radical selected in the group consisting of a methoxy, a trifluoromethoxy, a halogen, a hydroxy, a methyl, and a trifluromethyl.

In a particular embodiment, the compound of formula (I) or (I′) is such that n₃ is 1 and X₃ represents a radical selected in the group consisting of a (C₁-C₆)alkyl, a (C₁-C₆)alkoxy, a —C(O)—, a —SO₂—, a —NH—SO₂—, and a —NHCO—, said radical being substituted by a piperazinyl optionally substituted by a (C₁-C₆)alkyl, preferably a piperazinyl substituted by a methyl.

In a further particular embodiment, the compound of formula (I) or (I′) is such that n₃ is 1 and X₃ represents a (C₁-C₆)alkyl substituted by at least one radical A as defined herein, preferably, a piperazinyl substituted by a methyl, a —N(CH₃)₂, a 2,6-diazaspiro[3.3]heptanyl substituted by a methyl, and a —NHSO₂CH₃.

In a further particular embodiment, the compound of formula (I) or (I′) is such that n₃ is 1 and X₃ represents a —SO₂— substituted by at least one radical A as defined herein, preferably a piperazinyl substituted by a methyl, a —NR₁₁R₁₂ group with Rn and R₁₂ being independently a hydrogen or a methyl group, and a morpholinyl.

In a preferred embodiment, the compound of formula (I) or (I′) is such that:
R₁, R₂, and R₃ represent independently a hydrogen, or a —NR₇R₈ group with R₇ and R₈ being independently a hydrogen, preferably R₁ and R₃ represent NH₂ and R₂ represents H;
R₄ and R₅ represent independently a (C₁-C₆)alkyl group, preferably R₄ represents a propyl and R₅ a methyl;
R₆ represents a hydrogen;
X₂ represents a 5-6 membered ring selected in the group consisting of a phenyl, and a pyridinyl, said 5-6 membered ring being optionally substituted by at least one radical selected in the group consisting of a trifluoromethyl, a trifluoromethoxy, a halogen, a hydroxy, a methoxy, and a methyl;
X₃ represents a —SO₂— substituted by at least one radical selected in the group consisting of a piperazinyl optionally substituted by a (C₁-C₆)alkyl or by a (C₁-C₆)alkyl substituted by a —COOH, a piperidinyl optionally substituted by a —NH₂, and a —NH—(C₁-C₆)alkyl-NH₂;
n₁ is 0; and
n₂, and n₃ are 1.

A preferred compound of formula (I) is selected in the group consisting of:
N-(3-((4-(4-Aminopyrimidin-2-yl)thiazol-2-yl)amino)-4-methylphenyl)-4-((4-methylpiperazin-1-yl)methyl)benzamide dCKi-1;
N-(3-((4-(4,6-Diaminopyrimidin-2-yl)thiazol-2-yl)amino)-4-methylphenyl)-4-((4-methylpiperazin-1-yl)methyl)benzamide dCKi-2:
N-(3-((4-(4-Aminopyrimidin-2-yl)thiazol-2-yl)(propyl)amino)-4-methylphenyl)-4-((4-methylpiperazin-1-yl)methyl)benzamide OR005;
N-(3-((4-(4-Aminopyrimidin-2-yl)thiazol-2-yl)(propyl)amino)-4-methylphenyl)-2-((4-methylpiperazin-1-yl)methyl)pyrimidine-5-carboxamide OR025; 4-(4-Aminopyrimidin-2-yl)-N-(2-methyl-5-(4-((4-methylpiperazin-1-yl)methyl)phenoxy)phenyl)thiazol-2-amine OR043;
N-(3-((4-(4-Aminopyrimidin-2-yl)thiazol-2-yl)amino)-4-methylphenyl)-4-(2-(4-methylpiperazin-1-yl)ethyl)benzamide OR046;
4-(4-Aminopyrimidin-2-yl)-N-(2-methyl-5-((4-((4-methylpiperazin-1-yl)methyl)phenyl)ethynyl)phenyl)thiazol-2-amine OR053;
N-(3-((4-(4-Aminopyrimidin-2-yl)thiazol-2-yl)amino)-4-methylphenyl)-4-((dimethylamino)methyl)benzamide OR055;
N-(3-((4-(4-Aminopyrimidin-2-yl)thiazol-2-yl)amino)-4-methylphenyl)-4-((6-methyl-2,6-diazaspiro[3.3]heptan-2-yl)methyl)benzamide OR0156;
4-(4-Aminopyrimidin-2-yl)-N-(2-methyl-5-(4-((4-methylpiperazin-1-yl)methyl)benzyloxy)phenyl)thiazol-2-amine OR0232;
4-(4-Aminopyrimidin-2-yl)-N-(5-((4-((dimethylamino)methyl)phenyl)ethynyl)-2-methylphenyl)thiazol-2-amine OR0237;
N-(3-((4-(4-Amino-5-fluoropyrimidin-2-yl)thiazol-2-yl)amino)-4-methylphenyl)-4-((4-methylpiperazin-1-yl)methyl)benzamide OR0239;
N-(3-((4-(4-Aminopyrimidin-2-yl)thiazol-2-yl)amino)-4-methylphenyl)-5-((4-methylpiperazin-1-yl)methyl)thiophene-2-carboxamide OR0241;
4-(4-Aminopyrimidin-2-yl)-N-(4-methyl-4′-((4-methylpiperazin-1-yl)methyl)-[1,1′-biphenyl]-3-yl)thiazol-2-amine OR0274;
N-(1-(4-(4-Aminopyrimidin-2-yl)thiazol-2-yl)-2,3,4,5-tetrahydro-1H-benzo[b]azepin-8-yl)-4-((4-methylpiperazin-1-yl)methyl)benzamide OR0289;
3′-((4-(4-Aminopyrimidin-2-yl)thiazol-2-yl)amino)-4′-methyl-[1,1′-biphenyl]-4-ol OR0320;
3′-((4-(4-Aminopyrimidin-2-yl)thiazol-2-yl)amino)-4′-methyl-[1,1′-biphenyl]-4-ol OR0321;
4-(4-Aminopyrimidin-2-yl)-N-(4-methyl-4′-(2-(4-methylpiperazin-1-yl)ethyl)-[1,1′-biphenyl]-3-yl)thiazol-2-amine OR0325;
4-(4-Aminopyrimidin-2-yl)-N-(4-methyl-4′-(3-(4-methylpiperazin-1-yl)propyl)-[1,1′-biphenyl]-3-yl)thiazol-2-amine OR0331;
4-(4-Aminopyrimidin-2-yl)-N-(4-methyl-4′-(3-(4-methylpiperazin-1-yl)ethyl)-[1,1′-biphenyl]-3-yl)-N-propylthiazol-2-amine OR0345;
2-(2-(8-(4-(2-(4-Methylpiperazin-1-yl)ethyl)phenyl)-2,3,4,5-tetrahydro-1H-benzo[b]azepin-1-yl)thiazol-4-yl)pyrimidin-4-amine OR0402;
4-(4-Aminopyrimidin-2-yl)-N-(4-methyl-4′-(2-(4-methylpiperazin-1-yl)ethoxy)-[1,1′-biphenyl]-3-yl)-N-propylthiazol-2-amine OR0596;
4-(4-Aminopyrimidin-2-yl)-N-(4-methyl-4′-(2-(4-methylpiperazin-1-yl)-2-oxoethoxy)-[1,1′-biphenyl]-3-yl)-N-propylthiazol-2-amine OR0597;
4-(4-Aminopyrimidin-2-yl)-N-(4-methyl-4′-(3-(4-methylpiperazin-1-yl)propyl)-[1,1′-biphenyl]-3-yl)-N-propylthiazol-2-amine OR0598;
4-(4-Aminopyrimidin-2-yl)-N-(2-methyl-5-(6-(2-(4-methylpiperazin-1-yl)ethyl)pyridin-3-yl)phenyl)-N-propylthiazol-2-amine OR0599;
2-(2-((4-Methyl-4′-(2-(4-methylpiperazin-1-yl)ethyl)-[1,1′-biphenyl]-3-yl)(propyl)amino)thiazol-4-yl)pyrimidine-4,6-diamine OR0600;
2-(2-((4-Methyl-4′-(3-(4-methylpiperazin-1-yl)propyl)-[1,1′-biphenyl]-3-yl)(propyl)amino)thiazol-4-yl)pyrimidine-4,6-diamine OR0601;
2-(2-((2-Methyl-5-(6-(2-(4-methylpiperazin-1-yl)ethyl)pyridin-3-yl)phenyl)(propyl)amino)thiazol-4-yl)pyrimidine-4,6-diamine OR0602;
2-(2-(iso-Butyl(4-methyl-4′-(2-(4-methylpiperazin-1-yl)ethyl)-[1,1′-biphenyl]-3-yl)amino)thiazol-4-yl)pyrimidine-4,6-diamine OR0603;
2-(2-((Cyclopropylmethyl)(4-methyl-4′-(2-(4-methylpiperazin-1-yl)ethyl)-[1,1′-biphenyl]-3-yl)amino)thiazol-4-yl)pyrimidine-4,6-diamine OR0604;
2-(2-(iso-Pentyl(4-methyl-4′-(2-(4-methylpiperazin-1-yl)ethyl)-[1,1′-biphenyl]-3-yl)amino)thiazol-4-yl)pyrimidine-4,6-diamine OR0605;
2-(2-(Butyl(4-methyl-4′-(2-(4-methylpiperazin-1-yl)ethyl)-[1,1′-biphenyl]-3-yl)amino)thiazol-4-yl)pyrimidine-4,6-diamine OR0606;
2-(2-((4-Methyl-4′-((4-methylpiperazin-1-yl)sulfonyl)-[1,1′-biphenyl]-3-yl)(propyl)amino)thiazol-4-yl)pyrimidine-4,6-diamine OR0607;
2-(2-(iso-Butyl(2-methyl-5-(6-(2-(4-methylpiperazin-1-yl)ethoxy)pyridin-3-yl)phenyl)amino)thiazol-4-yl)pyrimidine-4,6-diamine OR0608;
N-((1-(3-((4-(4,6-Diaminopyrimidin-2-yl)thiazol-2-yl)(isobutyl)amino)-4-methylphenyl)-1H-1,2,3-triazol-4-yl)methyl) methanesulfonamide OR0609;
2-(2-((2-Aminoethyl)(4-methyl-4′-(2-(4-methylpiperazin-1-yl)ethyl)-[1,1′-biphenyl]-3-yl)amino)thiazol-4-yl)pyrimidine-4,6-diamine OR0610;
2-(2-((3-Aminopropyl)(4-methyl-4′-(2-(4-methylpiperazin-1-yl)ethyl)-[1,1′-biphenyl]-3-yl)amino)thiazol-4-yl)pyrimidine-4,6-diamine OR0611;
2-[2-({2-Methyl-5-[6-(4-methyl-piperazin-1-ylcarbonyl)-pyridin-3-yl]-phenyl}-propyl-amino)-thiazol-4-yl]-pyrimidine-4,6-diamine OR0612;
2-(2-((5-(2-Aminopyrimidin-5-yl)-2-methylphenyl)(propyl)amino)thiazol-4-yl)pyrimidine-4,6-diamine OR0613;
2-(2-((4-Methyl-4′-((4-methylpiperazin-1-yl)sulfonyl)-[1,1′-biphenyl]-3-yl)amino)thiazol-4-yl)pyrimidine-4,6-diamine OR0614;
3′-((4-(4,6-Diaminopyrimidin-2-yl)thiazol-2-yl)(propyl)amino)-4′-methyl-[1,1′-biphenyl]-4-sulfonamide OR0615;
N-(5-(3-((4-(4,6-Diaminopyrimidin-2-yl)thiazol-2-yl)(propyl)amino)-4-methylphenyl)pyridin-2-yl)-4-methylpiperazine-1-carboxamide OR0616;
2-(2-((4-Methyl-4′-(morpholinosulfonyl)-[1,1′-biphenyl]-3-yl)(propyl)amino)thiazol-4-yl)pyrimidine-4,6-diamine OR0617;
3′-((4-(4,6-Diaminopyrimidin-2-yl)thiazol-2-yl)(propyl)amino)-N,N,4′-trimethyl-[1,1′-biphenyl]-4-sulfonamide OR0618;
2-(2-((4′-((Dimethylamino)methyl)-4-methyl-[1,1′-biphenyl]-3-yl)(propyl)amino)thiazol-4-yl)pyrimidine-4,6-diamine OR0619;
2-(2-((2-Methyl-5-(pyridin-3-yl)phenyl)(propyl)amino)thiazol-4-yl)pyrimidine-4,6-diamine OR0620;
(5-(3-((4-(4,6-Diaminopyrimidin-2-yl)thiazol-2-yl)(propyl)amino)-4-methylphenyl)pyridin-2-yl)(morpholino) methanone OR0621;
Methyl 5-(3-((4-(4,6-diaminopyrimidin-2-yl)thiazol-2-yl)(propyl)amino)-4-methylphenyl)picolinate OR0622;
N-(5-(3-((4-(4,6-Diaminopyrimidin-2-yl)thiazol-2-yl)(propyl)amino)-4-methylphenyl)pyridin-2-yl)-4-methylpiperazine-1-sulfonamide OR0625;
2-(2-((5-(1H-Indol-5-yl)-2-methylphenyl)(propyl)amino)thiazol-4-yl)pyrimidine-4,6-diamine OR0626;
2-(2-((2-Methyl-5-(6-((4-methylpiperazin-1-yl)sulfonyl)pyridin-3-yl)phenyl)(propyl)amino)thiazol-4-yl)pyrimidine-4,6-diamine OR0627;
2-(2-((2-Methyl-5-(6-(2-(4-methylpiperazin-1-yl)ethoxy)pyridin-3-yl)phenyl)(propyl)amino)thiazol-4-yl)pyrimidine-4,6-diamine OR0629;
2-((5-(3-((4-(4,6-Diaminopyrimidin-2-yl)thiazol-2-yl)(propyl)amino)-4-methylphenyl)pyridin-2-yl)oxy)-1-(4-methylpiperazin-1-yl)ethan-1-one OR0630;
2-(2-(Methyl(4-methyl-4′-((4-methylpiperazin-1-yl)sulfonyl)-[1,1′-biphenyl]-3-yl)amino)thiazol-4-yl)pyrimidine-4,6-diamine OR0631;
2-(2-(Ethyl(4-methyl-4′-((4-methylpiperazin-1-yl)sulfonyl)-[1,1′-biphenyl]-3-yl)amino)thiazol-4-yl)pyrimidine-4,6-di-amine OR0632;
2-(2-((5-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0633;
2-(2-((3′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine OR0635;
2-(2-(iso-Propyl(4-methyl-4′-((4-methylpiperazin-1-yl)sulfonyl)-[1,1′-biphenyl]-3-yl)amino)thiazol-4-yl)pyrimidine-4,6-diamine OR0636;
2-(2-(iso-Butyl(4-methyl-4′-((4-methylpiperazin-1-yl)sulfonyl)-[1,1′-biphenyl]-3-yl)amino)thiazol-4-yl)pyrimidine-4,6-diamine OR0637;
2-(2-((4′-((4-Ethylpiperazin-1-yl)sulfonyl)-4-methyl-[1,1′-biphenyl]-3-yl)(propyl)amino)thiazol-4-yl)pyrimidine-4,6-diamine OR0638;
2-(2-((4-Methyl-4′-(piperazin-1-ylsulfonyl)-[1,1′-biphenyl]-3-yl)(propyl)amino)thiazol-4-yl)pyrimidine-4,6-diamine OR0639;
2-(2-((4-Methyl-4′-((4-methylpiperazin-1-yl)sulfonyl)-[1,1′-biphenyl]-3-yl)(propyl)amino)oxazol-4-yl)pyrimidine-4,6-diamine OR0640;
2-(2-((2′-Chloro-4-methyl-4′-((4-methylpiperazin-1-yl)sulfonyl)-[1,1′-biphenyl]-3-yl)(propyl)amino)thiazol-4-yl)pyrimidine-4,6-diamine OR0641;
2-(2-((4-Methyl-4′-((4-methylpiperazin-1-yl)sulfonyl)-2′-(trifluoromethyl)-[1,1′-biphenyl]-3-yl)(propyl)amino) thiazol-4-yl)pyrimidine-4,6-diamine OR0642;
2-(2-((2′-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0643;
2-(2-((2′-Fluoro-6′-methoxy-4-methyl-4′-((4-methylpiperazin-1-yl)sulfonyl)-[1,1′-biphenyl]-3-yl)(propyl)amino) thiazol-4-yl)pyrimidine-4,6-diamine OR0644;
2-(2-((2′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine OR0645;
2-(2-((4-Methyl-4′-(piperidin-4-ylsulfonyl)-[1,1′-biphenyl]-3-yl)(propyl)amino)thiazol-4-yl)pyrimidine-4,6-diamine OR0646;
2-(2-((2′,6′-Difluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0647;
2-(2-((4′-((4-Aminopiperidin-1-yl)sulfonyl)-4-methyl-[1,1′-biphenyl]-3-yl)(propyl)amino)thiazol-4-yl)pyrimidine-4,6-diamine OR0648;
2-(4-((3′-((4-(4,6-Diaminopyrimidin-2-yl)thiazol-2-yl)(propyl)amino)-4′-methyl-[1,1′-biphenyl]-4-yl)sulfonyl) piperazin-1-yl)acetic acid OR0649;
2-(2-((4-Methyl-4′-((4-methylpiperazin-1-yl)sulfonyl)-3′-(trifluoromethoxy)-[1,1′-biphenyl]-3-yl)(propyl)amino) thiazol-4-yl)pyrimidine-4,6-diamine OR0650;
N-(2-Aminoethyl)-3′-((4-(4,6-diaminopyrimidin-2-yl)thiazol-2-yl)(propyl)amino)-4′-methyl-[1,1′-biphenyl]-4-sulfonamide OR0651;
2-(2-((2-Methyl-5-(4-methyl-6-((4-methylpiperazin-1-yl)sulfonyl)pyridin-3-yl)phenyl)(propyl)amino)thiazol-4-yl)pyrimidine-4,6-diamine OR0652;
2-(2-((5-Bromo-2-methylphenyl)(isobutyl)amino)thiazol-4-yl)pyrimidine-4,6-diamine OR0637-1;
2-(2-((5-Bromo-2-methylphenyl)(propyl)amino)thiazol-4-yl)pyrimidine-4,6-diamine OR0652-1;
2-(2-((2-Methyl-5-(2-methyl-6-((4-methylpiperazin-1-yl)sulfonyl)pyridin-3-yl)phenyl)(propyl)amino)thiazol-4-yl)pyrimidine-4,6-diamine OR0653;
2-(2-((5-(6-Methoxy-4-(trifluoromethyl)pyridin-3-yl)-2-methylphenyl)(propyl)amino)thiazol-4-yl)pyrimidine-4,6-diamine OR0654;
2-(2-((5-(6-Hydroxy-4-(trifluoromethyl)pyridin-3-yl)-2-methylphenyl)(propyl)amino)thiazol-4-yl)pyrimidine-4,6-diamine OR0655;
2-(2-((2-Methyl-5-(6-((4-methylpiperazin-1-yl)sulfonyl)-4-(trifluoromethyl)pyridin-3-yl)phenyl)(propyl)amino)thiazol-4-yl)pyrimidine-4,6-diamine OR0656;
2-(2-((2-Methyl-5-(6-((4-methylpiperazin-1-yl)sulfonyl)-2-(trifluoromethyl)pyridin-3-yl)phenyl)(propyl)amino)thiazol-4-yl)pyrimidine-4,6-diamine OR0657; and
2-(2-((2-Methyl-5-(6-(piperazin-1-ylsulfonyl)-2-(trifluoromethyl)pyridin-3-yl)phenyl)(propyl)amino)thiazol-4-yl)pyrimidine-4,6-diamine OR0658.

A further object of the invention is a compound as defined herein for use as a drug.

A further object is a pharmaceutical composition comprising a compound as defined herein, and a pharmaceutically acceptable excipient. In a particular embodiment, the pharmaceutical composition further comprises an inhibitor of the De Novo nucleotide biosynthesis pathway, particularly a ribonucleotide reductase inhibitor, preferably thymidine.

Another object of the invention is a compound as defined herein or a pharmaceutical composition as defined herein for use for treating a cancer, preferably a liquid cancer, more preferably acute lymphoblastic leukemia, even more preferably T-cell acute lymphoblastic leukemia.

### LEGENDS OF THE FIGURES

FIG. 1: Scheme of the strategy for co-targeting deoxyribonucleotide triphosphate synthesis by inhibiting de novo and salvage pathways simultaneously. A dCK inhibitor in combination with a RNR inhibitor inhibits cancer cells proliferation, as both pathways are inhibited at the same time. The dTTP produced via TK1 from exogenously added dT acts as a RNR inhibitor for pyrimidines reduction by an allosteric regulation of the R₁ subunit. (RNR: ribonucleotide reductase, TK: thymidine kinase, dCK: deoxycytidine kinase).

FIG. 2: Effect of compounds of the invention on dCK thermal stabilization (determined by the binding assay by thermal shift assay) and cellular proliferation assay (on the CCRF-CEM cell line in presence of 200 μM dT and 1 μM dC). Grey square represents compound OR0642.

FIG. 3: Effect of selected compounds of the invention on cell proliferation of the CCRF-CEM cell line in presence of dT (200 μM) and dC (1 μM).

FIG. 4: Schedule of the vehicle and drugs administration (vehicle, dT 1.5 g/Kg, OR0642 40 mg/Kg, and dT+OR0642) (once/day (QD) and twice/day (BID)) against CCRF-CEM leukemia bearing mice.

FIG. 5: Effect of treatments on the quantification of whole-body radiance of CCRF-CEM leukemia bearing mice (bioluminescence). Mice were treated with vehicle (CTRL), dT, OR0642 or dT+OR0642. The arrow highlights the end of the treatment. Data were presented as the mean f SEM.

FIG. 6: Effect of treatments on the quantification of whole-body radiance of CCRF-CEM leukemia bearing mice (bioluminescence) at Day 21. Mice were treated with vehicle (CTRL), dT, OR0642 or dT+OR0642. Comparison of bioluminescence data at Day 21 was performed using One way ANOVA Kruskal-Wallis Test and Dunn's Multiple Comparison Test. (NI=non injected mouse).

FIG. 7: Effect of treatments of CCRF-CEM leukemia bearing mice on hCD45 populations in blood determined by flux cytometry at Day 21. Mice were treated with vehicle (CTRL), dT, OR0642 or dT+OR0642. Comparison of hCD45 populations was performed using One way ANOVA Kruskal-Wallis Test and Dunn's Multiple Comparison Test.

FIG. 8: Survival analysis of CCRF-CEM leukemia bearing mice treated with vehicle (CTRL), dT, OR0642 or dT+OR0642. The arrow highlights the end of the treatment. Median survival times were compared using Log-Rank (Mantel-Cox) Test.

### DETAILED DESCRIPTION OF THE INVENTION

### Definitions

According to the present invention, the terms below have the following meanings:

The terms mentioned herein with prefixes such as for example C₁-C₆, can also be used with lower numbers of carbon atoms such as C₁-C₂. If, for example, the term C₁-C₆ is used, it means that the corresponding hydrocarbon chain may comprise from 1 to 6 carbon atoms, especially 1, 2, 3, 4, 5, or 6 carbon atoms. If, for example, the term C₁-C₃ is used, it means that the corresponding hydrocarbon chain may comprise from 1 to 3 carbon atoms, especially 1, 2, or 3 carbon atoms.

The term “alkyl” refers to a saturated, linear or branched aliphatic group. The term “(C₁-C₆)alkyl” more specifically means methyl, ethyl, propyl, isopropyl, butyl, isobutyl, pentyl, isopentyl, or hexyl.

The term “alkoxy” or “alkyloxy” corresponds to the alkyl group as above defined bonded to the molecule by an —O— (ether) bond. (C₁-C₆)alkoxy includes methoxy or methyloxy, ethoxy or ethyloxy, propoxy or propyloxy, isopropoxy or isopropyloxy, butoxy or butyloxy, isobutoxy or isobutyloxy, pentoxy or pentyloxy, isopentoxy or isopentyloxy, and hexoxy or hexyloxy.

The term “3-20 membered ring” corresponds to a ring having between 3 and 20 atoms. Such a term includes the term “5-12 membered ring” having between 5 and 12 atoms. The term “ring” corresponds to a mono-, bi, or tricycle, which can be saturated or unsaturated, and optionally comprises at least one heteroatom. Particularly, the term “ring” includes a cycloalkyl, a heterocycloalkyl, an aryl, and a heteroaryl.

The term “cycloalkyl” corresponds to a saturated or unsaturated mono-, bi- or tri-cyclic alkyl group comprising between 3 and 20, preferably between 5 and 12 atoms of carbons. It also includes fused, bridged, or spiro-connected cycloalkyl groups. The term “cycloalkyl” includes for instance cyclopropyl, cyclobutyl, cyclopentyl, and cyclohexyl.

The term “heterocycloalkyl” corresponds to a saturated or unsaturated cycloalkyl group as above defined further comprising at least one heteroatom such as nitrogen, oxygen, or sulphur atom. It also includes fused, bridged, or spiro-connected heterocycloalkyl groups.

Representative heterocycloalkyl groups include, but are not limited to dioxolanyl, benzo[1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, azepanyl, 2,6-diazaspiro[3.3]heptanyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, and tetrahydrothiophenyl. In a preferred embodiment, the heterocycloalkyl group is azepanyl, piperazinyl, morpholinyl, piperidinyl, and 2,6-diazaspiro[3.3]heptanyl.

The term “aryl” corresponds to a mono- or bi-cyclic aromatic hydrocarbons having from 6 to 12 carbon atoms. For instance, the term “aryl” includes phenyl, naphtalenyl, or anthracenyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. As used herein, the term “heteroaryl” further includes the “fused arylheterocycloalkyl” and “fused heteroarylcycloalkyl”. The terms “fused arylheterocycloalkyl” and “fused heteroarylcycloalkyl” correspond to a bicyclic group in which an aryl as above defined or a heteroaryl is respectively bounded to the heterocycloalkyl or the cycloalkyl as above defined by at least two carbons. In other terms, the aryl or the heteroaryl shares a carbon bond with the heterocycloalkyl or the cycloalkyl. Examples of such mono- and poly-cyclic heteroaryl group, fused arylheterocycloalkyl and fused arylcycloalkyl may be: pyridinyl, thiophenyl, furanyl, pyrrolyl, pyrazolyl, imidazolyl, oxazolyl, isoxazolyl, thiazolyl, isothiazolyl, triazolyl, tetrazolyl, oxadiazolyl, furazanyl, thiadiazolyl, tetrazolyl, benzofuranyl, thianaphthalenyl, indolyl, indolinyl, indanyl, quinolinyl, isoquinolinyl, benzimidazolyl, tetrahydroquinolinyl, tetrahydroisoquinolinyl, triazinyl, thianthrenyl, benzofuranyl, dihydrobenzofuranyl, isobenzofuranyl, benzothiophenyl, isobenzothiophenyl, chromenyl, xanthenyl, phenoxanthinyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolyl, benzoxazolinyl, benzoxazinyl, benzothienyl, benzothiazolyl, benzodiazepinyl, benzazepinyl, benzoxazepinyl, isatinyl, dihydrobenzodioxepinyl, dihydropyridyl, s-triazinyl, oxazolyl, or thiofuranyl. In a preferred embodiment, the heteroaryl group is pyrimidinyl, thiophenyl, pyridinyl, triazolyl, and indolyl.

The term “halogen” corresponds to a fluorine, chlorine, bromine, or iodine atom, preferably a fluorine, chlorine or bromine atom, preferably a fluorine or a chlorine.

The expressions “a radical substituted by a” and “a radical substituted by at least” means that the radical is substituted by one or several groups of the list. For instance, the expression “a (C₁-C₆)alkyl substituted by at least one halogen, preferably a fluorine, may include a fluoromethyl (—CH₂F), a difluoromethyl (—CHF₂), or a trifluoromethyl (—CF₃).

The expression “optionally substituted” means that the radical is not substituted or substituted by one or several groups of the list.

The “tautomers” are isomeric compounds that differ only in the position of the protons and the electrons.

The “solvates” are compounds further comprising at least one molecule of solvent. The “hydrates” are compounds further comprising at least one molecule of water. For instance, if the compound comprises one molecule of water, it corresponds to a monohydrate form. If the compound comprises two molecules of water, it corresponds to a dihydrate form.

The “pharmaceutically salts” include inorganic as well as organic acids salts. Representative examples of suitable inorganic acids include hydrochloric, hydrobromic, hydroiodic, phosphoric, di- or tri-hydrochloric, di- or tri-hydrobromic, di- or tri-hydroiodic, di- or tri-phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. —COR may refer to —C(O)—R, —CO— may refer to —C(O)—, —CONHR may refer to —C(O)—NH—R, —NRR′ may refer to —N(R)R′, —NHCO— may refer to —NH—CO—, —O—CH₂— may refer to —CH₂—O—, and —CO₂R may refer to —C(O)—O—R.

As used herein, the terms “treatment”, “treat” or “treating” refer to any act intended to ameliorate the health status of patients such as therapy, prevention, prophylaxis and retardation of a disease, in particular a cancer. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to a mammal, even more preferably to a human, including adult, child, newborn and human at the prenatal stage. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient”, “active pharmaceutical ingredient”, and “drug” are equivalent and refer to a component of a pharmaceutical composition having a therapeutic effect.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease, particularly a cancer. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “pharmaceutically acceptable excipient” refers to any ingredient except active ingredients which are present in a pharmaceutical composition. Its addition may be aimed to confer a particular consistency or other physical or gustative properties to the final product.

A pharmaceutically acceptable excipient must be devoid of any interaction, in particular chemical, with the active ingredients.

### Compounds

The present invention provides new compounds of therapeutic interest.

According to the invention, a compound has the following formula (I):

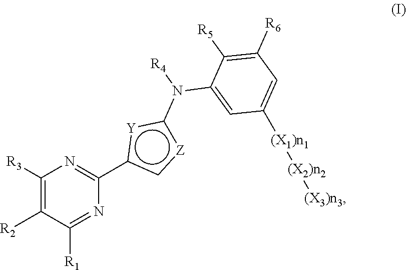

wherein:
Y and Z represent independently NH, N, O, or S;
R₁, R₂, and R₃ represent independently a radical selected in the group consisting of a hydrogen, a —NR₇R₈ group with R₇ and R₈ being independently a hydrogen or a (C₁-C₆)alkyl group, and a halogen;
R₄ and R₅ represent independently a hydrogen, a (C₁-C₆)alkyl group optionally substituted by a radical selected in the group consisting of a cycloalkyl, and a —NR₉R₁₀ group with R₉ and R₁₀ being independently a hydrogen or a (C₁-C₆)alkyl group, or R₄ and R₅ form together an azepanyl;
R₆ represents a hydrogen or a halogen;
X₁ represents a —NHCO— group, an oxygen atom, a halogen, a —C≡C— group, a —O—CH₂— group;
X₂ represents a 5-12 membered ring optionally substituted by at least one radical selected in the group consisting of a (C₁-C₆)alkyl group optionally substituted by at least one halogen, a (C₁-C₆)alkoxy group optionally substituted by at least one halogen, a halogen, and a hydroxy;
X₃ represents a radical selected in the group consisting of:
   a (C₁-C₆)alkyl substituted by at least one radical A
   a (C₁-C₆)alkoxy substituted by at least one radical A,
   a —C(O)— substituted by at least one radical A,
   a —SO₂— substituted by at least one radical A,
   a —NH—SO₂— substituted by at least one radical A,
   a —NHCO— substituted by at least one radical A,
in which said at least one radical A is selected in the group consisting of:
   a piperazinyl optionally substituted by a (C₁-C₆)alkyl or by a (C₁-C₆)alkyl substituted by a —COOH,
   a —CO-piperazinyl substituted by a (C₁-C₆)alkyl,
   a morpholinyl,
   a piperidinyl optionally substituted by a —NR₇R₈ group with R₇ and R₈ being independently a hydrogen or a (C₁-C₆)alkyl group, or
   a 2,6-diazaspiro[3.3]heptanyl substituted by a (C₁-C₆)alkyl,
   a —NR₁₁R₁₂ group with R₁₁ and R₁₂ being independently a hydrogen, a (C₁-C₆)alkyl group, or a —SO₂—CH₃ group,
   a —NH—(C₁-C₆)alkyl-NR₁₃R₁₄ group with R₁₃ and R₁₄ being independently a hydrogen, or a (C₁-C₆)alkyl group,
   a (C₁-C₆)alkoxy, and
   a hydroxy,
   a hydroxy, and
   a —NR₁₅R₁₆ group with R₁₅ and R₁₆ being independently a hydrogen or a (C₁-C₆)alkyl group; and
n₁, n₂, and n₃ are independently 0 or 1;
   and the pharmaceutically acceptable salts, the tautomers, and the solvates thereof.

According to a particular embodiment of the invention, a compound has the following formula (I):

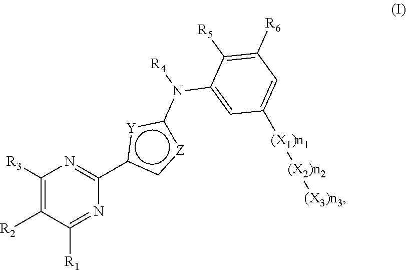

wherein:
Y and Z represent independently NH, N, O, or S;
R₁, R₂, and R₃ represent independently a radical selected in the group consisting of a hydrogen, a —NR₇R₈ group with R₇ and R₈ being independently a hydrogen or a (C₁-C₆)alkyl group, and a halogen;
R₄ and R₅ represent independently a hydrogen, a (C₁-C₆)alkyl group optionally substituted by a radical selected in the group consisting of a cycloalkyl, and a —NR₉R₁₀ group with R₉ and R₁₀ being independently a hydrogen or a (C₁-C₆)alkyl group, or R₄ and R₅ form together an azepanyl;
R₆ represents a hydrogen or a halogen;
X₁ represents a —NHCO— group, an oxygen atom, a —C≡C— group, or a —O—CH₂— group;
X₂ represents a 5-12 membered ring optionally substituted by at least one radical selected in the group consisting of a (C₁-C₆)alkyl group optionally substituted by at least one halogen, a (C₁-C₆)alkoxy group optionally substituted by at least one halogen, and a halogen;
X₃ represents a radical selected in the group consisting of:
   a (C₁-C₆)alkyl substituted by at least one radical A
   a (C₁-C₆)alkoxy substituted by at least one radical A,
   a —C(O)— substituted by at least one radical A,
   a —SO₂— substituted by at least one radical A,
   a —NH—SO₂— substituted by at least one radical A,
   a —NHCO— substituted by at least one radical A,
in which said at least one radical A is selected in the group consisting of:
   a piperazinyl optionally substituted by a (C₁-C₆)alkyl or by a (C₁-C₆)alkyl substituted by a —COOH,
   a —CO-piperazinyl substituted by a (C₁-C₆)alkyl,
   a morpholinyl,
   a piperidinyl optionally substituted by a —NR₇R₈ group with R₇ and R₈ being independently a hydrogen or a (C₁-C₆)alkyl group, or
   a 2,6-diazaspiro[3.3]heptanyl substituted by a (C₁-C₆)alkyl,
   a —NR₁₁R₁₂ group with R₁₁ and R₁₂ being independently a hydrogen, a (C₁-C₆)alkyl group, or a —SO₂—CH₃ group,
   a —NH—(C₁-C₆)alkyl-NR₁₃R₁₄ group with R₁₃ and R₁₄ being independently a hydrogen, or a (C₁-C₆)alkyl group,
   a (C₁-C₆)alkoxy, and
   a hydroxy,
   a hydroxy, and
   a —NR₁₅R₁₆ group with R₁₅ and R₁₆ being independently a hydrogen or a (C₁-C₆)alkyl group; and
n₁, n₂, and n₃ are independently 0 or 1;
   and the pharmaceutically acceptable salts, the tautomers, and the solvates thereof.

According to the invention, Y and Z represent independently NH, N, O, or S. Particularly, Y and Z represent independently N, O, or S. In a preferred embodiment, Y represents N and Z represents O or S. In a more preferred embodiment, Y represents N and Z represents S.

According to this more preferred embodiment, the compound has the following formula (I′):

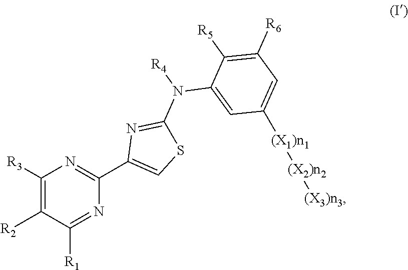

in which, R₁, R₂, R₃, R₄, R₅, R₆, X₁, X₂, X₃, n₁, n₂, and n₃ are such as defined herein.

According to the invention, R₁, R₂, and R₃ represent independently a radical selected in the group consisting of a hydrogen, a —NR₇R₈ group with R₇ and R₈ being independently a hydrogen or a (C₁-C₆)alkyl group, and a halogen. In a particular embodiment, R₁ represents a hydrogen or a —NR₇R₈ group with R₇ and R₈ being a hydrogen, i.e. an amino group —NH₂; R₂ represents a hydrogen or a halogen such as a fluorine, preferably a hydrogen; and R₃ represents a hydrogen or a —NR₇R₈ group with R₇ and R₈ being a hydrogen, preferably a —NR₇R₈ group with R₇ and R₈ being a hydrogen.

In a particular embodiment, at least one group chosen among R₁, R₂, and R₃ is not a hydrogen.

In a more particular embodiment, R₁, R₂, and R₃ represent independently a radical selected in the group consisting of a hydrogen, a —NR₇R₈ group with R₇ and R₈ being independently a hydrogen or a (C₁-C₆)alkyl group, and a halogen, provided that at least one group chosen among R₁, R₂, and R₃ is not a hydrogen. Preferably R₁ is not a hydrogen. In a preferred embodiment, R₁ represents a —NR₇R₈ group with R₇ and R₈ being a hydrogen; R₂ represents a hydrogen or a halogen, preferably a hydrogen; and R₃ represents a hydrogen or a —NR₇R₈ group with R₇ and R₈ being a hydrogen, preferably a —NR₇R₈ group with R₇ and R₈ being a hydrogen.

In a more preferred embodiment, R₁ represents a —NR₇R₈ group with R₇ and R₈ being a hydrogen; R₂ represents a hydrogen; and R₃ represents a —NR₇R₈ group with R₇ and R₈ being a hydrogen. According to this more preferred embodiment, R₁ is —NH₂, R₂ is H, and R₃ is —NH₂.

According to the invention, R₄ and R₅ represent independently a hydrogen, a (C₁-C₆)alkyl group optionally substituted by a radical selected in the group consisting of a cycloalkyl, and a —NR₉R₁₀ group with R₉ and R₁₀ being independently a hydrogen or a (C₁-C₆)alkyl group, or R₄ and R₅ form together an azepanyl.

In a particular embodiment, R₄ represents a hydrogen or a (C₁-C₆)alkyl group optionally substituted by a cycloalkyl or by a —NR₉R₁₀ group with R₉ and R₁₀ being a hydrogen. Preferably R₄ represents a hydrogen, a propyl, an isopropyl, an isobutyl, a —CH₂-cyclopropyl, an isopentyl, a butyl, a —(CH₂)₂—NH₂, a —(CH₂)₃—NH₂, a methyl, and an ethyl, preferably a propyl.

In a particular embodiment, R₅ represents a (C₁-C₆)alkyl group, preferably a methyl group.

In a more particular embodiment, R₄ represents a hydrogen or a (C₁-C₆)alkyl group optionally substituted by a cycloalkyl or by a —NR₉R₁₀ group with R₉ and R₁₀ being a hydrogen, preferably R₄ represents a (C₁-C₆)alkyl group; and R₅ represents a (C₁-C₆)alkyl group, preferably a methyl group.

In a preferred embodiment, R₄ represents a propyl group and R₅ represents a methyl group.

In a further particular embodiment, R₄ and R₅ form together an azepanyl.

According to the invention, R₆ represents a hydrogen or a halogen. Preferably, R₆ is a hydrogen.

According to the invention, n₁, n₂, and n₃ are independently 0 or 1. In a particular embodiment, n₁+n₂+n₃ is equal or superior to 1, preferably n₁+n₂+n₃ is 1, 2, or 3.

According to the invention, n₁ is 0 or 1. If n₁ is 1, then X₁ represents a —NHCO— group, an oxygen atom, a halogen, a —C≡C— group, or a —O—CH₂— group. In a particular embodiment, n₁ is 1 and X₁ represents a —NHCO— group.

According to the invention, n₂ is 0 or 1. If n₂ is 1, then X₂ represents a 5-12 membered ring optionally substituted by at least one radical selected in the group consisting of a (C₁-C₆)alkyl group optionally substituted by at least one halogen, a (C₁-C₆)alkoxy group optionally substituted by at least one halogen, a halogen, and a hydroxy.

In a particular embodiment, n₂ is 1 and X₂ represents a 5-12 membered ring selected in the group consisting of a phenyl, a pyrimidinyl, a thiophenyl, a pyridinyl, a triazolyl, and an indolyl, said 5-12 membered ring is optionally substituted by at least one radical selected in the group consisting of a methoxy, a trifluoromethoxy, a halogen, a hydroxy, a methyl, and a trifluromethyl.

According to the invention, n₃ is 0 or 1. If n is 1, then X₃ represents a radical selected in the group consisting of:
a (C₁-C₆)alkyl substituted by at least one radical A
a (C₁-C₆)alkoxy substituted by at least one radical A,
a —C(O)— substituted by at least one radical A,
a —SO₂— substituted by at least one radical A,
a —NH—SO₂— substituted by at least one radical A,
a —NHCO— substituted by at least one radical A,
in which said at least one radical A is selected in the group consisting of:
   a piperazinyl optionally substituted by a (C₁-C₆)alkyl or by a (C₁-C₆)alkyl substituted by a —COOH,
   a —CO-piperazinyl substituted by a (C₁-C₆)alkyl,
   a morpholinyl,
   a piperidinyl optionally substituted by a —NR₇R₈ group with R₇ and R₈ being independently a hydrogen or a (C₁-C₆)alkyl group, or
   a 2,6-diazaspiro[3.3]heptanyl substituted by a (C₁-C₆)alkyl,
   a —NR₁₁R₁₂ group with R₁₁ and R₁₂ being independently a hydrogen, a (C₁-C₆)alkyl group, or a —SO₂—CH₃ group,
   a —NH—(C₁-C₆)alkyl-NR₁₃R₁₄ group with R₁₃ and R₁₄ being independently a hydrogen, or a (C₁-C₆)alkyl group,
   a (C₁-C₆)alkoxy, and
   a hydroxy,
a hydroxy; and
a —NR₁₅R₁₆ group with R₁₅ and R₁₆ being independently a hydrogen or a (C₁-C₆)alkyl group.

In a particular embodiment, n₃ is 1 and X₃ represents a radical selected in the group consisting of a (C₁-C₆)alkyl, a (C₁-C₆)alkoxy, a —C(O)—, a —SO₂—, a —NH—SO₂—, and a —NHCO—, said radical being substituted by a piperazinyl optionally substituted by a (C₁-C₆)alkyl, preferably a piperazinyl substituted by a methyl.

In a particular embodiment, n₁ is 1. According to this particular embodiment, X₁ represents a —NHCO— group, an oxygen atom, a halogen, a —C≡C— group, or a —O—CH₂— group.

According to this particular embodiment, a compound of formula (I) or (I) is such that:
R₁ is NH₂;
R₂ is a hydrogen or a halogen, preferably a fluorine;
R₃ is a hydrogen or NH₂;
R₄ is a hydrogen or a (C₁-C₆)alkyl group, preferably a propyl, and R₅ represent a (C₁-C₆)alkyl group, preferably a methyl, or R₄ and R₅ form together an azepanyl;
R₆ represents a hydrogen;
X₂ represents a 5-6 membered ring selected in the group consisting of a phenyl, a pyrimidinyl, and a thiophenyl;
X₃ represents a (C₁-C₆)alkyl substituted by at least one radical A as defined herein, preferably at least one radical A selected in the group consisting of a piperazinyl substituted by a methyl, a —NH—(C₁-C₆)alkyl-NR₁₃R₁₄ group with R₁₃ and R₁₄ being independently a hydrogen or a methyl, and a 2,6-diazaspiro[3.3]heptanyl substituted by a methyl; and
n₂ and n₃ are 1.

In a preferred embodiment, n₁ is 1 and X₁ represents a —NHCO— group.

According to this preferred embodiment, a compound of formula (I) or (I′) is such that:
R₁ is NH₂;
R₂ is a hydrogen or a halogen, preferably a fluorine;
R₃ is a hydrogen or NH₂;
R₄ is a hydrogen or a (C₁-C₆)alkyl group, preferably a propyl, and R₅ represent a (C₁-C₆)alkyl group, preferably a methyl, or R₄ and R₅ form together an azepanyl;
R₆ represents a hydrogen;
X₂ represents a 5-6 membered ring selected in the group consisting of a phenyl, a pyrimidinyl, and a thiophenyl;
X₃ represents a (C₁-C₆)alkyl substituted by at least one radical A as defined herein, preferably at least one radical A selected in the group consisting of a piperazinyl substituted by a methyl, a —NH—(C₁-C₆)alkyl-NR₁₃R₁₄ group with R₁₃ and R₁₄ being independently a hydrogen or a methyl, and a 2,6-diazaspiro[3.3]heptanyl substituted by a methyl; and
n₂ and n₃ are 1.

In a further preferred embodiment, n₁ is 1 and X₁ represents an oxygen atom.

According to this preferred embodiment, a compound of formula (I′) is 4-(4-Aminopyrimidin-2-yl)-N-(2-methyl-5-(4-((4-methylpiperazin-1-yl)methyl)phenoxy)phenyl)thiazol-2-amine OR0143.

In a preferred embodiment, n₁ is 1 and X₁ represents a halogen, preferably a bromine. According to this preferred embodiment, a compound of formula (I′) is 2-(2-((5-Bromo-2-methylphenyl)(isobutyl)amino)thiazol-4-yl)pyrimidine-4,6-diamine OR0637-1; and 2-(2-((5-Bromo-2-methylphenyl)(propyl)amino)thiazol-4-yl)pyrimidine-4,6-diamine OR0652-1.

In a further preferred embodiment, n₁ is 1 and X₁ represents a —C≡C— group.
R₁ is NH₂;
R₂ is a hydrogen;
R₃ is a hydrogen;
R₄ is a hydrogen and R₅ represent a (C₁-C₆)alkyl group, preferably a methyl;
R₆ represents a hydrogen;
X₂ represents a phenyl;
X₃ represents a (C₁-C₆)alkyl substituted by at least one radical A as defined herein, preferably at least one radical A selected in the group consisting of a piperazinyl substituted by a methyl, a —NH—(C₁-C₆)alkyl-NR₁₃R₁₄ group with R₁₃ and R₁₄ being independently a hydrogen or a methyl; and
n₂ and n₃ are 1.

In a further preferred embodiment, n₁ is 1 and X₁ represents a —O—CH₂— group.

According to this preferred embodiment, a compound of formula (I) is OR0232.

In a further particular embodiment, n₁ is 0.

According to this particular embodiment, n₂ is 1 and X₂ represents a 5-12 membered ring selected in the group consisting of a phenyl, a pyrimidinyl, a thiophenyl, a pyridinyl, a triazolyl, and an indolyl, said 5-12 membered ring is optionally substituted by at least one radical selected in the group consisting of a methoxy, a trifluoromethoxy, a halogen, a hydroxy, a methyl, and a trifluromethyl.

According to this particular embodiment, n₃ is 0 or 1, preferably 1 and X₃ represents a radical selected in the group consisting of a (C₁-C₆)alkyl, a (C₁-C₆)alkoxy, a —C(O)—, a —SO₂—, a —NH—SO₂—, and a —NHCO—, said radical being substituted by at least one radical A as defined herein.

In a preferred embodiment, n₃ is 1 and X₃ represents a (C₁-C₆)alkyl substituted by at least one radical A as defined herein. Preferably said at least one radical A is a piperazinyl substituted by a methyl, a —N(CH₃)₂, and a —NHSO₂CH₃.

In a further preferred embodiment, n₃ is 1 and X₃ represents a —SO₂— substituted by at least one radical A as defined herein. Preferably said at least one radical A is a piperazinyl optionally substituted by a methyl, an ethyl or a —CH₂—COOH, a —NR₁₁R₁₂ group with Rn and R₁₂ being independently a hydrogen or a methyl group, a —NH—(CH₂)₂—NH₂, a morpholinyl, and a piperidinyl optionally substituted by a —NH₂, more preferably a piperazinyl unsubstituted or substituted by a methyl, a —NR₁₁R₁₂ group with R₁₁ and R₁₂ being independently a hydrogen or a methyl group, or a morpholinyl.

In a further preferred embodiment, n₃ is 1 and X₃ represents a (C₁-C₆)alkoxy substituted by at least one radical A as defined herein, preferably a piperazinyl optionally substituted by a methyl or a —CO-piperazinyl optionally substituted by a methyl.

In a further preferred embodiment, n₃ is 1 and X₃ represents a —C(O)— substituted by at least one radical A as defined herein, preferably a piperazinyl optionally substituted by a methyl, a morpholinyl, or a methoxy.

In a further preferred embodiment, n₃ is 1 and X₃ represents a —NH—SO₂— substituted by at least one radical A as defined herein, preferably a piperazinyl optionally substituted by a methyl.

In a further preferred embodiment, n₃ is 1 and X₃ represents a —NHCO— substituted by at least one radical A as defined herein, preferably a piperazinyl optionally substituted by a methyl.

In a further preferred embodiment, n₃ is 1 and X₃ represents a hydroxy.

In a further preferred embodiment, n₃ is 1 and X₃ represents a —NR₁₅R₁₆ group with R₁₅ and R₁₆ being independently a hydrogen or a (C₁-C₆)alkyl group, preferably a methyl.

A preferred compound of formula (I) or (I′) is such that:
R₁, R₂, and R₃ represent independently a hydrogen, or a —NR₇R₈ group with R₇ and R₈ being independently a hydrogen; preferably provided that at least one group chosen among R₁, R₂, and R₃ is not a hydrogen
R₄ and R₅ represent independently a hydrogen or a (C₁-C₆)alkyl group;
R₆ represents a hydrogen;
X₁ represents a —NHCO— group;
X₂ represents a 5-6 membered ring selected in the group consisting of a phenyl and a pyridinyl, said 5-6 membered ring being optionally substituted by at least one radical selected in the group consisting of a trifluoromethyl, a trifluoromethoxy, a halogen, a hydroxy, a methoxy, and a methyl;
X₃ represents a radical selected in the group consisting of a (C₁-C₆)alkyl and a —SO₂—, said radical being substituted by a piperazinyl substituted by a (C₁-C₆)alkyl
n₁ is 0 or 1; and
n₂ and n₃ are 1.

A further preferred compound of formula (I) or (I′) is such that:
R₁, R₂, and R₃ represent independently a hydrogen, or a —NR₇R₈ group with R₇ and R₈ being independently a hydrogen, preferably R₁ and R₃ represent NH₂ and R₂ represents H;
R₄ and R₅ represent independently a (C₁-C₆)alkyl group, preferably R₄ represents a propyl and R₅ a methyl;
R₆ represents a hydrogen;
X₂ represents a 5-6 membered ring selected in the group consisting of a phenyl, and a pyridinyl, said 5-6 membered ring being optionally substituted by at least one radical selected in the group consisting of a trifluoromethyl, a trifluoromethoxy, a halogen, a hydroxy, a methoxy, and a methyl;
X₃ represents a —SO₂— substituted by at least one radical selected in the group consisting of a piperazinyl optionally substituted by a (C₁-C₆)alkyl or by a (C₁-C₆)alkyl substituted by a —COOH, a piperidinyl optionally substituted by a —NH₂, and a —NH—(C₁-C₆)alkyl-NH₂;
n₁ is 0; and
n₂ and n₃ are 1.

An even more preferred compound of formula (I) or (I) is such that:
R₁ represents a —NH₂, R₂ represents a hydrogen, and R₃ represent —NH₂;
R₄ represents a propyl and R₅ a methyl;
R₆ represents a hydrogen;
X₂ represents a phenyl optionally substituted by at least one radical selected in the group consisting of a trifluoromethyl, a trifluoromethoxy, a halogen, a methoxy, and a methyl;
X₃ represents a —SO₂— substituted by piperazinyl optionally substituted by a methyl;
n₁ is 0; and
n₂ and n₃ are 1.

A particular compound of formula (I) is selected in the group consisting of:
N-(3-((4-(4-Aminopyrimidin-2-yl)thiazol-2-yl)amino)-4-methylphenyl)-4-((4-methylpiperazin-1-yl)methyl)benzamide dCKi-1;
N-(3-((4-(4,6-Diaminopyrimidin-2-yl)thiazol-2-yl)amino)-4-methylphenyl)-4-((4-methylpiperazin-1-yl)methyl)benzamide dCKi-2:
N-(3-((4-(4-Aminopyrimidin-2-yl)thiazol-2-yl)(propyl)amino)-4-methylphenyl)-4-((4-methylpiperazin-1-yl)methyl)benzamide OR005;
N-(3-((4-(4-Aminopyrimidin-2-yl)thiazol-2-yl)(propyl)amino)-4-methylphenyl)-2-((4-methylpiperazin-1-yl)methyl)pyrimidine-5-carboxamide OR025;
4-(4-Aminopyrimidin-2-yl)-N-(2-methyl-5-(4-((4-methylpiperazin-1-yl)methyl)phenoxy)phenyl)thiazol-2-amine OR043;
N-(3-((4-(4-Aminopyrimidin-2-yl)thiazol-2-yl)amino)-4-methylphenyl)-4-(2-(4-methylpiperazin-1-yl)ethyl)benzamide OR046;
4-(4-Aminopyrimidin-2-yl)-N-(2-methyl-5-((4-((4-methylpiperazin-1-yl)methyl)phenyl)ethynyl)phenyl)thiazol-2-amine OR053;
N-(3-((4-(4-Aminopyrimidin-2-yl)thiazol-2-yl)amino)-4-methylphenyl)-4-((dimethylamino)methyl)benzamide OR055;
N-(3-((4-(4-Aminopyrimidin-2-yl)thiazol-2-yl)amino)-4-methylphenyl)-4-((6-methyl-2,6-diazaspiro[3.3]heptan-2-yl)methyl)benzamide OR0156;
4-(4-Aminopyrimidin-2-yl)-N-(2-methyl-5-(4-((4-methylpiperazin-1-yl)methyl)benzyloxy)phenyl)thiazol-2-amine OR0232;
4-(4-Aminopyrimidin-2-yl)-N-(5-((4-((dimethylamino)methyl)phenyl)ethynyl)-2-methylphenyl)thiazol-2-amine OR0237;
N-(3-((4-(4-Amino-5-fluoropyrimidin-2-yl)thiazol-2-yl)amino)-4-methylphenyl)-4-((4-methylpiperazin-1-yl)methyl)benzamide OR0239;
N-(3-((4-(4-Aminopyrimidin-2-yl)thiazol-2-yl)amino)-4-methylphenyl)-5-((4-methylpiperazin-1-yl)methyl)thiophene-2-carboxamide OR0241;
4-(4-Aminopyrimidin-2-yl)-N-(4-methyl-4′-((4-methylpiperazin-1-yl)methyl)-[1,1′-biphenyl]-3-yl)thiazol-2-amine OR0274;
N-(1-(4-(4-Aminopyrimidin-2-yl)thiazol-2-yl)-2,3,4,5-tetrahydro-1H-benzo[b]azepin-8-yl)-4-((4-methylpiperazin-1-yl)methyl)benzamide OR0289;
3′-((4-(4-Aminopyrimidin-2-yl)thiazol-2-yl)amino)-4′-methyl-[1,1′-biphenyl]-4-ol OR0320;
3′-((4-(4-Aminopyrimidin-2-yl)thiazol-2-yl)amino)-4′-methyl-[1,1′-biphenyl]-4-ol OR0321;
4-(4-Aminopyrimidin-2-yl)-N-(4-methyl-4′-(2-(4-methylpiperazin-1-yl)ethyl)-[1,1′-biphenyl]-3-yl)thiazol-2-amine OR0325;
4-(4-Aminopyrimidin-2-yl)-N-(4-methyl-4′-(3-(4-methylpiperazin-1-yl)propyl)-[1,1′-biphenyl]-3-yl)thiazol-2-amine OR0331;
4-(4-Aminopyrimidin-2-yl)-N-(4-methyl-4′-(3-(4-methylpiperazin-1-yl)ethyl)-[1,1′-biphenyl]-3-yl)-N-propylthiazol-2-amine OR0345;
2-(2-(8-(4-(2-(4-Methylpiperazin-1-yl)ethyl)phenyl)-2,3,4,5-tetrahydro-1H-benzo[b]azepin-1-yl)thiazol-4-yl)pyrimidin-4-amine OR0402;
4-(4-Aminopyrimidin-2-yl)-N-(4-methyl-4′-(2-(4-methylpiperazin-1-yl)ethoxy)-[1,1′-biphenyl]-3-yl)-N-propylthiazol-2-amine OR0596;
4-(4-Aminopyrimidin-2-yl)-N-(4-methyl-4′-(2-(4-methylpiperazin-1-yl)-2-oxoethoxy)-[1,1′-biphenyl]-3-yl)-N-propylthiazol-2-amine OR0597;
4-(4-Aminopyrimidin-2-yl)-N-(4-methyl-4′-(3-(4-methylpiperazin-1-yl)propyl)-[1,1′-biphenyl]-3-yl)-N-propylthiazol-2-amine OR0598;
4-(4-Aminopyrimidin-2-yl)-N-(2-methyl-5-(6-(2-(4-methylpiperazin-1-yl)ethyl)pyridin-3-yl)phenyl)-N-propylthiazol-2-amine OR0599;
2-(2-((4-Methyl-4′-(2-(4-methylpiperazin-1-yl)ethyl)-[1,1′-biphenyl]-3-yl)(propyl)amino)thiazol-4-yl)pyrimidine-4,6-diamine OR0600;
2-(2-((4-Methyl-4′-(3-(4-methylpiperazin-1-yl)propyl)-[1,1′-biphenyl]-3-yl)(propyl)amino)thiazol-4-yl)pyrimidine-4,6-diamine OR0601;
2-(2-((2-Methyl-5-(6-(2-(4-methylpiperazin-1-yl)ethyl)pyridin-3-yl)phenyl)(propyl)amino)thiazol-4-yl)pyrimidine-4,6-diamine OR0602;
2-(2-(iso-Butyl(4-methyl-4′-(2-(4-methylpiperazin-1-yl)ethyl)-[1,1′-biphenyl]-3-yl)amino)thiazol-4-yl)pyrimidine-4,6-diamine OR0603;
2-(2-((Cyclopropylmethyl)(4-methyl-4′-(2-(4-methylpiperazin-1-yl)ethyl)-[1,1′-biphenyl]-3-yl)amino)thiazol-4-yl)pyrimidine-4,6-diamine OR0604;
2-(2-(iso-Pentyl(4-methyl-4′-(2-(4-methylpiperazin-1-yl)ethyl)-[1,1′-biphenyl]-3-yl)amino)thiazol-4-yl)pyrimidine-4,6-diamine OR0605;
2-(2-(Butyl(4-methyl-4′-(2-(4-methylpiperazin-1-yl)ethyl)-[1,1′-biphenyl]-3-yl)amino)thiazol-4-yl)pyrimidine-4,6-diamine OR0606;
2-(2-((4-Methyl-4′-((4-methylpiperazin-1-yl)sulfonyl)-[1,1′-biphenyl]-3-yl)(propyl)amino)thiazol-4-yl)pyrimidine-4,6-diamine OR0607;
2-(2-(iso-Butyl(2-methyl-5-(6-(2-(4-methylpiperazin-1-yl)ethoxy)pyridin-3-yl)phenyl)amino)thiazol-4-yl)pyrimidine-4,6-diamine OR0608;
N-((1-(3-((4-(4,6-Diaminopyrimidin-2-yl)thiazol-2-yl)(isobutyl)amino)-4-methylphenyl)-1H-1,2,3-triazol-4-yl)methyl) methanesulfonamide OR0609;
2-(2-((2-Aminoethyl)(4-methyl-4′-(2-(4-methylpiperazin-1-yl)ethyl)-[1,1′-biphenyl]-3-yl)amino)thiazol-4-yl)pyrimidine-4,6-diamine OR0610;
2-(2-((3-Aminopropyl)(4-methyl-4′-(2-(4-methylpiperazin-1-yl)ethyl)-[1,1′-biphenyl]-3-yl)amino)thiazol-4-yl)pyrimidine-4,6-diamine OR0611;
2-[2-({2-Methyl-5-[6-(4-methyl-piperazin-1-ylcarbonyl)-pyridin-3-yl]-phenyl}-propyl-amino)-thiazol-4-yl]-pyrimidine-4,6-diamine OR0612;
2-(2-((5-(2-Aminopyrimidin-5-yl)-2-methylphenyl)(propyl)amino)thiazol-4-yl)pyrimidine-4,6-diamine OR0613;
2-(2-((4-Methyl-4′-((4-methylpiperazin-1-yl)sulfonyl)-[1,1′-biphenyl]-3-yl)amino)thiazol-4-yl)pyrimidine-4,6-diamine OR0614;
3′-((4-(4,6-Diaminopyrimidin-2-yl)thiazol-2-yl)(propyl)amino)-4′-methyl-[1,1′-biphenyl]-4-sulfonamide OR0615;
N-(5-(3-((4-(4,6-Diaminopyrimidin-2-yl)thiazol-2-yl)(propyl)amino)-4-methylphenyl)pyridin-2-yl)-4-methylpiperazine-1-carboxamide OR0616;
2-(2-((4-Methyl-4′-(morpholinosulfonyl)-[1,1′-biphenyl]-3-yl)(propyl)amino)thiazol-4-yl)pyrimidine-4,6-diamine OR0617;
3′-((4-(4,6-Diaminopyrimidin-2-yl)thiazol-2-yl)(propyl)amino)-N,N,4′-trimethyl-[1,1′-biphenyl]-4-sulfonamide OR0618;
2-(2-((4′-((Dimethylamino)methyl)-4-methyl-[1,1′-biphenyl]-3-yl)(propyl)amino)thiazol-4-yl)pyrimidine-4,6-diamine OR0619;
2-(2-((2-Methyl-5-(pyridin-3-yl)phenyl)(propyl)amino)thiazol-4-yl)pyrimidine-4,6-diamine OR0620;
(5-(3-((4-(4,6-Diaminopyrimidin-2-yl)thiazol-2-yl)(propyl)amino)-4-methylphenyl)pyridin-2-yl)(morpholino) methanone OR0621;
Methyl 5-(3-((4-(4,6-diaminopyrimidin-2-yl)thiazol-2-yl)(propyl)amino)-4-methylphenyl)picolinate OR0622;
N-(5-(3-((4-(4,6-Diaminopyrimidin-2-yl)thiazol-2-yl)(propyl)amino)-4-methylphenyl)pyridin-2-yl)-4-methylpiperazine-1-sulfonamide OR0625;
2-(2-((5-(1H-Indol-5-yl)-2-methylphenyl)(propyl)amino)thiazol-4-yl)pyrimidine-4,6-diamine OR0626;
2-(2-((2-Methyl-5-(6-((4-methylpiperazin-1-yl)sulfonyl)pyridin-3-yl)phenyl)(propyl)amino)thiazol-4-yl)pyrimidine-4,6-diamine OR0627;
2-(2-((2-Methyl-5-(6-(2-(4-methylpiperazin-1-yl)ethoxy)pyridin-3-yl)phenyl)(propyl)amino)thiazol-4-yl)pyrimidine-4,6-diamine OR0629;
2-((5-(3-((4-(4,6-Diaminopyrimidin-2-yl)thiazol-2-yl)(propyl)amino)-4-methylphenyl)pyridin-2-yl)oxy)-1-(4-methylpiperazin-1-yl)ethan-1-one OR0630;
2-(2-(Methyl(4-methyl-4′-((4-methylpiperazin-1-yl)sulfonyl)-[1,1′-biphenyl]-3-yl)amino)thiazol-4-yl)pyrimidine-4,6-diamine OR0631;
2-(2-(Ethyl(4-methyl-4′-((4-methylpiperazin-1-yl)sulfonyl)-[1,1′-biphenyl]-3-yl)amino)thiazol-4-yl)pyrimidine-4,6-di-amine OR0632;
2-(2-((5-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0633;
2-(2-((3′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine OR0635;
2-(2-(iso-Propyl(4-methyl-4′-((4-methylpiperazin-1-yl)sulfonyl)-[1,1′-biphenyl]-3-yl)amino)thiazol-4-yl)pyrimidine-4,6-diamine OR0636;
2-(2-(iso-Butyl(4-methyl-4′-((4-methylpiperazin-1-yl)sulfonyl)-[1,1′-biphenyl]-3-yl)amino)thiazol-4-yl)pyrimidine-4,6-diamine OR0637;
2-(2-((4′-((4-Ethylpiperazin-1-yl)sulfonyl)-4-methyl-[1,1′-biphenyl]-3-yl)(propyl)amino)thiazol-4-yl)pyrimidine-4,6-diamine OR0638;
2-(2-((4-Methyl-4′-(piperazin-1-ylsulfonyl)-[1,1′-biphenyl]-3-yl)(propyl)amino)thiazol-4-yl)pyrimidine-4,6-diamine OR0639;
2-(2-((4-Methyl-4′-((4-methylpiperazin-1-yl)sulfonyl)-[1,1′-biphenyl]-3-yl)(propyl)amino)oxazol-4-yl)pyrimidine-4,6-diamine OR0640;
2-(2-((2′-Chloro-4-methyl-4′-((4-methylpiperazin-1-yl)sulfonyl)-[1,1′-biphenyl]-3-yl)(propyl)amino)thiazol-4-yl)pyrimidine-4,6-diamine OR0641;
2-(2-((4-Methyl-4′-((4-methylpiperazin-1-yl)sulfonyl)-2′-(trifluoromethyl)-[1,1′-biphenyl]-3-yl)(propyl)amino) thiazol-4-yl)pyrimidine-4,6-diamine OR0642;
2-(2-((2′-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0643;
2-(2-((2′-Fluoro-6′-methoxy-4-methyl-4′-((4-methylpiperazin-1-yl)sulfonyl)-[1,1′-biphenyl]-3-yl)(propyl)amino) thiazol-4-yl)pyrimidine-4,6-diamine OR0644;
2-(2-((2′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine OR0645;
2-(2-((4-Methyl-4′-(piperidin-4-ylsulfonyl)-[1,1′-biphenyl]-3-yl)(propyl)amino)thiazol-4-yl)pyrimidine-4,6-diamine OR0646;
2-(2-((2′,6′-Difluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0647;
2-(2-((4′-((4-Aminopiperidin-1-yl)sulfonyl)-4-methyl-[1,1′-biphenyl]-3-yl)(propyl)amino)thiazol-4-yl)pyrimidine-4,6-diamine OR0648;
2-(4-((3′-((4-(4,6-Diaminopyrimidin-2-yl)thiazol-2-yl)(propyl)amino)-4′-methyl-[1,1′-biphenyl]-4-yl)sulfonyl) piperazin-1-yl)acetic acid OR0649;
2-(2-((4-Methyl-4′-((4-methylpiperazin-1-yl)sulfonyl)-3′-(trifluoromethoxy)-[1,1′-biphenyl]-3-yl)(propyl)amino) thiazol-4-yl)pyrimidine-4,6-diamine OR0650;
N-(2-Aminoethyl)-3′-((4-(4,6-diaminopyrimidin-2-yl)thiazol-2-yl)(propyl)amino)-4′-methyl-[1,1′-biphenyl]-4-sulfonamide OR0651;
2-(2-((2-Methyl-5-(4-methyl-6-((4-methylpiperazin-1-yl)sulfonyl)pyridin-3-yl)phenyl)(propyl)amino)thiazol-4-yl)pyrimidine-4,6-diamine OR0652;
2-(2-((5-Bromo-2-methylphenyl)(isobutyl)amino)thiazol-4-yl)pyrimidine-4,6-diamine OR0637-1;
2-(2-((5-Bromo-2-methylphenyl)(propyl)amino)thiazol-4-yl)pyrimidine-4,6-diamine OR0652-1;
2-(2-((2-Methyl-5-(2-methyl-6-((4-methylpiperazin-1-yl)sulfonyl)pyridin-3-yl)phenyl)(propyl)amino)thiazol-4-yl)pyrimidine-4,6-diamine OR0653;
2-(2-((5-(6-Methoxy-4-(trifluoromethyl)pyridin-3-yl)-2-methylphenyl)(propyl)amino)thiazol-4-yl)pyrimidine-4,6-diamine OR0654;
2-(2-((5-(6-Hydroxy-4-(trifluoromethyl)pyridin-3-yl)-2-methylphenyl)(propyl)amino)thiazol-4-yl)pyrimidine-4,6-diamine OR0655;
2-(2-((2-Methyl-5-(6-((4-methylpiperazin-1-yl)sulfonyl)-4-(trifluoromethyl)pyridin-3-yl)phenyl)(propyl)amino)thiazol-4-yl)pyrimidine-4,6-diamine OR0656;
2-(2-((2-Methyl-5-(6-((4-methylpiperazin-1-yl)sulfonyl)-2-(trifluoromethyl)pyridin-3-yl)phenyl)(propyl)amino)thiazol-4-yl)pyrimidine-4,6-diamine OR0657; and
2-(2-((2-Methyl-5-(6-(piperazin-1-ylsulfonyl)-2-(trifluoromethyl)pyridin-3-yl)phenyl)(propyl)amino)thiazol-4-yl)pyrimidine-4,6-diamine OR0658.

### Applications

As illustrated by examples, the inventors have demonstrated the therapeutic interest of the compounds of the invention. Indeed, the inventors have shown that the compounds according to the invention are able to bind dCK and inhibit its activity, demonstrating thereby the therapeutic interest of such compounds in therapies, more particularly in cancer therapies. They have also demonstrated that such compounds, in combination with an antitumor drug, such as an inhibitor of the De Novo nucleotide biosynthesis pathway, significantly improved survival rate in a murine leukemia model.

Accordingly, the present invention relates to a compound of formula (I) or (I′) as defined herein, for use as a drug or a medicine. The present invention further relates to a pharmaceutical or veterinary composition comprising a compound according to the invention. Preferably, the pharmaceutical composition further comprises a pharmaceutically or veterinary acceptable carrier or excipient. The present invention relates to the use of a compound according to the invention as a drug or a medicine. The invention further relates to a method for treating a disease in a subject, wherein a therapeutically effective amount of a compound according to the invention, is administered to said subject in need thereof. The invention also relates to the use of a compound according to the invention, for the manufacture of a medicine. The invention also relates to a pharmaceutical composition comprising a compound according to the invention for use as a drug.

The present invention also concerns:
a compound of formula (I) or (I′) as defined above including anyone of the disclosed embodiments, or a pharmaceutical composition comprising such a compound for preventing and/or treating or for use for preventing and/or treating a cancer; and/or
a pharmaceutical composition comprising a compound of formula (I) or (I′) as defined above including anyone of the disclosed embodiments, and an antitumor drug, for the prevention and/or the treatment of cancer or for use in the prevention and/or the treatment of cancer; and/or
a compound of formula (I) or (I′) including anyone of the disclosed embodiments, or a pharmaceutical composition comprising such a compound, for preventing and/or treating a cancer or for use for preventing and/or treating a cancer in combination with radiotherapy, hyperthermia and/or other antitumor therapies, optionally before, simultaneously and/or after surgery (e.g., tumor resection); and/or
a kit comprising (a) a compound of formula (I) or (I′) as defined above including anyone of the disclosed embodiments; and (b) an antitumor drug as a combined preparation for simultaneous, separate or sequential use, for preventing and/or treating cancer or for use for preventing and/or treating a cancer; and/or
the use of a compound of formula (I) or (I′) as defined above including anyone of the disclosed embodiments, or a pharmaceutical composition comprising such a compound, for the manufacture of a medicament, a medicine or a drug for the prevention and/or the treatment of a cancer; and/or
the use of a pharmaceutical composition comprising a compound of formula (I) or (I′) as defined above including anyone of the disclosed embodiments, and an additional antitumor drug, for the manufacture of a medicament, a medicine or a drug for the prevention and/or the treatment of a cancer; and/or
the use of a compound of formula (I) or (I′) as defined above including anyone of the disclosed embodiments, or a pharmaceutical composition comprising such a compound, for the manufacture of a medicament, a medicine or a drug for the prevention and/or the treatment of a cancer in combination with radiotherapy, hyperthermia and/or other antitumor therapies, optionally before, simultaneously and/or after surgery (e.g., tumor resection); and/or
a method for treating a cancer, in a subject in need thereof, comprising administering an effective amount of a compound of formula (I) or (I′) as defined herein, or a pharmaceutical composition comprising such a compound;
a method for treating a cancer, in a subject in need thereof, comprising administering an effective amount of a compound of formula (I) or (I′) as defined herein, or a pharmaceutical composition comprising such a compound, and an additional antitumor drug;
a method for treating a cancer, in a subject in need thereof, comprising administering an effective amount of a compound of formula (I) or (I′) as defined herein, or a pharmaceutical composition comprising such a compound; the method further comprises radiotherapy, hyperthermia and/or other antitumor therapies, optionally before, simultaneously and/or after surgery (e.g., tumor resection).

The term “cancer”, as used herein, refers to the presence of cells possessing characteristics typical of cancer-causing cells, such as uncontrolled proliferation, immortality, metastatic potential, rapid growth and proliferation rate, and certain characteristic morphological features. The cancer may be a solid cancer, such as a solid tumor or a liquid cancer, such as a hematopoietic tumor. Examples of cancer include, for example, leukemia, lymphoma, blastoma, carcinoma, such as cholangiocarcinoma, and sarcoma. More particular examples of such cancers include chronic myeloid leukemia, acute lymphoblastic leukemia, such as T-cell acute lymphoblastic leukemia, Philadelphia chromosome positive acute lymphoblastic leukemia (Ph+ALL), squamous cell carcinoma, lung cancer, small-cell lung cancer, non-small cell lung cancer, glioma, gastrointestinal cancer, renal cancer, ovarian cancer, liver cancer, colorectal cancer, endometrial cancer, kidney cancer, prostate cancer, melanoma, skin cancer, thyroid cancer, neuroblastoma, osteosarcoma, pancreatic cancer, glioblastoma multiforme, cervical cancer, stomach cancer, bladder cancer, hepatoma, breast cancer, oesophagal cancer, colon cancer, head and neck cancer, brain cancer, gastric cancer, germ cell tumor, pediatric sarcoma, sinonasal natural killer, multiple myeloma, acute myelogenous leukemia (AML), chronic lymphocytic leukemia, mastocytosis and any symptom associated with mastocytosis. In a particular embodiment, the cancer is a liquid cancer, preferably acute lymphoblastic leukemia, more preferably T-cell acute lymphoblastic leukemia.

The term “therapy”, as used herein, refers to any type of treatment of cancer (i.e., antitumor therapy), including an adjuvant therapy and a neoadjuvant therapy. Therapy comprises radiotherapy and therapies, preferably systemic therapies such as hormone therapy, chemotherapy, immunotherapy and monoclonal antibody therapy.

The term “adjuvant therapy”, as used herein, refers to any type of treatment of cancer given as additional treatment, usually after surgical resection of the primary tumor, in a patient affected with a cancer that is at risk of metastasizing and/or likely to recur. The aim of such an adjuvant treatment is to improve the prognosis. Adjuvant therapies comprise radiotherapy and therapy, preferably systemic therapy, such as hormone therapy, chemotherapy, immunotherapy and monoclonal antibody therapy.

The term “hormone therapy” or “hormonal therapy” refers to a cancer treatment having for purpose to block, add or remove hormones. For instance, in breast cancer, the female hormones oestrogen and progesterone can promote the growth of some breast cancer cells. So, in these patients, hormone therapy is given to block oestrogen and a non-exhaustive list commonly used drugs includes: tamoxifen, toremifene, anastrozole, exemestane, letrozole, goserelin, leuprolide, megestrol acetate, and fluoxymesterone.

As used herein, the term “chemotherapeutic treatment” or “chemotherapy” refers to a cancer therapeutic treatment using chemical or biological substances, in particular using one or several antineoplastic agents.

The term “radiotherapeutic treatment” or “radiotherapy” is a term commonly used in the art to refer to multiple types of radiation therapy including internal and external radiation therapies or radioimmunotherapy, and the use of various types of radiations including X-rays, gamma rays, alpha particles, beta particles, photons, electrons, neutrons, radioisotopes, and other forms of ionizing radiations.

The term “therapeutical antibody” refers to any antibody having an anti-tumoral effect.

Preferably, the therapeutical antibody is a monoclonal antibody. Therapeutic antibodies are generally specific for surface antigens, e.g., membrane antigens. Most preferred therapeutic antibodies are specific for tumor antigens (e.g., molecules specifically expressed by tumor cells), such as CD20, CD52, ErbB2 (or HER2/Neu), CD33, CD22, CD25, MUC-1, CEA, KDR, aVb3, and the like. The therapeutical antibody includes, but is not limited to, antibodies such as trastuzumab (anti-HER2 antibody), rituximab (anti-CD20 antibody), alemtuzumab, gemtuzamab, cetuximab, pertuzumab, epratuzumab, basiliximab, daclizumab, labetuzumab, sevirumab, tuvurimab, palivizumab, infliximab, omalizumab, efalizumab, natalizumab, clenoliximab, and bevacizumab.

Hyperthermia is a medical treatment in which is exposed to high temperatures to damage and kill cancer cells or to make cancer cells more sensitive to the effects of radiation and certain anti-cancer drugs. There are many techniques, well-known by the one skilled in the art, by which heat may be delivered. Some of the most common involve the use of focused ultrasound (FUS or HIFU), infrared sauna, microwave heating, induction heating, magnetic hyperthermia, infusion of warmed liquids, or direct application of heat such as through sitting in a hot room or wrapping a patient in hot blankets.

The administration route can be topical, transdermal, oral, rectal, sublingual, intranasal, intrathecal, intratumor or parenteral (including subcutaneous, intramuscular, intravenous and/or intradermal). Preferably, the administration route is parental, oral or topical. The pharmaceutical composition is adapted for one or several of the above-mentioned routes. The pharmaceutical composition, kit, product or combined preparation is preferably administered by injection or by intravenous infusion or suitable sterile solutions, or in the form of liquid or solid doses via the alimentary canal.

The pharmaceutical composition can be formulated as solutions in pharmaceutically compatible solvents or as emulsions, suspensions or dispersions in suitable pharmaceutical solvents or vehicles, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations for rectal administration may be in the form of a suppository incorporating the active ingredient and carrier such as cocoa butter, or in the form of an enema. Formulations suitable for parenteral administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavoring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

Pharmaceutical compositions according to the invention may be formulated to release the active drug substantially immediately upon administration or at any predetermined time or time period after administration. Preferably, the treatment with the compound according to the invention or the pharmaceutical composition according to the invention starts no longer than a month, preferably no longer than a week, after the diagnosis of the disease. In a most preferred embodiment, the treatment starts the day of the diagnosis.

The compound according to the invention or the pharmaceutical composition according to the invention may be administered as a single dose or in multiple doses.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with the compound according to the invention or the pharmaceutical composition according to the invention is preferably comprised between 1 day and 50 weeks, more preferably between 1 day and 30 weeks, still more preferably between 1 day and 15 weeks, even more preferably between 1 day and 10 weeks. In a particular embodiment, the duration of the treatment is of about 1 week. Alternatively, the treatment may last as long as the disease persists.

The amount of compound according to the invention or of pharmaceutical composition according to the invention to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a preferred embodiment, the total compound dose for each administration of the compound according to the invention or of the pharmaceutical composition according to the invention is comprised between 0.00001 and 1 g, preferably between 0.01 and 10 mg. The form of the pharmaceutical compositions, the route of administration and the dose of administration of the compound according to the invention, or the pharmaceutical composition according to the invention can be adjusted by the man skilled in the art according to the type and severity of the disease, and to the patient, in particular its age, weight, sex, and general physical condition.

### Antitumor Drug

In an embodiment, the compound of the invention can be used in combination with another antitumor drug or antineoplastic agent. The additional antitumor drug can be selected in the non-exhaustive list of antitumor agents consisting of an inhibitor of topoisomerases I or II, an anti-mitotic agent, a DNA alkylating agent, an agent causing crosslinking of DNA, an anti-metabolic agent, a targeted agent such as a kinase inhibitor, a histone deacetylase inhibitor and an anti-EGFR agent and/or a therapeutical antibody designed to mediate cytotoxicity against the cancer cells or to modulate one of their key biological functions.

Antimitotic agents include, but are not limited to, paclitaxel, docetaxel and analogs such as larotaxel (also called XRP9881; Sanofi-Aventis), XRP6258 (Sanofi-Aventis), BMS-184476 (Bristol-Meyer-Squibb), BMS-188797 (Bristol-Meyer-Squibb), BMS-275183 (Bristol-Meyer-Squibb), ortataxel (also called IDN 5109, BAY 59-8862 or SB-T-101131; Bristol-Meyer-Squibb), RPR 109881A (Bristol-Meyer-Squibb), RPR 116258 (Bristol-Meyer-Squibb), NBT-287 (TAPESTRY), PG-paclitaxel (also called CT-2103, PPX, paclitaxel poliglumex, paclitaxel polyglutamate or Xyotax™), ABRAXANE® (also called Nab-paclitaxel; ABRAXIS BIOSCIENCE), tesetaxel (also called DJ-927), IDN 5390 (INDENA), taxoprexin (also called docosahexanoic acid-paclitaxel; PROTARGA), DHA-paclitaxel (also called Taxoprexin®), and MAC-321 (WYETH). Preferably, antimitotic agents are docetaxel, paclitaxel, and is more preferably docetaxel.

Inhibitors of topoisomerases I and/or II include, but are not limited to etoposide, topotecan, camptothecin, irinotecan, amsacrine, intoplicin, anthracyclines such as doxorubicin, epirubicin, daunorubicin, idarubicin and mitoxantrone. Inhibitors of topoisomerase I and II include, but are not limited to intoplicin.

The additional antitumor agent can be alkylating agents including, without limitation, nitrogen mustards, ethylenimine derivatives, alkyl sulfonates, nitrosoureas, metal salts and triazenes. Non-exhaustive examples thereof include uracil mustard, chlormethine, cyclophosphamide (CYTOXAN®), ifosfamide, melphalan, chlorambucil, pipobroman, triethylenemelamine, triethylenethiophosphoramine, busulfan, carmustine, lomustine, cisplatin, carboplatin, fotemustine, oxaliplatin, thiotepa, streptozocin, dacarbazine, and temozolomide. In a preferred embodiment, the DNA alkylating agent is preferably cisplatin, carboplatin, temozolomide, fotemustine or dacarbazine.

Anti-metabolic agents block the enzymes responsible for nucleic acid synthesis or become incorporated into DNA, which produces an incorrect genetic code and leads to apoptosis. Non-exhaustive examples thereof include, without limitation, folic acid antagonists, pyrimidine analogs, purine analogs and adenosine deaminase inhibitors, and more particularly methotrexate, floxuridine, cytarabine, 6-mercaptopurine, 6-thioguanine, fludarabine phosphate, pentostatine, 5-fluorouracil, gemcitabine and capecitabine. In a preferred embodiment, such an agent is gemcitabine.

The additional anti-tumor agent can also be a targeted agent, in particular a kinase inhibitor. The kinase may be selected from the group consisting of intracellular tyrosine or serine/threonine kinases, receptors tyrosine or serine/threonine kinase. The kinase could be selected among EGFR family, ALK, B-Raf, MEK, and mTOR. For instance, the agents may have ability to inhibit angiogenesis based on the inhibitory activities on VEGFR and PDGFR kinases. In particular, the targeted agent can be selected among the multiple kinase inhibitor drugs which are already approved: Gleevec®, which inhibits Bcr-Abl and c-Kit, and Iressa® and Tarceva®, which both inhibit EGFR, sorafenib (Nexavar®, BAY 43-9006) which inhibits Raf, dasatinib (BMS-354825) and nilotinib (AMN-107, Tasigna®) which also inhibits Bcr-Abl, lapatinib which also inhibits EGFR, temsirolimus (Torisel®, CCI-779) which targets the mTOR pathway, sunitinib (Student, SU11248) which inhibits several targets including VEGFR as well as specific antibodies inactivating kinase receptors: Herceptin® and Avastin®. The anti-EGFR agent can be selected among gefitinib, erlotinib, lapatinib, vandetanib, afatinib, osimertinib, neratinib, dacomitinib, brigatinib, canertinib, naquotinib, nazartinib, pelitinib, rociletinib, icotinib, AZD3759, AZ5104 (CAS No 1421373-98-9), poziotinib, WZ4002, preferably is erlotinib or cetuximab. The ALK inhibitor can be selected among crizotinib, entrectinib, ceritinib, alectinib, brigatinib, lorlatinib, TSR-011, CEP-37440, and ensartinib. The B-Raf inhibitor can be selected among vemurafenib, dabrafenib, regorafenib, and PLX4720. The MEK inhibitor can be selected among cobimetinib, trametinib, binimetinib, selumetinib, PD-325901, CI-1040, PD035901, U0126, TAK-733.

The additional drug can also be a checkpoint inhibitor, for instance an antibody targeting PD-1, PD-L1, CTLA-4 and the like.

### De Novo Pathway Inhibitor

In a preferred embodiment, the compound of the invention can be used in combination with an inhibitor of the De Novo nucleotide biosynthesis pathway. In De Novo (from scratch) pathways, the nucleotide bases are assembled from simpler compounds. The framework for a pyrimidine base is assembled first and then attached to ribose. In contrast, the framework for a purine base is synthesized piece by piece directly onto a ribose-based structure. De novo pathways synthesize pyrimidines and purine nucleotides from amino acids, carbon dioxide, folate derivatives, and phosphoribosyl pyrophosphate (PRPP).

As used herein the terms “De Novo nucleotide biosynthesis pathway inhibitor”, “inhibitor of the De Novo nucleotide biosynthesis pathway” or simply “De Novo pathway inhibitor” or “inhibitor of the De Novo pathway” refers to any agent (e.g., compound, antibody, protein, or nucleic acid) capable of reducing the level of a protein component of the De Novo nucleotide biosynthesis pathway, an mRNA of a protein component of the De Novo nucleotide biosynthesis pathway, or the activity of a component of the De novo nucleotide biosynthesis pathway, relative to a control (e.g., comparison of level in the absence of the De Novo pathway inhibitor). Preferably, the De Novo Pathway inhibitor is a compound (e.g., molecule). The De Novo pathway inhibitor may reduce the level of production of dCTP, and/or dATP and/or dGTP and dTTP compared to control (e.g., absence of the De Novo Pathway inhibitor). Non-limiting examples of De Novo Pathway inhibitor are listed below:
Methotrexate, ((2S)-2-[(4-{[(2,4-Diaminopteridin-6-yl)methyl](methyl)amino}benzoyl)amino]pentanedioic acid) (CAS Number 59-05-2);
Mercaptopurine (MP) (3,7-dihydropurine-6-thione), (CAS Number 50-44-2);
5-fluorouracil (5-FU) (CAS Number 51-21-8;
Mycophenolic acid (MPA) (CAS Number 24280-93-1);
Mizoribine (1-[(2R,3R,4S,5R)-3,4-dihydroxy-5-(hydroxymethyl)oxolan-2-yl]-5-hydroxyimidazole-4-carboxamide) (CAS Number 50924-49-7);
Teriflunomide ((Z)-2-cyano-3-hydroxy-N-[4-(trifluoromethyl)phenyl]but-2-enamide) (CAS Number 163451-81-8);
Brequinar (6-fluoro-2-(2′-fluoro-1,1′-biphenyl-4-yl)-3-methyl-4-quinolinecarboxylic acid) (CAS Number 96187-53-0);
Vidofludimus (2-[[2-fluoro-4-(3-methoxyphenyl)phenyl]carbamoyl]cyclopentene-1-carboxylic acid) (CAS Number 717824-30-1);
BAY2402234 ((S)—N-(2-chloro-6-fluorophenyl)-4-(4-ethyl-3-(hydroxymethyl)-5-oxo-4,5-dihydro-1H-1,2,4-triazol-1-yl)-5-fluoro-2-((1,1,1-trifluoropropan-2-yl)oxy)benzamide) (CAS Number 2225819-06-5); and
Leflunomide (5-methyl-N-[4-(trifluoromethyl)phenyl]-isoxazole-4-carboxamide) (CAS Number: 75706-12-6).

In a more preferred embodiment, the inhibitor of the De Novo pathway is a ribonucleotide reductase (RNR) inhibitor. As used herein the terms “inhibitor of ribonucleotide reductase” or “ribonucleotide reductase inhibitor refer to an agent (e.g., chemical compound, antibody, protein, or nucleic acid) capable of reducing the level of RNR protein, RNR mRNA, or RNR activity, relative to a control (e.g., comparison of level in the absence of the RNR inhibitor). Preferably, the RNR inhibitor is a chemical compound (e.g., a molecule). The RNR inhibitor may reduce the level of activity of RNR. The RNR inhibitor may reduce the level of activity of RNR when the RNR inhibitor binds RNR. Non-limiting examples of RNR inhibitors are listed below:
Hydroxycarbamide, also known as hydroxyurea (CAS Number 127-07-1);
Motexafin gadolinium, (CAS Number: 156436-89-4);
Fludarabine ((2R,3S,4S,5R)-2-(6-amino-2-fluoropurin-9-yl)-5-(hydroxymethyl)oxolane-3,4-diol), (CAS Number 21679-14-1);
Cladribine (5-(6-Amino-2-chloro-purin-9-yl)-2-(hydroxymethyl)oxolan-3-ol), (CAS Number 4291-63-8);
Gemcitabine (4-Amino-1-(2-deoxy-2,2-difluoro-β-D-erythro pentofuranosyl)pyrimidin-2(1H)-on), (CAS Number 95058-81-4);
Tezacitabine (4-Amino-1-[(2R,3E,4S,5R)-3-(fluoromethylidene)-4-hydroxy-5-(hydroxymethyl)oxolan-2-yl]pyrimidin-2(1H)-one), (CAS Number 130306-02-4);
DMDC (Deoxy-2′-methylidenecytidine) (CAS Number 119804-96-5);
Cytarabine (Cytosine arabinoside) (CAR Number 147-94-4);
Triapine (3-aminopyridine-2-carbaldehyde thiosemicarbazone), (CAS Number 236392-56-6);
Dp44mT (2-(di-2-pyridinylmethylene)-N,N-dimethyl-hydrazinecarbothioamide) (CAS Number 152095-12-0);
DpT (Di(2-pyridyl) ketone thiosemicarbazone) (CAS Number 6839-91-4);
DpC (Di-2-pyridylketone 4-cyclohexyl-4-methyl-3-thiosemicarbazone hydrochloride) (CAS Number 1382469-40-0);
Dp2mT (Di-2-pyridylketone-2-methyl-3-thiosemicarbazone) (CAS Number 741250-22-6);
Gallium maltolate (Tris(3-hydroxy-2-methyl-4H-pyran-4-one)gallium), (CAS Number 108560-70-9);
Gallium nitrate, (CAS Number 13494-90-1);
Clofarabine (5-(6-amino-2-chloro-purin-9-yl)-4-fluoro-2-(hydroxymethyl)oxolan-3-ol), (CAS Number 123318-82-1);
Didox (3,4-Dihydroxybenzohydroxamic acid) (CAS Number 69839-83-4);
Trimidox (3,4,5-Trihydroxybenzamidoxime) (CAS Number 95933-74-7);
COH29 (N-(4-(3,4-dihydroxyphenyl)-5-phenylthiazol-2-yl)-3,4-dihydroxybenzamide) (CAS Number 1190932-38-7);
DFOA (Desferrioxamine) (CAS Number 70-51-9);
PIH (Pyridoxal isonicotinoyl hydrazine) (CAS Number 737-86-0); and
Thymidine (dT), also known as deoxythymidine, deoxyribosylthymine (1-[(2R,4S,5R)-4-Hydroxy-5-(hydroxymethyl)oxolan-2-yl]-5-methylpyrimidine-2,4(1H,3H)-dione), (CAS Number 50-89-5).

In an even more preferred embodiment, the ribonucleotide reductase inhibitor is thymidine.

Further aspects and advantages of the invention will be disclosed in the following experimental section.

### EXAMPLES

### Example A—Chemistry

General Methods. Commercially available reagents and solvents were used without further additional purification. Thin layer chromatography (TLC) was performed on precoated aluminum sheets of silica (60 F254 nm, Merck) and visualized using short-wave UV light. Reaction monitoring and purity of compounds were recorded by using analytical Agilent Infinity high performance liquid chromatography with DAD at 254 nm column Agilent Poroshell 120 EC-C18 2.7 μm (4.6×50 mm), mobile phase (A: 0.1% FA H₂O, B: 0.1% FA MeCN), method (A) flow rate 0.3 mL/min, time/% B 0/10, 4/90, 7/90, 9/10, 10/10; method (B) flow rate 0.5 mL/min, time/% B 0/10, 4/90, 7/90, 9/10, 13/10); mobile phase (A: 0.1% TFA H₂O, B: 0.1% TFA MeCN), method (C) flow rate 1 mL/min, time/% B 0/10, 5/100, 8/100; column Thermo Scientific Hypersil Gold 12 μm (4.6×250 mm), mobile phase (A: 0.1% TFA H₂O, B: 0.1% TFA MeCN), method (D) flow rate 2 mL/min, time/% B 0/10, 6/100, 11/100. Column chromatography was performed on a Reveleris purification system using Reveleris Flash silica cartridges or C18 40 μM cartridges. Petroleum refers to the fraction with distillation range 40-65° C. ¹H and ¹³C NMR spectra were recorded by using a Bruker AC 400, AC300 or AC250 spectrometer. Chemical shifts, (δ) are reported in ppm and coupling values (J) in hertz. Abbreviations for peaks are, br: broad, s: singlet, d: doublet, t: triplet, q: quadruplet, quint: quintuplet, sext: sextuplet, sept: septuplet and m: multiplet). The spectra recorded are consistent with the proposed structures. Low-resolution mass spectra were obtained with Agilent SQ G6120B mass spectrometer in positive and negative electrospray mode.

General methods for preparing compounds of the invention are illustrated by Schemes 1-7.

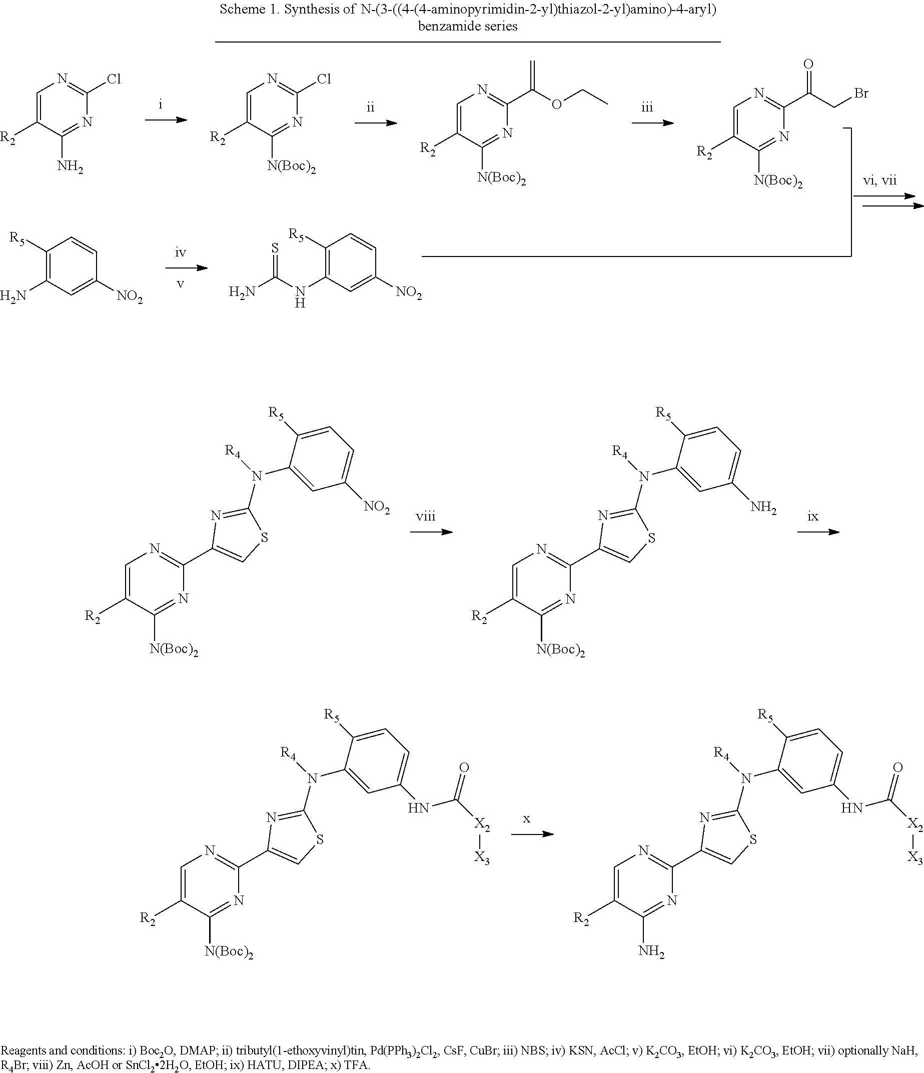

N-(3-((4-(4-aminopyrimidin-2-yl)thiazol-2-yl)amino)-4-aryl)benzamide derivatives were prepared by convergent synthesis, from commercially available 4-amino-2-chloropyrimidine and appropriate nitroaniline (Scheme 1). The 4-amino-2-chloropyrimidine was protected using Boc₂O to afford corresponding bis-carbamate. A Stille cross-coupling reaction with tributyl(1-ethoxyvinyl)tin gives the enol ether which was then turned into the corresponding α-bromoketone using N-bromosuccinimide. Starting from appropriate nitroaniline, condensation with acetylisothiocyanate followed by saponification gave the corresponding thiourea. The α-bromoketone was engaged in the Hantzsch thiazole synthesis with the thiourea leading to the corresponding thiazole. Optionally R₄ as (C₁-C₆)alkyl group can be introduced before reduction of the nitro group followed by peptide coupling with appropriate carboxylic acid affording expected amides. Finally, deprotection with TFA led to corresponding N-(3-((4-(4-aminopyrimidin-2-yl)thiazol-2-yl)amino)-4-aryl)benzamides.

General Procedure for the Synthesis of N-(3-((4-(4-aminopyrimidin-2-yl)thiazol-2-yl)amino)-4-aryl)benzamide Derivatives. A solution of N-(3-((4-(4-(di-tert-butoxycarbonylamino)pyrimidin-2-yl)thiazol-2-yl)amino)-4-aryl)benzamide derivative (0.1 mmol) in a mixture of dichloromethane-trifluoroacetic acid (3:1, 4 mL) was stirred at room temperature for 2 hrs. The solvent was distillated off under reduced pressure and the residue was purified by flash chromatography to afford the corresponding N-(3-((4-(4-aminopyrimidin-2-yl)thiazol-2-yl)amino)-4-aryl)benzamide.

N-(3-((4-(4-Aminopyrimidin-2-yl)thiazol-2-yl)amino)-4-methylphenyl)-4-((4-methylpiperazin-1-yl)methyl)benzamide dCKi-1 (78%) as a white powder. ¹H NMR (400 MHz, MeOD) δ 8.40 (d, J=1.7 Hz, 1H), 8.13 (d, J=6.0 Hz, 1H), 7.96 (d, J=8.2 Hz, 2H), 7.66 (dd, J=8.2, 1.7 Hz, 1H), 7.60 (s, 1H), 7.49 (d, J=8.2 Hz, 2H), 7.21 (d, J=8.2 Hz, 1H), 6.44 (d, J=6.0 Hz, 1H), 3.61 (brs, 2H), 2.53 (brs, 8H), 2.32 (s, 3H), 2.30 (s, 3H); ¹³C NMR (100 MHz, MeOD) δ 168.4, 167.5, 165.5, 161.2, 155.8, 151.1, 142.9, 140.7, 138.8, 135.4, 131.9, 130.6, 128.7, 125.5, 117.0, 113.9, 112.7, 104.6, 63.3, 55.7, 53.5, 45.9, 17.6; LCMS C₂₇H₃₀FN₈OS method (D) Rₜ=3.579 min, ESI+ m/z=515.2 (M+H).

N-(1-(4-(4-Aminopyrimidin-2-yl)thiazol-2-yl)-2,3,4,5-tetrahydro-1H-benzo[b]azepin-8-yl)-4-((4-methylpiperazin-1-yl)methyl)benzamide OR0289 (66%) as a white solid. Rf=0.40 (DCM-MeOH—NH₄OH, 85:13.5:1.5); ¹H NMR (400 MHz, MeOD) δ 8.08 (d, J=6.0 Hz, 1H), 7.89 (d, J=8.2 Hz, 2H), 7.83 (d, J=2.0 Hz, 1H), 7.62 (dd, J=8.2, 2.1 Hz, 1H), 7.44 (d, J=8.2 Hz, 2H), 7.37 (s, 1H), 7.30 (d, J=8.3 Hz, 1H), 6.41 (d, J=6.0 Hz, 1H), 3.95 (m, 2H), 3.58 (s, 2H), 2.78-2.67 (m, 2H), 2.57 (s, 8H), 2.34 (s, 3H), 1.93 (m, 2H), 1.69 (m, 2H); ¹³C NMR (100 MHz, MeOD) δ 170.78, 168.40, 165.47, 161.24, 155.30, 151.56, 146.10, 142.94, 139.56, 138.08, 135.09, 132.21, 130.50, 128.78, 121.77, 121.45, 112.18, 104.50, 63.08, 55.57, 53.20, 52.20, 45.63, 34.87, 29.52, 27.58; LC method (C) Rₜ=2.822 min.

N-(3-((4-(4-Aminopyrimidin-2-yl)thiazol-2-yl)(propyl)amino)-4-methylphenyl)-4-((4-methylpiperazin-1-yl)methyl)benzamide OR0105 (86%) as a white solid. Rf=0.18 (DCM-MeOH—NH₄OH, 90:9:1). ¹H NMR (400 MHz, MeOD) δ 8.14 (d, J=6.0 Hz, 1H), 7.93 (d, J=8.3 Hz, 2H), 7.78 (d, J=2.0 Hz, 1H), 7.72 (dd, J=8.3, 2.2 Hz, 1H), 7.51 (d, J=8.2 Hz, 2H), 7.41 (d, J=8.4 Hz, 1H), 7.38 (s, 1H), 6.45 (d, J=6.0 Hz, 1H), 4.01 (brs, 2H), 3.64 (s, 2H), 2.57 (brs, 8H), 2.34 (s, 3H), 2.26 (s, 3H), 1.86-1.69 (m, 2H), 1.02 (t, J=7.4 Hz, 3H); LC method (C) Rₜ=3.967 min.

N-(3-((4-(4-Aminopyrimidin-2-yl)thiazol-2-yl)(propyl)amino)-4-methylphenyl)-2-((4-methylpiperazin-1-yl)methyl)pyrimidine-5-carboxamide OR0125 (81%) as a light orange powder. Rf=0.23 (DCM-MeOH—NH₄OH, 80:20:0.2); ¹H NMR (400 MHz, MeOD) δ 9.33 (s, 2H), 8.54 (d, J=1.9 Hz, 1H), 8.14 (d, J=6.0 Hz, 1H), 7.70 (dd, J=8.2, 1.9 Hz, 1H), 7.63 (s, 1H), 7.21 (d, J=8.2 Hz, 1H), 6.45 (d, J=6.0 Hz, 1H), 3.91 (s, 2H), 2.82-2.46 (m, 8H), 2.33 (s, 6H); LC method (D) Rₜ=3.967 min.

N-(3-((4-(4-Aminopyrimidin-2-yl)thiazol-2-yl)amino)-4-methylphenyl)-4-(2-(4-methylpiperazin-1-yl)ethyl)benzamide OR0146 (49%) as orange crystals. Rf=0.55 (DCM-MeOH—NH₄OH, 80:18:2). ¹H NMR (400 MHz, MeOD) δ 8.42 (d, J=2.0 Hz, 1H), 8.10 (d, J=6.0 Hz, 1H), 7.88 (d, J=8.3 Hz, 2H), 7.61 (dd, J=8.5, 2.0 Hz, 1H), 7.59 (s, 1H), 7.32 (d, J=8.3 Hz, 2H), 7.16 (d, J=8.5 Hz, 1H), 6.41 (d, J=6.0 Hz, 1H), 2.88-2.81 (m, 2H), 2.65-2.59 (m, 2H), 2.59 (s, 8H), 2.32 (s, 3H), 2.29 (s, 3H); ¹³C NMR (100 MHz, MeOD) δ 168.39, 167.34, 165.46, 160.96, 155.39, 150.83, 145.51, 140.60, 138.79, 134.16, 131.85, 129.92, 128.86, 125.26, 116.90, 113.78, 112.99, 104.63, 60.58, 55.45, 53.28, 45.69, 33.83, 17.63; LC method (D) Rₜ=3.592 min.

N-(3-((4-(4-Amino-5-fluoropyrimidin-2-yl)thiazol-2-yl)amino)-4-methylphenyl)-4-((4-methylpiperazin-1-yl)methyl)benzamide OR0239 (57%) as a white powder. ¹H NMR (400 MHz, MeOD) δ 8.33 (d, J=1.8 Hz, 1H), 8.10 (d, J=3.6 Hz, 1H), 7.95 (d, J=8.2 Hz, 2H), 7.64 (dd, J=8.3, 1.8 Hz, 1H), 7.54 (s, 1H), 7.49 (d, J=8.2 Hz, 2H), 7.21 (d, J=8.3 Hz, 1H), 3.62 (brs, 2H), 2.52 (brs, 8H), 2.32 (s, 3H), 2.29 (s, 3H); LC method (D) Rₜ=3.548 min N-(3-((4-(4-Aminopyrimidin-2-yl)thiazol-2-yl)amino)-4-methylphenyl)-4-((dimethylamino)methyl)benzamide OR0155. (77%) as a white solid. Rf=0.20 (DCM-MeOH—NH₄OH, 90:9:1); ¹H NMR (400 MHz, MeOD) δ8.39 (s, 1H), 8.11 (s, 1H), 7.96 (d, J=8.1 Hz, 2H), 7.62 (m, 1H), 7.60 (s, 1H), 7.47 (d, J=8.21 Hz, 2H), 7.20 (d, J=8.01 Hz, 1H), 6.43 (d, J=6.1 Hz, 1H), 3.60 (s, 2H), 2.31 (s, 3H), 2.30 (s, 6H); ¹³C NMR (100 MHz, MeOD) S 168.32, 167.53, 165.52, 161.13, 155.60, 152.54, 142.42, 140.58, 138.80, 135.63, 131.92, 130.83, 128.81, 125.52, 117.08, 113.97, 112.80, 104.67, 64.30, 45.19, 17.59; LC method (D) Rₜ=3.703 min.

N-(3-((4-(4-Aminopyrimidin-2-yl)thiazol-2-yl)amino)-4-methylphenyl)-4-((6-methyl-2,6-diazaspiro[3.3]heptan-2-yl)methyl)benzamide OR0156 (10% over 2 steps) as a white solid. Rf=0.18 (DCM-MeOH—NH₄OH, 80:20:2). ¹H NMR (400 MHz, MeOD) δ 8.42 (d, J=2.0 Hz, 1H), 8.13 (d, J=6.0 Hz, 1H), 7.96 (d, J=8.1 Hz, 2H), 7.63 (s, 1H), 7.52 (dd, J=8.2, 2.0 Hz, 1H), 7.43 (d, J=8.1 Hz, 2H), 7.21 (d, J=8.3 Hz, 1H), 6.46 (d, J=6.1 Hz, 1H), 3.98 (s, 4H), 3.72 (s, 2H), 3.49 (s, 4H), 2.69 (s, 3H), 2.33 (s, 3H).

N-(3-((4-(4-Aminopyrimidin-2-yl)thiazol-2-yl)amino)-4-methylphenyl)-5-((4-methylpiperazin-1-yl)methyl)thiophene-2-carboxamide OR0241 (83%) as a white powder. ¹H NMR (400 MHz, DMSO-d₆) δ 10.17 (s, 1H), 9.37 (brs, 1H), 8.08 (d, J=5.8 Hz, 1H), 7.98 (d, J=1.8 Hz, 1H), 7.83 (d, J=3.8 Hz, 1H), 7.47 (s, 1H), 7.45 (dd, J=8.4, 1.8 Hz, 1H), 7.20 (d, J=8.4 Hz, 1H), 7.02 (d, J=3.8 Hz, 1H), 6.79 (brs, 2H), 6.31 (d, J=5.8 Hz, 1H), 3.68 (s, 2H), 2.42 (brs, 4H), 2.33 (brs, 4H), 2.23 (s, 3H), 2.15 (s, 3H); LC method (D) Rₜ=3.725 min.

General Procedure for the Synthesis of di-tert-butyl (2-(2-((3-benzamidoaryl)amino)thiazol-4-yl)pyrimidin-4-yl)carbamate Derivatives. To a solution of di-tert-butyl (2-(2-((3-aminoaryl)amino)thiazol-4-yl)pyrimidin-4-yl)carbamate derivative (0.37 mmol) and appropriate acid (0.56 mmol) in dry dimethyformamide (6 mL) were successively added 0-(7-azabenzotriazol-1-yl)-N,N,N′,N′-tetramethyluronium hexafluorophosphate (170 mg, 0.44 mmol), in one portion, and diisopropylethylamine (330 μL, 1.85 mmol). The resulting mixture was stirred overnight at room temperature and then concentrated under reduced pressure. The residue was diluted with EtOAc (100 mL) and successively washed twice with water (20 mL) and saturated aqueous Na₂CO₃ (20 mL). The organic layer was dried over Na₂SO₄, and the solvent was distillated off under reduced pressure. The residue was purified by flash chromatography to afford the corresponding di-tert-butyl (2-(2-((3-benzamidoaryl)amino)thiazol-4-yl)pyrimidin-4-yl)carbamate.

Di-tert-butyl (2-(2-((2-methyl-5-(4-(2-(4-methylpiperazin-1-yl)methyl)benzamido)phenyl)amino)thiazol-4-yl)pyrimidin-4-yl)carbamate dCKi-1-1 (38%) as a white powder. ¹H NMR (300 MHz, CDCl₃) 6.68 (d, J=5.7, 1H), 8.19 (s, 1H), 7.93 (brs, 1H), 7.84 (d, J=8.0, 2H), 7.55 (s, 1H), 7.54 (d, J=5.6, 1H), 7.45-7.41 (m, 3H), 7.22-7.18 (m, 2H), 3.57 (s, 2H), 2.51 (brs, 8H), 2.33 (s, 3H), 2.29 (s, 3-H), 1.55 (s, 18H); ¹³C NMR (75 MHz, CDCl₃) δ 165.7, 165.5, 159.9, 158.6, 150.3, 149.7, 142.8, 138.9, 137.4, 133.8, 131.6, 129.5, 127.2, 124.2, 115.3, 112.5, 110.9, 110.6, 84.4, 77.4, 62.6, 52.2, 53.0, 46.0, 28.0, 17.6; LCMS C₃₇H₄₆N₈O₅S method (D) Rₜ=4.908 min, ESI+ m/z=715.3 (M+H).

Di-tert-butyl (2-(2-(8-(4-((4-methylpiperazin-1-yl)methyl)benzamido)-2,3,4,5-tetrahydro-1H-benzo[b]azepin-1-yl)thiazol-4-yl)pyrimidin-4-yl)carbamate OR0289-1 (45%) as a white foamy solid. Rf=0.30 (DCM-MeOH—NH₄OH, 90:9:1); ¹H NMR (400 MHz, MeOD) S 8.74 (d, J=5.7 Hz, 1H), 7.91 (d, J=8.2 Hz, 2H), 7.85 (d, J=2.0 Hz, 1H), 7.64 (dd, J=8.3, 2.1 Hz, 1H), 7.56 (d, J=5.7 Hz, 1H), 7.49 (d, J=8.3 Hz, 2H), 7.46 (s, 1H), 7.35 (d, J=8.3 Hz, 1H), 4.05 (brs, 2H), 3.67 (s, 2H), 2.95-2.82 (brs, 4H), 2.69-2.50 (brs, 4H), 2.75 (m, 2H), 2.59 (s, 3H), 1.98 (s, 2H), 1.74 (m, 2H), 1.54 (s, 18H); ¹³C NMR (100 MHz, MeOD) δ 171.20, 168.46, 161.55, 160.25, 159.48, 151.57, 151.18, 146.12, 142.77, 139.61, 138.22, 135.24, 132.29, 130.45, 128.82, 121.86, 121.46, 113.68, 113.03, 85.64, 62.68, 55.31, 52.29, 52.19, 44.76, 34.89, 29.57, 28.07, 27.62; LC method (C) Rₜ=4.143 min.

Di-tert-butyl (2-(2-((2-methyl-5-(4-((4-methylpiperazin-1-yl)methyl)benzamido)phenyl)(propyl)amino)thiazol-4-yl)pyrimidin-4-yl)carbamate OR0105-1 (55%) as a white powder used in the next step without further purification.

Di-tert-butyl (2-(2-((2-methyl-5-(2-((4-methylpiperazin-1-yl)methyl)pyrimidine-5-carboxamido)phenyl)(propyl)amino)thiazol-4-yl)pyrimidin-4-yl)carbamate OR0125-1 (26%) as an orange powder. Rf=0.42 (DCM-MeOH—NH₄OH, 90:9:1); ¹H NMR (400 MHz, MeOD) δ 9.38 (s, 2H), 8.69 (d, J=5.7 Hz, 1H), 8.34 (d, J=1.3 Hz, 1H), 7.58 (dd, J=8.3, 1.3 Hz, 1H), 7.52 (d, J=5.7 Hz, 1H), 7.49 (s, 1H), 7.16 (d, J=8.3 Hz, 1H), 3.91 (s, 2H), 3.09 (brs, 8H), 2.31 (s, 6H), 1.53 (s, 18H).

Di-tert-butyl (2-(2-((2-methyl-5-(4-(2-(4-methylpiperazin-1-yl)ethyl)benzamido)phenyl)amino)thiazol-4-yl)pyrimidin-4-yl)carbamate OR0146-1 (28%) as a white foamy solid. Rf=0.36 (DCM-MeOH—NH₄OH, 90:9:1); ¹H NMR (400 MHz, MeOD) δ 8.73 (d, J=5.6 Hz, 1H), 8.36 (d, J=1.4 Hz, 1H), 7.92 (d, J=8.1 Hz, 2H), 7.64 (s, 1H), 7.61 (dd, J=8.3, 1.5 Hz, 1H), 7.59 (d, J=5.5 Hz, 1H), 7.39 (d, J=8.1 Hz, 2H), 7.19 (d, J=8.3 Hz, 1H), 3.09 (brs, 4H), 2.91 (m, 2H), 2.85 (brs, 4H), 2.80 (m, 2H), 2.72 (s, 3H), 2.30 (s, 3H), 1.53 (s, 18H); ¹³C NMR (100 MHz, MeOD) δ 168.40, 168.16, 161.12, 160.25, 159.55, 151.40, 150.24, 145.04, 140.44, 138.76, 134.14, 131.98, 130.06, 128.93, 125.90, 117.41, 114.48, 114.29, 113.00, 85.75, 59.58, 54.71, 51.67, 44.15, 33.59, 28.09, 17.64; LC method (D) Rₜ=4.960 min.

Di-tert-butyl (5-fluoro-2-(2-((2-methyl-5-(4-((4-methylpiperazin-1-yl)methyl)benzamido)phenyl)amino)thiazol-4-yl)pyrimidin-4-yl)carbamate OR0239-1 (31%) as a light brown powder. ¹H NMR (400 MHz, CDCl₃) δ 8.65 (d, J=1.3 Hz, 1H), 8.19 (d, J=1.8 Hz, 1H), 7.84 (d, J=8.2 Hz, 2H), 7.78 (dd, J=8.2, 1.3 Hz, 1H), 7.61 (s, 1H), 7.45 (d, J=8.2 Hz, 2H), 7.21 (d, J=8.2 Hz, 1H), 3.57 (s, 2H), 2.48 (brs, 8H), 2.31 (s, 3H), 2.29 (s, 3H), 1.45 (s, 18H).

Di-tert-butyl (2-(2-((2-methyl-5-(5-((4-methylpiperazin-1-yl)methyl)thiophene-2-carboxamido)phenyl)amino)thiazol-4-yl)pyrimidin-4-yl)carbamate OR0241-1 (31%) as a yellow powder. Rf=0.53 (DCM-MeOH—NH₄OH, 95:5:0.5); ¹H NMR (400 MHz, CDCl₃) δ 8.67 (d, J=5.7 Hz, 1H), 8.18 (d, J=1.8 Hz, 1H), 8.07 (brs, 1H), 7.60-7.50 (m, 3H), 7.39 (brs, 1H), 7.26-7.18 (m, 2H), 6.90 (d, J=3.6 Hz, 1H), 3.74 (s, 2H), 2.67 (brs, 8H), 2.44 (s, 3H), 2.27 (s, 3H), 1.56 (s, 18H).

Di-tert-butyl (2-(2-((5-(4-((dimethylamino)methyl)benzamido)-2-methylphenyl)amino)thiazol-4-yl)pyrimidin-4-yl)carbamate OR0155-1.

Under argon, at 0° C., to a solution of di-tert-butyl (2-(2-((5-amino-2-methylphenyl)amino)thiazol-4-yl)pyrimidin-4-yl)carbamate dCKi-1-2 (195 mg, 0.391 mmol), in a mixture of tetrahydrofuran-dimethylformamide (4:1, 5 mL) was successively added N,N-diisopropylethylamine (DIPEA) (400 μL, 2.30 mmol) and a freshly prepared suspension of 4-((dimethylamino)methyl)benzoyl chloride hydrochloride (108 mg, 0.463 mmol) in tetrahydrofuran (4 mL). After addition, the resulting mixture was stirred overnight at room temperature. The solvent was distillated off under reduced pressure and the residue was taken up into saturated Na₂CO₃ aqueous solution (20 mL) and extracted with EtOAc (2×50 mL). The combined organic layers were dried over Na₂SO₄ and concentrated under reduced pressure. The residue was purified by flash chromatography, gradient (DCM-MeOH—NH₄OH, 90:10:1) to afford 4-((dimethylamino)methyl)-N-(3-((4-(4-(di-tertbutoxycarbonylamino)pyrimidin-2-yl)thiazol-2-yl)amino)-4-methylphenyl)benzamide OR0155-1 (75 mg, 29%) as a white solid. Rf=0.10 (DCM-MeOH—NH₄OH, 95:5:1). ¹H NMR (400 MHz, CDCl₃) δ 9.09 (s, 1H), 8.54 (s, 1H), 8.17 (s, 1H), 7.88 (d, J=6.8 Hz, 2H), 7.53-7.34 (m, 5H), 7.09 (d, J=7.6 Hz, 1H), 3.70 (s, 2H), 2.37 (s, 6H), 2.20 (s, 3H), 1.52 (s, 18H). ¹³C NMR (100 MHz, CDCl₃) δ 166.19, 165.81, 159.74, 158.44, 158.32, 150.21, 149.51, 138.81, 137.52, 134.84, 131.38, 130.06, 127.88, 124.86, 116.21, 114.15, 112.39, 111.95, 110.69, 84.44, 62.65, 44.26, 27.90, 17.52; LC method (D) Rₜ=5.229 min.

General Procedure for the Synthesis of di-tert-butyl (2-(2-((3-aminoaryl)amino)thiazol-4-yl)pyrimidin-4-yl)carbamate derivatives. To a solution of di-tert-butyl (2-(2-((3-nitroaryl)amino)thiazol-4-yl)pyrimidin-4-yl)carbamate derivative (0.5 mmol) in a mixture of tetrahydrofuran-ethanol (10:1, 22 mL) was added tin (II) chloride dihydrate (1.14 g, 5.06 mmol). The reaction mixture was stirred overnight at room temperature until the reaction was complete as indicated by TLC or LCMS monitoring. The reaction was quenched with a saturated aqueous Na₂CO₃ solution (20 mL) and stirred for further 15 min. After dilution with EtOAc (70 mL) and filtration through a short pad of celite, the aqueous layer was extracted twice with EtOAc (70 mL). The combined organic layers were dried over Na₂SO₄ and the solvent was distillated off under reduced pressure. The residue was purified by flash chromatography to afford the corresponding di-tert-butyl (2-(2-((3-aminoaryl)amino)thiazol-4-yl)pyrimidin-4-yl)carbamate.

Di-tert-butyl (2-(2-((5-amino-2-methylphenyl)amino)thiazol-4-yl)pyrimidin-4-yl)carbamate dCKi-1-2 (77%) as an orange foamy solid. Rf=0.40 (DCM-MeOH, 95:5); ¹H NMR (400 MHz, CDCl₃) δ 8.65 (d, J=5.7 Hz, 1H), 7.51 (d, J=5.7 Hz, 1H), 7.48 (s, 1H), 7.00 (d, J=8.1 Hz, 1H), 6.89 (d, J=2.3 Hz, 1H), 6.43 (dd, J=8.0, 2.3 Hz, 1H), 3.67 (s, 2H), 2.18 (s, 3H), 1.55 (s, 18H); ¹³C NMR (75 MHz, CDCl₃) δ 166.6, 158.6, 150.3, 149.9, 145.8, 139.2, 132.0, 119.3, 111.8, 111.7, 111.0, 107.1, 84.4, 77.4, 28.0, 17.1; LCMS C₂₄H₃₀N₆O₄S method (D) Rₜ=5.154 min, ESI+ m/z=499.2 (M+H).

Di-tert-butyl (2-(2-(8-amino-2,3,4,5-tetrahydro-1H-benzo[b]azepin-1-yl)thiazol-4-yl)pyrimidin-4-yl)carbamate OR0289-2 (71%) as a yellow foamy solid. Rf=0.20 (DCM-MeOH, 95:5); ¹H NMR (400 MHz, CDCl₃) δ 8.70 (d, J=5.7 Hz, 1H), 7.47 (d, J=5.7 Hz, 1H), 7.37 (s, 1H), 7.04 (d, J=8.1 Hz, 1H), 6.72 (d, J=2.1 Hz, 1H), 6.57 (dd, J=8.0, 2.3 Hz, 1H), 3.97 (brs, 2H), 3.45 (brs, 2H), 2.70-2.55 (m, 2H), 1.90 (m, 2H), 1.63 (s, 2H), 1.53 (s, 18H); ¹³C NMR (100 MHz, CDCl₃) δ 169.99, 160.40, 158.51, 158.39, 150.45, 150.32, 145.79, 145.53, 131.66, 130.85, 115.07, 114.64, 112.63, 110.45, 84.25, 51.01, 33.46, 28.48, 27.92, 26.77; LC method (C) Rₜ=4.122 min.

Di-tert-butyl (2-(2-((5-amino-2-methylphenyl)(propyl)amino)thiazol-4-yl)pyrimidin-4-yl)carbamate OR0105-2 (78%) as a yellow foamy solid. Rf=0.30 (DCM-MeOH, 95:5); ¹H NMR (400 MHz, CDCl₃) δ 8.72 (d, J=5.7 Hz, 1H), 7.47 (d, J=5.7 Hz, 1H), 7.37 (s, 1H), 7.11 (d, J=8.2 Hz, 1H), 6.68 (dd, J=8.1, 2.2 Hz, 1H), 6.59 (s, 1H), 3.96 (brs, 2H), 2.12 (s, 3H), 2.04 (s, 3H), 1.69-1.59 (m, 2H), 1.54 (s, 18H), 0.92 (t, J=7.3 Hz, 2H); LC method (D) Rₜ=5.050 min.

Di-tert-butyl (2-(2-((5-amino-2-methylphenyl)amino)thiazol-4-yl)-5-fluoropyrimidin-4-yl)carbamate OR0239-2 (70%) as a yellow powder. ¹H NMR (400 MHz, CDCl₃) δ 8.62 (s, 1H), 7.55 (s, 1H), 7.02 (d, J=8.0 Hz, 1H), 6.88 (d, J=2.0, 1H), 6.45 (dd, J=8.0, 2.0 Hz, 1H), 3.67 (brs, 2H), 2.19 (s, 3H), 1.45 (s, 18H); LC method (D) Rₜ=5.101 min.

Di-tert-butyl (2-(2-((5-nitro-2-methylphenyl)(propyl)amino)thiazol-4-yl)pyrimidin-4-yl)carbamate OR0105-3.

Under argon, at 0° C., to a stirred solution of di-tert-butyl (2-(2-((2-methyl-5-nitrophenyl)amino)thiazol-4-yl)pyrimidin-4-yl)carbamate dCKi-1-3 (400 mg, 0.757 mmol) in a mixture of tetrahydrofuran-dimethylformamide (5:2, 14 mL) was added sodium hydride (60% in mineral oil, 38 mg, 0.95 mmol). After addition, the reaction mixture was stirred during 30 minutes at 0° C., allowed to warm to room temperature and stirred for further 30 minutes at this temperature. Then, propyl bromide (90 μL, 0.99 mmol) was added at 0° C. and the resulting suspension was heated to 60° C. for 3 days. The reaction was carefully quenched at 0° C. with methanol (10 ml) and concentrated under reduced pressure. The residue was purified by flash chromatography, gradient Cyclohexane-EtOAc (95:5 to 85:15) to afford di-tert-butyl (2-(2-((5-nitro-2-methylphenyl)(propyl)amino)thiazol-4-yl)pyrimidin-4-yl)carbamate OR0105-3 (142 mg, 33%) as a white solid. Rf=0.67 (PE-EtOAc, 1:1); ¹H NMR (400 MHz, CDCl₃) δ 8.72 (d, J=5.7 Hz, 1H), 8.18 (dd, J=8.4, 2.2 Hz, 1H), 8.16 (d, J=2.2 Hz, 1H), 7.53 (d, J=8.4 Hz, 1H), 7.52 (d, J=5.7 Hz, 1H), 7.43 (s, 1H), 4.09-3.96 (m, 2H), 2.37 (s, 3H), 1.72-1.59 (m, 2H), 1.54 (s, 18H), 0.95 (t, J=7.4 Hz, 3H); LCMS C₂H₃₄N₆O₆S method (B) Rₜ=8.987 min, ESI+ m/z=571.3 (M+H).

General Procedure for the Synthesis of di-tert-butyl (2-(2-((3-nitroaryl)amino)thiazol-4-yl)pyrimidin-4-yl)carbamate derivatives. To a stirred solution of appropriate di-tert-butyl (2-(2-bromoacetyl)-pyrimidin-4-yl)carbamate (1.3 mmol) and 1-(3-nitroaryl)thiourea (1.22 mmol) in ethanol (40 mL) was added potassium carbonate (258 mg, 1.87 mmol). The resulting mixture was stirred at temperature and for time indicated below. The solvent was distillated off under reduced pressure. The residue was purified by flash chromatography to afford the corresponding di-tert-butyl (2-(2-((3-nitroaryl)amino)thiazol-4-yl)pyrimidin-4-yl)carbamate.

Di-tert-butyl (2-(2-((2-methyl-5-nitrophenyl)amino)thiazol-4-yl)pyrimidin-4-yl)carbamate dCKi-1-3 (96%) as a dark orange foamy solid. Rf=0.10 (PE-EtOAc, 1:1); ¹H NMR (400 MHz, CDCl₃) δ 8.58 (d, J=5.7 Hz, 1H), 8.47 (d, J=2.2 Hz, 1H), 7.90 (dd, J=8.3, 2.2 Hz, 1H), 7.60 (s, 1H), 7.55 (d, J=5.7 Hz, 1H), 7.38 (d, J=8.3 Hz, 1H), 2.40 (s, 3H), 1.56 (s, 18H); LCMS C₂₄H₂₈N₆O₆S, method (B) Rₜ=8.197 min, ESI+ m/z=529.2 (M+H).

Di-tert-butyl (2-(2-(8-nitro-2,3,4,5-tetrahydro-1H-benzo[b]azepin-1-yl)thiazol-4-yl)pyrimidin-4-yl)carbamate OR0289-3 (50%) as a yellow foamy solid. Rf=0.20 (DCM-MeOH, 98:2); ¹H NMR (400 MHz, CDCl₃) δ 8.73 (d, J=5.8 Hz, 1H), 8.32 (d, J=2.2 Hz, 1H), 8.10 (dd, J=8.4, 2.2 Hz, 1H), 7.54 (d, J=5.8 Hz, 1H), 7.47 (d, J=8.5 Hz, 1H), 7.46 (s, 1H), 4.06 (brs, 2H), 2.91-2.82 (m, 2H), 2.01-1.88 (m, 2H), 1.75 (brs, 2H), 1.54 (s, 18H). ¹³C NMR (100 MHz, CDCl₃) δ 168.86, 159.95, 158.57, 158.35, 150.25, 148.70, 147.50, 145.45, 131.81, 123.56, 123.51, 122.87, 113.21, 110.52, 84.37, 51.08, 34.24, 28.24, 27.92, 25.42; LCMS C₂₇H₃₂N₆O₆S, method (B) Rₜ=8.694 min, ESI+ m/z=569.3 (M+H).

Di-tert-butyl (5-fluoro-2-(2-((2-methyl-5-nitrophenyl)amino)thiazol-4-yl)pyrimidin-4-yl)carbamate OR0239-3 (83%) as a deep orange solid. ¹H NMR (400 MHz, CDCl₃) δ 8.67 (s, 1H), 8.52 (d, J=2.2 Hz, 1H), 7.92 (dd, J=8.3, 2.2 Hz, 1H), 7.71 (s, 1H), 7.40 (d, J=8.3 Hz, 1H), 2.42 (s, 3H), 1.46 (s, 18H); LCMS C₂₄H₂₇FN₆O₆S, method (A) Rₜ=7.871 min, ESI− m/z=545.2 (M−H).

General Procedure for the Synthesis of 1-(3-nitroaryl)thiourea Derivatives. A suspension of appropriate N-((3-nitroaryl)carbamothioyl)acetamide (2 mmol) and potassium carbonate (1.53 g, 11.1 mmol), in methanol (10 mL) was stirred at room temperature, upon complete consumption of the starting material monitored by TLC or LCMS. The solvent was distillated off under reduced pressure and the residue was poured onto water (30 mL), and then extracted with DCM (3×50 mL). The combined organic layers were dried over Na₂SO₄, and the solvent was distillated off under reduced pressure. The crude product was purified by flash chromatography to afford corresponding 1-(3-nitroaryl)thiourea.

1-(2-Methyl-5-nitrophenyl)thiourea dCKi-1-4 (90%) as a pale yellow solid. Rf=0.20 (PE-EtOAc, 1:1); ¹H NMR (300 MHz, DMSO-d₆) δ 9.41 (s, 1H), 8.27 (d, J=2.4 Hz, 1H), 7.98 (dd, J=8.4, 2.4 Hz, 1H), 7.70 (s, 2H), 7.51 (d, J=8.5 Hz, 1H), 2.31 (s, 3H); LCMS C₈H₉N₃O₂S method (D) Rₜ=4.675 min, ESI+ m/z=212.1 (M+H).

N-8-Nitro-2,3,4,5-tetrahydro-1H-benzo[b]azepine-1-carbothioamide OR0289-4 (79%) as a white solid. Rf=0.31 (DCM-MeOH, 98:2); ¹H NMR (400 MHz, CDCl₃) δ 8.20-8.12 (m, 2H), 7.51 (d, J=7.9 Hz, 1H), 5.62 (brs, 2H), 5.31 (d, J=12.8 Hz, 1H), 2.99 (t, J=12.8 Hz, 1H), 2.86 (m, 2H), 2.33 (m, 1H), 2.09 (d, J=14.0 Hz, 1H), 1.83 (d, J=13.9 Hz, 1H), 1.41 (m, 1H); LCMS C₁₁H₁₃N₃O₂S method (B) Rₜ=5.782 min, ESI+ m/z=252.1 (M+H).

General Procedure for the Synthesis of N-((3-nitroaryl)carbamothioyl)acetamide Derivatives. Under argon, to a suspension of potassium thiocyanate (982 mg, 10 mmol) in acetone (30 mL) was added dropwise acetyl chloride (0.60 mL, 7.95 mmol). The resulting solution was refluxed for 3 hrs. After to cooling to room temperature, a solution of appropriate 3-nitroaniline (7.68 mmol) in acetone (20 mL) was added dropwise. The mixture was stirred overnight at room temperature, upon complete consumption of the starting material monitored by TLC or LCMS. The solvent was distillated off under reduced pressure and the residue was poured onto water (100 mL) and stirred for 1 hr. The resulting precipitate was collected by filtration, to afford corresponding N-((3-nitroaryl)carbamothioyl)acetamide.

N-((2-Methyl-5-nitrophenyl)carbamothioyl)acetamide dCKi-1-5 (99%) as a dark orange powder. Rf=(PE-EtOAc, 85:15); ¹H NMR (300 MHz, DMSO-d₆) δ12.37 (s, 1H), 11.67 (s, 1H), 8.67 (d, J=2.4 Hz, 1H), 8.06 (dd, J=8.4, 2.4 Hz, 1H), 7.58 (d, J=8.5 Hz, 1H), 2.33 (s, 3H), 2.18 (s, 3H); LCMS C₁₀H₁₁N₃O₃S method (B) Rₜ=6.036 min, ESI+ m/z=254.1 (M+H).

N-(8-Nitro-2,3,4,5-tetrahydro-1H-benzo[b]azepine-1-carbonothioyl)acetamide OR0289-5 (93%) as a light brown solid. LCMS C₁₃H₁₅N₃O₃S method (B) Rₜ=6.061 min, ESI+ m/z=294.1 (M+H).

8-Nitro-2,3,4,5-tetrahydro-1H-benzo[b]azepine OR0289-6

At −5° C., under stirring, to concentrated sulfuric acid (30 mL) was added by solid fraction over 30 min, a mixture of 2,3,4,5-tetrahydro-1H-benzo[b]azepine (2.94 g, 20 mmol) and potassium nitrate (2.02 g, 20 mmol). After addition, the reaction mixture was poured onto ice (100 g), then Na₂CO₃ was added until pH=8-9, and extracted with DCM (3×20 mL). The combined organic layers were dried over Na₂SO₄ and the solvent was distillated off under reduced pressure. The residue was purified by flash chromatography, DCM-PE (8:2) to afford 8-nitro-2,3,4,5-tetrahydro-1H-benzo[b]azepine OR0289-6 (2.70 g, 71%) as an orange oil which crystallized from a mixture of MeOH-Et₂O-PE as orange plates. Rf=0.35 (DCM-PE, 8:2); ¹H NMR (400 MHz, CDCl₃) δ 7.67 (dd, J=8.2, 2.3 Hz, 1H), 7.64 (d, J=2.3 Hz, 1H), 7.22 (d, J=8.2 Hz, 1H), 3.15-3.08 (m, 2H), 2.91-2.82 (m, 2H), 1.89-1.83 (m, 2H), 1.74-1.62 (m, 2H); LCMS C₁₀H₁₂N₂O₂ method (B) Rₜ=6.522 min, ESI+ m/z=193.1 (M+H).

General Procedure for the Synthesis of di-tert-butyl (2-(2-bromoacetyl)-pyrimidin-4-yl)carbamate Derivatives. To a stirred solution of appropriate di-tert-butyl (2-(1-ethoxyvinyl)pyrimidin-4-yl)carbamate (4 mmol) in a mixture of tetrahydrofuran-water (3:1, 8 mL) was added in one portion N-bromosuccinimide (844 mg, 4.74 mmol). The resulting solution was stirred for 3 hrs at room temperature, upon complete consumption of the starting material. The reaction mixture was concentrated under reduced pressure without heating (T≤30° C.). The crude product was crystallized from Et₂O to afford corresponding di-tert-butyl (2-(2-bromoacetyl)-pyrimidin-4-yl)carbamate.

Di-tert-butyl (2-(2-bromoacetyl)-pyrimidin-4-yl)carbamate dCKi-1-6 (95%) as a white crystals. Rf=0.35 (PE-EtOAc, 8:2); ¹H NMR (400 MHz, CDCl₃) δ 8.78 (d, J=5.8 Hz, 1H), 7.93 (d, J=5.8 Hz, 1H), 4.68 (s, 2H), 1.57 (s, 18H); LCMS C₁₆H₂₂BrN₃O₅ method (B) Rₜ=7.009 min, ESI+ m/z=418.2 (M+H).

Di-tert-butyl (2-(2-bromoacetyl)-5-fluoropyrimidin-4-yl)carbamate OR0239-4 (quantitative) as a white crystals engaged in the next step without further purification. LCMS C₁₆H₂₁BrFN₃O₅ method (A) Rₜ=7.256 min, ESI+ m/z=234.1 (M-(Boc)₂).

General Procedure for the Synthesis of di-tert-butyl (2-(1-ethoxyvinyl)pyrimidin-4-yl)carbamate Derivatives. Under argon, to a solution of appropriate di-tert-butyl (2-chloropyrimidin-4-yl)carbamate (5.0 mmol) in dry 1,4-dioxane or toluene (50 mL) were added successively tributyl(1-ethoxyvinyl)tin (2.52 g, 7.0 mmol) and cesium fluoride (1.51 g, 10.0 mmol). The resulting mixture was thoroughly degassed several times under argon fillings before addition of bis(triphenylphosphine)palladium dichloride (351 mg, 0.5 mmol, 10 mol %). The resulting mixture was warmed until complete consumption of starting material. After cooling to room temperature the resulting mixture was filtrated through a short pad of Celite, rinsing with EtOAc (50 mL). The filtrate was concentrated under reduced pressure and the residue was purified by flash chromatography to afford corresponding di-tert-butyl (2-(1-ethoxyvinyl)pyrimidin-4-yl)carbamate.

Di-tert-butyl (2-(1-ethoxyvinyl)pyrimidin-4-yl)carbamate dCKi-1-7. Under argon, to a solution of di-tert-butyl (2-chloropyrimidin-4-yl)carbamate dCKi-1-8 (25.5 g, 77.4 mmol) in dry 1,4-dioxane (450 mL) were added successively tributyl(1-ethoxyvinyl)tin (38.4 g, 106.2 mmol) and cesium fluoride (23.3 g, 153.0 mmol). The resulting mixture was thoroughly degassed several times under argon fillings before addition of bis(triphenylphosphine)palladium dichloride (5.5 g, 7.75 mmol, 10 mol %). The resulting mixture was stirred at 90° C. overnight, and then allowed to cool to room temperature before filtration through a pad of Celite, washing with EtOAc (800 mL). The filtrate was concentrated under reduced pressure and the residue was purified by flash chromatography, PE-EtOAc (8:2) to afford di-tert-butyl (2-(1-ethoxyvinyl)pyrimidin-4-yl)carbamate dCKi-1-7 (20.7 g, 73%) as yellow crystals. Rf=0.35 (PE-EtOAc, 8:2); ¹H NMR (400 MHz, CDCl₃) δ 8.66 (d, J=5.7 Hz, 1H), 7.56 (d, J=5.7 Hz, 1H), 5.58 (d, J=1.9 Hz, 1H), 4.57 (d, J=1.9 Hz, 1H), 3.99 (q, J=7.0 Hz, 2H), 1.55 (s, 18H), 1.46 (t, J=7.0 Hz, 3H); LCMS C₁₈H₂₇N₃O₅ method (A) R_{c}=7.608 min, ESI+ m/z=366.3 (M+H).

Di-tert-butyl (2-(1-ethoxyvinyl)-5-fluoropyrimidin-4-yl)carbamate OR0239-5. A stirring solution of di-tert-butyl (2-chloro-5-fluoropyrimidin-4-yl)carbamate OR0239-6 (400 mg, 1.15 mmol), tributyl(1-ethoxyvinyl)tin (550 μL, 1.61 mmol), copper(I) bromide (25 mg, 0.17 mmol) and cesium fluoride (350 mg, 2.3 mmol) in dry toluene (10 mL) was degassed under argon fillings for 15 min. PdCl₂(PPh₃)₂ (41 mg, 0.06 mmol) and triphenylphosphine (45 mg, 0.17 mmol) were successively added. Argon was bubbled through for another 5 min and the mixture was refluxed during 4 hrs upon complete conversion monitored by TLC. The resulting mixture was allowed to cool to room temperature, filtered through a pad of celite and rinsed with DCM. The crude product was purified by flash chromatography, DCM-EtOAc to afford di-tert-butyl (2-(1-ethoxyvinyl)-5-fluoropyrimidin-4-yl)carbamate OR0239-5 as a colorless oil (401 mg, 91%). ¹H NMR (400 MHz, CDCl₃) δ 8.62 (d, J=1.4 Hz, 1H), 5.60 (d, J=2.2 Hz, 1H), 4.62 (d, J=2.2 Hz, 1H), 4.02 (q, J=7.0 Hz, 2H), 1.48 (t, J=7.0 Hz, 3H), 1.44 (s, 18H); LCMS C₁₈H₂₆FN₃O₅ method (A) Rₜ=7.568 min, ESI+ m/z=384.2 (M+H).

General Procedure for the Synthesis of di-tert-butyl (2-chloropyrimidin-4-yl)carbamate Derivatives. To a stirred solution of appropriate 2-chloro-4-aminopyrimidine (1.95 g, 15 mmol) in tetrahydrofuran (50 mL), was added di-tert-butyl dicarbonate (8.0 g, 37 mmol) and 4-dimethylaminopyridine (124 mg, 1 mmol). The resulting solution was stirred at room temperature overnight upon completion of the reaction. The solvent was distillated off under reduced pressure and the residue was diluted with DCM (50 mL) and washed with H₂O (2×10 mL). The organic layer was dried over Na₂SO₄ and concentrated under reduced pressure to afford corresponding di-tert-butyl (2-chloropyrimidin-4-yl)carbamate.

Di-tert-butyl (2-chloropyrimidin-4-yl)carbamate dCKi-1-8 (quantitative) as a white solid. Rf=0.70 (PE-EtOAc, 85:15). ¹H NMR (300 MHz, CDCl₃) δ 8.46 (d, J=5.7 Hz, 1H), 7.71 (d, J=5.7 Hz, 1H), 1.56 (s, 18H); LCMS C₁₄H₂₀ClN₃O₄ method (B) Rₜ=7.407 min, ESI+ m/z=330.2 (M+H).

Di-tert-butyl (2-chloro-5-fluoropyrimidin-4-yl)carbamate OR0239-6 (28%) as a white powder. ¹H NMR (400 MHz, CDCl₃) δ 8.50 (s, 1H), 1.46 (s, 18H); LCMS C₁₄H₁₉ClFN₃O₄ method (A) Rₜ=7.759 min, ESI+ m/z=192.1 (M-(Boc)₂+HCO₂H).

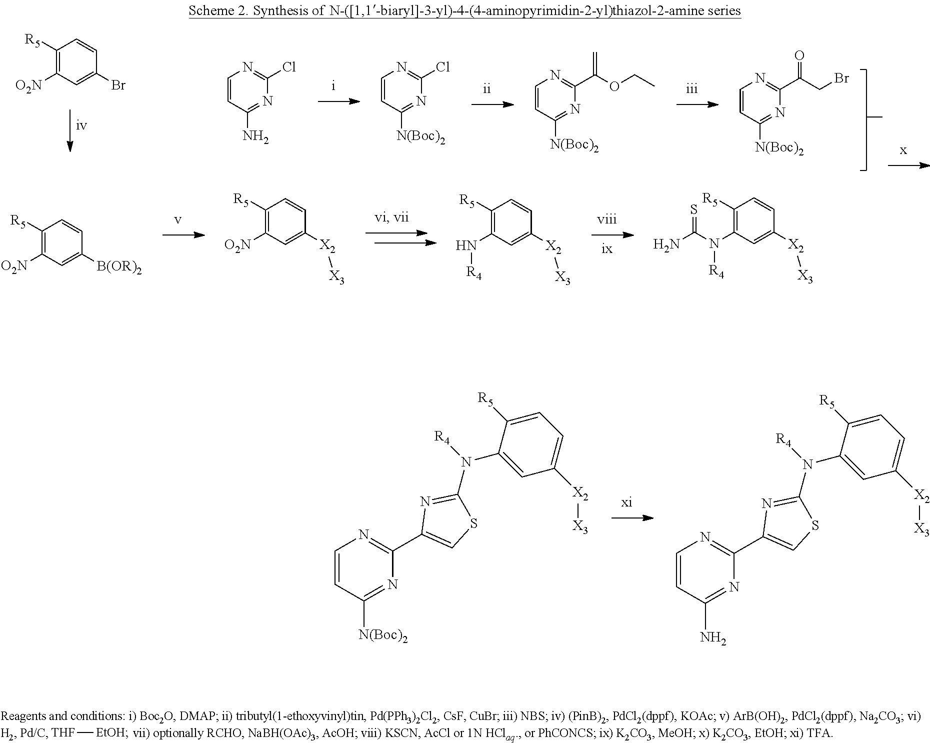

N-([1,1′-biaryl]-3-yl)-4-(4-aminopyrimidin-2-yl)thiazol-2-amine derivatives were prepared by convergent synthesis, in eight steps, from commercially available 4-amino-2-chloropyrimidine and appropriate bromo-3-nitrobenzene (Scheme 2). The 4-amino-2-chloropyrimidine was protected using Boc₂O to afford corresponding bis-carbamate. A Stille cross-coupling reaction with tributyl(1-ethoxyvinyl)tin gave the enol ether which was then turned into the corresponding α-bromoketone using N-bromosuccinimide. Starting from appropriate bromo-3-nitrobenzene, a Suzuki cross-coupling reaction with corresponding aryl boronate allowed introducing the key biaryl scaffold. Hydrogenation of the nitro group followed by condensation with potassium thiocyanate, after optional (C₁-C₆)alkylation, gave the corresponding thiourea. As previously described, the α-bromoketone was engaged in the Hantzsch thiazole synthesis with the thiourea leading to the corresponding thiazole. Finally, deprotection with TFA led to expected N-([1,1′-biaryl]-3-yl)-4-(4-aminopyrimidin-2-yl)thiazol-2-amines.

General Procedure for the Synthesis of N-([1,1′-biaryl]-3-yl)-4-(4-aminopyrimidin-2-yl)thiazol-2-amine. As previously described for N-(3-((4-(4-aminopyrimidin-2-yl)thiazol-2-yl)amino)-4-aryl)benzamide derivatives.

4-(4-Aminopyrimidin-2-yl)-N-(4-methyl-4′-((4-methylpiperazin-1-yl)methyl)-[1,1′-biphenyl]-3-yl)thiazol-2-amine OR0274 (57%) as a white powder. ¹H NMR (400 MHz, CDCl₃) δ 8.31 (d, J=5.7 Hz, 1H), 7.76 (s, 1H), 7.59 (s, 1H), 7.55 (d, J=8.1 Hz, 2H), 7.39 (d, J=8.1 Hz, 2H), 7.32-7.27 (m, 2H), 6.34 (d, J=5.7 Hz, 1H), 4.98 (brs, 2H), 3.55 (s, 2H), 2.50 (brs, 8H), 2.34 (s, 3H), 2.30 (s, 3H); ¹³C NMR (100 MHz, CDCl₃) δ 166.65, 163.11, 160.54, 156.49, 150.57, 140.32, 139.31, 139.17, 137.56, 131.73, 129.81, 128.70, 126.88, 123.25, 118.96, 110.94, 103.41, 62.75, 55.20, 53.11, 46.07, 17.69. LC method (D) Rₜ=3.558 min.

4-(4-Aminopyrimidin-2-yl)-N-(4-methyl-4′-(2-(4-methylpiperazin-1-yl)ethyl)-[1,1′-biphenyl]-3-yl)thiazol-2-amine OR0325 (45%) as a white powder. ¹H NMR (400 MHz, CDCl₃) δ 8.30 (d, J=5.7 Hz, 1H), 7.74 (s, 1H), 7.58 (s, 1H), 7.52 (d, J=8.2 Hz, 2H), 7.35 (brs, 1H), 7.29-7.27 (m, 4H), 6.33 (d, J=5.7 Hz, 1H), 4.99 (s, 2H), 2.85 (dd, J=9.9, 6.4 Hz, 1H), 2.65 (dd, J=9.9, 6.4 Hz, 1H), 2.52 (brs, 8H), 2.33 (s, 3H), 2.32 (s, 3H). ¹³C NMR (100 MHz, CDCl₃) δ 166.66, 163.10, 160.53, 156.48, 150.57, 140.34, 139.73, 139.16, 138.35, 131.71, 129.32, 128.62, 127.05, 123.19, 118.92, 110.92, 103.40, 60.47, 55.19, 53.20, 46.11, 33.31, 17.67; LC method (D) Rₜ=3.602 min. 3′-((4-(4-Aminopyrimidin-2-yl)thiazol-2-yl)amino)-4′-methyl-[1,1′-biphenyl]-4-ol OR0320 (58%) as a white powder. ¹H NMR (400 MHz, MeOD) δ 8.12 (d, J=6.0 Hz, 1H), 7.76 (d, J=1.6 Hz, 1H), 7.49 (s, 1H), 7.46 (d, J=8.6 Hz, 2H), 7.32 (dd, J=8.1, 1.6 Hz, 1H), 7.29 (d, J=8.1 Hz, 1H). 6.84 (d, J=8.6 Hz, 2H), 6.43 (d, J=6.0 Hz, 1H), 2.34 (s, 3H). ¹³C NMR (100 MHz, MeOD) δ 169.65, 165.60, 160.77, 158.24, 154.87, 150.72, 141.44, 140.88, 133.12, 132.59, 130.83, 128.88, 124.35, 122.07, 116.64, 112.10, 104.58, 17.61; LC method (D) Rₜ=4.369 min.

3′-((4-(4-Aminopyrimidin-2-yl)thiazol-2-yl)amino)-4′-methyl-[1,1′-biphenyl]-4-ol OR0321 (53%) as a white powder. ¹H NMR (400 MHz, CDCl₃) δ 8.28 (s, 1H), 7.70 (s, 1H), 7.57 (s, 1H), 7.28-7.26 (m, 2H), 7.12-6.96 (m, 3H), 6.33 (d, J=5.5 Hz, 1H), 5.03 (brs, 2H), 3.94 (s, 3H), 2.32 (s, 3H); LC method (D) Rₜ=4.389 min.

4-(4-Aminopyrimidin-2-yl)-N-(4-methyl-4′-(3-(4-methylpiperazin-1-yl)propyl)-[1,1′-biphenyl]-3-yl)thiazol-2-amine OR0331 (46%) as a light yellow powder. ¹H NMR (400 MHz, CDCl₃) δ 8.26 (d, J=5.7 Hz, 1H), 7.74 (brs, 1H), 7.56 (s, 1H), 7.51-7.49 (m, 3H), 7.31-7.24 (m, 4H), 6.31 (d, J=5.7 Hz, 1H), 5.05 (brs, 2H), 2.66 (t, J=7.8 Hz, 2H), 2.47 (brs, 8H), 2.39 (t, J=7.8 Hz, 2H), 2.31 (s, 3H), 2.28 (s, 3H), 1.81 (quint, J=7.8 Hz, 2H); ¹³C NMR (100 MHz, CDCl₃) δ 166.67, 163.10, 160.55, 156.51, 150.56, 141.65, 140.45, 139.12, 138.04, 131.71, 129.02, 128.51, 126.97, 123.20, 118.89, 110.97, 103.40, 58.08, 55.26, 53.29, 46.16, 33.47, 28.71, 17.68; LCMS C₂₈H₃₃N₇S method (B) Rₜ=3.881 min, ESI+ m/z=500.1 (M+H).

4-(4-Aminopyrimidin-2-yl)-N-(4-methyl-4′-(3-(4-methylpiperazin-1-yl)ethyl)-[1,1′-biphenyl]-3-yl)-N-propylthiazol-2-amine OR0345 (42%), as a colorless solid; ¹H NMR (400 MHz, CDCl₃) δ 8.26 (d, J=5.7 Hz, 1H), 7.53-7.46 (m, 4H), 7.39-7.38 (m, 2H), 7.28-7.25 (m, 2H), 6.25 (d, J=5.7 Hz, 1H), 5.3 (brs, 2H), 4.01 (brs, 2H), 2.86-2.82 (m, 2H), 2.87-2.51 (m, 2H), 2.52 (brs, 8H), 2.31 (s, 3H), 2.26 (s, 3H), 1.66 (sext, J=7.3 Hz, 2H), 0.92 (t, J=7.3 Hz, 3H); ¹³C NMR (100 MHz, CDCl₃) δ 171.27, 163.14, 160.88, 156.41, 151.19, 143.27, 140.74, 139.81, 137.84, 136.17, 132.49, 129.33, 127.97, 126.96, 111.21, 103.18, 60.39, 55.13, 53.61, 53.11, 46.05, 33.25, 21.27, 17.45, 11.40; LCMS C₃₀H₃₇N₇S method (B) Rₜ=4.152 min, ESI+m/z=528.3 (M+H).

4-(4-Aminopyrimidin-2-yl)-N-(4-methyl-4′-(3-(4-methylpiperazin-1-yl)propyl)-[1,1′-biphenyl]-3-yl)-N-propylthiazol-2-amine OR0598 (70%), as a pale yellow solid. Rf=0.40 (DCM-MeOH—NH₄OH, 90:9:1); ¹H NMR (400 MHz, CDCl₃) δ 8.29 (d, J=5.8 Hz, 1H), 7.53 (dd, J=7.9, 1.7 Hz, 1H), 7.49 (d, J=8.1 Hz, 2H), 7.46 (d, J=1.7 Hz, 1H), 7.39 (d, J=7.6 Hz, 1H), 7.37 (s, 1H), 7.24 (d, J=8.1 Hz, 2H), 6.29 (d, J=5.8 Hz, 1H), 5.10 (s, 2H), 4.02 (brs, 2H), 2.72-2.65 (m, 2H), 3.11-2.55 (m, 8H), 2.54-2.45 (m, 2H), 2.43 (s, 3H), 2.27 (s, 3H), 1.89 (quint, J=7.6 Hz, 2H), 1.67 (sext, J=7.5 Hz, 2H), 0.93 (t, J=7.5 Hz, 3H). ¹³C NMR (100 MHz, CDCl₃) δ 171.41, 163.09, 160.94, 156.53, 151.17, 143.29, 141.14, 140.78, 137.78, 136.24, 132.54, 129.04, 128.02, 127.00, 111.31, 103.19, 57.48, 54.33, 53.53, 52.03, 45.29, 33.18, 28.07, 21.32, 17.47, 11.44; LCMS C₃₁H₃₉N₇S method (B) Rₜ=4.253 min, ESI+ m/z=542.3 (M+H).

2-(2-(8-(4-(2-(4-Methylpiperazin-1-yl)ethyl)phenyl)-2,3,4,5-tetrahydro-1H-benzo[b]azepin-1-yl)thiazol-4-yl)pyrimidin-4-amine OR0402 (87%), as a pale yellow solid. Rf=0.18 (DCM-MeOH—NH₄OH, 90:9:1); ¹H NMR (400 MHz, MeOD) δ 8.12 (d, J=6.0 Hz, 1H), 7.60 (d, J=1.8 Hz, 1H), 7.56-7.47 (m, 3H), 7.38 (d, J=7.9 Hz, 1H), 7.35 (s, 1H), 7.27 (d, J=8.2 Hz, 2H), 6.43 (d, J=6.0 Hz, 1H), 4.03 (brs, 2H), 2.85-2.79 (m, 2H), 2.78-2.72 (m, 2H), 2.66-2.58 (m, 2H), 2.55 (brs, 8H), 2.30 (s, 3H), 1.95-1.93 (m, 2H), 1.75-7.73 (m, 2H). ¹³C NMR (100 MHz, MeOD) δ 170.97, 165.49, 161.54, 155.76, 151.89, 146.59, 142.09, 141.04, 140.70, 139.12, 132.64, 130.35, 127.88, 127.59, 127.25, 111.86, 104.49, 61.16, 55.53, 53.55, 52.04, 45.89, 35.03, 33.63, 29.59, 27.58; LCMS C₃₀H₃₅N₇S method (B) Rₜ=4.064 min, ESI+m/z=526.3 (M+H).

4-(4-Aminopyrimidin-2-yl)-N-(4-methyl-4′-(2-(4-methylpiperazin-1-yl)ethoxy)-[1,1′-biphenyl]-3-yl)-N-propylthiazol-2-amine OR0596 (63%), as a white solid. ¹H NMR (400 MHz, CDCl₃) δ 8.29 (d, J=5.8 Hz, 1H), 7.50 (d, J=8.8 Hz, 2H), 7.49 (dd, J=7.7, 1.8 Hz, 1H), 7.43 (d, J=2.0 Hz, 1H), 7.38 (s, 1H), 7.37 (d, J=7.7 Hz, 1H), 6.97 (d, J=8.8 Hz, 2H), 6.27 (d, J=5.8 Hz, 1H), 5.12 (s, 2H), 4.14 (t, J=5.8 Hz, 2H), 4.02 (brs, 2H), 2.83 (t, J=5.8 Hz, 2H), 2.63 (brs, 4H), 2.48 (brs, 4H), 2.29 (s, 3H), 2.26 (s, 3H), 1.67 (sext, J=7.4 Hz, 2H), 0.93 (t, J=7.4 Hz, 3H). ¹³C NMR (100 MHz, CDCl₃) δ 171.39, 163.09, 160.98, 158.62, 156.58, 151.19, 143.28, 140.59, 135.71, 132.73, 132.49, 128.02, 127.70, 126.75, 115.12, 111.27, 103.16, 66.17, 57.28, 55.16, 53.73, 53.56, 46.16, 21.31, 17.44, 11.44; LCMS C₃₀H₃₇N₇S method (B) Rₜ=4.304 min, ESI+ m/z=544.2 (M+H).

4-(4-Aminopyrimidin-2-yl)-N-(4-methyl-4′-(2-(4-methylpiperazin-1-yl)-2-oxoethoxy)-[1,1′-biphenyl]-3-yl)-N-propylthiazol-2-amine OR0597 (49%), as a white solid. ¹H NMR (400 MHz, CDCl₃) δ 8.30 (d, J=5.8 Hz, 1H), 7.51 (d, J=8.8 Hz, 2H), 7.48 (dd, J=8.0, 1.9 Hz, 1H), 7.42 (d, J=1.9 Hz, 1H), 7.38 (s, 1H), 7.38 (d, J=8.0 Hz, 1H), 7.01 (d, J=8.8 Hz, 2H), 6.29 (d, J=5.8 Hz, 1H), 5.04 (s, 2H), 4.72 (s, 2H), 4.03 (brs, 2H), 3.63 (m, 4H), 2.40 (m, 4H), 2.29 (s, 3H), 2.27 (s, 3H), 1.67 (sext, J=7.4 Hz, 2H), 0.94 (t, J=7.4 Hz, 3H). ¹³C NMR (100 MHz, CDCl₃) δ 171.41, 166.40, 163.07, 161.00, 157.69, 156.63, 151.19, 143.30, 140.40, 135.97, 133.65, 132.55, 128.22, 127.81, 126.83, 115.16, 111.31, 103.16, 67.85, 55.27, 54.74, 53.51, 46.15, 45.39, 42.18, 21.33, 17.45, 11.45; LCMS C₃₀H₃₅N₇O₂S method (B) Rₜ=4.296 min, ESI+ m/z=558.2 (M+H).

4-(4-Aminopyrimidin-2-yl)-N-(2-methyl-5-(6-(2-(4-methylpiperazin-1-yl)ethyl)pyridin-3-yl)phenyl)-N-propylthiazol-2-amine OR0599 (10%) as a yellow solid. Rf=0.40 (DCM-MeOH—NH₄OH, 90:9:1); ¹H NMR (400 MHz, CDCl₃) δ 8.74 (s, 1H), 8.30 (d, J=5.3 Hz, 1H), 7.77 (d, J=7.9 Hz, 1H), 7.52 (d, J=7.9 Hz, 1H), 7.45 (s, 1H), 7.43 (d, J=7.9 Hz, 1H), 7.39 (s, 1H), 7.24 (d, J=7.9 Hz, 1H), 6.29 (d, J=5.3 Hz, 1H), 5.07 (s, 2H), 4.04 (brs, 2H), 3.03 (m, 2H), 2.80 (m, 2H), 2.60 (brs, 4H), 2.48 (brs, 4H), 2.29 (s, 6H), 1.66 (sext, J=7.2 Hz, 2H), 0.94 (t, J=7.2 Hz, 3H). ¹³C NMR (100 MHz, CDCl₃) δ 171.20, 163.08, 160.97, 159.62, 156.65, 151.28, 147.57, 143.60, 137.68, 137.31, 134.70, 133.15, 132.92, 128.14, 127.01, 123.28, 111.35, 103.20, 58.43, 55.24, 53.54, 53.16, 46.15, 35.53, 21.36, 17.56, 11.44; LCMS C₂₉H₃₆N₈S method (B) Rₜ=3.950 min, ESI+ m/z=529.4 (M+H).

General Procedure for the Synthesis of di-tert-butyl (2-(2-([1,1′-biaryl]-3-ylamino)thiazol-4-yl)pyrimidin-4-yl)carbamate Derivatives. As previously described for di-tert-butyl (2-(2-((3-nitroaryl)amino)thiazol-4-yl)pyrimidin-4-yl)carbamate derivatives.

Di-tert-butyl (2-(2-((4-methyl-4′-((4-methylpiperazin-1-yl)methyl)-[1,1′-biphenyl]-3-yl)amino)thiazol-4-yl)pyrimidin-4-yl)carbamate OR0274-1 (37%) as a light brown powder. 1H NMR (400 MHz, CDCl₃) δ 8.70 (d, J=5.7 Hz, 1H), 7.75 (s, 1H), 7.57-7.50 (m, 4H), 7.39 (d, J=8.1 Hz, 2H), 7.31 (s, 2H), 3.55 (s, 2H), 2.50 (brs, 8H), 2.34 (s, 3H), 2.30 (s, 3H), 1.56 (s, 18H).

Di-tert-butyl (2-(2-((4-methyl-4′-(2-(4-methylpiperazin-1-yl)ethyl)-[1,1′-biphenyl]-3-yl)amino)thiazol-4-yl)pyrimidin-4-yl)carbamate OR0325-1 (46%) as a light brown powder. ¹H NMR (400 MHz, CDCl₃) δ 8.70 (d, J=5.7 Hz, 1H), 7.73 (s, 1H), 7.53-7.51 (m, 4H), 7.33-7.26 (m, 4H), 2.85 (dd, J=9.9, 6.4 Hz, 2H), 2.65 (dd, J=9.9, 6.4 Hz, 1H), 2.53 (brs, 8H), 2.34 (s, 3H), 2.32 (s, 3H), 1.56 (s, 18H); LC method (C) Rₜ=3.933 min.

Di-tert-butyl (2-(2-((4′-hydroxy-4-methyl-[1,1′-biphenyl]-3-yl)amino)thiazol-4-yl)pyrimidin-4-yl)carbamate OR0320-1 (48%) as a light brown powder. ¹H NMR (400 MHz, CDCl₃) δ 8.68 (d, J=5.7 Hz, 1H), 7.66 (s, 1H), 7.53 (d, J=5.7 Hz, 1H), 7.50 (s, 1H), 7.46 (d, J=8.6 Hz, 2H), 7.27-7.25 (m, 1H), 6.92 (d, J=8.6 Hz, 2H), 2.30 (s, 3H), 1.56 (s, 18H); LC method (A) Rₜ=8.406 min.

Di-tert-butyl (2-(2-((4′-hydroxy-3′-methoxy-4-methyl-[1,1′-biphenyl]-3-yl)amino)thiazol-4-yl)pyrimidin-4-yl)carbamate OR0321-1 (52%) as a light brown powder. ¹H NMR (400 MHz, CDCl₃) δ 8.70 (d, J=5.6 Hz, 1H), 7.70 (s, 1H), 7.54 (d, J=5.6 Hz, 1H), 7.51 (s, 1H), 7.28-7.26 (m 2H), 7.13-7.05 (m, 2H), 6.99 (d, J=8.1 Hz, 1H), 5.65 (brs, 1H), 3.97 (s, 3H), 2.34 (s, 3H), 1.56 (s, 18H); LCMS C₃₁H₃₅N₅O₆S method (A) Rₜ=8.452 min, ESI+ m/z=606.3 (M+H).

Di-tert-butyl (2-(2-((4-methyl-4′-(2-(4-methylpiperazin-1-yl)propyl)-[1,1′-biphenyl]-3-yl)amino)thiazol-4-yl)pyrimidin-4-yl)carbamate OR0331-1 (58%) as a yellow powder. LCMS C₃₈H₄₉N₇O₄S method (B) Rₜ=5.470 min, ESI+ m/z=700.4 (M+H).

Di-tert-butyl (2-(2-((4-methyl-4′-(3-(4-methylpiperazin-1-yl)ethyl)-[1,1′-biphenyl]-3-yl)(propyl)amino)thiazol-4-yl)pyrimidin-4-yl)carbamate OR0345-1 (47%) as a yellow powder. Rf=0.42 (DCM-MeOH—NH₄OH, 90:9:1). ¹H NMR (400 MHz, CDCl₃) δ 8.73 (d, J=5.7 Hz, 1H), 7.55-7.46 (m, 4H), 7.41-7.39 (m, 2H), 7.31-7.24 (m, 3H), 5.3 (brs, 2H), 3.99 (brs, 2H), 2.86-2.50 (m, 12H), 2.28 (s, 3H), 2.17 (s, 3H), 1.69 (sext, J=7.4 Hz, 2H), 1.56 (s, 18H), 0.94 (t, J=7.4 Hz, 3H); LCMS C₄₀H₅₃N₇O₄S method (B) Rₜ=5.040 min, ESI+ m/z=728.4 (M+H).

Di-tert-butyl (2-(2-((4-methyl-4′-(3-(4-methylpiperazin-1-yl)propyl)-[1,1′-biphenyl]-3-yl)(propyl)amino)thiazol-4-yl)pyrimidin-4-yl)carbamate OR0598-1 (85%) as a light brown powder. Rf=0.42 (DCM-MeOH—NH₄OH., 90:9:1). ¹H NMR (400 MHz, CDCl₃) δ 8.73 (d, J=5.7 Hz, 1H), 7.54 (dd, J=7.9, 1.7 Hz, 1H), 7.50 (d, J=8.6 Hz, 2H), 7.46-7.48 (m, 2H), 7.40 (d, J=8.0 Hz, 1H), 7.37 (s, 1H), 7.24 (d, J=8.2 Hz, 2H), 4.03 (brs, 2H), 2.94 (s, 8H), 2.96-2.87 (m, 2H), 2.70 (t, J=7.4 Hz, 2H), 2.57 (s, 3H), 2.28 (s, 3H), 1.98-1.94 (m, 2H), 1.68 (sext, J=7.4 Hz, 2H), 1.54 (s, 18H), 0.94 (t, J=7.4 Hz, 3H); ¹³C NMR (100 MHz, CDCl₃) δ 171.50, 160.50, 158.62, 158.55, 150.61, 150.37, 143.29, 140.75, 137.98, 136.32, 132.62, 129.04, 128.10, 127.23, 127.10, 112.49, 110.62, 84.24, 57.02, 55.86, 53.56, 53.36, 51.06, 32.95, 28.00, 27.96, 21.32, 17.46, 11.48; LCMS C₄₁H₅₅N₇O₄S method (B) Rₜ=5.956 min, ESI+ m/z=742.4 (M+H).

Di-tert-butyl (2-(2-(8-(4-(2-(4-methylpiperazin-1-yl)ethyl)phenyl)-2,3,4,5-tetrahydro-1H-benzo[b]azepin-1-yl)thiazol-4-yl)pyrimidin-4-yl)carbamate OR0402-1 (60%) as a light brown powder. Rf=0.42 (DCM-MeOH—NH₄OH, 90:9:1); LCMS C₄₀H₅₁N₇O₄S method (B) Rₜ=5.603 min, ESI+ m/z=726.3 (M+H).

Di-tert-butyl (2-(2-((4-methyl-4′-(2-(4-methylpiperazin-1-yl)ethoxy)-[1,1′-biphenyl]-3-yl)(propyl)amino)thiazol-4-yl)pyrimidin-4-yl)carbamate OR0596-1 (94%) as a colorless oil. Rf=0.42 (DCM-MeOH—NH₄OH, 90:9:1); LCMS C₄₀H₅₃N₇O₅S method (B) Rₜ=5.949 min, ESI+ m/z=744.4 (M+H).

Di-tert-butyl (2-(2-((4-methyl-4′-(2-(4-methylpiperazin-1-yl)-2-oxoethoxy)-[1,1′-biphenyl]-3-yl)(propyl)amino)thiazol-4-yl)pyrimidin-4-yl)carbamate OR0597-1 (82%) as a light brown solid. Rf=0.42 (DCM-MeOH—NH₄OH, 90:9:1); LCMS C₄₀H₅₃N₇O₅S method (B) Rₜ=5.889 min, ESI+ m/z=758.2 (M+H).

Di-tert-butyl (2-(2-((2-methyl-5-(6-(2-(4-methylpiperazin-1-yl)ethyl)pyridin-3-yl)phenyl)(propyl)amino)thiazol-4-yl)pyrimidin-4-yl)carbamate OR0599-1 (67%) as a light brown powder. Rf=0.42 (DCM-MeOH—NH₄OH, 90:9:1); LCMS C₃₉H₅₂NSO₄S method (B) Rₜ=5.489 min, ESI+ m/z=729.6 (M+H).

General Procedure for the Synthesis of 1-([1,1′-biaryl]-3-yl)thiourea Derivatives. Method (A): as previously described for 1-(3-nitroaryl)thiourea derivatives. Method (B): to a solution of appropriate [1,1′-biaryl]-3-amine (0.70 mmol) in 1N aqueous hydrochloric acid solution (7 mL) was added potassium thiocyanate (312 mg, 3.2 mmol). The resulting solution was successively stirred at 90° C. for 48 hours, allowed to cool to room temperature, then saturated aqueous NaHCO₃ solution was added until pH=8, and extracted with EtOAc (3×50 mL). The combined organic layers were dried over Na₂SO₄, and the solvent was distillated off under reduced pressure. The residue was purified by chromatography to afford corresponding 1-([1,1′-biaryl]-3-yl)thiourea.

1-(4-Methyl-4′-((4-methylpiperazin-1-yl)methyl)-[1,1′-biphenyl]-3-yl)thiourea OR0274-2 (method A, 39%) as a white powder. ¹H NMR (400 MHz, MeOD) δ 7.59 (d, J=8.3 Hz, 2H), 7.52 (dd, J=7.9, 1.9 Hz, 1H), 7.49 (d, J=1.9 Hz, 1H), 7.40 (d, J=8.3 Hz, 2H), 7.37 (d, J=7.9 Hz, 1H), 4.58 (s, 2H), 2.54 (brs, 8H), 2.31 (s, 3H), 2.30 (s, 3H).

1-(4-Methyl-4′-(2-(4-methylpiperazin-1-yl)ethyl)-[1,1′-biphenyl]-3-yl)thiourea OR0325-2 (method A, 77%) as a white powder. ¹H NMR (400 MHz, MeOD) δ 7.59-7.46 (m, 4H), 7.40-7.35 (m, 1H), 7.30-7.27 (m, 2H), 2.88-2.82 (m, 2H), 2.66-2.62 (m, 2H), 2.56 (brs, 8H), 2.31 (s, 6H); LC method (D) Rₜ=3.256 min.

1-(4′-Hydroxy-4-methyl-[1,1′-biphenyl]-3-yl)thiourea OR0320-2 (method A, 86%) as a white powder. ¹H NMR (400 MHz, MeOD) δ 7.46-7.42 (m, 3H), 7.40 (d, J=1.4 Hz, 1H), 7.32 (d, J=8.0 Hz, 1H), 6.85 (d, J=8.7 Hz, 2H), 2.29 (s, 3H); LCMS C₁₄H₁₄N₂OS method (A) Rₜ=5.366 min, ESI− m/z=257.4 (M−H).

1-(4′-Hydroxy-3′-methoxy-4-methyl-[1,1′-biphenyl]-3-yl)thiourea OR0321-2 (method A, 74%) as a white powder. ¹H NMR (400 MHz, MeOD) δ 7.47 (dd, J=7.9, 1.9 Hz, 1H), 7.42 (d, J=1.9 Hz, 1H), 7.33 (d, J=7.9 Hz, 1H), 7.16 (d, J=2.1 Hz, 1H), 7.06 (dd, J=8.2, 2.1 Hz, 1H), 6.85 (d, J=8.2 Hz, 1H), 3.92 (s, 3H), 2.30 (s, 3H); LCMS C₁₅H₁₆N₂O₂S method (A) Rₜ=5.448 min, ESI+ m/z=289.1 (M+H).

1-(4-Methyl-4′-(2-(4-methylpiperazin-1-yl)propyl)-[1,1′-biphenyl]-3-yl)thiourea OR0331-2 (method B, 73%) as a pale yellow solid. LCMS C₂₂H₃₀N₄S method (B) Rₜ=7.139 min, ESI+m/z=383.3 (M+H).

1-(4-Methyl-4′-(2-(4-methylpiperazin-1-yl)ethyl)-[1,1′-biphenyl]-3-yl)-1-propylthiourea OR0345-2 (method B, quantitative) as a yellow powder. ¹H NMR (400 MHz, CDCl₃) δ 7.53 (dd, J=7.9, 1.9 Hz, 1H), 7.46 (d, J=8.2 Hz, 2H), 7.39 (d, J=7.9 Hz, 1H), 7.34 (d, J=1.9 Hz, 1H), 7.29 (d, J=8.2 Hz, 2H), 5.47 (brs, 2H), 4.46-4.42 (m, 1H), 3.86-3.79 (m, 1H), 2.87-2.82 (m, 2H), 2.65-2.59 (m, 2H), 2.51 (brs, 8H), 2.33 (s, 3H), 2.31 (s, 3H), 1.87-1.78 (m, 2H), 0.92 (t, J=7.4 Hz, 3H); LCMS C₂₄H₃₄N₄S method (C) Rₜ=3.455 min, ESI+ m/z=411.3 (M+H).

1-(4-Methyl-4′-(3-(4-methylpiperazin-1-yl)propyl)-[1,1′-biphenyl]-3-yl)-1-propylthiourea OR0598-2 (method A, 90%) as a pale yellow solid. Rf=0.36 (DCM-MeOH—NH₄OH, 90:9:1); ¹H NMR (400 MHz, CDCl₃) δ 7.53 (dd, J=7.9, 1.8 Hz, 1H), 7.46 (d, J=8.1 Hz, 2H), 7.38 (d, J=7.9 Hz, 1H), 7.34 (d, J=1.8 Hz, 1H), 7.26 (d, J=8.1 Hz, 2H), 5.51 (brs, 2H), 4.51-4.41 (m, 1H), 3.72-3.61 (m, 1H), 2.72-2.64 (m, 2H), 2.56 (brs, 8H), 2.46-2.40 (m, 2H), 2.33 (s, 3H), 2.26 (s, 3H), 1.87 (quint, J=7.7 Hz, 2H), 1.86-1.62 (m, 2H), 0.92 (t, J=7.4 Hz, 3H); LCMS C₂₅H₃₆N₄S method (B) Rₜ=4.417 min, ESI+ m/z=425.3 (M+H). 8-(4-(2-(4-Methylpiperazin-1-yl)ethyl)phenyl)-2,3,4,5-tetrahydro-1H-benzo[b]azepine-1-carbothioamide OR0402-2 (method A, 92%) used in the next step without neither work-up nor purification. LCMS C₂₄H₃₂N₄S method (B) Rₜ=3.405 min, ESI+ m/z=409.3 (M+H).

1-(4-Methyl-4′-(2-(4-methylpiperazin-1-yl)ethoxy)-[1,1′-biphenyl]-3-yl)-1-propylthiourea OR0596-2 (method A, 61%) as a white solid. Rf=0.36 (DCM-MeOH—NH₄OH, 90:9:1); ¹H NMR (400 MHz, CDCl₃) δ 7.50 (dd, J=8.0, 1.8 Hz, 1H), 7.46 (d, J=8.8 Hz, 2H), 7.36 (d, J=8.0 Hz, 1H), 7.31 (d, J=1.8 Hz, 1H), 6.98 (d, J=8.8 Hz, 2H), 5.49 (brs, 2H), 4.49-4.41 (m, 1H), 4.15 (t, J=5.8 Hz, 2H), 3.68-3.61 (m, 1H), 2.84 (t, J=5.8 Hz, 2H), 2.64 (brs, 4H), 2.49 (brs, 4H), 2.30 (s, 3H), 2.25 (s, 3H), 1.84-1.75 (m, 1H), 1.72-1.62 (m, 1H), 0.91 (t, J=7.4 Hz, 3H); LCMS C₂₄H₃₄N₄OS method (B) Rₜ=4.535 min, ESI+ m/z=427.3 (M+H).

1-(4-Methyl-4′-(2-(4-methylpiperazin-1-yl)-2-oxoethoxy)-[1,1′-biphenyl]-3-yl)-1-propylthiourea OR0597-2 (method A, 47%) as a white solid. ¹H NMR (400 MHz, CDCl₃) δ 7.49 (dd, J=8.0, 1.9 Hz, 2H), 7.47 (d, J=8.8 Hz, 2H), 7.39 (s, 1H), 7.37 (d, J=8.0 Hz, 1H), 7.30 (d, J=1.9 Hz, 1H), 7.02 (d, J=8.8 Hz, 2H), 5.50 (brs, 2H), 4.73 (s, 2H), 3.67-3.56 (m, 4H), 2.44-2.36 (m, 4H), 2.29 (s, 3H), 2.25 (s, 3H), 1.86-1.74 (m, 1H), 1.74-1.62 (m, 1H), 0.91 (t, J=7.4 Hz, 3H); LCMS C₂₄H₃₂N₄O₂S method (B) Rₜ=4.498 min, ESI+ m/z=441.3 (M+H). 1-(2-Methyl-5-(6-(2-(4-methylpiperazin-1-yl)ethyl) pyridin-3-yl)phenyl)-1-propylthiourea OR0599-2 (method A, 81%) as a white solid. ¹H NMR (400 MHz, CDCl₃) δ 8.69 (d, J=2.0 Hz, 1H), 7.74 (dd, J=8.0, 2.0 Hz, 1H), 7.52 (dd, J=8.0, 2.0 Hz, 1H), 7.43 (d, J=8.0 Hz, 1H), 7.32 (d, J=2.0 Hz, 1H), 7.27 (d, J=8.0 Hz, 1H), 5.53 (brs, 2H), 4.48-4.41 (m, 1H), 3.69-3.61 (m, 1H), 3.05-3.01 (m, 2H), 2.81-2.77 (m, 2H), 2.59 (brs, 4H), 2.48 (brs, 4H), 2.29 (s, 3H), 2.28 (s, 3H), 1.82-1.63 (m, 2H), 0.91 (t, J=7.4 Hz, 3H); LCMS C₂₃H₃₃N₅S method (B) Rₜ=3.891 min, ESI+ m/z=412.3 (M+H).

General Procedure for the Synthesis of N-([1,1′-biaryl]-3-ylcarbamothioyl)acetamide Derivatives. As previously described for N-((3-nitroaryl)carbamothioyl)acetamide derivatives.

N-((4-Methyl-4′-((4-methylpiperazin-1-yl)methyl)-[1,1′-biphenyl]-3-yl)carbamothioyl)acetamide OR0274-3 (method A, 67%) as a light brown solid, engaged in the next step without further purification.

N-((4-Methyl-4′-(2-(4-methylpiperazin-1-yl)ethyl)-[1,1′-biphenyl]-3-yl)carbamothioyl)acetamide OR0325-3 (method A, 83%) as a light brown solid used in the next step without further purification.

N-((4′-Hydroxy-4-methyl-[1,1′-biphenyl]-3-yl)carbamothioyl)acetamide OR0320-3 (72%) as a white solid. ¹H NMR (400 MHz, MeOD) δ 7.88 (d, J=1.9 Hz, 1H), 7.45 (d, J=8.7 Hz, 2H), 7.39 (dd, J=7.9, 1.9 Hz, 1H), 7.28 (d, J=7.9 Hz, 1H), 6.83 (d, J=8.7 Hz, 2H), 2.27 (s, 3H), 2.18 (s, 3H); LCMS C₁₆H₁₆N₂O₂S method (A) Rₜ=6.114 min, ESI+ m/z=301.0 (M+H).

N-((4′-Hydroxy-3′-methoxy-4-methyl-[1,1′-biphenyl]-3-yl)carbamothioyl)acetamide OR0321-3 (method A, 88%), as a light brown solid. ¹H NMR (400 MHz, MeOD) δ 7.90 (d, J=1.8 Hz, 1H), 7.41 (dd, J=7.9, 1.8 Hz, 1H), 7.29 (d, J=7.9 Hz, 1H), 7.16 (d, J=2.0 Hz, 1H), 7.06 (dd, J=8.2, 2.0 Hz, 1H), 6.85 (d, J=8.2 Hz, 1H), 3.91 (s, 3H), 2.28 (s, 3H), 2.19 (s, 3H); LCMS C₁₇H₁₈N₂O₃S method (A) Rₜ=6.215 min, ESI+ m/z=331.1 (M+H).

General Procedure for the Synthesis of N-([1,1′-biaryl]-3-ylcarbamothioyl)benzamide derivatives. Under argon, at 0° C., to a solution of appropriate [1,1′-biaryl]-3-N-alkylamine (1.0 mmol) in dry acetone (25 mL) was added dropwise benzoyl isothiocyanate (1.1 mmol). The resulting solution was stirred at 0° C. for additional 1 hour and then allowed to warm to room temperature. After stirring overnight, upon complete consumption of the starting material monitored by TLC and LCMS, the solvent was distillated off under reduced pressure. The crude residue of corresponding N-([1,1′-biaryl]-3-ylcarbamothioyl)benzamide was used in the next step without further purification.

N-((4-Methyl-4′-(3-(4-methylpiperazin-1-yl)propyl)-[1,1′-biphenyl]-3-yl)(propyl)carbamothioyl)benzamide OR0598-3 (quantitative) as a pale yellow solid. Rf=0.40 (DCM-MeOH—NH₄OH, 90:9:1); ¹H NMR (400 MHz, CDCl₃) δ 7.57-7.20 (m, 12H), 4.48-4.42 (m, 1H), 3.86-3.80 (m, 1H), 2.72-2.64 (m, 2H), 2.58 (brs, 8H), 2.48-2.40 (m, 2H), 2.35 (s, 3H), 2.33 (s, 3H), 1.91-1.71 (m, 4H), 0.95 (t, J=7.4 Hz, 3H); LCMS C₃₂H₄₀N₄OS method (B) Rₜ=4.647 min, ESI+ m/z=529.3 (M+H).

N-(8-(4-(2-(4-Methylpiperazin-1-yl)ethyl)phenyl)-2,3,4,5-tetrahydro-1H-benzo[b]azepine-1-carbonothioyl)benzamide OR0402-3 (96%) as a pale yellow solid, used in the next step without further purification; LCMS C₃₁H₃₆N₄OS method (B) Rₜ=3.787 min, ESI+ m/z=513.3 (M+H).

N-((4-Methyl-4′-(2-(4-methylpiperazin-1-yl)ethoxy)-[1,1′-biphenyl]-3-yl)(propyl)carbamothioyl)benzamide OR0596-3 (99%) as a pale yellow solid. Rf=0.36 (DCM-MeOH—NH₄OH, 90:9:1); LCMS C₃₁H₃₅N₄O₂S method (B) Rₜ=4.982 min, ESI+ m/z=531.3 (M+H).

N-((4-Methyl-4′-(2-(4-methylpiperazin-1-yl)-2-oxoethoxy)-[1,1′-biphenyl]-3-yl)(propyl)carbamothioyl)benzamide OR0597-3 (quantitative) as a white solid. Rf=0.42 (DCM-MeOH—NH₄OH, 90:9:1); LCMS C₃₁H₃₆N₄O₃S method (B)_{R}=4.907 min, ESI+ m/z=545.3 (M+H).

N-((2-Methyl-5-(6-(2-(4-methylpiperazin-1-yl)ethyl)pyridin-3-yl)phenyl)(propyl)carbamothioyl)benzamide OR0599-3 (quantitative) as a white solid was used in the next step without further purification. Rf=0.40 (DCM-MeOH—NH₄OH, 90:9:1); LCMS C₃₀H₃₇N₅OS method (B) Rₜ=4.461 min, ESI+ m/z=516.3 (M+H).

General Procedure for the Synthesis of [1,1′-biaryl]-3-N-alkylamine Derivatives. To a stirred solution of appropriate [1,1′-biaryl]-3-amine (1.0 mmol) and aldehyde (1.5 mmol) in anhydrous tetrahydrofuran (8 mL) was added dropwise glacial acetic acid (1.5 mmol). After stirring for 2 hours, sodium triacetoxyborohydride (2.7 mmol) was added portionwise. The resulting suspension was stirred at room temperature, upon complete consumption of starting material monitored by TLC and LCMS. The reaction was cautiously quenched with a saturated aqueous solution of Na₂CO₃ (20 mL) and extracted with DCM (3×50 mL). The combined organic layers were dried over Na₂SO₄ and the solvent was distillated off under reduced pressure. The residue was purified by flash chromatography to afford corresponding [1,1′-biaryl]-3-N-alkylamine.

4-Methyl-4′-(2-(4-methylpiperazin-1-yl)ethyl)-N-propyl-[1,1′-biphenyl]-3-amine OR0345-3 (71%) as a yellow oil. ¹H NMR (400 MHz, CDCl₃) δ 7.51 (d, J=8.2 Hz, 2H), 7.25 (d, J=8.2 Hz, 2H), 7.10 (d, J=7.6 Hz, 1H), 6.85 (dd, J=7.6, 1.7 Hz, 1H), 6.79 (d, J=1.7 Hz, 1H), 3.19 (t, J=7.1 Hz, 2H), 2.89-2.81 (m, 2H), 2.69-2.62 (m, 2H), 2.51 (brs, 8H), 2.32 (s, 3H), 2.17 (s, 3H), 1.73 (sext, J=7.4 Hz, 2H), 1.03 (t, J=7.4 Hz, 3H); LCMS C₂₃H₃₃N₃ method (B) Rₜ=4.429 min, ESI+ m/z=352.3 (M+H).

4-Methyl-4′-(3-(4-methylpiperazin-1-yl)propyl)-N-propyl-[1,1′-biphenyl]-3-amine OR0598-4 (64%) as a yellow foam. Rf=0.38 (DCM-MeOH—NH₄OH, 90:9:1); ¹H NMR (400 MHz, CDCl₃) δ 7.50 (d, J=8.2 Hz, 2H), 7.23 (d, J=8.2 Hz, 2H), 7.10 (d, J=7.7 Hz, 1H), 6.86 (dd, J=7.7, 1.7 Hz, 1H), 6.80 (d, J=1.7 Hz, 1H), 3.52 (brs, 1H), 3.20 (t, J=7.1 Hz, 2H), 2.69-2.65 (m, 2H), 2.49 (brs, 8H), 2.43-2.39 (m, 2H), 2.31 (s, 3H), 2.17 (s, 3H), 1.86 (quint, J=7.7 Hz, 2H), 1.72 (sext, J=7.4 Hz, 2H), 1.04 (t, J=7.4 Hz, 3H). ¹³C NMR (100 MHz, CDCl₃) δ 146.57, 140.76, 140.30, 139.76, 130.32, 128.62, 127.06, 120.70, 115.46, 108.44, 58.01, 55.10, 53.09, 46.00, 45.81, 33.37, 28.61, 22.79, 17.15, 11.75; LCMS C₂₄H₃₅N₃ method (B) Rₜ=4.475 min, ESI+ m/z=366.3 (M+H).

4-Methyl-4′-(2-(4-methylpiperazin-1-yl)ethoxy)-N-propyl-[1,1′-biphenyl]-3-amine OR0596-4 (63%) as a pale yellow solid. Rf=0.36 (DCM-MeOH—NH₄OH, 90:9:1); ¹H NMR (400 MHz, CDCl₃) δ 7.50 (d, J=8.8 Hz, 2H), 7.08 (d, J=7.8 Hz, 1H), 6.95 (d, J=8.8 Hz, 2H), 6.82 (dd, J=7.8, 1.8 Hz, 1H), 6.77 (d, J=1.8 Hz, 1H), 4.15 (t, J=5.9 Hz, 2H), 3.52 (brs, 1H), 3.19 (t, J=7.1 Hz, 2H), 2.84 (t, J=5.9 Hz, 2H), 2.64 (brs, 4H), 2.50 (brs, 4H), 2.30 (s, 3H), 2.16 (s, 3H), 1.72 (sext, J=7.4 Hz, 2H), 1.04 (t, J=7.4 Hz, 3H); LCMS C₂₃H₃₃N₃O method (B) Rₜ=4.536 min, ESI+ m/z=368.3 (M+H).

2-((4′-Methyl-3′-(propylamino)-[1,1′-biphenyl]-4-yl)oxy)-1-(4-methylpiperazin-1-yl)ethan-1-one OR0597-4 (82%) as a colorless oil. ¹H NMR (400 MHz, CDCl₃) δ 7.52 (d, J=8.8 Hz, 2H), 7.09 (d, J=7.6 Hz, 1H), 6.99 (d, J=8.8 Hz, 2H), 6.81 (dd, J=7.6, 1.7 Hz, 1H), 6.76 (d, J=1.7 Hz, 1H), 4.72 (s, 2H), 3.71-3.59 (m, 4H), 3.19 (t, J=7.4 Hz, 2H), 2.46-2.35 (m, 4H), 2.30 (s, 3H), 2.16 (s, 3H), 1.72 (sext, J=7.4 Hz, 2H), 1.04 (t, J=7.4 Hz, 3H); LCMS C₂₃H₃₁N₃O₂ method (B) Rₜ=4.433 min, ESI+ m/z=382.3 (M+H).

2-Methyl-5-(6-(2-(4-methylpiperazin-1-yl)ethyl)pyridin-3-yl)-N-propylaniline OR0599-4 (71%) as a colorless oil. Rf=0.38 (DCM-MeOH—NH₄OH, 90:9:1); ¹H NMR (400 MHz, CDCl₃) δ 8.74 (d, J=1.8 Hz, 1H), 7.77 (dd, J=8.0, 2.4 Hz, 1H), 7.22 (d, J=8.0 Hz, 1H), 7.12 (d, J=7.7 Hz, 1H), 6.82 (dd, J=7.7, 1.8 Hz, 1H), 6.75 (d, J=1.8 Hz, 1H), 3.58 (brs, 1H), 3.18-3.17 (m, 2H), 3.02-3.00 (m, 2H), 2.83-2.80 (m, 2H), 2.61 (brs, 4H), 2.49 (brs, 4H), 2.30 (s, 3H), 2.17 (s, 3H), 1.73 (sext, J=7.4 Hz, 2H), 1.03 (t, J=7.4 Hz, 3H); LCMS C₂₂H₃₂N₄ method (B) Rₜ=3.941 min, ESI+ m/z=353.3 (M+H).

General Procedure for the Synthesis of [1,1′-biaryl]-3-amine Derivatives. Under hydrogen atmosphere, a solution of appropriate 3-nitro-1,1′-biaryl (1 mmol) and 10% palladium on charcoal (10% w/w) in a mixture of tetrahydrofuran-ethanol (1:1, 20 mL) was stirred at room temperature until the reaction was complete as indicated by TLC monitoring. The reaction mixture was filtrated through a short pad of celite and rinsed with EtOH. The solvent was distillated off under reduced pressure and the residue was purified by flash chromatography to afford the corresponding [1,1′-biaryl]-3-amine.

4-Methyl-4′-((4-methylpiperazin-1-yl)methyl)-[1,1′-biphenyl]-3-amine OR0274-4 (52%) as a yellow oil. ¹H NMR (400 MHz, CDCl₃) δ 7.49 (d, J=8.2 Hz, 2H), 7.35 (d, J=8.2 Hz, 2H), 7.11 (d, J=7.7 Hz, 1H), 6.94 (dd, J=7.7, 1.8 Hz, 1H), 6.90 (d, J=1.7 Hz, 1H), 6.90 (d, J=1.7 Hz, 1H), 3.67 (brs, 2H), 3.54 (s, 2H), 2.49 (s, 8H), 2.30 (s, 3H), 2.21 (s, 3H).

4-Methyl-4′-(2-(4-methylpiperazin-1-yl)ethyl)-[1,1′-biphenyl]-3-amine OR0325-4 (95%) as a white powder. ¹H NMR (400 MHz, CDCl₃) δ 7.47 (d, J=8.2 Hz, 2H), 7.24 (d, J=8.2 Hz, 2H), 7.10 (d, J=7.7 Hz, 1H), 6.92 (dd, J=7.7, 1.8 Hz, 1H), 6.89 (d, J=1.8 Hz, 1H), 3.67 (brs, 2H), 2.88-2.80 (m, 2H), 2.68-2.60 (m, 2H), 2.51 (brs, 8H), 2.31 (s, 3H), 2.20 (s, 3H); LC method (C) Rₜ=2.077 min.

3′-Amino-4′-methyl-[1,1′-biphenyl]-4-oI OR0320-4 (quantitative) as a yellow oil, without further purification. ¹H NMR (400 MHz, MeOD) δ 7.38 (d, J=8.7 Hz, 2H), 7.00 (d, J=7.7 Hz, 1H), 6.93 (d, J=1.7 Hz, 1H), 6.82 (dd, J=7.7, 1.7 Hz, 1H), 6.80 (d, J=8.7 Hz, 2H), 2.17 (s, 3H); LCMS C₁₃H₁₃NO method (A) Rₜ=5.325 min, ESI+ m/z=200.2 (M+H).

3′-Amino-3-methoxy-4′-methyl-[1,1′-biphenyl]-4-oI OR0321-4 (81%) as a white powder. ¹H NMR (400 MHz, CDCl₃) δ 7.10-7.04 (m, 3H), 6.95 (d, J=8.0 Hz, 1H), 6.89 (dd, J=7.7, 1.8 Hz, 1H), 6.86 (d, J=1.8 Hz, 1H), 5.59 (s, 1H), 3.94 (s, 3H), 3.67 (brs, 2H), 2.20 (s, 3H); LCMS C₁₄H₁₅NO₂ method (A) Rₜ=5.721 min, ESI+ m/z=230.2 (M+H).

4-Methyl-4′-(3-(4-methylpiperazin-1-yl)propyl)-[1,1′-biphenyl]-3-amine OR0598-5 (86%) as a yellow foam. Rf=0.35 (DCM-MeOH—NH₄OH, 90:9:1). ¹H NMR (400 MHz, CDCl₃) δ 7.47 (d, J=8.2 Hz, 2H), 7.22 (d, J=8.2 Hz, 2H), 7.10 (d, J=7.7 Hz, 1H), 6.93 (dd, J=7.7, 1.8 Hz, 1H), 6.89 (d, J=1.8 Hz, 1H), 3.66 (brs, 2H), 2.68-2.63 (m, 2H), 2.48 (brs, 8H), 2.42-2.38 (m, 2H), 2.29 (s, 3H), 2.20 (s, 3H), 1.85 (quint, J=7.7 Hz, 2H); LCMS C₂₁H₂₉N₃ method (B) Rₜ=3.659 min, ESI+ m/z=324.3 (M+H).

4-Methyl-4′-(2-(4-methylpiperazin-1-yl)ethoxy)-[1,1′-biphenyl]-3-amine OR0596-5 (quantitative) as a yellow oil. LCMS C₂₀H₂₇N₃O method (B) Rₜ=3.669 min, ESI+ m/z=326.3 (M+H).

2-((3′-Amino-4′-methyl-[1,1′-biphenyl]-4-yl)oxy)-1-(4-methylpiperazin-1-yl)ethan-1-one OR0597-5 (quantitative) as a pinky solid. LCMS C₂₀H₂₅N₃O₂ method (B) R=3.586 min, ESI+m/z=340.4 (M+H).

2-Methyl-5-(6-(2-(4-methylpiperazin-1-yl)ethyl)pyridin-3-yl)aniline OR0599-5 (76%) as a white solid, which was used in the next step without further purification. ¹H NMR (400 MHz, CDCl₃) δ 8.70 (d, J=2.0 Hz, 1H), 7.74 (dd, J=8.0, 2.0 Hz, 1H), 7.21 (d, J=8.0 Hz, 1H), 7.13 (d, J=7.7 Hz, 1H), 6.89 (dd, J=7.7, 2.0 Hz, 1H), 6.85 (d, J=2.0 Hz, 1H), 3.72 (brs, 2H), 3.04-3.02 (m, 2H), 2.83-2.81 (m, 2H), 2.64 (brs, 4H), 2.53 (brs, 4H), 2.32 (s, 3H), 2.20 (s, 3H); LCMS C₁₉H₂₆N₄ method (B) Rₜ=1.680 min, ESI+ m/z=311.2 (M+H).

8-(4-(2-(4-Methylpiperazin-1-yl)ethyl)phenyl)-2,3,4,5-tetrahydro-1H-benzo[b]azepine OR0402-4.

A suspension of 1-methyl-4-(4-(4,4,5,5-tetramethyl-1,3,2-dioxaborolan-2-yl)phenethyl)piperazine OR0402-5 (330 mg, 1.0 mmol), 8-bromo-2,3,4,5-tetrahydro-1H-benzo[b]azepine hydrochloride (262 mg, 1.0 mmol) PdCl₂(dppf) (109 mg, 0.149 mmol), and K₂CO₃ (839 mg, 6.07 mmol) in a mixture of 1,4-dioxane-water (5:1, 30 mL) was thoroughly degassed several times under argon fillings. The reaction mixture was heated at 80° C. for 1.5 hours. The solvent was distillated off and the residue was triturated with EtOAc-DCM (1:1, 250 mL) and filtered through a pad of celite. The filtrate was concentrated under reduced pressure, and the residue was purified by flash chromatography, gradient DCM-MeOH—NH₄OH (100:0:0 to 90:9:1) to afford 8-(4-(2-(4-methylpiperazin-1-yl)ethyl)phenyl)-2,3,4,5-tetrahydro-1H-benzo[b]azepine OR0402-4 (215 mg, 61%) as a yellow foam. Rf=0.27 (DCM-MeOH—NH₄OH, 95:5:0.5); ¹H NMR (400 MHz, CDCl₃) δ 7.49 (d, J=8.2 Hz, 2H), 7.26 (d, J=8.2 Hz, 2H), 7.16 (d, J=7.7 Hz, 1H), 7.04 (dd, J=7.7, 1.8 Hz, 1H), 6.94 (d, J=1.8 Hz, 1H), 3.88 (brs, 1H), 3.12-3.06 (m, 2H), 2.87-2.83 (m, 2H), 2.82-2.78 (m, 2H), 2.70-2.64 (m, 2H), 2.60 (brs, 8H), 2.35 (s, 3H), 1.85-1.81 (m, 2H), 1.70-1.66 (m, 2H)). ¹³C NMR (100 MHz, CDCl₃) δ 150.79, 139.67, 139.09, 139.05, 132.76, 131.38, 129.11, 127.11, 119.56, 118.02, 60.44, 55.10, 53.03, 49.06, 46.01, 35.85, 33.27, 32.10, 27.10; LCMS C₂₃H₃₁N₃ method (B) Rₜ=2.662 min, ESI+ m/z=350.3 (M+H).

General Procedure for the Synthesis of 3-nitro-1,1′-biaryl Derivatives. Under argon, a suspension of appropriate aryl halide (1.0 mmol), 4,4,5,5-tetramethyl-2-(4-methyl-3-nitrophenyl)-1,3,2-dioxaborolane (264 mg, 1.03 mmol), PdCl₂(dppf) (36 mg, 0.05 mmol) and Na₂CO₃ (212 mg, 2 mmol) in a degassed mixture of 1,4-dioxane-water (5:1, 12 mL) was refluxed for 3 hrs, upon complete consumption of starting material. The solvent was distillated off under reduced pressure and the residue purified by flash chromatography to afford corresponding 3-nitro-1,1′-biaryl.

1-Methyl-4-(4′-methyl-3′-nitro-[1,1′-biphenyl]-4-yl)methyl)piperazine OR0274-5 (74%) as a dark oil. ¹H NMR (400 MHz, CDCl₃) δ 8.19 (d, J=1.9 Hz, 1H), 7.71 (dd, J=8.0, 1.9 Hz, 1H), 7.54 (d, J=8.2 Hz, 2H), 7.42 (d, J=8.2 Hz, 2H), 7.39 (d, J=8.0 Hz, 1H), 3.56 (s, 2H), 2.63 (s, 3H), 2.48 (brs, 8H), 2.30 (s, 3H); LC method (A) Rₜ=6.840 min.

1-Methyl-4-(2′(4′-methyl-3′-nitro-[1,1′-biphenyl]-4-yl)ethyl)piperazine OR0325-5 (89%) as a dark oil. ¹H NMR (400 MHz, CDCl₃) δ 8.18 (d, J=1.9 Hz, 1H), 7.70 (dd, J=7.9, 1.9 Hz, 1H), 7.52 (d, J=8.2 Hz, 2H), 7.39 (d, J=7.9 Hz, 1H), 7.31 (d, J=8.2 Hz, 2H), 2.90-2.82 (m, 2H), 2.68-2.63 (m, 2H), 2.63 (s, 3H), 2.51 (s, 8H), 2.31 (s, 3H); LCMS C₂₀H₂₅N₃O₂ method (B) Rₜ=4.532 min. ESI+ m/z=340.2 (M+H).

4′-Methyl-3′-nitro-[1,1′-biphenyl]-4-ol OR0320-5 (73%) as a yellow powder. ¹H NMR (400 MHz, CDCl₃) δ 8.14 (d, J=2.0 Hz, 1H)), 7.67 (dd, J=7.9, 2.0 Hz, 1H), 7.49 (d, J=8.7 Hz, 2H), 7.37 (d, J=7.9 Hz, 1H), 6.93 (d, J=8.7 Hz, 2H), 4.94 (s, 1H), 2.62 (s, 3H); LCMS C₁₃H₁₁NO₃ method (A) Rₜ=6.516 min, ESI+ m/z=230.1 (M+H).

3-Methoxy-4′-methyl-3′-nitro-[1,1′-biphenyl]-4-ol OR0321-5 (69%) as a yellow powder. ¹H NMR (400 MHz, CDCl₃) δ 8.14 (d, J=1.6 Hz, 1H), 7.67 (dd, J=7.9, 1.6 Hz, 1H), 7.37 (d, J=7.9 Hz, 1H), 7.11 (dd, J=8.2, 1.9 Hz, 1H), 7.06 (d, J=1.9 Hz, 1H), 7.01 (d, J=8.2 Hz, 1H), 5.71 (s, 1H), 3.98 (s, 3H), 2.62 (s, 3H); LCMS C₁₄H₁₃NO₄ method (A) Rₜ=6.637 min, ESI+m/z=260.1 (M+H).

1-Methyl-4-(3′(4′-methyl-3′-nitro-[1,1′-biphenyl]-4-yl)propyl)piperazine OR0598-6 (73%) as a yellow foam. Rf=0.42 (DCM-MeOH—NH₄OH, 90:9:1). ¹H NMR (400 MHz, CDCl₃) δ 8.18 (d, J=1.9 Hz, 1H), 7.70 (dd, J=7.9, 1.9 Hz, 1H), 7.51 (d, J=8.2 Hz, 2H), 7.38 (d, J=8.0 Hz, 1H), 7.28 (d, J=8.2 Hz, 2H), 2.71-2.66 (m, 2H), 2.62 (s, 3H), 2.47 (brs, 8H), 2.42-2.36 (m, 2H), 2.29 (s, 3H), 1.85 (quint, J=7.7 Hz, 2H); LCMS C₂₁H₂₇N₃O₂ method (B) R=4.576 min, ESI+ m/z=354.2 (M+H).

1-Methyl-4-(2-((4′-methyl-3′-nitro-[1,1′-biphenyl]-4-yl)oxy)ethyl)piperazine OR0596-6 (72%) as a yellow oil. ¹H NMR (400 MHz, CDCl₃) δ 8.15 (d, J=2.0 Hz, 1H), 7.67 (dd, J=8.0, 2.0 Hz, 1H), 7.52 (d, J=8.8 Hz, 2H), 7.36 (d, J=8.0 Hz, 1H), 6.99 (d, J=8.8 Hz, 2H), 4.15 (t, J=5.8 Hz, 2H), 2.85 (t, J=5.8 Hz, 2H), 2.61 (s, 3H), 2.60 (brs, 4H), 2.50 (brs, 4H), 2.30 (s, 3H); LCMS C₂₀H₂₅N₃O₃ method (B) Rₜ=4.655 min, ESI+ m/z=356.3 (M+H).

2-((4′-Methyl-3′-nitro-[1,1′-biphenyl]-4-yl)oxy)-1-(4-methylpiperazin-1-yl)ethan-1-one OR0597-6 (72%) as a yellow solid. ¹H NMR (400 MHz, CDCl₃) δ 8.14 (d, J=1.8 Hz, 1H), 7.66 (dd, J=7.9, 1.8 Hz, 1H), 7.52 (d, J=8.8 Hz, 2H), 7.37 (d, J=7.9 Hz, 1H), 7.04 (d, J=8.8 Hz, 2H), 4.74 (s, 2H), 3.68-3.58 (m, 4H), 2.61 (s, 3H), 2.44-2.37 (m, 4H), 2.30 (s, 3H); LCMS C₂₀H₂₃N₃O₄ method (B) Rₜ=4.555 min, ESI+ m/z=370.4 (M+H).

1-Methyl-4-(2-(5-(4-methyl-3-nitrophenyl)pyridin-2-yl)ethyl)piperazine OR0599-6 (79%) as a white solid. ¹H NMR (400 MHz, CDCl₃) δ 8.75 (d, J=2.4 Hz, 1H), 8.16 (d, J=1.9 Hz, 1H), 7.80 (dd, J=8.1, 2.4 Hz, 1H), 7.70 (dd, J=7.9, 1.9 Hz, 1H), 7.44 (d, J=7.9 Hz, 1H), 7.30 (d, J=8.1 Hz, 1H), 3.06-3.03 (m, 2H), 2.82-2.80 (m, 2H), 2.64 (s, 3H), 2.60 (brs, 4H), 2.48 (brs, 4H), 2.29 (s, 3H); LCMS C₁₉H₂₄N₄O₂ method (B) Rₜ=3.942 min, ESI+ m/z=341.2 (M+H).

General Procedure for the Synthesis of 1-(Arylalkyl)-4-methylpiperazine Derivatives. Under argon, to a solution of appropriate arylalkyl methanesulfonate (10 mmol) in acetonitrile (30 mL) were successively added potassium carbonate (4.13 g, 30 mmol) and N-methyl-piperazine (1.40 mL, 15 mmol). The reaction mixture was heated at 60° C., until complete conversion monitored by TLC. The resulting mixture was allowed to cool to room temperature, then poured into H₂O (100 mL) and extracted with DCM (3×30 mL). The combined organic layers were washed with brine and dried over Na₂SO₄. The solvent was distillated off and the residue was purified by flash chromatography, to afford corresponding 1-(arylalkyl)-4-methylpiperazine.

1-(4-Bromophenethyl)-4-methylpiperazine OR0325-6 (68%) as a colorless oil. ¹H NMR (400 MHz, CDCl₃) δ 7.39 (d, J=8.3 Hz, 2H), 7.07 (d, J=8.3 Hz, 2H), 2.79-2.71 (m, 2H), 2.62-2.54 (m, 2H), 2.53 (brs, 8H), 2.32 (s, 3H). LC method (D) Rₜ=5.648 min.

1-(3-(4-Bromophenyl)propyl)-4-methylpiperazine OR0598-7 (98%) was used in the next step without further purification. Rf=0.40 (DCM-MeOH—NH₄OH, 90:9:1). ¹H NMR (400 MHz, CDCl₃) δ 7.38 (d, J=8.4 Hz, 2H), 7.05 (d, J=8.4 Hz, 2H), 2.62-2.54 (m, 2H), 2.44 (brs, 8H), 2.38-2.30 (m, 2H), 2.28 (s, 3H), 1.78 (quint, J=7.7 Hz, 2H); LCMS C₁₄H₂₁BrN₂ method (B) Rₜ=1.174 min, ESI+ m/z=297.1 (M+H).

1-(2-(5-Bromopyridin-2-yl)ethyl)-4-methylpiperazine OR0599-7 (81%) was used in the next step without further purification. ¹H NMR (400 MHz, CDCl₃) δ 8.55 (d, J=2.2 Hz, 1H), 7.69 (dd, J=8.3, 2.2 Hz, 1H), 7.08 (d, J=8.3 Hz, 1H), 2.96-2.88 (m, 2H), 2.76-2.68 (m, 2H), 2.54 (brs, 4H), 2.43 (brs, 4H), 2.27 (s, 3H); LCMS C₁₂H₁₅BrN₃ method (B) Rₜ=1.921 min, ESI+ m/z=284.0 (M+H). 1-(2-(4-Bromophenoxy)ethyl)-4-methylpiperazine OR0596-7 (88%) as a colorless oil. ¹H NMR (400 MHz, CDCl₃) δ 7.35 (d, J=9.0 Hz, 2H), 6.78 (d, J=9.0 Hz, 2H), 4.06 (t, J=5.8 Hz, 2H), 2.79 (t, J=5.8 Hz, 2H), 2.60 (brs, 4H), 2.47 (brs, 4H), 2.28 (s, 3H); LCMS C₁₃H₁₉BrN₂O method (B) Rₜ=4.004 min. ESI+ m/z=299.1 (M+H).

1-Methyl-4-(4-(4,4,5,5-tetramethyl-1,3,2-dioxaborolan-2-yl)phenethyl)piperazine OR0402-5 (65%) as a white solid. Rf=0.40 (EP/EA, 5:5); ¹H NMR (400 MHz, CDCl₃) δ 7.73 (d, J=8.0 Hz, 2H), 7.21 (d, J=8.0 Hz, 2H), 2.86-2.78 (m, 2H), 2.67-2.59 (m, 2H), 2.61 (brs, 8H), 2.34 (s, 3H), 1.33 (s, 12H); ¹³C NMR (100 MHz, CDCl₃) δ 143.63, 135.09, 128.27, 83.82, 60.11, 55.00, 52.81, 45.89, 33.78, 24.98; LCMS C₁₉H₃₁BN₂O₂ method (B) Rₜ=3.392 min, ESI+ m/z=331.3 (M+H).

2-(4-Bromophenoxy)-1-(4-methylpiperazin-1-yl)ethan-1-one OR0597-7.

By peptide coupling procedure, previously described for the synthesis of di-tert-butyl (2-(2-((3-benzamidoaryl)amino)thiazol-4-yl)pyrimidin-4-yl)carbamate derivatives, (88%) as a yellow oil. ¹H NMR (400 MHz, CDCl₃) δ 7.37 (d, J=9.1 Hz, 2H), 6.83 (d, J=9.1 Hz, 2H), 4.66 (s, 2H), 3.66-3.60 (m, 2H), 3.59-3.53 (m, 2H), 2.41-2.33 (m, 4H), 2.28 (s, 3H); LCMS C₁₃H₁₇BrN₂O₂ method (B) Rₜ=3.939 min, ESI+ m/z=313.1 (M+H).

General Procedure for the Synthesis of 1-(Arylalkyl) Methanesulfonate Derivatives. Under argon, at −5° C., to a solution of appropriate commercially available alcohol (10 mmol) and triethylamine (2.8 mL, 20 mmol) in dry dichloromethane (30 mL) was added dropwise methanesulfonyl chloride (1.15 mL, 14.9 mmol). After addition, the reaction mixture was stirred at −5° C. for 45 min, until complete conversion monitored by TLC, then poured into a saturated NaHCO₃ aqueous solution (50 mL) and extracted twice with DCM (20 mL). The combined organic layers were washed with brine and dried over Na₂SO₄. The solvent was distillated off to afford corresponding arylalkyl methanesulfonate, used in the next step without further purification. 4-Bromophenethyl methanesulfonate OR0325-7 (quantitative) as a white solid. ¹H NMR (400 MHz, CDCl₃) δ 7.45 (d, J=8.4 Hz, 2H), 7.13 (d, J=8.4 Hz, 2H), 4.39 (t, J=6.8 Hz, 2H), 3.01 (t, J=6.8 Hz, 2H), 2.89 (s, 3H); LCMS C₉H₁₁BrO₃S method (B) Rₜ=6.025 min, ESI+m/z=301.0 (M+Na).

3-(4-Bromophenyl)propyl methanesulfonate OR0598-8 (95%) as a white solid. Rf=0.55 (DCM). ¹H NMR (400 MHz, CDCl₃) δ 7.42 (d, J=8.4 Hz, 2H), 7.07 (d, J=8.4 Hz, 2H), 4.21 (t, J=6.3 Hz, 2H), 2.99 (s, 3H), 2.75-2.67 (m, 2H), 2.10-1.99 (m, 2H); LC method (B) Rₜ=5.642 min.

2-(4-Bromophenoxy)ethyl methanesulfonate OR0596-8 (quantitative) as a colorless oil. LC method (B) Rₜ=6.010 min. 2-(5-Bromopyridin-2-yl)ethyl methanesulfonate OR0599-8 (quantitative) as a colorless oil, was used in the next step without further purification. LCMS C₈H₁₀BrNO₃S method (B) Rₜ=5.280 min, ESI+ m/z=280.0 (M+H).

4-(4,4,5,5-tetramethyl-1,3,2-dioxaborolan-2-yl)phenethyl methanesulfonate OR0402-6 (91%) as a white solid, used in the next step without further purification. Rf=0.30 (PE/EtOAc, 1:1); ¹H NMR (400 MHz, CDCl₃) δ 7.77 (d, J=7.9 Hz, 2H), 7.24 (d, J=7.9 Hz, 2H), 4.42 (t, J=6.9 Hz, 2H), 3.06 (t, J=6.9 Hz, 2H), 2.83 (s, 3H), 1.34 (s, 12H); LCMS C₁₅H₂₃BO₅S method (B) Rₜ=5.498 min, ESI+ m/z=327.2 (M+H).

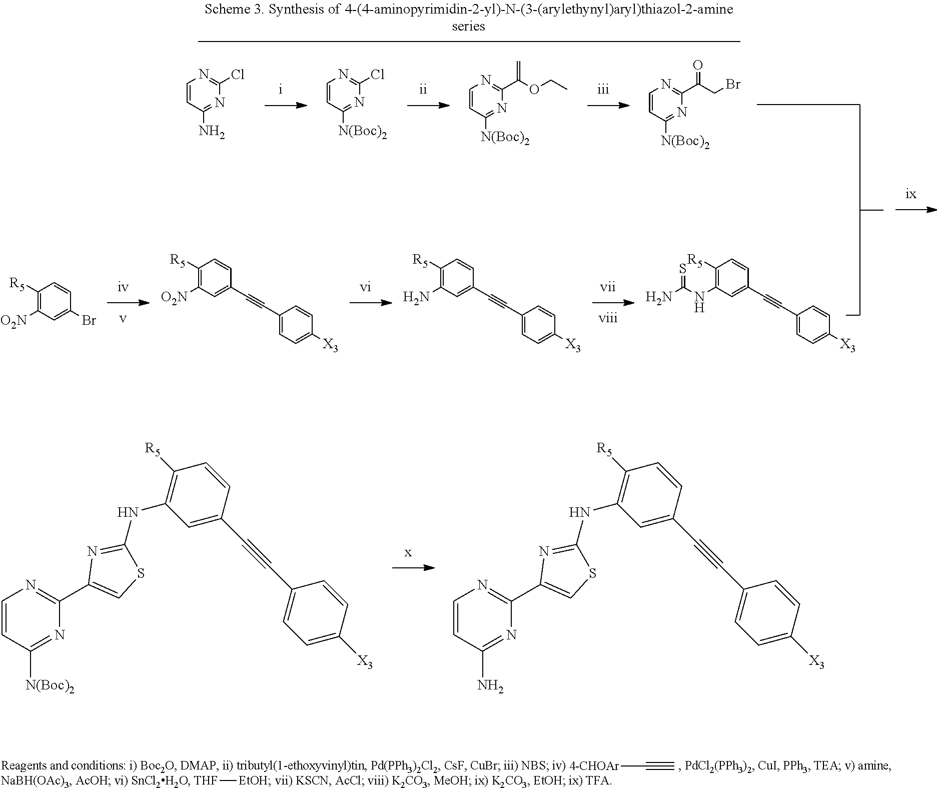

4-(4-Aminopyrimidin-2-yl)-N-(3-(arylethynyl)aryl)thiazol-2-amine derivatives were prepared by convergent synthesis, from commercially available 4-amino-2-chloropyrimidine and appropriate bromo-3-nitrobenzene (Scheme 3). The 4-amino-2-chloropyrimidine was protected using Boc₂O to afford corresponding bis-carbamate. A Stille cross-coupling reaction with tributyl(1-ethoxyvinyl)tin gives the enol ether which was then turned into the corresponding α-bromoketone using N-bromosuccinimide. Starting from appropriate bromo-3-nitrobenzene, a Sonogashira cross-coupling reaction with appropriate 4-ethynylbenzaldehyde allowed to introduce the key 1,2-diarylethyne scaffold. Reductive amination afforded the corresponding benzylamine, while reduction of the nitro group followed by condensation with acetylisothiocyanate and saponification gave the corresponding thiourea. The thiourea was then engaged in a Hantzsch thiazole synthesis with the α-bromoketone leading to the corresponding thiazole. Finally, deprotection with TFA led to expected 4-(4-aminopyrimidin-2-yl)-N-(3-(arylethynyl)aryl)thiazol-2-amines.

General Procedure for the Synthesis of 4-(4-Aminopyrimidin-2-yl)-N-(3-(arylethynyl)aryl)thiazol-2-amine Derivatives. As previously described for N-(3-((4-(4-aminopyrimidin-2-yl)thiazol-2-yl)amino)-4-aryl)benzamide derivatives.

4-(4-Aminopyrimidin-2-yl)-N-(5-((4-((dimethylamino)methyl)phenyl)ethynyl)-2-methylphenyl)thiazol-2-amine OR0237 (30%) as a white powder. ¹H NMR (400 MHz, CDCl₃) δ 8.30 (d, J=5.8 Hz, 1H), 7.67 (d, J=1.2 Hz, 1H), 7.60 (s, 1H), 7.50 (d, J=8.1 Hz, 2H), 7.29 (d, J=8.1 Hz, 2H), 7.24 (dd, J=7.8, 1.2 Hz, 1H), 7.22 (d, J=7.8 Hz, 1H), 6.34 (d, J=5.8 Hz, 1H), 4.98 (s, 2H), 3.43 (s, 2H), 2.32 (s, 3H), 2.24 (s, 6H); ¹³C NMR (100 MHz, CDCl₃) δ 166.21, 163.10, 160.53, 156.60, 150.57, 139.37, 138.76, 131.71, 131.45, 130.23, 129.24, 128.02, 123.03, 122.49, 121.96, 111.40, 103.46, 89.63, 88.85, 64.23, 45.49, 18.11; LC method (D) Rₜ=4.030 min.

4-(4-Aminopyrimidin-2-yl)-N-(2-methyl-5-((4-((4-methylpiperazin-1-yl)methyl)phenyl)ethynyl)phenyl)thiazol-2-amine OR0153 (45%) as a white powder. ¹H NMR (400 MHz, CDCl₃) δ 8.30 (d, J=5.7 Hz, 1H), 7.67 (d, J=1.2 Hz, 1H), 7.60 (s, 1H), 7.48 (d, J=8.0 Hz, 2H), 7.31 (d, J=8.0 Hz, 2H), 7.24 (dd, J=7.8, 1.2 Hz, 1H), 7.22 (d, J=8.0 Hz), 6.34 (d, J=5.7 Hz, 1H), 4.98 (s, 2H), 3.51 (s, 2H), 2.47 (brs, 8H), 2.32 (s, 3H), 2.29 (s, 3H); ¹³C NMR (100 MHz, CDCl₃) δ 166.37, 163.13, 160.55, 156.55, 150.59, 138.84, 131.65, 131.45, 130.48, 129.24, 128.10, 123.30, 122.45, 121.91, 114.19, 111.32, 103.50, 89.60, 88.81, 62.82, 55.17, 53.10, 46.05, 18.08; LC method (D) R_{c}=3.865 min.

General Procedure for the Synthesis of di-tert-butyl (2-(2-((3-(arylethynyl)aryl)amino)thiazol-4-yl)pyrimidin-4-yl)carbamate Derivatives. As previously described for di-tert-butyl (2-(2-((3-nitroaryl)amino)thiazol-4-yl)pyrimidin-4-yl)carbamate derivatives.

Di-tert-butyl (2-(2-((5-((4-((dimethylamino)methyl)phenyl)ethynyl)-2-methylphenyl)amino)thiazol-4-yl)pyrimidin-4-yl)carbamate OR0237-1 (60%) as a light brown powder. ¹H NMR (400 MHz, CDCl₃) δ 8.67 (d, J=5.7 Hz, 1H), 7.67 (d, J=1.3 Hz, 1H), 7.53 (d, J=5.1 Hz, 1H), 7.53 (s, 1H), 7.50 (d, J=8.2 Hz, 2H), 7.31 (d, J=8.2 Hz, 2H), 7.26 (dd, J=7.8, 1.3 Hz, 1H), 7.23 (d, J=7.8 Hz, 1H), 3.47 (s, 2H), 2.32 (s, 3H), 2.27 (s, 6H), 1.56 (s, 18H).

Di-tert-butyl (2-(2-((2-methyl-5-((4-((4-methylpiperazin-1-yl)methyl)phenyl)ethynyl)phenyl)amino)thiazol-4-yl)pyrimidin-4-yl)carbamate OR0153-1(44%) as a light brown powder. ¹H NMR (400 MHz, CDCl₃) δ 8.68 (d, J=5.7 Hz, 1H), 7.67 (d, J=1.4 Hz, 1H), 7.53 (d, J=5.9 Hz, 1H), 7.53 (s, 1H), 7.48 (d, J=8.2 Hz, 2H), 7.31 (d, J=8.2 Hz, 2H), 7.25 (dd, J=7.9, 1.4 Hz, 1H), 7.22 (d, J=7.9 Hz, 1H), 3.53 (s, 2H), 2.55 (brs, 8H), 2.36 (s, 3H), 2.32 (s, 3H), 1.56 (s, 18H).

General Procedure for the Synthesis of 1-(3-(arylethynyl)aryl)thiourea Derivatives. As previously described for 1-(3-nitroaryl)thiourea derivatives.

1-(5-((4-((Dimethylamino)methyl)phenyl)ethynyl)-2-methylphenyl)thiourea OR0237-2 (72%) as a white powder. ¹H NMR (400 MHz, MeOD) δ 7.53-7.47 (m, 3H), 7.40-7.30 (m, 4H), 3.50 (s, 2H), 2.30 (s, 3H), 2.25 (s, 6H).

1-(2-Methyl-5-((4-((4-methylpiperazin-1-yl)methyl)phenyl)ethynyl)phenyl)thiourea OR0153-2 (65%) as a white powder. ¹H NMR (400 MHz, MeOD) δ 7.53-7.21 (m, 7H), 3.55 (s, 2H), 2.51 (brs, 8H), 2.28 (s, 6H).

General Procedure for the Synthesis of N-((3-(arylethynyl)aryl)carbamothioyl)acetamide Derivatives. As previously described for N-((3-nitroaryl)carbamothioyl)acetamide derivatives.

N-((5-((4-((Dimethylamino)methyl)phenyl)ethynyl)-2-methylphenyl)carbamothioyl)acetamide OR0237-3 as a light brown solid, engaged in the next step without further purification. ¹H NMR (400 MHz, CDCl₃) δ 12.01 (s, 1H), 9.10 (s, 1H), 7.86 (d, J=1.3 Hz, 1H), 7.57 (d, J=8.3 Hz, 2H), 7.53 (d, J=8.3 Hz, 2H), 7.39 (dd, J=7.9, 1.3 Hz, 1H), 7.24 (d, J=7.9 Hz, 1H), 4.09 (s, 2H), 2.70 (s, 6H), 2.32 (s, 3H), 2.26 (s, 3H).

N-((2-Methyl-5-((4-((4-methylpiperazin-1-yl)methyl)phenyl)ethynyl)phenyl) carbamothioyl)acetamide OR0153-3 as a light brown solid, engaged in the next step without further purification. ¹H NMR (400 MHz, CDCl₃) δ 11.99 (s, 1H), 9.07 (s, 1H), 7.83 (d, J=1.3 Hz, 1H), 7.47 (d, J=8.1 Hz, 2H), 7.37 (dd, J=7.8, 1.3 Hz, 1H), 7.28 (d, J=8.1 Hz, 2H), 7.24 (d, J=7.8 Hz, 1H), 3.60 (s, 2H), 2.90 (brs, 8H), 2.66 (s, 3H), 2.31 (s, 3H), 2.25 (s, 3H).

General Procedure for the Synthesis of 3-(arylethynyl)aniline Derivatives. As previously described for di-tert-butyl (2-(2-((3-aminoaryl)amino)thiazol-4-yl)pyrimidin-4-yl)carbamate derivatives.

5-((4-((Dimethylamino)methyl)phenyl)ethynyl)-2-methylaniline OR0237-4 engaged in the next step without further purification. ¹H NMR (400 MHz, CDCl₃) δ 7.45 (d, J=8.1 Hz, 2H), 7.29 (d, J=8.1 Hz, 2H), 7.01 (d, J=7.7 Hz, 1H), 6.88 (dd, J=7.7, 1.9 Hz, 1H), 6.85 (d, J=1.9 Hz, 1H), 3.62 (brs, 2H), 3.50 (s, 2H), 2.46 (s, 2H), 2.25 (s, 6H), 2.18 (s, 3H).

2-Methyl-5-((4-((4-methylpiperazin-1-yl)methyl)phenyl)ethynyl)aniline OR0153-4 engaged in the next step without further purification. ¹H NMR (400 MHz, CDCl₃) δ 7.45 (d, J=8.1 Hz, 2H), 7.29 (d, J=8.1 Hz, 2H), 7.01 (d, J=7.7 Hz, 1H), 6.88 (dd, J=7.7, 1.1 Hz, 1H), 6.85 (d, J=1.1 Hz, 1H), 3.62 (brs, 2H), 3.50 (s, 2H), 2.46 (brs, 8H), 2.29 (s, 3H), 2.18 (s, 3H).

General Procedure for the Synthesis of (4-((3-nitroaryl)ethynyl)aryl)methanamine Derivatives. To a solution of appropriate 4-((3-nitroaryl)ethynyl)benzaldehyde (1 mmol) and amine (3 mmol) in a mixture of 1,2-dichloroethane-tetrahydrofuran (10:1, 11 mL) was added few drops of acetic acid. The reaction mixture was stirred 5 min, before portion wise addition of sodium triacetoxyborohydride (424 mg, 2 mmol). The resulting suspension was stirred at room temperature overnight, upon complete consumption of starting material monitored by TLC. The reaction was slowly quenched with saturated aqueous NaHCO₃ (10 mL) and extracted with DCM (3×10 mL). The combined organic layers were washed with brine (10 mL) and dried over Na₂SO₄. The solvent was distillated off under reduced pressure and the residue was purified by flash chromatography to afford corresponding (4-((3-nitroaryl)ethynyl)aryl)methanamine.

N,N-Dimethyl-1-(4-((4-methyl-3-nitrophenyl)ethynyl)phenyl)methanamine OR0237-5 (51%) as a yellow oil. ¹H NMR (400 MHz, CDCl₃) δ 8.12 (d, J=1.6 Hz, 1H), 7.61 (dd, J=7.9, 1.6 Hz, 1H), 7.49 (d, J=8.2 Hz, 2H), 7.33-7.31 (m, 3H), 3.44 (s, 2H), 2.61 (s, 3H), 2.25 (s, 6H).

1-Methyl-4-(4-((4-methyl-3-nitrophenyl)ethynyl)benzyl)piperazine OR0153-5 (53%) as a light brown powder. ¹H NMR (400 MHz, CDCl₃) δ 8.11 (d, J=1.7 Hz, 1H), 7.61 (dd, J=7.9, 1.7 Hz, 1H), 7.48 (d, J=8.2 Hz, 2H), 7.33 (d, J=8.2 Hz, 2H), 7.32 (d, J=7.9 Hz, 1H), 3.52 (s, 2H), 2.61 (s, 3H), 2.49 (brs, 8H), 2.31 (s, 3H).

General Procedure for the Synthesis of 4-((3-nitroaryl)ethynyl)benzaldehyde Derivatives. A solution of appropriate bromo-3-nitrobenzene (1 mmol) and 4-ethynylbenzaldehyde (1.4 mmol) in a mixture of dimethylformamide-triethylamine (1:1, 6 mL) was thoroughly degassed several times under argon fillings. Bis(triphenylphosphine)palladium (II) dichloride (35 mg, 0.05 mmol), copper iodide (10 mg, 0.05 mmol) and triphenylphosphine (26 mg, 0.1 mmol) were successively added. The reaction mixture was heated at 70° C. until complete conversion monitored by TLC. The solvent was distillated off under reduced pressure and the residue was purified by flash chromatography to afford corresponding 4-((3-nitroaryl)ethynyl)benzaldehyde.

4-((4-Methyl-3-nitrophenyl)ethynyl)benzaldehyde OR0153-6 (quantitative) as a light brown powder. ¹H NMR (400 MHz, CDCl₃) δ 10.03 (s, 1H), 8.15 (d, J=1.6 Hz, 1H), 7.89 (d, J=8.2 Hz, 2H), 7.69 (d, J=8.2 Hz, 2H), 7.65 (dd, J=7.9, 1.6 Hz, 1H), 7.36 (d, J=7.9 Hz, 1H), 2.63 (s, 3H); LC method (A) Rₜ=7.467 min.

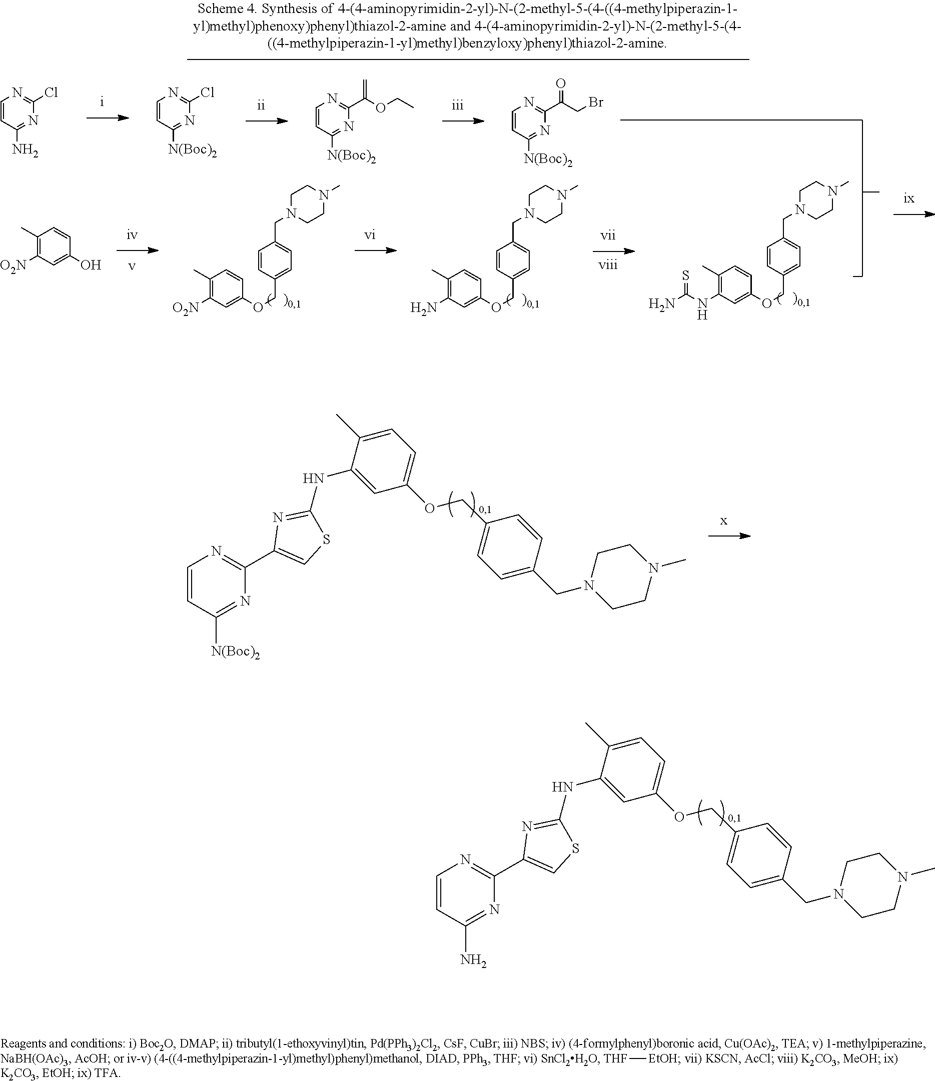

4-(4-Aminopyrimidin-2-yl)-N-(2-methyl-5-(4-((4-methylpiperazin-1-yl)methyl)phenoxy)phenyl)thiazol-2-amine and benzyloxyphenyl analogue were prepared by convergent synthesis, in ten steps, from commercially available 4-amino-2-chloropyrimidine and 4-methyl-3-nitrophenol (Scheme 4). The 4-amino-2-chloropyrimidine was protected using Boc₂O to afford corresponding bis-carbamate. A Stille cross-coupling reaction with tributyl(1-ethoxyvinyl)tin gave the enol ether which was then turned into the corresponding α-bromoketone using N-bromosuccinimide. Starting from 4-methyl-3-nitrophenol, O-arylation in the Chan-Lam coupling conditions, with (4-formylphenyl)boronic acid allowed to construct the diphenyl ether moiety while Mistunobu reaction with appropriate arylmethanol led to corresponding benzyloxyphenyl derivative. Reductive amination afforded the corresponding amine, while reduction of the nitro group followed by condensation with acetylisothiocyanate and saponification gave the corresponding thiourea. The thiourea was then engaged in a Hantzsch thiazole synthesis with the α-bromoketone leading to the corresponding thiazole. Finally, deprotection with TFA led to expected 4-(4-aminopyrimidin-2-yl)-N-(2-methyl-5-(4-((4-methylpiperazin-1-yl)methyl)phenoxy)phenyl)thiazol-2-amine and 4-(4-aminopyrimidin-2-yl)-N-(2-methyl-5-(4-((4-methylpiperazin-1-yl)methyl)benzyloxy)phenyl)thiazol-2-amine, respectively.

4-(4-Aminopyrimidin-2-yl)-N-(2-methyl-5-(4-((4-methylpiperazin-1-yl)methyl)phenoxy)phenyl)thiazol-2-amine OR0143

As previously described for N-(3-((4-(4-aminopyrimidin-2-yl)thiazol-2-yl)amino)-4-aryl)benzamide derivatives, (62%) as a white powder. ¹H NMR (400 MHz, CDCl₃) δ 8.29 (d, J=5.7 Hz, 1H), 7.56 (s, 1H), 7.29 (d, J=8.5 Hz, 2H), 7.18 (d, J=2.4 Hz, 1H), 7.16 (d, J=8.3 Hz, 1H), 6.98 (d, J=8.5 Hz, 2H), 6.70 (dd, J=8.3, 2.4 Hz, 1H), 6.32 (d, J=5.7 Hz, 1H), 4.98 (brs, 2H), 3.48 (s, 2H), 2.47 (brs, 8H), 2.28 (s, 3H), 2.26 (s, 3H); ¹³C NMR (100 MHz, CDCl₃) δ 165.76, 163.07, 160.44, 156.67, 156.47, 156.14, 150.46, 139.61, 133.30, 132.05, 130.67, 123.45, 118.79, 114.33, 111.23, 110.01, 103.42, 62.45, 55.21, 53.07, 46.11, 17.32; LC method (C) Rₜ=3.476 min.

4-(4-Aminopyrimidin-2-yl)-N-(2-methyl-5-(4-((4-methylpiperazin-1-yl)methyl)benzyloxy)phenyl)thiazol-2-amine OR0232

As previously described for N-(3-((4-(4-aminopyrimidin-2-yl)thiazol-2-yl)amino)-4-aryl)benzamide derivatives, (70%) as a white powder. ¹H NMR (400 MHz, CDCl₃) δ 8.29 (d, J=5.7 Hz, 1H), 7.56 (s, 1H), 7.37 (d, J=8.1 Hz, 2H), 7.33 (d, J=8.1 Hz, 2H), 7.16 (d, J=2.5 Hz, 1H), 7.12 (d, J=8.3 Hz, 1H), 6.69 (dd, J=8.3, 2.5 Hz, 1H), 6.32 (d, J=5.7 Hz, 1H), 5.04 (s, 2H), 5.00 (brs, 2H), 3.51 (s, 2H), 2.46 (brs, 8H), 2.28 (s, 3H), 2.22 (s, 3H); ¹³C NMR (100 MHz, CDCl₃) δ 13C NMR (100 MHz, CDCl₃) δ 166.07, 163.09, 160.50, 158.10, 156.47, 150.47, 139.35, 138.16, 135.72, 131.82, 129.52, 127.55, 121.33, 111.08, 110.84, 106.67, 103.39, 70.19, 62.81, 55.20, 53.15, 46.10, 17.14; LC method (C) Rₜ=3.679 min.

Di-tert-butyl (2-(2-((2-methyl-5-(4-((4-methylpiperazin-1-yl)methyl)phenoxy)phenyl)amino)thiazol-4-yl)pyrimidin-4-yl)carbamate OR0143-1

As previously described for di-tert-butyl (2-(2-((3-nitroaryl)amino)thiazol-4-yl)pyrimidin-4-yl)carbamate derivatives, (48%) as a light brown powder. ¹H NMR (400 MHz, CDCl₃) δ 8.68 (d, J=5.7 Hz, 1H), 7.52 (d, J=5.7 Hz, 1H), 7.49 (s, 1H), 7.36 (brs, 1H), 7.28 (d, J=8.6 Hz, 2H), 7.20 (d, J=2.4 Hz, 1H), 7.17 (d, J=8.3 Hz, 1H), 6.97 (d, J=8.6 Hz, 2H), 6.70 (dd, J=8.3, 2.4 Hz, 1H), 3.49 (s, 2H), 2.49 (brs, 8H), 2.31 (s, 3H), 2.27 (s, 3H), 1.55 (s, 18H).

Di-tert-butyl (2-(2-((2-methyl-5-(4-((4-methylpiperazin-1-yl)methyl)benzyloxy)phenyl)amino)thiazol-4-yl)pyrimidin-4-yl)carbamate OR0232-1

As previously described for di-tert-butyl (2-(2-((3-nitroaryl)amino)thiazol-4-yl)pyrimidin-4-yl)carbamate derivatives, (76%) as a light brown powder. ¹H NMR (400 MHz, CDCl₃) δ 8.69 (d, J=5.7 Hz, 1H), 7.53 (d, J=5.7 Hz, 1H), 7.50 (s, 1H), 7.38 (d, J=8.2 Hz, 2H), 7.33 (d, J=8.2 Hz, 2H), 7.17 (d, J=2.5 Hz, 1H), 7.13 (d, J=8.3 Hz, 1H), 6.69 (dd, J=8.3, 2.5 Hz, 1H), 5.04 (s, 2H), 3.51 (s, 2H), 2.47 (brs, 8H), 2.29 (s, 3H), 2.23 (s, 3H), 1.56 (s, 18H).

1-(2-Methyl-5-(4-((4-methylpiperazin-1-yl)methyl)phenoxy)phenyl)thiourea OR0143-2

As previously described for 1-(3-nitroaryl)thiourea derivatives, (56%) as a white powder. ¹H NMR (400 MHz, CDCl₃) δ 7.63 (brs, 1H), 7.30 (d, J=8.6 Hz, 2H), 7.24 (d, J=8.3 Hz, 1H), 6.93 (d, J=8.6 Hz, 2H), 6.92 (dd, J=8.3, 2.4 Hz, 1H), 6.84 (d, J=2.4 Hz, 1H), 5.94 (brs, 2H), 3.50 (s, 2H), 2.48 (brs, 8H), 2.29 (s, 3H), 2.27 (s, 3H).

1-(2-Methyl-5-(4-((4-methylpiperazin-1-yl)methyl)benzyloxy)phenyl)thiourea OR0232-2

As previously described for 1-(3-nitroaryl)thiourea derivatives, (70%) as a white powder. ¹H NMR (400 MHz, MeOD) δ 7.40 (d, J=8.1 Hz, 2H), 7.34 (d, J=8.1 Hz, 2H), 7.19 (d, J=8.5 Hz, 1H), 6.90 (dd, J=8.5, 2.5 Hz, 1H), 6.85 (d, J=2.5 Hz, 1H), 5.06 (s, 2H), 3.54 (s, 2H), 2.50 (brs, 8H), 2.28 (s, 3H), 2.19 (s, 3H).

N-((2-Methyl-5-(4-((4-methylpiperazin-1-yl)methyl)phenoxy)phenyl)carbamothioyl)acetamide OR0143-3

As previously described for N-((3-nitroaryl)carbamothioyl)acetamide derivatives, engaged in the next step without further purification.

N-((2-Methyl-5-(4-((4-methylpiperazin-1-yl)methyl)benzyloxy)phenyl)carbamothioyl)acetamide OR0232-3

As previously described for N-((3-nitroaryl)carbamothioyl)acetamide derivatives, engaged in the next step without further purification.

2-Methyl-5-(4-((4-methylpiperazin-1-yl)methyl)phenoxy)aniline OR0143-4

As previously described for di-tert-butyl (2-(2-((3-aminoaryl)amino)thiazol-4-yl)pyrimidin-4-yl)carbamates, (70%) as a colorless oil. ¹H NMR (400 MHz, CDCl₃) δ 7.24 (d, J=8.6 Hz, 2H), 6.97 (d, J=8.5 Hz, 1H), 6.93 (d, J=8.6 Hz, 2H), 6.36 (dd, J=8.5, 2.4 Hz, 1H), 6.33 (d, J=2.4 Hz, 1H), 3.62 (brs, 2H), 3.47 (s, 2H), 2.46 (brs, 8H), 2.29 (s, 3H), 2.13 (s, 3H).

2-Methyl-5-(4-((4-methylpiperazin-1-yl)methyl)benzyloxy)aniline OR0232-4

As previously described for di-tert-butyl (2-(2-((3-aminoaryl)amino)thiazol-4-yl)pyrimidin-4-yl)carbamate derivatives, (77%) as a colorless oil. ¹H NMR (400 MHz, CDCl₃) δ 7.36 (d, J=8.2 Hz, 2H), 7.32 (d, J=8.2 Hz, 2H), 6.93 (d, J=8.5 Hz, 1H), 6.35 (dd, J=8.5, 2.5 Hz, 1H), 6.33 (d, J=2.5 Hz, 1H), 4.98 (s, 2H), 3.59 (brs, 2H), 3.50 (s, 2H), 2.46 (brs, 8H), 2.29 (s, 3H), 2.10 (s, 3H).

1-Methyl-4-(4-(4-methyl-3-nitrophenoxy)benzyl)piperazine OR0143-5

As previously described for (4-((3-nitroaryl)ethynyl)aryl)methanamine derivatives, (84%) as a colorless oil. ¹H NMR (400 MHz, CDCl₃) δ 7.57 (d, J=2.6 Hz, 1H), 7.32 (d, J=8.6 Hz, 2H), 7.27 (d, J=8.4 Hz, 1H), 7.15 (dd, J=8.4, 2.6 Hz, 1H), 6.97 (d, J=8.6 Hz, 2H), 3.50 (s, 2H), 2.55 (s, 3H), 2.48 (brs, 8H), 2.30 (s, 3H).

1-Methyl-4-(4-((4-methyl-3-nitrophenoxy)methyl)benzyl)piperazine OR0232-5

Under argon, at 0° C., to a solution of 4-methyl-3-nitrophenol (230 mg, 1.5 mmol), (4-((4-methylpiperazin-1-yl)methyl)phenyl)methanol (300 mg, 1.36 mmol) and triphenylphosphine (536 mg, 2.04 mmol) in tetrahydrofuran (68 mL), was added dropwise a solution of diisopropyl azodicarboxylate (430 μL, 2.04 mmol) in tetrahydrofuran (35 mL). The resulting mixture was allowed to warm to room temperature and stirred overnight. The solvent was distillated off under reduced pressure and the residue was purified by flash chromatography, eluent DCM-MeOH—NH₄OH (99:1:0.1), to afford 1-methyl-4-(4-((4-methyl-3-nitrophenoxy)methyl)benzyl)piperazine OR0232-5 (368 g, 76%) as a colorless oil. ¹H NMR (400 MHz, CDCl₃) δ 7.59 (d, J=2.7 Hz, 1H), 7.38-7.34 (m, 4H), 7.23 (d, J=8.5 Hz, 1H), 7.12 (dd, J=8.5, 2.7 Hz, 1H), 5.07 (s, 2H), 3.52 (s, 2H), 2.53 (s, 3H), 2.47 (brs, 8H), 2.29 (s, 3H).

4-(4-Methyl-3-nitrophenoxy)benzaldehyde OR0143-6

To a solution of 4-methyl-3-nitrophenol (1 g, 6.5 mmol), (4-formylphenyl)boronic acid (1.96 g, 13.0 mmol) and copper(II) acetate (1.19 g, 6.5 mmol) in dichloromethane (65 mL), charged with activated molecular sieves, was added triethylamine (4.6 mL, 35 mmol). The reaction mixture was vigorously stirred under air atmosphere until complete conversion monitored by LCMS. The solvent was distillated off under reduced pressure and the residue was purified by flash chromatography to afford 4-(4-methyl-3-nitrophenoxy)benzaldehyde OR0143-6 (0.57 g, 34%) as a light yellow powder. ¹H NMR (400 MHz, CDCl₃) δ 9.96 (s, 1H), 7.90 (d, J=8.6 Hz, 2H), 7.70 (d, J=2.5 Hz, 1H), 7.38 (d, J=8.4 Hz, 1H), 7.25 (dd, J=8.4, 2.5 Hz, 1H), 7.11 (d, J=8.6 Hz, 2H), 2.61 (s, 3H); LCMS C₁₄H₁₁NO₄ method (A) Rₜ=6.953 min, ESI+ m/z=258.1 (M+H).

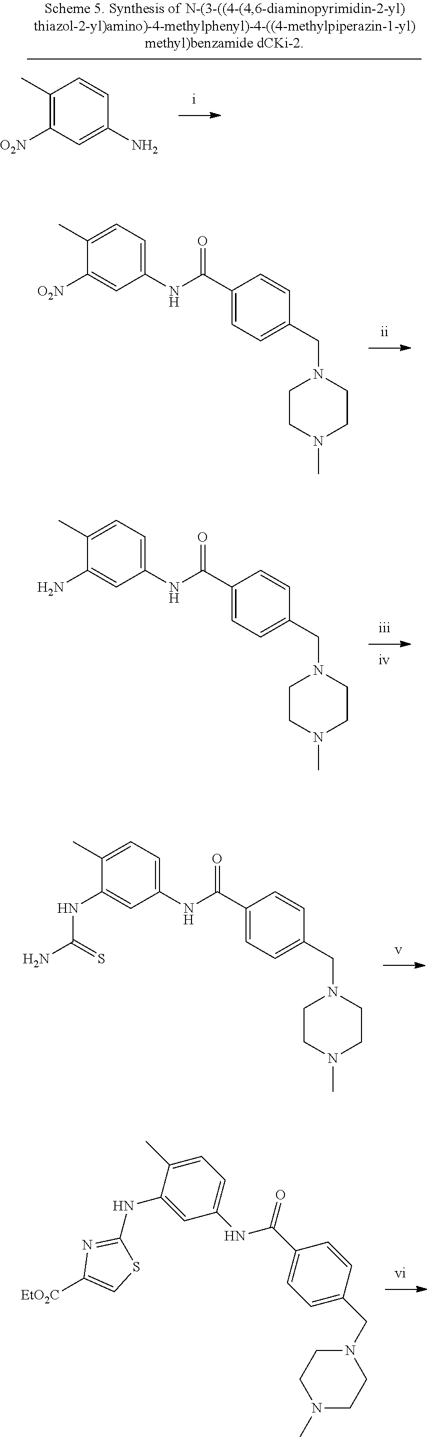 [SMILES: Cc1ccc(N)cc1[N+](=O)[O-] ; Cc1ccc(NC(=O)c2ccc(CN3CCN(C)CC3)cc2)cc1[N+](=O)[O-] ; Cc1ccc(NC(=O)c2ccc(CN3CCN(C)CC3)cc2)cc1N ; Cc1ccc(NC(=O)c2ccc(CN3CCN(C)CC3)cc2)cc1NC(N)=S ; CCOC(=O)c1csc(Nc2cc(NC(=O)c3ccc(CN4CCN(C)CC4)cc3)ccc2C)n1]

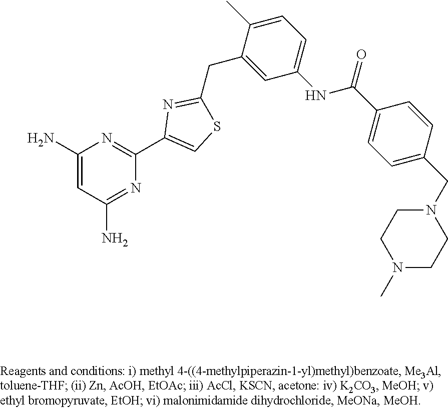 [SMILES: Cc1ccc(NC(=O)c2ccc(CN3CCN(C)CC3)cc2)cc1Cc1nc(-c2nc(N)cc(N)n2)cs1]

The condensation of commercially available 4-methyl-3-nitroaniline and methyl 4-((4-methylpiperazin-1-yl)methyl)benzoate was carried out in the presence of trimethylaluminum, to yield the corresponding amide quantitatively. After reduction of the nitro group, the amine was subjected to acetylisothiocyanate, then saponification gave the corresponding thiourea. Ethyl bromopyruvate was engaged in a Hantzsch thiazole synthesis with the thiourea leading to the corresponding thiazole. Finally, tandem addition/cyclization with malonimidamide led to expected N-(3-((4-(4,6-diaminopyrimidin-2-yl)thiazol-2-yl)amino)-4-methylphenyl)-4-((4-methylpiperazin-1-yl)methyl)benzamide dCKi-2.

N-(3-((4-(4,6-Diaminopyrimidin-2-yl)thiazol-2-yl)amino)-4-methylphenyl)-4-((4-methylpiperazin-1-yl)methyl)benzamide dCKi-2

To a suspension of ethyl 2-((2-methyl-5-(4-((4-methylpiperazin-1-yl)methyl)benzamido)phenyl)amino)thiazole-4-carboxylate dCKi-2-1 (2.4 g, 4.8 mmol), malonimidamide dihydrochloride (2.5 g, 14.4 mmol) in methanol (50 mL) was added sodium methoxide solution 25 wt. % in MeOH (14.9 mL, 65.2 mmol). The reaction mixture was heated at 70° C. for 2 hours and concentrated under vacuum. The crude product was triturated with THF (3×50 mL) and combined organic layers were dried over Na₂SO₄. The solvent was distillated off under reduced pressure and the residue was purified by flash chromatography, gradient DCM-MeOH—NH₄OH (100:0:0 to 90:9:1) to afford N-(3-((4-(4,6-diaminopyrimidin-2-yl)thiazol-2-yl)amino)-4-methylphenyl)-4-((4-methylpiperazin-1-yl)methyl)benzamide dCKi-2 (940 mg, 37%) as a light brown powder. Rf=0.10 (DCM-MeOH—NH₄OH, 92:7:1); ¹H NMR (300 MHz, DMSO-d₆) δ 10.22 (brs, 1H), 9.37 (brs, 1H), 8.84 (d, J=1.7 Hz, 1H), 7.90 (d, J=8.2 Hz, 2H), 7.51 (dd, J=8.3, 1.7 Hz, 1H), 7.42 (d, J=8.2 Hz, 2H), 7.31 (s, 1H), 7.20 (d, J=8.3 Hz, 1H), 6.10 (brs, 4H), 5.34 (s, 1H), 3.52 (s, 2H), 2.48-2.30 (m, 8H), 2.22 (s, 3H), 2.19 (s, 3H); ¹³C NMR (75 MHz, DMSO-d₆) δ 165.56, 165.27, 163.66, 159.29, 151.32, 142.01, 139.60, 137.78, 133.60, 130.66, 128.59, 127.59, 125.97, 116.39, 114.39, 109.48, 81.27, 61.45, 54.46, 52.23, 45.38, 17.41; LCMS C₂H₃₁N₉OS method (A) Rₜ=6.085 min, ESI+ m/z=530.2 (M+H).

Ethyl 2-((2-methyl-5-(4-((4-methylpiperazin-1-yl)methyl)benzamido)phenyl)amino)thiazole-4-carboxylate dCKi-2-1 To a stirred solution of dCKi-2-2 (4.0 g, 10.0 mmol) in ethanol (100 mL) was added ethyl bromopyruvate (1.5 mL, 12.0 mmol). The resulting mixture was refluxed for 3 hrs and then concentrated under reduced pressure. The residue was purified by flash chromatography, gradient DCM-MeOH (100:0 to 80:20) to afford ethyl 2-((2-methyl-5-(4-((4-methylpiperazin-1-yl)methyl)benzamido)phenyl)amino)thiazole-4-carboxylate dCKi-2-1 (4.2 g, 85%) as a light brown solid. Rf=0.32 (DCM-MeOH, 9:1); ¹H NMR (250 MHz, MeOD) δ 8.21 (d, J=2.1 Hz, 1H), 7.93 (d, J=8.2 Hz, 2H), 7.64 (s, 1H), 7.58 (dd, J=8.3, 2.1 Hz, 1H), 7.51 (d, J=8.2 Hz, 2H), 7.23 (d, J=8.3 Hz, 1H), 4.34 (q, J=7.1 Hz, 2H), 3.72 (s, 2H), 2.47-3.10 (m, 4H), 2.97-2.60 (m, 4H), 2.84 (s, 3H), 2.30 (s, 3H), 1.37 (t, J=7.1 Hz, 3H); ¹³C NMR (63 MHz, MeOD) δ 168.32, 168.21, 163.33, 163.33, 143.77, 142.36, 140.26, 138.70, 135.52, 132.06, 130.31, 128.87, 127.00, 119.17, 118.19, 115.40, 62.29, 62.19, 54.92, 51.12, 50.02, 49.68, 49.34, 49.00, 48.66, 48.32, 47.98, 43.74, 17.60, 14.58; LCMS C₂₆H₃₁N₅O₃S method (A) R=6.325 min, ESI+m/z=494.0 (M+H).

N-[3-(Carbamothioylamino)-4-methyl-phenyl]-4-[(4-methylpiperazin-1-yl)methyl]benzamide dCKi-2-2

As previously described for the synthesis of 1-(3-nitroaryl)thiourea derivatives, (94%) as a light brown solid, which was used directly in the next step without further purification. Rf=0.04 (DCM-MeOH 90:10); ¹H NMR (250 MHz, DMSO-d₆) δ 10.17 (brs, 1H), 9.32 (brs, 1H), 7.89 (d, J=8.1 Hz, 2H), 7.66 (d, J=2.0 Hz, 1H), 7.62 (dd, J=8.3, 2.0 Hz, 1H), 7.43 (d, J=8.1 Hz, 2H), 7.20 (d, J=8.3 Hz, 1H), 3.52 (s, 2H), 2.35 (brs, 8H), 2.14 (s, 6H); ¹³C NMR (63 MHz, DMSO-d₆) δ 181.46, 165.27, 142.23, 137.59, 137.00, 133.61, 130.43, 129.65, 128.65, 127.58, 119.25, 118.54, 61.62, 54.72, 52.60, 45.76, 17.13; LCMS C₂₁H₂₇N₅O₂S method (A) Rₜ=6.259 min, ESI+ m/z=398.0 (M+H).

N-[3-(Acetylcarbamothioylamino)-4-methyl-phenyl]-4-[(4-methylpiperazin-1-yl)methyl]benzamide dCKi-2-3

As previously described for the synthesis of N-((3-nitroaryl)carbamothioyl)acetamide derivatives, (59%) as a light brown solid, which was used directly in the next step without further purification. Rf=0.12 (DCM-MeOH 90:10); ¹H NMR (250 MHz, DMSO-d₆) δ 12.13 (brs, 1H), 11.52 (brs, 1H), 10.27 (brs, 1H), 7.99 (d, J=1.7 Hz, 1H), 7.94 (d, J=8.2 Hz, 2H), 7.62 (dd, J=8.1, 1.7 Hz, 1H), 7.47 (d, J=8.2 Hz, 2H), 7.25 (d, J=8.1 Hz, 1H), 3.65 (s, 2H), 3.40 (brs, 4H), 3.10 (brs, 4H), 2.70 (s, 3H), 2.17 (s, 6H); ¹³C NMR (63 MHz, DMSO-d₆) δ 179.75, 172.77, 165.15, 140.92, 137.30, 136.65, 133.84, 130.30, 128.87, 128.63, 127.74, 119.11, 118.48, 60.39, 52.75, 49.41, 42.35, 23.79, 17.13; LCMS C₂₃H₂₉N₅O₂S method (A) Rₜ=6.074 min, ESI+ m/z=440.0 (M+H).

N-(3-Amino-4-methylphenyl)-4-((4-methylpiperazin-1-yl)methyl)benzamide dCKi-2-4

To a solution of dCKi-2-5 (9.3 g, 25.3 mmol) in a mixture of ethyl acetate and acetic acid (2:1, 300 mL), zinc dust (24.4 g, 25.3 mmol) was added carefully. The resulting mixture was stirred at room temperature for 3 hrs, then filtered through Celite pad and washed with EtOAc (200 mL). The filtrate was concentrated under reduced pressure and the crude residue was successively dissolved in water, then Na₂CO₃ was added until pH=9, and extracted with EtOAc. The combined organic layer were dried over Na₂SO₄, and the solvent was distillated off under reduced pressure to afford N-(3-amino-4-methylphenyl)-4-((4-methylpiperazin-1-yl)methyl)benzamide dCKi-2-4 (8.5 g, quantitative) as a light brown solid, used in the next step without further purification. Rf=0.11 (DCM-MeOH—NH₄OH 92:7:1); ¹H NMR (250 MHz, DMSO-d₆, δ) 9.86 (brs, 1H), 7.86 (d, J=8.0 Hz, 2H), 7.40 (d, J=8.0 Hz, 2H), 7.11 (d, J=1.4 Hz, 1H), 6.91-6.73 (m, 2H), 4.83 (brs, 2H), 3.52 (s, 2H), 2.47-2.25 (brs, 8H), 2.19 (s, 3H), 2.01 (s, 3H); LCMS C₂₀H₂₆N₄O method (A) Rₜ=4.346 min, ESI+ m/z=339.0 (M+H).

N-(4-Methyl-3-nitrophenyl)-4-((4-methylpiperazin-1-yl)methyl)benzamide dCKi-2-5

Under argon, at 0° C., to a stirred solution of 4-methyl-3-nitroaniline (4.2 g, 27.6 mmol) and methyl 4-((4-methylpiperazin-1-yl)methyl)benzoate (6.3 g, 25.3 mmol) in a mixture of toluene-tetrahydrofuran (3:1, 320 mL) was added dropwise a 2M solution of trimethylaluminium in toluene (40 mL, 80 mmol). The resulting mixture was allowed to warm to room temperature and heated to 50° C. for 6 hrs. The mixture was poured carefully onto ice (200 g) and quenched by the addition of 10% NaOH aqueous solution (150 mL). After stirring for 30 min, the aqueous phase was extracted with DCM (3×200 mL). The combined organic layers were concentrated to dryness under reduced pressure to afford N-(4-methyl-3-nitrophenyl)-4-((4-methylpiperazin-1-yl)methyl)benzamide dCKi-2-5 (9.3 g, quantitative) as a light brown solid, used in the next step without further purification. Rf=0.27 (DCM-MeOH—NH₄OH 92:7:1); ¹H NMR (250 MHz, CDCl₃) δ 8.25 (d, J=2.3 Hz, 1H), 8.04 (brs, 1H), 7.91 (dd, J=8.3, 2.3 Hz, 2H), 7.83 (d, J=8.2 Hz, 2H), 7.47 (d, J=8.2 Hz, 2H), 7.34 (d, J=8.3 Hz, 1H), 3.58 (s, 2H), 2.59 (s, 3H), 2.70-2.25 (m, 8H), 2.30 (s, 3H); ¹³C NMR (63 MHz, CDCl₃) δ 166.03, 149.07, 143.32, 137.06, 133.33, 132.91, 129.49, 129.33, 127.25, 124.93, 116.28, 62.56, 55.18, 53.22, 46.14, 20.13; LCMS C₂₀H₂₄N₄O₃ method (A) Rₜ=6.479 min, ESI+ m/z=369.0 (M+H).

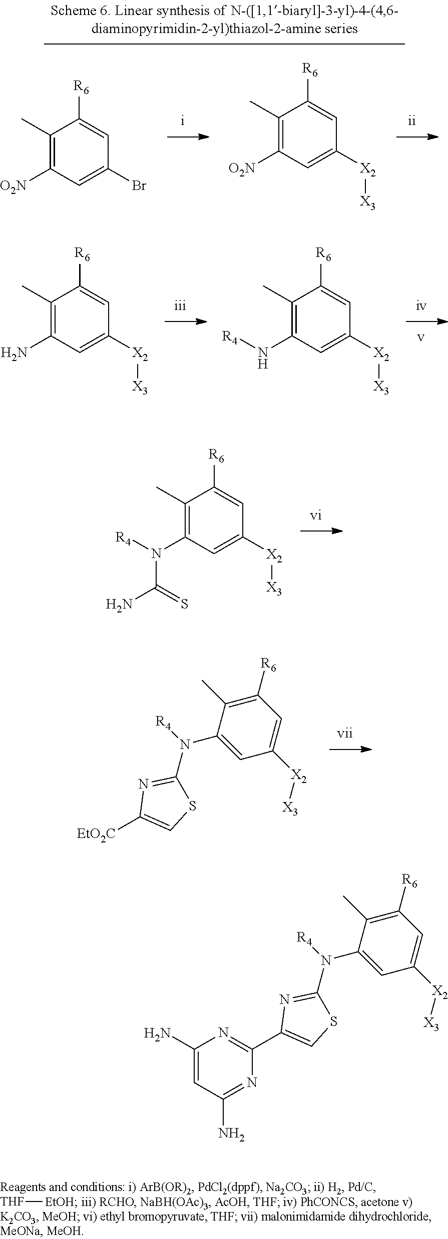

N-([1,1′-biaryl]-3-yl)-4-(4,6-diaminopyrimidin-2-yl)thiazol-2-amine derivatives were prepared in seven steps (Scheme 6). Starting from appropriate bromo-3-nitrobenzene, a Suzuki cross-coupling reaction with arylboronic acid derivatives allowed introducing the key biphenyl scaffold. Reduction of the nitro group successively followed by alkylation, then condensation with benzoyl isothiocyanate and saponification gave the corresponding thiourea. The ethyl bromopyruvate was engaged in a Hantzsch thiazole synthesis with the thiourea leading to the corresponding thiazole. Finally, tandem addition/cyclization with malonimidamide led to expected structurally diverse N-([1,1′-biaryl]-3-yl)-4-(4,6-diaminopyrimidin-2-yl)thiazol-2-amine.

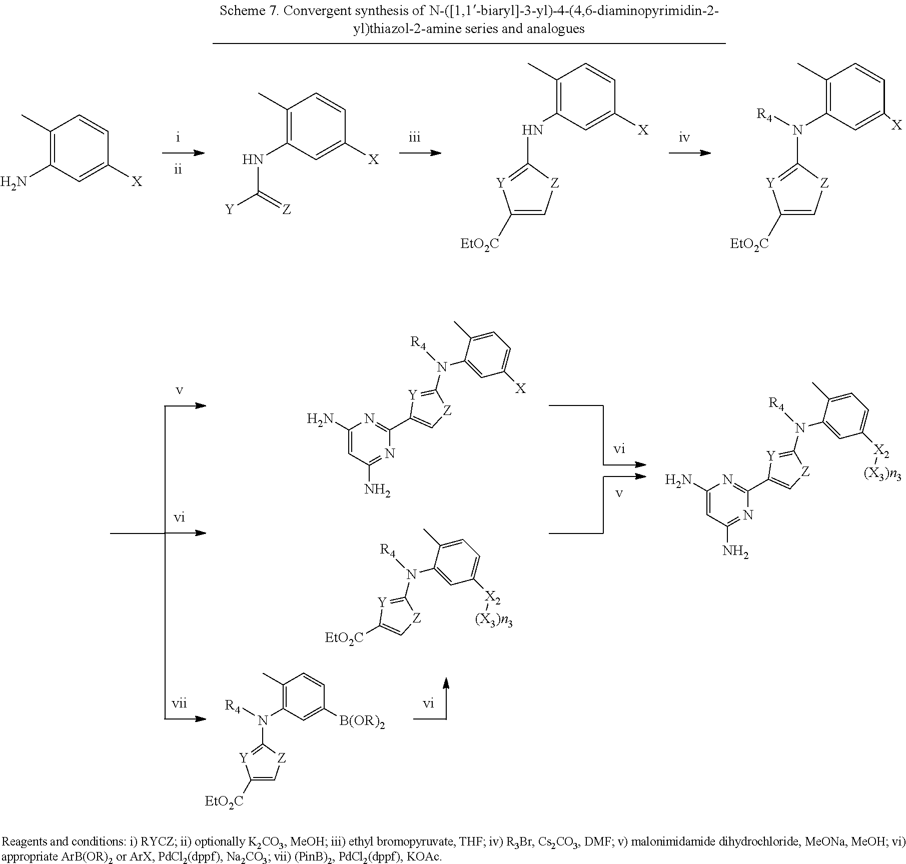

Alternatively, N-([1,1′-biaryl]-3-yl)-4-(4,6-diaminopyrimidin-2-yl)thiazol-2-amine derivatives and analogues, were prepared by convergent approach from commercially available aminohalogenobenzene (Scheme 7). For instance, starting from 5-bromo- or 5-iodo-2-methylaniline, condensation with benzoyl isothiocyanate and saponification gave the corresponding thiourea. Ethyl bromopyruvate was engaged in a Hantzsch thiazole synthesis with the thiourea leading to the corresponding thiazole. The thiazole derivative was then turned into expected N-([1,1′-biaryl]-3-yl)-4-(4,6-diaminopyrimidin-2-yl)thiazol-2-amine by Suzuki cross-coupling reaction via either arylboronate or halogenoaryl precursors.

General Procedure for the Synthesis of 2-[2-(biaryl-3-ylamino)-thiazol-4-yl]-pyrimidine-4,6-diamine hydrochloride salt. To a solution of appropriate 2-[2-(biaryl-3-ylamino)-thiazol-4-yl]-pyrimidine-4,6-diamine (1.5 mmol) in a mixture of methanol-dichloromethane (5:2, 35 mL) was added dropwise a 1N hydrogen chloride solution in diethyl ether (3.2 mL, 3.2 mmol). The resulting solution was stirred at room temperature for 30 minutes and concentrated under reduced pressure until few volumes. The residue was crystallized from Et₂O, under vigorous stirring, to afford expected 2-[2-(biaryl-3-ylamino)-thiazol-4-yl]-pyrimidine-4,6-diamine hydrochloride salt.

2-(2-((4-Methyl-4′-((4-methylpiperazin-1-yl)sulfonyl)-2′-(trifluoromethyl)-[1,1′-biphenyl]-3-yl)(propyl)amino) thiazol-4-yl)pyrimidine-4,6-diamine hydrochloride salt OR0642 (93%) as a light yellow powder. H NMR (400 MHz, MeOD) δ 8.17 (s, 1H), 8.15 (dd, J=8.1, 1.7 Hz, 1H), 7.74 (d, J=8.0 Hz, 1H), 7.69 (s, 1H), 7.57 (d, J=8.0 Hz, 1H), 7.41 (dd, J=7.9, 1.6 Hz, 1H), 7.34 (s, 1H), 5.67 (s, 1H), 4.28 (brs, 1H), 4.01 (brs, 2H), 3.85 (brs, 1H), 3.61 (brs, 2H), 3.30 (brs, 2H), 2.93 (brs, 2H), 2.92 (s, 3H), 2.34 (s, 3H), 1.72 (sext, J=7.4 Hz, 2H), 1.02 (t, J=7.4 Hz, 3H). ¹³C NMR (100 MHz, MeOD) δ 172.73, 152.34, 146.43, 144.00, 143.15, 139.26, 138.98, 136.95, 135.12, 133.51, 132.38, 130.94, 130.62, 126.74, 126.04, 123.32, 115.99, 80.46, 54.70, 53.95, 44.53, 43.49, 22.09, 17.51, 11.75.

General Procedure for the Synthesis of 2-[2-(biaryl-3-ylamino)-thiazol-4-yl]-pyrimidine-4,6-diamine and analogues. Method (A): as previously described for synthesis of dCKi-2, briefly to a suspension of ethyl 2-([1,1′-aryl]-3-yl-amino)thiazole-4-carboxylate derivative or analogue (0.76 mmol), malonimidamide dihydrochloride (225 mg, 1.28 mmol) in methanol (8 mL) was added sodium methoxide solution 25 wt. % in MeOH (1.56 mL, 6.80 mmol). The reaction mixture was heated at 70° C. for 4 hours and concentrated under vacuum. The crude product was triturated with THF (3×50 mL) and combined organic layers were dried over Na₂SO₄. The solvent was distillated off under reduced pressure and the residue was purified by flash chromatography to afford the corresponding 2-[2-(biaryl-3-ylamino)-thiazol-4-yl]-pyrimidine-4,6-diamine or analogue. Method (B): under argon, a suspension of appropriate 2-(2-(5-halogenophenylxamino)thiazol-4-yl)pyrimidine-4,6-diamine (0.214 mmol), arylboronate or arylboronic acid (0.286 mmol), PdCl₂(dppf) (21 mg, 0.028 mmol) and K₂CO₃ (103 mg, 0.745 mmol) in a degassed mixture of 1,4-dioxane-water (5:1, 12 mL) was heated at 80° C. for 1.5 hrs, upon complete consumption of starting material. The solvent was distillated off under reduced pressure and the residue purified by flash chromatography to afford corresponding 2-[2-(biaryl-3-ylamino)-thiazol-4-yl]-pyrimidine-4,6-diamine. Method (C): from appropriate tert-butyl(biaryl-3-yl-[4-(4,6-diamino-pyrimidin-2-yl)-thiazol-2-yl]-aminoalkyl)carbamate, as previously described for the synthesis of N-(3-((4-(4-aminopyrimidin-2-yl)thiazol-2-yl)amino)-4-aryl)benzamide derivatives.

2-(2-((4-Methyl-4′-(2-(4-methylpiperazin-1-yl)ethyl)-[1,1′-biphenyl]-3-yl)(propyl)amino)thiazol-4-yl)pyrimidine-4,6-diamine OR0600 (method A, 25%) as a light brown solid. Rf=0.25 (DCM-MeOH—NH₄OH, 90:9:1); ¹H NMR (400 MHz, CDCl₃) δ 7.52 (dd, J=8.2, 2.0 Hz, 1H), 7.50 (d, J=8.4 Hz, 2H), 7.45 (d, J=2.0 Hz, 1H), 7.38 (d, J=8.2 Hz, 1H), 7.31 (s, 1H), 7.27 (d, J=8.4 Hz, 2H), 5.42 (s, 1H), 4.71 (brs, 4H), 4.01 (brs, 2H), 2.86-2.81 (m, 2H), 2.66-2.61 (m, 2H), 2.50 (brs, 8H), 2.30 (s, 3H), 2.26 (s, 3H), 1.66 (sext, J=7.4 Hz, 2H), 0.92 (t, J=7.4 Hz, 3H); ¹³C NMR (100 MHz, CDCl₃) δ 171.22, 163.93, 160.87, 151.29, 143.34, 140.77, 139.91, 137.91, 136.29, 132.50, 129.38, 128.02, 127.00, 126.96, 110.80, 83.01, 60.52, 55.27, 53.62, 53.30, 46.20, 33.35, 21.33, 17.49, 11.42; LCMS C₃₀H₃₈N₈S method (B) R_{c}=4.243 min, ESI+ m/z=543.3 (M+H).

2-(2-((4-Methyl-4′-(3-(4-methylpiperazin-1-yl)propyl)-[1,1′-biphenyl]-3-yl)(propyl)amino)thiazol-4-yl)pyrimidine-4,6-diamine OR0601 (method A, 40%) as a light yellow solid. Rf=0.25 (DCM-MeOH—NH₄OH, 90:9:1); ¹H NMR (400 MHz, CDCl₃) δ 7.52 (dd, J=8.0, 1.8 Hz, 1H), 7.49 (d, J=8.1 Hz, 2H), 7.45 (d, J=1.8 Hz, 1H), 7.38 (d, J=8.0 Hz, 1H), 7.31 (s, 1H), 7.25 (d, J=8.1 Hz, 2H), 5.42 (s, 1H), 4.82 (brs, 4H), 4.01 (brs, 2H), 2.66 (t, J=7.7 Hz, 2H), 2.51 (brs, 8H), 2.44-2.38 (m, 2H), 2.31 (s, 3H), 2.25 (s, 3H), 1.85 (quint, J=7.7 Hz, 2H), 1.66 (sext, J=7.5 Hz, 2H), 0.92 (t, J=7.5 Hz, 3H); ¹³C NMR (100 MHz, CDCl₃) δ 171.19, 163.80, 160.57, 151.07, 143.31, 141.64, 140.80, 137.61, 136.18, 132.48, 129.06, 127.93, 126.92, 110.87, 82.95, 57.95, 55.10, 53.67, 53.07, 46.02, 33.41, 28.61, 21.32, 17.49, 11.41. 1; LCMS C₃₁H₄₀N₈S method (B) R_{c}=4.279 min, ESI+ m/z=557.3 (M+H).

2-(2-((2-Methyl-5-(6-(2-(4-methylpiperazin-1-yl)ethyl)pyridin-3-yl)phenyl)(propyl)amino)thiazol-4-yl)pyrimidine-4,6-diamine OR0602 (method A, 34%) as a white powder. Rf=0.25 (DCM-MeOH—NH₄OH, 90:9:1); ¹H NMR (400 MHz, CDCl₃) δ 8.74 (d, J=2.0 Hz, 1H), 7.77 (dd, J=8.1, 2.0 Hz, 1H), 7.51 (dd, J=7.9, 1.8 Hz, 1H), 7.44 (d, J=1.8 Hz, 1H), 7.43 (d, J=7.9 Hz, 1H), 7.32 (s, 1H), 7.25 (d, J=8.1 Hz, 1H), 5.44 (s, 1H), 4.71 (brs, 4H), 4.01 (brs, 2H), 3.05-3.00 (m, 2H), 2.82-2.77 (m, 2H), 2.60 (brs, 4H), 2.48 (brs, 4H), 2.29 (s, 3H), 2.28 (s, 3H), 1.66 (sext, J=7.4 Hz, 2H), 0.93 (t, J=7.4 Hz, 3H); ¹³C NMR (100 MHz, CDCl₃) δ 171.00, 163.92, 160.82, 159.60, 151.33, 147.56, 143.64, 137.64, 137.33, 134.71, 133.18, 132.87, 128.11, 126.94, 123.29, 110.86, 83.02, 58.44, 55.26, 53.65, 53.19, 46.17, 35.53, 21.36, 17.57, 11.41; LCMS C₂₉H₃₇N₉S method (B) Rₜ=3.952 min, ESI+ m/z=544.3 (M+H).

2-(2-(iso-Butyl(4-methyl-4′-(2-(4-methylpiperazin-1-yl)ethyl)-[1,1′-biphenyl]-3-yl)amino)thiazol-4-yl)pyrimidine-4,6-diamine OR0603 (method A, 23%) as a light brown powder. Rf=0.5 (DCM-MeOH—NH₄OH, 90:9:1); ¹H NMR (400 MHz, CDCl₃) δ 7.52 (dd, J=7.6, 1.9 Hz, 1H), 7.50 (d, J=1.9 Hz, 1H), 7.49 (d, J=8.2 Hz, 2H), 7.38 (d, J=7.6 Hz, 1H), 7.32 (s, 1H), 7.28 (d, J=8.2 Hz, 2H), 5.41 (s, 1H), 4.70 (brs, 4H), 3.84 (brs, 2H), 2.88-2.80 (m, 2H), 2.67-2.61 (m, 2H), 2.56 (brs, 8H), 2.30 (s, 3H), 2.26 (s, 3H), 2.04 (sept, J=6.8 Hz, 1H), 1.00 (d, J=6.8 Hz, 6H). ¹³C NMR (100 MHz, CDCl₃) δ 171.69, 163.92, 160.89, 151.35, 144.07, 140.59, 139.89, 138.00, 135.94, 132.71, 129.39, 127.87, 127.03, 126.81, 111.00, 83.02, 60.53, 59.79, 55.27, 53.30, 46.19, 33.35, 27.43, 20.78, 17.73; LCMS C₃₁H₄₀N₈S method (B) Rₜ=4.368 min, ESI+ m/z=557.2 (M+H).

2-(2-((Cyclopropylmethyl)(4-methyl-4′-(2-(4-methylpiperazin-1-yl)ethyl)-[1,1′-biphenyl]-3-yl)amino)thiazol-4-yl)pyrimidine-4,6-diamine OR0604 (method A, 8%) as a light brown powder. Rf=0.5 (DCM-MeOH—NH₄OH, 90:9:1); ¹H NMR (400 MHz, CDCl₃) δ 7.53 (dd, J=8.7, 1.9 Hz, 1H), 7.52 (d, J=1.9 Hz, 1H), 7.50 (d, J=8.2 Hz, 2H), 7.38 (d, J=8.7 Hz, 1H), 7.31 (s, 1H), 7.28 (d, J=8.2 Hz, 2H), 5.44 (s, 1H), 4.68 (brs, 4H), 3.96 (brs, 2H), 2.89-2.80 (m, 2H), 2.68-2.60 (m, 2H), 2.53 (brs, 8H), 2.32 (s, 3H), 2.29 (s, 3H), 1.17-1.05 (m, 1H), 0.44-0.37 (m, 2H), 0.19-0.11 (m, 2H); ¹³C NMR (100 MHz, CDCl₃) δ 171.25, 163.90, 160.90, 151.20, 143.55, 140.63, 139.80, 138.01, 136.48, 132.27, 129.37, 128.44, 126.97, 110.95, 83.01, 60.48, 56.51, 55.21, 53.19, 46.12, 33.32, 17.48, 9.88, 3.97; LCMS C₃₁H₃₈N₈S method (B) Rₜ=4.235 min, ESI+ m/z=555.3 (M+H).

2-(2-(iso-Pentyl(4-methyl-4′-(2-(4-methylpiperazin-1-yl)ethyl)-[1,1′-biphenyl]-3-yl)amino)thiazol-4-yl)pyrimidine-4,6-diamine OR0605 (method A, 28%) as a light yellow solid. Rf=0.5 (DCM-MeOH—NH₄OH, 90:9:1); ¹H NMR (400 MHz, CDCl₃) δ 7.52 (dd, J=7.9, 1.8 Hz, 1H), 7.50 (d, J=8.2 Hz, 2H), 7.43 (d, J=1.8 Hz, 1H), 7.38 (d, J=7.9 Hz, 1H), 7.33 (s, 1H), 7.27 (d, J=8.2 Hz, 2H), 5.45 (s, 1H), 4.85 (brs, 4H), 4.07 (brs, 2H), 2.90-2.82 (m, 2H), 2.73-2.66 (m, 2H), 2.65 (brs, 8H), 2.39 (s, 3H), 2.25 (s, 3H), 1.71-1.59 (m, 1H), 1.50 (q, J=7.3 Hz, 2H), 0.90 (d, J=6.6 Hz, 6H); ¹³C NMR (100 MHz, CDCl₃) δ 171.26, 163.56, 143.30, 140.69, 139.52, 138.03, 136.34, 132.51, 129.38, 127.90, 127.05, 126.97, 111.15, 82.88, 60.12, 54.82, 52.55, 50.66, 45.67, 36.64, 33.12, 26.43, 22.83, 17.54; LCMS C₃₂H₄₀N₈S method (B) Rₜ=4.452 min, ESI+ m/z=571.4 (M+H).

2-(2-(Butyl(4-methyl-4′-(2-(4-methylpiperazin-1-yl)ethyl)-[1,1′-biphenyl]-3-yl)amino)thiazol-4-yl)pyrimidine-4,6-diamine OR0606 (method A, 19%) as a light brown solid. Rf=0.5 (DCM-MeOH—NH₄OH, 90:9:1); ¹H NMR (400 MHz, CDCl₃) δ 7.53 (dd, J=8.0, 1.7 Hz, 1H), 7.50 (d, J=8.2 Hz, 2H), 7.44 (d, J=1.7 Hz, 1H), 7.38 (d, J=8.0 Hz, 1H), 7.32 (s, 1H), 7.27 (d, J=8.2 Hz, 2H), 5.45 (s, 1H), 4.82 (brs, 4H), 4.05 (brs, 2H), 2.87-2.83 (m, 2H), 2.69-2.65 (m, 2H), 2.57 (brs, 8H), 2.37 (s, 3H), 2.25 (s, 3H), 1.60-1.58 (m, 2H), 1.38-1.35 (m, 2H), 0.89 (t, J=7.3 Hz, 3H); ¹³C NMR (100 MHz, CDCl₃) δ 171.28, 163.65, 150.79, 143.30, 140.73, 139.64, 137.99, 136.32, 132.52, 129.38, 127.98, 127.03, 126.98, 111.06, 82.91, 60.24, 52.78, 51.86, 45.84, 33.20, 30.17, 29.84, 20.34, 17.51, 14.12; LCMS C₃₁H₄₀N₈S method (B) Rₜ=4.394 min, ESI+ m/z=557.3 (M+H).

2-(2-((4-Methyl-4′-((4-methylpiperazin-1-yl)sulfonyl)-[1,1′-biphenyl]-3-yl)(propyl)amino)thiazol-4-yl)pyrimidine-4,6-diamine OR0607 (method B, 76%) as a light brown solid. Rf=0.45 (DCM-MeOH—NH₄OH, 90:9:1); ¹H NMR (400 MHz, MeOD) δ 7.89 (d, J=8.7 Hz, 2H), 7.85 (d, J=8.7 Hz, 2H), 7.71 (dd, J=8.0, 1.8 Hz, 1H), 7.66 (d, J=1.8 Hz, 1H), 7.54 (d, J=8.0 Hz, 1H), 7.25 (s, 1H), 5.54 (s, 1H), 4.01 (brs, 2H), 3.08-3.04 (m, 4H), 2.53-2.49 (m, 4H), 2.31 (s, 3H), 2.26 (s, 3H), 1.74 (sext, J=7.4 Hz, 2H), 0.99 (t, J=7.4 Hz, 3H); ¹³C NMR (100 MHz, MeOD) δ 171.99, 165.62, 161.25, 152.08, 145.86, 145.04, 140.31, 139.04, 135.59, 134.14, 129.71, 129.44, 128.58, 128.51, 111.01, 82.96, 55.09, 54.87, 46.95, 45.75, 22.31, 17.61, 11.61; LCMS C₂₈H₃₄N₈O₂S₂ method (B) Rₜ=4.496 min, ESI+ m/z=579.3 (M+H).

2-(2-(iso-Butyl(2-methyl-5-(6-(2-(4-methylpiperazin-1-yl)ethoxy)pyridin-3-yl)phenyl)amino)thiazol-4-yl)pyrimidine-4,6-diamine OR0608 (method A, 9%) as a light yellow solid. Rf=0.5 (DCM-MeOH—NH₄OH, 90:9:1); ¹H NMR (400 MHz, CDCl₃) δ 8.34 (d, J=2.5 Hz, 1H), 7.77 (dd, J=8.6, 2.5 Hz, 1H), 7.46 (dd, J=7.8, 1.7 Hz, 1H), 7.43 (d, J=1.7 Hz, 1H), 7.40 (d, J=7.8 Hz, 1H), 7.35 (s, 1H), 6.83 (d, J=8.6 Hz, 1H), 5.50 (s, 1H), 5.04 (brs, 4H), 4.49 (t, J=5.7 Hz, 2H), 3.80 (brs, 2H), 2.87 (t, J=5.7 Hz, 2H), 2.80-2.60 (m, 8H), 2.43 (s, 3H), 2.26 (s, 3H), 2.10-1.96 (m, 1H), 1.00 (d, J=6.6 Hz, 6H). ¹³C NMR (100 MHz, CDCl₃) δ 171.42, 163.70, 163.30, 160.26, 151.03, 144.88, 144.16, 137.42, 137.33, 136.17, 132.96, 129.13, 127.42, 126.42, 111.36, 111.04, 82.90, 63.71, 59.87, 57.15, 55.06, 53.43, 46.02, 27.42, 20.67, 17.73; LCMS C₃₀H₃₉N₉OS method (B) Rₜ=5.303 min, ESI+ m/z=574.3 (M+H).

N-((1-(3-((4-(4,6-Diaminopyrimidin-2-yl)thiazol-2-yl)(isobutyl)amino)-4-methylphenyl)-1H-1,2,3-triazol-4-yl)methyl) methanesulfonamide OR0609 (method A, 18%) as a light brown powder. Rf=0.5 (DCM-MeOH—NH₄OH, 90:9:1); ¹H NMR (400 MHz, MeOD) δ 8.51 (s, 1H), 7.88 (d, J=2.2 Hz, 1H), 7.82 (dd, J=8.3, 2.2 Hz, 1H), 7.60 (d, J=8.3 Hz, 1H), 7.30 (s, 1H), 5.53 (s, 1H), 4.45 (s, 2H), 3.91 (brs, 2H), 2.99 (s, 3H), 2.33 (s, 3H), 2.08-1.96 (m, 1H), 1.03 (d, J=6.7 Hz, 6H); ¹³C NMR (100 MHz, MeOD) δ 172.12, 165.62, 161.25, 152.19, 147.17, 145.86, 139.43, 137.65, 134.79, 122.65, 122.30, 121.25, 111.43, 83.02, 60.94, 40.62, 39.00, 28.74, 20.78, 17.85; LCMS C₂₂H₂₈N₁₀O₂S₂ method (B) Rₜ=4.585 min, ESI+ m/z=529.2 (M+H).

2-(2-((2-Aminoethyl)(4-methyl-4′-(2-(4-methylpiperazin-1-yl)ethyl)-[1,1′-biphenyl]-3-yl)amino)thiazol-4-yl)pyrimidine-4,6-diamine OR0610 (method C, 74%), as a pale yellow solid. Rf=0.40 (DCM-MeOH—NH₄OH, 80:18:2); ¹H NMR (400 MHz, CDCl₃) δ 7.49-7.47 (m, 2H), 7.43 (d, J=8.1 Hz, 2H), 7.31 (d, J=8.5 Hz, 1H), 7.25 (s, 1H), 7.20 (d, J=8.1 Hz, 2H), 5.71 (brs, 4H), 4.16 (brs, 1H), 3.80 (brs, 1H), 3.48-3.20 (m, 2H), 2.79-2.75 (m, 2H), 2.59-2.56 (m, 2H), 2.48 (brs, 8H), 2.33 (s, 3H), 2.14 (s, 3H); ¹³C NMR (100 MHz, CDCl₃) δ 170.65, 163.08, 162.63, 162.29, 143.80, 141.26, 139.88, 137.34, 134.63, 132.79, 129.41, 127.59, 126.98, 118.35, 115.44, 82.75, 60.07, 58.49, 54.87, 52.69, 45.72, 40.62, 33.08, 18.56, 17.41; LCMS C₂₉H₃₇N₉S method (B) Rₜ=3.649 min, ESI+ m/z=544.2 (M+H).

tert-Butyl (2-((4-(4,6-diaminopyrimidin-2-yl)thiazol-2-yl)(4-methyl-4′-(2-(4-methylpiperazin-1-yl)ethyl)-[1,1′-bi-phenyl]-3-yl)amino)ethyl)carbamate OR0610-1 (method A, 31%) as a light brown solid. Rf=0.33 (DCM-MeOH—NH₄OH, 90:9:1); LCMS C₃₄H₄₅N₉O₂S method (B) Rₜ=4.360 min, ESI+ m/z=644.4 (M+H).

2-(2-((3-Aminopropyl)(4-methyl-4′-(2-(4-methylpiperazin-1-yl)ethyl)-[1,1′-biphenyl]-3-yl)amino)thiazol-4-yl)pyrimidine-4,6-diamine OR0611 (method C, 50%) as a pale yellow solid. Rf=0.55 (DCM-MeOH—NH₄OH, 80:18:2); ¹H NMR (400 MHz, CDCl₃) δ 7.47 (dd, J=8.1, 1.2 Hz, 1H), 7.41 (d, J=8.2 Hz, 2H), 7.31 (d, J=8.1 Hz, 1H), 7.30 (d, J=1.2 Hz, 1H), 7.21 (d, J=8.2 Hz, 2H), 7.15 (s, 1H), 5.51 (brs, 4H), 5.29 (s, 1H), 4.15 (brs, 1H), 3.81 (brs, 1H), 3.18-3.16 (m, 2H), 2.84-2.76 (m, 2H), 2.63-2.53 (m, 2H), 2.47 (brs, 8H), 2.28 (s, 3H), 2.13 (s, 3H), 2.01-1.97 (m, 2H); ¹³C NMR (100 MHz, CDCl₃) δ 171.39, 164.09, 159.70, 150.42, 143.27, 141.36, 140.09, 137.45, 135.13, 132.80, 129.41, 127.47, 127.01, 110.34, 83.57, 60.45, 55.24, 53.56, 53.26, 50.17, 46.18, 33.31, 29.84, 17.44; LCMS C₃₀H₃₉N₉S method (B) Rₜ=3.671 min, ESI+ m/z=558.3 (M+H).

tert-Butyl (3-((4-(4,6-diaminopyrimidin-2-yl)thiazol-2-yl)(4-methyl-4′-(2-(4-methylpiperazin-1-yl)ethyl)-[1,1′-biphenyl]-3-yl)amino)propyl)carbamate OR0611-1 (method A, 39%) as a light brown solid. Rf=0.50 (DCM-MeOH—NH₄OH, 90:9:1); LCMS C₃₅H₄₇N₉O₂S method (B) Rₜ=4.450 min, ESI+ m/z=658.4 (M+H).

2-[2-({2-Methyl-5-[6-(4-methyl-piperazin-1-ylcarbonyl)-pyridin-3-yl]-phenyl}-propyl-amino)-thiazol-4-yl]-pyrimidine-4,6-diamine OR0612 (method A, 2%) as a light yellow solid. Rf=0.5 (DCM-MeOH—NH₄OH, 90:9:1); ¹H NMR (400 MHz, CDCl₃) δ 8.78 (d, J=2.0 Hz, 1H), 7.96 (dd, J=8.1, 2.0 Hz, 1H), 7.72 (d, J=8.1 Hz, 1H), 7.54 (dd, J=7.9, 1.4 Hz, 1H), 7.47 (d, J=1.4 Hz, 1H), 7.45 (d, J=7.9 Hz, 1H), 7.35 (s, 1H), 5.53 (s, 1H), 5.26 (brs, 4H), 3.98 (brs, 2H), 3.87-3.83 (m, 2H), 3.70-3.66 (m, 2H), 2.55-2.51 (m, 2H), 2.45-2.41 (m, 2H), 2.33 (s, 3H), 2.27 (s, 3H), 1.66 (sext, J=7.3 Hz, 2H), 0.93 (t, J=7.3 Hz, 3H); LCMS C₂₈H₃₃N₉OS method (B) Rₜ=4.071 min, ESI+ m/z=544.3 (M+H).

2-(2-((5-(2-Aminopyrimidin-5-yl)-2-methylphenyl)(propyl)amino)thiazol-4-yl)pyrimidine-4,6-diamine OR0613 (method B, 67%) as a light brown solid. Rf=0.45 (DCM-MeOH—NH₄OH, 95:4.5:0.5); ¹H NMR (400 MHz, MeOD) δ 8.54 (s, 2H), 7.54 (dd, J=8.0, 1.7 Hz, 1H), 7.50 (d, J=1.7 Hz, 1H), 7.47 (d, J=8.0 Hz, 1H), 7.29 (s, 1H), 5.54 (s, 1H), 3.98 (brs, 2H), 2.27 (s, 3H), 1.72 (sext, J=7.5 Hz, 2H), 0.97 (t, J=7.5 Hz, 3H); ¹³C NMR (100 MHz, MeOD) δ 200.27, 193.20, 192.10, 188.30, 185.51, 179.17, 173.11, 165.67, 164.59, 162.29, 155.80, 155.25, 152.22, 139.76, 110.84, 83.11, 50.40, 45.71, 39.78; LCMS C₂₁H23N9S method (B) Rₜ=4.620 min, ESI+ m/z=434.2 (M+H).

2-(2-((4-Methyl-4′-((4-methylpiperazin-1-yl)sulfonyl)-[1,1′-biphenyl]-3-yl)amino)thiazol-4-yl)pyrimidine-4,6-diamine OR0614 (method B, 35%) as a light yellow solid. Rf=0.22 (DCM-MeOH—NH₄OH, 90:9:1); ¹H NMR (400 MHz, CDCl₃) δ 7.74 (d, J=8.2 Hz, 2H), 7.67 (s, 1H), 7.62 (d, J=8.2 Hz, 2H), 7.40 (s, 1H), 7.23 (d, J=8.5 Hz, 1H), 7.19 (d, J=8.5 Hz, 1H), 5.39 (s, 1H), 5.13 (brs, 4H), 3.03-2.99 (m, 4H), 2.46-2.42 (m, 4H), 2.21 (s, 6H); ¹³C NMR (100 MHz, CDCl₃) δ 166.59, 163.71, 159.74, 150.31, 145.04, 139.70, 138.20, 133.70, 131.96, 130.67, 128.45, 127.45, 123.37, 119.60, 110.58, 83.12, 67.13, 58.30, 54.04, 46.02, 45.71, 18.47, 17.66; LCMS C₂₅H₂₈N₈O₂S₂ method (B) Rₜ=4.109 min, ESI+ m/z=537.2 (M+H).

3′-((4-(4,6-Diaminopyrimidin-2-yl)thiazol-2-yl)(propyl)amino)-4′-methyl-[1,1′-biphenyl]-4-sulfonamide OR0615 (method B, 36%) as a light brown solid. Rf=0.27 (DCM-MeOH—NH₄OH, 90:9:1); ¹H NMR (400 MHz, MeOD) δ 7.97 (d, J=8.4 Hz, 2H), 7.78 (d, J=8.4 Hz, 2H), 7.66 (dd, J=8.1, 1.8 Hz, 1H), 7.60 (d, J=1.8 Hz 1H), 7.50 (d, J=8.1 Hz, 1H), 7.29 (s, 1H), 5.55 (s, 1H), 3.98 (brs, 2H), 2.28 (s, 3H), 1.71 (sext, J=7.5 Hz, 2H), 0.97 (t, J=7.5 Hz, 3H); ¹³C NMR (100 MHz, MeOD) δ 172.10, 165.00, 160.09, 150.99, 144.82, 144.72, 143.93, 140.59, 138.61, 134.05, 129.19, 128.50, 128.26, 127.88, 111.66, 82.69, 54.91, 22.25, 17.60, 11.63; LCMS C₂₃H₂₅N₇O₂S₂ method (B) Rₜ=4.933 min, ESI+ m/z=496.2 (M+H).

N-(5-(3-((4-(4,6-Diaminopyrimidin-2-yl)thiazol-2-yl)(propyl)amino)-4-methylphenyl)pyridin-2-yl)-4-methylpiperazine-1-carboxamide OR0616 (method A, 19%) as a light yellow solid. Rf=0.5 (DCM-MeOH—NH₄OH, 90:9:1); ¹H NMR (400 MHz, DMSO-d₆) δ 9.28 (brs, 1H), 8.59 (d, J=2.3 Hz, 1H), 8.03 (dd, J=8.8, 2.3 Hz, 1H), 7.86 (d, J=8.8 Hz, 1H), 7.68 (dd, J=7.9, 1.8 Hz, 1H), 7.67 (d, J=1.8 Hz, 1H), 7.49 (d, J=7.9 Hz, 1H), 7.17 (s, 1H), 6.03 (brs, 4H), 5.34 (s, 1H), 3.89 (brs, 2H), 3.50-3.42 (m, 4H), 2.33-2.26 (m, 4H), 2.21 (s, 3H), 2.18 (s, 3H), 1.62 (sext, J=7.4 Hz, 2H), 0.90 (t, J=7.4 Hz, 3H); LCMS C₂₈H₃₄N₁₀OS method (B) Rₜ=4.237 min, ESI+ m/z=559.3 (M+H).

2-(2-((4-Methyl-4′-(morpholinosulfonyl)-[1,1′-biphenyl]-3-yl)(propyl)amino)thiazol-4-yl)pyrimidine-4,6-diamine OR0617 (method B, 61%) as a light yellow solid. Rf=0.50 (DCM-MeOH—NH₄OH, 90:9:1); ¹H NMR (400 MHz, MeOD) δ 7.90 (d, J=8.5 Hz, 2H), 7.85 (d, J=8.5 Hz, 2H), 7.71 (dd, J=8.0, 1.7 Hz, 1H), 7.67 (d, J=1.7 Hz, 1H), 7.54 (d, J=8.0 Hz, 1H), 7.26 (s, 1H), 5.54 (s, 1H), 4.02 (brs, 2H), 3.73-3.69 (m, 4H), 3.02-2.98 (m, 4H), 2.31 (s, 3H), 1.63 (sext, J=7.3 Hz, 2H), 0.99 (t, J=7.3 Hz, 3H); ¹³C NMR (100 MHz, MeOD) S 172.00, 165.60, 161.21, 152.04, 145.94, 145.04, 140.32, 139.04, 135.37, 134.13, 129.78, 129.46, 128.61, 128.55, 111.03, 82.95, 67.23, 54.90, 47.46, 22.31, 17.62, 11.61; LCMS C₂₇H₃₁N₇O₃S₂ method (B) Rₜ=5.538 min, ESI+ m/z=566.2 (M+H).

3′-((4-(4,6-Diaminopyrimidin-2-yl)thiazol-2-yl)(propyl)amino)-N,N,4′-trimethyl-[1,1′-biphenyl]-4-sulfonamide OR0618 (method B, 67%) as a light brown solid. Rf=0.55 (DCM-MeOH—NH₄OH, 90:9:1); ¹H NMR (400 MHz, MeOD) δ 7.88 (d, J=8.7 Hz, 2H), 7.85 (d, J=8.7 Hz, 2H), 7.71 (dd, J=7.9, 1.4 Hz, 1H), 7.66 (J=1.4 Hz, 1H), 7.53 (J=7.9 Hz, 1H), 7.25 (s, 1H), 5.54 (s, 1H), 4.02 (brs, 2H), 2.71 (s, 6H), 2.31 (s, 3H), 1.74 (sext, J=7.4 Hz, 2H), 0.99 (t, J=7.4 Hz, 3H). ¹³C NMR (100 MHz, MeOD) δ 172.00, 165.57, 161.16, 151.99, 145.63, 145.01, 140.38, 138.95, 135.44, 134.11, 132.26, 129.65, 129.41, 128.51, 111.05, 82.94, 54.88, 38.30, 22.30, 17.61, 11.61; LCMS C₂₅H₂₉N₇O₂S₂ method (B) Rₜ=5.395 min, ESI+ m/z=524.2 (M+H).

2-(2-((4′-((Dimethylamino)methyl)-4-methyl-[1,1′-biphenyl]-3-yl)(propyl)amino)thiazol-4-yl)pyrimidine-4,6-diamine OR0619 (method B, 61%) as a light yellow solid. Rf=0.50 (DCM-MeOH—NH₄OH, 90:9:1); ¹H NMR (400 MHz, MeOD) δ 7.63 (dd, J=8.0, 1.8 Hz, 1H), 7.60 (d, J=8.1 Hz, 2H), 7.54 (d, J=1.8 Hz, 1H), 7.47 (d, J=8.0 Hz, 1H), 7.40 (d, J=8.1 Hz, 2H), 7.23 (s, 1H), 5.54 (s, 1H), 4.01 (brs, 2H), 3.51 (s, 2H), 2.28 (s, 3H), 2.26 (s, 6H), 1.73 (sext, J=7.4 Hz, 2H), 0.99 (t, J=7.4 Hz, 3H); ¹³C NMR (100 MHz, MeOD) δ 172.18, 165.64, 161.34, 152.03, 144.75, 141.99, 140.42, 138.13, 137.33, 133.79, 131.38, 128.89, 128.18, 127.75, 110.91, 82.96, 64.53, 54.76, 45.22, 22.30, 17.50, 11.61; LCMS C₂₆H₃₁N₇S method (B) Rₜ=4.385 min, ESI+ m/z=474.3 (M+H).

2-(2-((2-Methyl-5-(pyridin-3-yl)phenyl)(propyl)amino)thiazol-4-yl)pyrimidine-4,6-diamine OR0620 (method B, 86%) as a light brown solid. Rf=0.50 (DCM-MeOH—NH₄OH, 90:9:1); ¹H NMR (400 MHz, MeOD) δ 8.81 (d, J=1.8 Hz, 1H), 8.52 (dd, J=4.9, 1.8 Hz, 1H), 8.10 (dt, J=8.0, 1.8 Hz, 1H), 7.67 (dd, J=7.8, 1.9 Hz, 1H), 7.63 (d, J=1.9 Hz, 1H), 7.55 (d, J=7.8 Hz, 1H), 7.52 (dd, J=8.0, 4.9 Hz, 1H), 7.25 (s, 1H), 5.54 (s, 1H), 4.01 (brs, 2H), 2.31 (s, 3H), 1.74 (sext, J=7.4 Hz, 2H), 0.99 (t, J=7.4 Hz, 3H); ¹³C NMR (100 MHz, MeOD) δ 171.98, 165.61, 161.25, 152.05, 149.00, 148.19, 145.12, 138.79, 138.49, 137.44, 136.37, 134.21, 129.17, 128.33, 125.57, 111.01, 82.96, 54.92, 22.30, 17.61, 11.60; LCMS C₂₂H23N7S method (B) Rₜ=4.544 min, ESI+ m/z=418.2 (M+H).

(5-(3-((4-(4,6-Diaminopyrimidin-2-yl)thiazol-2-yl)(propyl)amino)-4-methylphenyl)pyridin-2-yl)(morpholino) methanone OR0621 (method B, 71%) as a light yellow solid. Rf=0.50 (DCM-MeOH—NH₄OH, 90:9:1); ¹H NMR (400 MHz, MeOD) δ 8.88 (d, J=2.0 Hz, 1H), 8.21 (dd, J=8.2, 2.0 Hz, 1H), 7.73 (dd, J=8.0, 1.8 Hz, 1H), 7.72 (d, J=8.2 Hz, 1H), 7.69 (d, J=1.8 Hz, 1H), 7.56 (d, J=8.0 Hz, 1H), 7.25 (s, 1H), 5.53 (s, 1H), 4.02 (brs, 2H), 3.79 (brs, 4H), 3.70-3.64 (m, 2H), 3.62-3.56 (m, 2H), 2.32 (s, 3H), 1.74 (sext, J=7.4 Hz, 2H), 0.99 (t, J=7.4 Hz, 3H); ¹³C NMR (100 MHz, MeOD) δ 171.91, 169.24, 165.61, 161.24, 153.30, 152.08, 147.72, 145.20, 139.29, 138.03, 137.87, 136.88, 134.30, 129.36, 128.41, 124.96, 111.02, 82.97, 67.93, 54.94, 44.00, 22.30, 17.65, 11.60; LCMS C₂₇H₃₀N₈O₂S method (B) Rₜ=4.835 min, ESI+ m/z=531.2 (M+H).

Methyl 5-(3-((4-(4,6-diaminopyrimidin-2-yl)thiazol-2-yl)(propyl)amino)-4-methylphenyl)picolinate OR0622 (method B, 42%) as a light brown solid. Rf=0.50 (DCM-MeOH—NH₄OH, 90:9:1); ¹H NMR (400 MHz, MeOD) δ 8.95 (dd, J=2.0, 0.6 Hz, 1H), 8.27 (dd, J=8.2, 2.0 Hz, 1H), 8.22 (dd, J=8.2, 0.6 Hz, 1H), 7.75 (dd, J=8.0, 1.8 Hz, 1H), 7.73 (d, J=1.8 Hz, 1H), 7.58 (d, J=8.0 Hz, 1H), 7.26 (s, 1H), 5.54 (s, 1H), 4.02 (brs, 2H), 4.00 (s, 3H) 2.33 (s, 3H), 1.74 (sext, J=7.4 Hz, 2H), 0.99 (t, J=7.4 Hz, 3H); ¹³C NMR (100 MHz, MeOD) δ 171.88, 166.44, 165.59, 161.19, 152.06, 148.65, 147.36, 145.28, 140.31, 139.80, 137.46, 136.88, 134.38, 129.52, 128.54, 126.61, 111.07, 82.96, 54.99, 53.24, 22.30, 17.69, 11.60; LCMS C₂₄H₂₅N₇O₂S method (B) Rₜ=5.062 min, ESI+ m/z=476.3 (M+H).

N-(5-(3-((4-(4,6-Diaminopyrimidin-2-yl)thiazol-2-yl)(propyl)amino)-4-methylphenyl)pyridin-2-yl)-4-methylpiperazine-1-sulfonamide OR0625 (method A, 18%) as a white powder. Rf=0.5 (DCM-MeOH—NH₄OH, 90:9:1); ¹H NMR (400 MHz, DMSO-d₆) ¹H NMR (400 MHz, DMSO-d₆) δ 8.51 (d, J=2.0 Hz, 1H), 8.12 (dd, J=8.8, 2.0 Hz, 1H), 7.71 (dd, J=8.4, 1.8 Hz, 1H), 7.70 (d, J=1.8 Hz, 1H), 7.52 (d, J=8.8 Hz, 1H), 7.41 (s, 1H), 7.21 (d, J=8.4 Hz, 1H), 7.03 (s, 4H), 5.48 (s, 1H), 3.97 (brs, 2H), 3.25-3.21 (m, 4H), 2.56-2.52 (m, 4H), 2.28 (s, 3H), 2.22 (s, 3H), 1.62 (sext, J=7.4 Hz, 2H), 0.93 (t, J=7.4 Hz, 3H); LCMS C₂₇H₃₄N₁₀O₂S₂ method (B) Rₜ=4.182 min, ESI+ m/z=595.3 (M+H). 2-(2-((5-(1H-Indol-5-yl)-2-methylphenyl)(propyl)amino)thiazol-4-yl)pyrimidine-4,6-diamine OR0626 (method B, 82%) as a light yellow solid. Rf=0.50 (DCM-MeOH—NH₄OH, 90:9:1); ¹H NMR (400 MHz, MeOD) δ 7.78 (d, J=1.8 Hz, 1H), 7.63 (dd, J=7.9, 1.8 Hz, 1H), 7.53 (d, J=1.6 Hz, 1H), 7.44 (d, J=7.9 Hz, 1H), 7.43 (d, J=8.2 Hz, 1H), 7.36 (dd, J=8.2, 1.6 Hz, 1H), 7.25 (s, 1H), 7.24 (d, J=2.8 Hz, 1H), 6.50 (d, J=2.8 Hz, 1H), 5.54 (s, 1H), 4.00 (brs, 2H), 2.27 (s, 3H), 1.75 (sext, J=7.4 Hz, 2H), 0.99 (t, J=7.4 Hz, 3H); ¹³C NMR (100 MHz, MeOD) δ 172.44, 165.37, 160.86, 151.48, 144.48, 144.22, 137.37, 135.56, 133.49, 132.36, 130.07, 128.85, 128.40, 126.44, 121.60, 119.39, 112.58, 111.16, 102.85, 82.81, 58.32, 54.72, 22.30, 18.36, 17.40, 11.64; LCMS C₂₅H₂₅N₇S method (B) Rₜ=5.452 min, ESI+ m/z=456.2 (M+H).

2-(2-((2-Methyl-5-(6-((4-methylpiperazin-1-yl)sulfonyl)pyridin-3-yl)phenyl)(propyl)amino)thiazol-4-yl)pyrimidine-4,6-diamine OR0627 (method A, 22% or method B, 71%), as a light yellow powder. Rf=0.50 (DCM-MeOH—NH₄OH, 90:10:1); ¹H NMR (400 MHz, MeOD) δ 9.00 (d, J=1.8 Hz, 1H), 8.31 (dd, J=8.2, 1.8 Hz, 1H), 8.01 (d, J=8.2 Hz, 1H), 7.76 (dd, J=7.8, 1.9 Hz, 1H), 7.74 (d, J=1.9 Hz, 1H), 7.58 (d, J=7.8 Hz, 1H), 7.26 (s, 1H), 5.53 (s, 1H), 4.02 (brs, 2H), 3.35-3.30 (m, 4H), 2.54-2.46 (m, 4H), 2.33 (s, 3H), 2.28 (s, 3H), 1.74 (sext, J=7.4 Hz, 2H), 0.99 (t, J=7.4 Hz, 3H); ¹³C NMR (100 MHz, MeOD) δ 171.84, 165.64, 161.29, 155.57, 152.17, 149.29, 145.31, 140.03, 139.92, 137.40, 137.14, 134.41, 129.64, 128.60, 124.55, 111.02, 82.99, 55.38, 55.00, 47.30, 45.87, 22.30, 17.70, 11.60; LCMS C₂₇H₃₃N₉O₂S₂ method (B) Rₜ=4.346 min, ESI+ m/z=580.3 (M+H).

2-(2-((2-Methyl-5-(6-(2-(4-methylpiperazin-1-yl)ethoxy)pyridin-3-yl)phenyl)(propyl)amino)thiazol-4-yl)pyrimidine-4,6-diamine OR0629 (method A, 31%) as a white powder. Rf=0.5 (DCM-MeOH—NH₄OH, 90:9:1); ¹H NMR (400 MHz, CDCl₃) δ 8.34 (d, J=2.3 Hz, 1H), 7.75 (dd, J=8.6, 2.3 Hz, 1H), 7.45 (dd, J=7.9, 1.8 Hz, 1H), 7.39 (d, J=7.9 Hz, 1H), 7.38 (d, J=1.8 Hz, 1H), 7.31 (s, 1H), 6.81 (d, J=8.6 Hz, 1H), 5.42 (s, 1H), 4.89 (brs, 4H), 4.47 (t, J=5.8 Hz, 2H), 3.98 (brs, 2H), 2.82 (t, J=5.8 Hz, 2H), 2.64 (brs, 4H), 2.51 (brs, 4H), 2.30 (s, 3H), 2.25 (s, 3H), 1.65 (sext, J=7.4 Hz, 2H), 0.91 (t, J=7.4 Hz, 3H); ¹³C NMR (100 MHz, CDCl₃) δ 170.95, 163.76, 163.31, 160.39, 151.04, 144.88, 143.52, 137.64, 137.33, 136.53, 132.76, 129.09, 127.63, 126.59, 111.33, 110.87, 82.92, 63.71, 57.15, 55.05, 53.72, 53.40, 46.01, 21.32, 17.50, 11.39; LCMS C₂₉H₃₇N₉OS method (B) Rₜ=4.379 min, ESI+m/z=560.3 (M+H).

2-((5-(3-((4-(4,6-Diaminopyrimidin-2-yl)thiazol-2-yl)(propyl)amino)-4-methylphenyl)pyridin-2-yl)oxy)-1-(4-methylpiperazin-1-yl)ethan-1-one OR0630 (method A, 3%) as a white powder. Rf=0.5 (DCM-MeOH—NH₄OH, 90:9:1); ¹H NMR (400 MHz, CDCl₃) δ 8.30 (d, J=2.4 Hz, 1H), 7.80 (dd, J=8.6, 2.4 Hz, 1H), 7.45 (dd, J=7.9, 1.7 Hz, 1H), 7.38 (d, J=7.9 Hz, 1H), 7.37 (d, J=1.7 Hz, 1H), 7.36 (s, 1H), 6.96 (d, J=8.6 Hz, 1H), 5.56 (s, 1H), 5.47 (brs, 4H), 5.05 (s, 2H), 3.97 (brs, 2H), 3.70-3.62 (m, 2H), 3.58-3.50 (m, 2H), 2.51-2.43 (m, 2H), 2.45-2.37 (m, 2H), 2.32 (s, 3H), 2.24 (s, 3H), 1.65 (sext, J=7.4 Hz, 2H), 0.93 (t, J=7.4 Hz, 3H); LCMS C₂₉H₃₅N₉O₂S method (B) Rₜ=4.361 min, ESI+ m/z=574.3 (M+H).

2-(2-(Methyl(4-methyl-4′-((4-methylpiperazin-1-yl)sulfonyl)-[1,1′-biphenyl]-3-yl)amino)thiazol-4-yl)pyrimidine-4,6-diamine OR0631 (method B, 67%) as a light yellow solid. Rf=0.47 (DCM-MeOH—NH₄OH, 90:9:1); ¹H NMR (400 MHz, MeOD) δ 7.89 (d, J=8.6 Hz, 2H), 7.84 (d, J=8.6 Hz, 2H), 7.71 (d, J=1.9 Hz, 1H), 7.69 (dd, J=7.7, 1.9 Hz, 1H), 7.53 (d, J=7.7 Hz, 1H), 7.29 (s, 1H), 5.54 (s, 1H), 3.60 (s, 3H), 3.09-3.01 (m, 4H), 2.54-2.46 (m, 4H), 2.32 (s, 3H), 2.25 (s, 3H); ¹³C NMR (100 MHz, CDCl₃) δ 171.88, 165.64, 161.18, 152.13, 146.67, 145.82, 140.49, 138.39, 135.59, 134.02, 129.67, 128.56, 128.49, 128.31, 111.39, 82.98, 55.08, 46.95, 45.75, 40.15, 17.37; LCMS C₂₆H₃₀N₈O₂S₂ method (B) Rₜ=4.294 min, ESI+ m/z=551.2 (M+H).

2-(2-(Ethyl(4-methyl-4′-((4-methylpiperazin-1-yl)sulfonyl)-[1,1′-biphenyl]-3-yl)amino)thiazol-4-yl)pyrimidine-4,6-di-amine OR0632 (method B, 58%) as a light brown solid. Rf=0.36 (DCM-MeOH—NH₄OH, 90:9:1); ¹H NMR (400 MHz, MeOD) δ 7.89 (d, J=8.6 Hz, 2H), 7.84 (d, J=8.6 Hz, 2H), 7.71 (dd, J=8.0, 1.8 Hz, 1H), 7.66 (d, J=1.8 Hz, 1H), 7.54 (d, J=8.0 Hz, 1H), 7.26 (s, 1H), 5.54 (s, 1H), 4.13 (brs, 2H), 3.09-3.01 (m, 4H), 2.54-2.46 (m, 4H), 2.31 (s, 3H), 2.25 (s, 3H), 1.28 (t, J=7.2 Hz, 3H); ¹³C NMR (100 MHz, MeOD) δ 171.66, 165.65, 161.30, 152.12, 145.84, 144.65, 140.31, 139.18, 135.58, 134.06, 129.70, 129.56, 128.58, 128.55, 110.99, 82.98, 55.08, 47.75, 46.95, 45.75, 17.58, 13.55, LCMS C₂₇H₃₂N₈O₂S₂ method (B) Rₜ=4.418 min, ESI+ m/z=565.2 (M+H).

2-(2-((5-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0633 (method A, 11%) as a light brown solid. Rf=0.5 (DCM-MeOH—NH₄OH, 90:9:1); ¹H NMR (400 MHz, CDCl₃) δ 7.81 (d, J=8.4 Hz, 2H), 7.68 (d, J=8.4 Hz, 2H), 7.40 (s, 1H), 7.32 (dd, J=10.2, 1.6 Hz, 1H), 7.31 (d, J=1.6 Hz, 1H), 5.57 (s, 1H), 5.40 (brs, 4H), 4.01 (brs, 2H), 3.10-3.02 (m, 4H), 2.53-2.45 (m, 4H), 2.26 (s, 3H), 2.19 (s, 3H), 1.66 (sext, J=7.4 Hz, 2H), 0.95 (t, J=7.4 Hz, 3H); ¹⁹F NMR (376 MHz, CDCl₃) δ −110.83; LCMS C₂SH₃₃FN₈O₂S₂ method (B) Rₜ=4.384 min, ESI+ m/z=597.2 (M+H).

2-(2-((3′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine OR0635 (method A, 36%) as light brown powder. Rf=0.5 (DCM-MeOH—NH₄OH, 90:9:1); ¹H NMR (400 MHz, CDCl₃) δ 7.91 (d, J=8.1 Hz, 1H), 7.53 (dd, J=8.1, 1.9 Hz, 1H), 7.45 (d, J=1.9 Hz, 1H), 7.43 (d, J=8.2 Hz, 1H), 7.35 (s, 1H), 7.20 (dd, J=8.2, 1.4 Hz, 1H), 7.12 (d, J=1.4 Hz, 1H), 5.49 (s, 1H), 5.10 (brs, 4H), 3.99 (brs, 2H), 3.96 (s, 3H), 3.31-3.22 (m, 4H), 2.50-2.42 (m, 4H), 2.29 (s, 3H), 2.28 (s, 3H), 1.66 (sext, J=7.4 Hz, 2H), 0.94 (t, J=7.4 Hz, 3H); ¹³C NMR (100 MHz, CDCl₃) δ 170.91, 163.31, 159.37, 157.46, 150.25, 146.51, 143.59, 139.34, 138.09, 132.85, 132.48, 128.36, 127.30, 125.02, 119.05, 111.33, 110.73, 82.80, 56.20, 54.84, 53.94, 46.02, 45.98, 21.34, 17.64, 11.42; LCMS C₂₉H₃₆FN₈O₃S₂ method (B) Rₜ=4.258 min, ESI+ m/z=609.3 (M+H).

2-(2-(iso-Propyl(4-methyl-4′-((4-methylpiperazin-1-yl)sulfonyl)-[1,1′-biphenyl]-3-yl)amino)thiazol-4-yl)pyrimidine-4,6-diamine OR0636 (method B, 29%) as a light brown solid. Rf=0.41 (DCM-MeOH—NH₄OH, 90:9:1); ¹H NMR (400 MHz, MeOD) δ 7.88 (d, J=8.0 Hz, 2H), 7.84 (d, J=8.0 Hz, 2H), 7.73 (dd, J=8.0, 1.9 Hz, 1H), 7.57 (d, J=1.9 Hz, 1H), 7.55 (d, J=8.0 Hz, 1H), 7.24 (s, 1H), 5.54 (s, 1H), 5.21 (sept, J=6.7 Hz, 1H), 3.09-3.01 (m, 4H), 2.54-2.46 (m, 4H), 2.32 (s, 3H), 2.25 (s, 3H), 1.29-1.28 (m, 6H); ¹³C NMR (100 MHz, MeOD) δ 171.77, 165.62, 161.37, 152.12, 145.82, 142.12, 140.70, 140.05, 135.57, 134.11, 130.95, 129.73, 128.86, 128.57, 110.77, 82.97, 55.07, 52.86, 46.94, 45.75, 21.46, 18.24; LCMS C₂₈H₃₄FN₈O₂S₂ method (B) Rₜ=4.311 min, ESI+ m/z=579.3 (M+H).

2-(2-(iso-Butyl(4-methyl-4′-((4-methylpiperazin-1-yl)sulfonyl)-[1,1′-biphenyl]-3-yl)amino)thiazol-4-yl)pyrimidine-4,6-diamine OR0637 (method B, 70%) as a light brown solid. Rf=0.45 (DCM-MeOH—NH₄OH, 90:9:1); ¹H NMR (400 MHz, MeOD) δ 7.88 (d, J=8.8 Hz, 2H), 7.85 (d, J=8.8 Hz, 2H), 7.69 (dd, J=8.0, 1.7 Hz, 1H), 7.67 (d, J=1.7 Hz, 1H), 7.53 (d, J=8.0 Hz, 1H), 7.26 (s, 1H), 5.53 (s, 1H), 3.92 (brs, 2H), 3.09-3.01 (m, 4H), 2.54-2.46 (m, 4H), 2.31 (s, 3H), 2.25 (s, 3H), 2.06-1.96 (m, 1H), 1.03 (d, J=6.6 Hz, 6H); ¹³C NMR (100 MHz, MeOD) δ 172.66, 165.63, 161.33, 152.13, 145.90, 145.49, 140.15, 138.81, 135.57, 134.36, 129.73, 129.26, 128.58, 128.41, 111.14, 83.00, 60.51, 55.08, 46.95, 45.75, 28.72, 20.82, 17.80; LCMS C₂₉H₃₆FN₈O₂S₂ method (B) Rₜ=4.369 min, ESI+ m/z=593.3 (M+H).

2-(2-((4′-((4-Ethylpiperazin-1-yl)sulfonyl)-4-methyl-[1,1′-biphenyl]-3-yl)(propyl)amino)thiazol-4-yl)pyrimidine-4,6-diamine OR0638 (method A, 36%) as a white solid. Rf=0.5 (DCM-MeOH—NH₄OH, 90:9:1); ¹H NMR (400 MHz, CDCl₃) δ 7.80 (d, J=8.5 Hz, 2H), 7.69 (d, J=8.5 Hz, 2H), 7.53 (dd, J=7.9, 1.9 Hz, 1H), 7.45 (d, J=1.9 Hz, 1H), 7.44 (d, J=7.9 Hz, 1H), 7.35 (s, 1H), 5.49 (s, 1H), 5.09 (brs, 4H), 4.00 (brs, 2H), 3.10-3.02 (m, 4H), 2.57-2.49 (m, 4H), 2.39 (q, J=7.2 Hz, 2H), 2.28 (s, 3H), 1.67 (sext, J=7.4 Hz, 2H), 1.02 (t, J=7.2 Hz, 3H), 0.94 (t, J=7.4 Hz, 3H); ¹³C NMR (100 MHz, CDCl₃) δ 170.91, 163.32, 159.40, 150.31, 144.63, 143.58, 139.05, 138.01, 133.88, 132.96, 128.63, 128.55, 127.56, 127.30, 111.30, 82.81, 53.83, 52.02, 51.91, 46.23, 21.35, 17.61, 12.03, 11.42; LCMS C₂₉H₃₆FN₈O₂S₂ method (B) Rₜ=4.316 min, ESI+ m/z=593.3 (M+H).

2-(2-((4-Methyl-4′-(piperazin-1-ylsulfonyl)-[1,1′-biphenyl]-3-yl)(propyl)amino)thiazol-4-yl)pyrimidine-4,6-diamine OR0639 (method C, 53%) as a white solid. Rf=0.18 (DCM-MeOH—NH₄OH, 90:9:1); ¹H NMR (400 MHz, CDCl₃) δ 7.83 (d, J=8.2 Hz, 2H), 7.74 (d, J=8.2 Hz, 2H), 7.57 (dd, J=7.9, 1.9 Hz, 1H), 7.51 (d, J=1.9 Hz, 1H), 7.47 (d, J=7.9 Hz, 1H), 7.38 (s, 1H), 5.50 (s, 1H), 5.00 (brs, 4H), 4.01 (brs, 2H), 3.10-3.02 (m, 4H), 3.01-2.94 (m, 4H), 2.30 (s, 3H), 1.69 (sext, J=7.2 Hz, 2H), 0.96 (t, J=7.2 Hz, 3H); ¹³C NMR (100 MHz, CDCl₃) δ 179.89, 170.95, 163.53, 159.84, 150.64, 144.57, 143.59, 139.00, 138.05, 134.28, 132.93, 128.59, 128.52, 127.57, 127.28, 111.18, 82.92, 53.89, 46.84, 45.32, 35.43, 21.34, 17.63, 11.43; LCMS C₂₇H₃₂N₈O₂S₂ method (B) Rₜ=4.272 min, ESI+ m/z=565.3 (M+H).

tert-Butyl 4-((3′-((4-(4,6-diaminopyrimidin-2-yl)thiazol-2-yl)(propyl)amino)-4′-methyl-[1,1′-biphenyl]-4-yl)sulfonyl) piperazine-1-carboxylate OR0639-1 (method A, 30%) as a light brown solid. ¹H NMR (400 MHz, CDCl₃) δ 7.80 (d, J=8.5 Hz, 2H), 7.72 (d, J=8.5 Hz, 2H), 7.56 (dd, J=7.9, 1.9 Hz, 1H), 7.48 (d, J=1.9 Hz, 1H), 7.46 (d, J=7.9 Hz, 1H), 7.40 (s, 1H), 5.76 (brs, 4H), 5.66 (s, 1H), 4.04 (brs, 2H), 3.55-3.47 (m, 4H), 3.05-2.97 (m, 4H), 2.29 (s, 3H), 1.69 (sext, J=7.4 Hz, 2H), 1.39 (s, 9H), 0.97 (t, J=7.4 Hz, 3H); LCMS C₃₂H₄₀N₈O₄S₂ method (B) Rₜ=5.632 min, ESI+ m/z=665.3 (M+H).

2-(2-((4-Methyl-4′-((4-methylpiperazin-1-yl)sulfonyl)-[1,1′-biphenyl]-3-yl)(propyl)amino)oxazol-4-yl)pyrimidine-4,6-diamine OR0640 (method A, 34%) as a light brown solid. Rf=0.50 (DCM-MeOH—NH₄OH, 90:10:1); ¹H NMR (400 MHz, MeOD) δ 7.86 (d, J=8.4 Hz, 2H), 7.83 (s, 1H), 7.82 (d, J=8.4 Hz, 2H), 7.61 (d, J=1.6 Hz, 1H), 7.59 (dd, J=8.4, 1.6 Hz, 1H), 7.45 (d, J=8.4 Hz, 1H), 5.48 (s, 1H), 3.89-3.81 (m, 2H), 3.08-3.00 (m, 4H), 2.53-2.45 (m, 4H), 2.25 (s, 3H), 2.24 (s, 3H), 1.74 (sext, J=7.4 Hz, 2H), 0.97 (t, J=7.4 Hz, 3H); ¹³C NMR (100 MHz, MeOD) δ 165.48, 162.13, 159.85, 146.19, 142.81, 141.16, 139.57, 138.48, 135.45, 134.97, 133.31, 129.64, 128.61, 128.57, 127.55, 82.92, 55.09, 54.80, 46.95, 45.75, 22.39, 17.74, 11.51; LCMS C₂₈H₃₄N₈O₃S method (B) Rₜ=4.676 min, ESI+ m/z=564.3 (M+H).

2-(2-((2′-Chloro-4-methyl-4′-((4-methylpiperazin-1-yl)sulfonyl)-[1,1′-biphenyl]-3-yl)(propyl)amino)thiazol-4-yl)pyrimidine-4,6-diamine OR0641 (method A, 28%) as a light yellow solid. Rf=0.50 (DCM-MeOH—NH₄OH, 90:10:1); ¹H NMR (400 MHz, MeOD) δ 7.90 (d, J=1.7 Hz, 1H), 7.77 (dd, J=8.1, 1.7 Hz, 1H), 7.67 (d, J=8.1 Hz, 1H), 7.54 (d, J=7.6 Hz, 1H), 7.46 (dd, J=7.6, 1.8 Hz, 1H), 7.44 (d, J=1.8 Hz, 1H), 7.26 (s, 1H), 5.53 (s, 1H), 3.99 (brs, 2H), 3.13-3.05 (m, 4H), 2.56-2.48 (m, 4H), 2.32 (s, 3H), 2.27 (s, 3H), 1.73 (sext, J=7.4 Hz, 2H), 0.98 (t, J=7.4 Hz, 3H). ¹³C NMR (100 MHz, MeOD) δ 171.97, 165.58, 161.20, 152.02, 145.23, 144.10, 139.09, 138.78, 137.57, 134.54, 133.48, 133.36, 131.77, 130.59, 130.21, 127.71, 111.10, 82.96, 55.07, 54.72, 46.94, 45.76, 22.26, 17.64, 11.61; LCMS C₂₈H₃₃ClN₈O₂S₂ method (B) Rₜ=4.434 min, ESI+ m/z=613.2 (M+H).

2-(2-((4-Methyl-4′-((4-methylpiperazin-1-yl)sulfonyl)-2′-(trifluoromethyl)-[1,1′-biphenyl]-3-yl)(propyl)amino)thiazol-4-yl)pyrimidine-4,6-diamine OR0642 (method A, 31% or method B, 56%) as a light yellow powder. Rf=0.27 (DCM-MeOH—NH₄OH, 95:5:0.5); ¹H NMR (400 MHz, MeOD) δ 8.11 (d, J=1.5 Hz, 1H), 8.07 (dd, J=8.0, 1.5 Hz, 1H), 7.70 (d, J=8.0 Hz, 1H), 7.52 (d, J=7.9 Hz, 1H), 7.35 (dd, J=7.9, 1.4 Hz, 1H), 7.31 (d, J=1.4 Hz, 1H), 7.26 (s, 1H), 5.53 (s, 1H), 4.00 (brs, 2H), 3.14-3.06 (m, 4H), 2.57-2.49 (m, 4H), 2.33 (s, 3H), 2.27 (s, 3H), 1.70 (sext, J=7.4 Hz, 2H), 0.98 (t, J=7.4 Hz, 3H); ¹³C NMR (100 MHz, MeOD) δ 171.96, 165.63, 161.29, 152.08, 146.18, 143.87, 139.13, 137.11, 134.77, 133.23, 132.28, 131.14, 130.53, 130.22, 130.12, 126.56, 111.07, 82.98, 55.05, 54.64, 46.90, 45.75, 22.20, 17.60, 11.58; LCMS C₂₉H₃₃F₃N₈O₂S₂ method (B) Rₜ=4.470 min, ESI+ m/z=647.2 (M+H).

2-(2-((2′-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0643 (method A, 32%) as a light brown powder. Rf=0.55 (DCM-MeOH—NH₄OH, 90:10:1); ¹H NMR (400 MHz, MeOD) δ 7.81-7.75 (m, 1H), 7.68-7.52 (m, 5H), 7.26 (s, 1H), 5.53 (s, 1H), 4.00 (brs, 2H), 3.13-3.05 (m, 4H), 2.56-2.48 (m, 4H), 2.32 (s, 3H), 2.27 (s, 3H), 1.73 (sext, J=7.4 Hz, 2H), 0.99 (t, J=7.4 Hz, 3H); ¹³C NMR (100 MHz, MeOD) δ 171.92, 165.63, 161.29, 159.37, 152.12, 144.59, 139.43, 137.77, 135.04, 133.97, 133.78, 132.79, 131.41, 130.35, 125.29, 117.08, 111.03, 82.98, 55.08, 54.81, 46.94, 45.76, 22.26, 17.67, 11.59; LCMS C₂₈H₃₃FN₈O₂S₂ method (B) Rₜ=4.322 min, ESI+ m/z=597.3 (M+H).

2-(2-((2′-Fluoro-6′-methoxy-4-methyl-4′-((4-methylpiperazin-1-yl)sulfonyl)-[1,1′-biphenyl]-3-yl)(propyl)amino)thiazol-4-yl)pyrimidine-4,6-diamine OR0644 (method A, 24%) as a light yellow solid. Rf=0.55 (DCM-MeOH—NH₄OH, 90:10:1); ¹H NMR (400 MHz, MeOD) δ 7.49 (d, J=7.9 Hz, 1H), 7.39 (d, J=7.9 Hz, 1H), 7.35 (s, 1H), 7.28-7.20 (m, 3H), 5.53 (s, 1H), 3.99 (brs, 2H), 3.88 (s, 3H), 3.15-3.07 (m, 4H), 2.57-2.49 (m, 4H), 2.31 (s, 3H), 2.28 (s, 3H), 1.72 (sext, J=7.4 Hz, 2H), 0.98 (t, J=7.4 Hz, 3H); ¹³C NMR (100 MHz, MeOD) δ 172.13, 165.61, 162.39, 161.30, 159.88, 152.01, 143.84, 138.53, 137.89, 133.05, 132.91, 131.87, 130.92, 123.26, 111.03, 108.99, 107.48, 82.96, 57.18, 55.11, 54.57, 46.98, 45.77, 22.18, 17.62, 11.61; LCMS C₂₉H₃₅FN₈O₃S₂ method (B) Rₜ=4.388 min, ESI+ m/z=627.3 (M+H).

2-(2-((2′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine OR0645 (method A, 28%) as a light brown solid. Rf=0.68 (DCM-MeOH—NH₄OH, 90:10:1); ¹H NMR (400 MHz, MeOD) δ 7.57 (d, J=7.9 Hz, 1H), 7.52 (dd, J=7.9, 1.7 Hz, 1H), 7.51 (d, J=1.7 Hz, 1H), 7.47 (d, J=7.9 Hz, 1H), 7.44 (dd, J=7.9, 1.7 Hz, 1H), 7.37 (d, J=1.7 Hz, 1H), 7.25 (s, 1H), 5.54 (s, 1H), 4.02 (brs, 2H), 3.89 (s, 3H), 3.12-3.04 (m, 4H), 2.56-2.50 (m, 4H), 2.29 (s, 3H), 2.26 (s, 3H), 1.73 (sext, J=7.6 Hz, 2H), 0.99 (t, J=7.4 Hz, 3H); ¹³C NMR (100 MHz, MeOD) δ 172.16, 165.50, 161.08, 158.24, 151.83, 143.86, 137.99, 137.83, 136.89, 135.50, 133.12, 132.12, 131.97, 130.75, 121.54, 111.54, 111.08, 82.90, 56.59, 55.11, 54.63, 46.98, 45.76, 22.21, 17.57, 11.63; LCMS C₂₉H₃₆FN₈O₃S₂ method (B) Rₜ=4.359 min, ESI+ m/z=609.3 (M+H).

2-(2-((4-Methyl-4′-(piperidin-4-ylsulfonyl)-[1,1′-biphenyl]-3-yl)(propyl)amino)thiazol-4-yl)pyrimidine-4,6-diamine OR0646 (method A, 28%) as a light yellow solid. Rf=0.20 (DCM-MeOH—NH₄OH, 90:10:1); ¹H NMR (400 MHz, MeOD) δ 7.95 (d, J=8.6 Hz, 2H), 7.91 (d, J=8.6 Hz, 2H), 7.71 (dd, J=8.0, 1.9 Hz, 1H), 7.66 (d, J=1.9 Hz, 1H), 7.55 (d, J=8.0 Hz, 1H), 7.25 (s, 1H), 5.54 (s, 1H), 4.01 (brs, 2H), 3.32-3.24 (m, 1H), 3.12-3.04 (m, 2H), 2.59-2.51 (m, 2H), 2.31 (s, 3H), 1.99-1.91 (m, 2H), 1.72 (sext, J=7.4 Hz, 2H), 1.64-1.53 (m, 2H), 0.99 (t, J=7.4 Hz, 3H); ¹³C NMR (100 MHz, MeOD) δ 171.95, 165.63, 161.27, 152.11, 146.75, 145.05, 140.20, 139.23, 136.81, 134.17, 130.94, 129.54, 128.66, 128.59, 111.00, 82.99, 62.42, 54.89, 45.52, 26.62, 22.30, 17.63, 11.61; LCMS C₂₈H₃₃N₇O₂S₂ method (B) Rₜ=4.266 min, ESI+ m/z=564.2 (M+H).

2-(2-((2′,6′-Difluoro-4-methyl-4′-((4-methylpiperazin-1-yl)sulfonyl)-[1,1′-biphenyl]-3-yl)(propyl)amino)thiazol-4-yl)pyrimidine-4,6-diamine OR0647 (method A, 27%) as a light yellow solid. Rf=0.70 (DCM-MeOH—NH₄OH, 90:10:1); ¹H NMR (400 MHz, MeOD) δ 7.57 (d, J=8.0 Hz, 1H), 7.54-7.48 (m, 3H), 7.48 (s, 1H), 7.26 (s, 1H), 5.53 (s, 1H), 3.99 (brs, 2H), 3.16-3.08 (m, 4H), 2.57-2.49 (m, 4H), 2.33 (s, 3H), 2.28 (s, 3H), 1.72 (sext, J=7.4 Hz, 2H), 0.98 (t, J=7.4 Hz, 3H); ¹³C NMR (100 MHz, MeOD) δ 171.90, 165.62, 162.49, 161.27, 159.90, 152.09, 144.38, 139.94, 138.60, 133.52, 132.70, 131.55, 128.50, 112.64, 111.10, 82.98, 55.07, 54.73, 46.92, 45.77, 22.21, 17.72, 11.58; LCMS C₂₈H₃₂N₈O₂S₂ method (B) Rₜ=4.396 min, ESI+ m/z=615.2 (M+H).

2-(2-((4′-((4-Aminopiperidin-1-yl)sulfonyl)-4-methyl-[1,1′-biphenyl]-3-yl)(propyl)amino)thiazol-4-yl)pyrimidine-4,6-diamine OR0648 (method C, 90%) as a light yellow solid. Rf=0.18 (DCM-MeOH—NH₄OH, 90:9:1). ¹H NMR (400 MHz, MeOD) δ 7.84 (d, J=8.6 Hz, 2H), 7.81 (d, J=8.6 Hz, 2H), 7.67 (dd, J=8.0, 1.5 Hz, 1H), 7.62 (d, J=1.5 Hz, 1H), 7.51 (d, J=8.0 Hz, 1H), 7.24 (s, 1H), 5.54 (s, 1H), 3.99 (brs, 2H), 3.74-3.66 (m, 2H), 2.60-2.52 (m, 1H), 2.43-2.35 (m, 2H), 2.28 (s, 3H), 1.89-1.81 (m, 2H), 1.71 (sext, J=7.4 Hz, 2H), 1.46-1.34 (m, 2H), 0.96 (t, J=7.4 Hz, 3H); ¹³C NMR (100 MHz, MeOD) δ 171.92, 165.62, 161.29, 152.14, 145.55, 144.99, 140.27, 138.95, 136.32, 134.13, 129.50, 129.38, 128.54, 128.48, 111.01, 83.03, 54.86, 48.72, 46.43, 34.80, 22.29, 17.64, 11.63; LCMS C₂₈H₃₄N₈O₂S₂ method (B) Rₜ=4.317 min, ESI+ m/z=579.3 (M+H).

tert-Butyl (1-((3′-((4-(4,6-diaminopyrimidin-2-yl)thiazol-2-yl)(propyl)amino)-4′-methyl-[1,1′-biphenyl]-4-yl)sulfonyl) piperidin-4-yl)carbamate OR0648-1 (method A, 20%) as a light brown solid. Rf=0.20 (DCM-MeOH—NH₄OH, 95:5:0.5); ¹H NMR (400 MHz, CDCl₃) δ 7.80 (d, J=8.5 Hz, 2H), 7.72 (d, J=8.5 Hz, 2H), 7.57 (dd, J=7.9, 1.8 Hz, 1H), 7.50 (d, J=1.8 Hz, 1H), 7.46 (d, J=7.9 Hz, 1H), 7.41 (s, 1H), 5.70 (brs, 4H), 5.65 (s, 1H), 4.42 (brs, 1H), 4.05 (brs, 2H), 3.73 (brs, 1H), 3.40 (brs, 1H), 2.53-2.45 (m, 2H), 2.29 (s, 3H), 2.02-1.96 (m, 2H), 1.71 (sext, J=7.4 Hz, 2H), 1.55-1.47 (m, 2H), 1.40 (s, 9H), 0.98 (t, J=7.4 Hz, 3H); LCMS C₃₃H₄₂N₈O₄S₂ method (B) Rₜ=5.495 min, ESI+ m/z=679.3 (M+H).

2-(4-((3′-((4-(4,6-Diaminopyrimidin-2-yl)thiazol-2-yl)(propyl)amino)-4′-methyl-[1,1′-biphenyl]-4-yl)sulfonyl) piperazin-1-yl)acetic acid OR0649 (method A, 20%) as a white solid. Rf=0.20 (DCM-MeOH—NH₄OH, 80:18:2); ¹H NMR (400 MHz, MeOD) δ 7.89 (d, J=8.7 Hz, 2H), 7.85 (d, J=8.7 Hz, 2H), 7.74 (dd, J=8.0, 1.9 Hz, 1H), 7.66 (d, J=1.9 Hz, 1H), 7.57 (s, 1H), 7.56 (d, J=8.0 Hz, 1H), 5.61 (s, 1H), 4.01 (brs, 2H), 3.17 (s, 2H), 3.16-3.12 (m, 4H), 2.86-2.80 (m, 4H), 2.31 (s, 3H), 1.74 (sext, J=7.4 Hz, 2H), 1.01 (t, J=7.4 Hz, 3H); ¹³C NMR (100 MHz, MeOD) δ 174.37, 172.57, 162.36, 154.84, 146.30, 145.71, 144.52, 140.49, 138.81, 135.64, 134.31, 129.76, 129.26, 128.85, 128.64, 114.62, 81.17, 61.32, 54.98, 53.18, 46.41, 22.21, 17.56, 11.73; LCMS C₂₉H₃₄N₈O₄S₂ method (B) Rₜ=4.505 min, ESI+ m/z=623.3 (M+H).

2-(2-((4-Methyl-4′-((4-methylpiperazin-1-yl)sulfonyl)-3′-(trifluoromethoxy)-[1,1′-biphenyl]-3-yl)(propyl)amino) thiazol-4-yl)pyrimidine-4,6-diamine OR0650 (method A, 40%) as a light brown solid. Rf=0.20 (DCM-MeOH—NH₄OH, 95:5:0.5); ¹H NMR (400 MHz, MeOD) δ 8.04 (d, J=8.3 Hz, 1H), 7.81 (dd, J=8.3, 1.7 Hz, 1H), 7.73-7.72 (m, 1H), 7.69 (dd, J=7.9, 2.0 Hz, 1H), 7.66 (d, J=2.0 Hz, 1H), 7.56 (d, J=7.9 Hz, 1H), 7.26 (s, 1H), 5.54 (s, 1H), 4.01 (brs, 2H), 3.27-3.19 (m, 4H), 2.53-2.45 (m, 4H), 2.31 (s, 3H), 2.28 (s, 3H), 1.73 (sext, J=7.4 Hz, 2H), 0.99 (t, J=7.4 Hz, 3H); ¹³C NMR (100 MHz, MeOD) δ 171.84, 165.58, 161.18, 152.07, 148.09, 147.72, 145.17, 139.95, 138.92, 134.35, 133.68, 130.03, 129.48, 128.48, 126.65, 122.98, 120.66, 111.09, 82.97, 55.37, 54.97, 46.59, 45.86, 22.29, 17.70, 11.60; LCMS C₂₉H₃₃F₃N₈O₃S₂ method (B) Rₜ=4.530 min, ESI+ m/z=663.2 (M+H).

N-(2-Aminoethyl)-3′-((4-(4,6-diaminopyrimidin-2-yl)thiazol-2-yl)(propyl)amino)-4′-methyl-[1,1′-biphenyl]-4-sulfonamide OR0651 (method C, 100%) as a light yellow solid. Rf=0.22 (DCM-MeOH—NH₄OH, 90:9:1); ¹H NMR (400 MHz, MeOD) δ 7.97 (d, J=8.5 Hz, 2H), 7.87 (d, J=8.5 Hz, 2H), 7.75 (dd, J=8.0, 1.7 Hz, 1H), 7.69 (s, 1H), 7.66 (d, J=1.7 Hz, 1H), 7.58 (d, J=8.0 Hz, 1H), 5.65 (s, 1H), 4.22 (brs, 1H), 3.84 (brs, 1H), 3.18-3.12 (m, 2H), 3.12-3.04 (m, 2H), 2.32 (s, 3H), 1.75 (sext, J=7.4 Hz, 2H), 1.04 (t, J=7.4 Hz, 3H); ¹³C NMR (100 MHz, MeOD) δ 172.81, 152.43, 145.44, 144.32, 144.07, 140.64, 139.90, 138.72, 134.38, 129.13, 129.01, 128.94, 128.69, 115.92, 80.43, 54.92, 41.39, 40.67, 22.17, 17.50, 11.74. 1; LCMS C₂₆H₃₄F₃N₈O₂S₂ method (B) Rₜ=4.102 min, ESI+ m/z=539.3 (M+H).

tert-Butyl (2-((3′-((4-(4,6-diaminopyrimidin-2-yl)thiazol-2-yl)(propyl)amino)-4′-methyl-[1,1′-biphenyl])-4-sulfonamido)ethyl)carbamate OR0651-1 (method A, 29%) as a white powder. Rf=0.6 (DCM-MeOH—NH₄OH, 90:9:1); ¹H NMR (400 MHz, CDCl₃) δ 7.90 (d, J=8.4 Hz, 2H), 7.66 (d, J=8.4 Hz, 2H), 7.51 (dd, J=8.0, 1.7 Hz, 1H), 7.45 (d, J=1.7 Hz, 1H), 7.40 (d, J=8.0 Hz, 1H), 7.33 (s, 1H), 5.77 (brs, 1H), 5.50 (s, 1H), 5.12 (brs, 4H), 5.08 (brs, 1H), 3.93 (brs, 2H), 3.25-3.21 (m, 2H), 3.11-3.05 (m, 2H), 2.24 (s, 3H), 1.62 (sext, 7.3 Hz, 2H), 1.40 (s, 9H), 0.90 (t, J=7.3 Hz, 3H); ¹³C NMR (100 MHz, CDCl₃) δ 170.92, 163.17, 156.74, 144.15, 143.47, 143.21, 139.00, 138.82, 137.80, 132.86, 128.31, 127.80, 127.63, 127.58, 127.25, 111.41, 82.86, 80.00, 54.02, 43.88, 40.48, 28.49, 21.28, 17.63, 11.41; LCMS C₃₀H₃₈N₈O₄S₂ method (B) Rₜ=4.890 min, ESI+ m/z=639.3 (M+H).

2-(2-((2-Methyl-5-(4-methyl-6-((4-methylpiperazin-1-yl)sulfonyl)pyridin-3-yl)phenyl)(propyl)amino)thiazol-4-yl)pyrimidine-4,6-diamine OR0652 (method B, 31%) as a light yellow powder. Rf=0.23 (DCM-MeOH, 90:10); ¹H NMR (400 MHz, CDCl₃) δ 8.51 (s, 1H), 7.82 (s, 1H), 7.48 (d, J=7.9 Hz, 1H), 7.42 (s, 1H), 7.28 (dd, J=7.9, 1.4 Hz, 1H), 7.20 (d, J=1.4 Hz, 1H), 5.95 (brs, 4H), 5.69 (s, 1H), 4.04 (brs, 2H), 3.40-3.36 (m, 4H), 2.54-2.50 (m, 4H), 2.38 (s, 3H), 2.31 (s, 3H), 2.30 (s, 3H), 1.67 (sext, J=7.4 Hz, 2H), 0.97 (t, J=7.4 Hz, 3H); ¹³C NMR (100 MHz, CDCl₃) δ 170.90, 161.56, 158.18, 154.50, 150.25, 147.16, 142.97, 140.78, 139.57, 137.91, 136.05, 132.93, 130.55, 129.41, 124.70, 112.64, 82.10, 54.50, 53.80, 46.57, 45.93, 21.40, 20.43, 17.62, 11.50; LCMS C₂₈H₃₅N₉O₂S₂ method (B) Rₜ=4.434 min, ESI+ m/z=594.2 (M+H).

2-(2-((2-Methyl-5-(2-methyl-6-((4-methylpiperazin-1-yl)sulfonyl)pyridin-3-yl)phenyl)(propyl)amino)thiazol-4-yl)pyrimidine-4,6-diamine OR0653 (method B, 48%) as a light yellow powder. Rf=0.23 (DCM-MeOH, 90:10); ¹H NMR (400 MHz, CDCl₃) δ 7.80 (d, J=8.0 Hz, 1H), 7.71 (d, J=8.0 Hz, 1H), 7.45 (d, J=7.9 Hz, 1H), 7.36 (s, 1H), 7.24 (dd, J=7.9, 1.8 Hz, 1H), 7.19 (d, J=1.8 Hz, 1H), 5.53 (s, 1H), 5.20 (brs, 4H), 3.99 (brs, 2H), 3.42-3.34 (m, 4H), 2.55 (s, 3H), 2.59-2.51 (m, 4H), 2.30 (s, 3H), 2.29 (s, 3H), 1.65 (sext, J=7.4 Hz, 2H), 0.94 (t, J=7.4 Hz, 3H); ¹³C NMR (100 MHz, CDCl₃) δ 170.87, 163.09, 157.39, 154.07, 149.83, 143.05, 139.16, 138.69, 138.00, 137.84, 132.69, 130.21, 129.04, 120.72, 111.51, 82.70, 54.55, 53.65, 46.76, 46.00, 23.78, 21.37, 17.60, 11.43; LCMS C₂₈H₃₅N₉O₂S₂ method (B) Rₜ=4.306 min, ESI+ m/z=594.5 (M+H).

2-(2-((5-(6-Methoxy-4-(trifluoromethyl)pyridin-3-yl)-2-methylphenyl)(propyl)amino)thiazol-4-yl)pyrimidine-4,6-diamine OR0654 (method A, 25%) as a light yellow powder. Rf=0.23 (DCM-MeOH, 90:10); ¹H NMR (400 MHz, CDCl₃) δ 8.17 (s, 1H), 7.39 (d, J=7.9 Hz, 1H), 7.34 (s, 1H), 7.24 (dd, J=7.9, 1.7 Hz, 1H), 7.20 (d, J=1.7 Hz, 1H), 7.07 (s, 1H), 5.52 (s, 1H), 5.10 (brs, 4H), 4.00 (s, 5H), 2.28 (s, 3H), 1.61 (sext, J=7.4 Hz, 2H), 0.92 (t, J=7.4 Hz, 3H); ¹³C NMR (100 MHz, CDCl₃) δ 171.18, 164.02, 163.30, 149.81, 142.43, 138.91, 138.60, 137.51, 135.57, 132.00, 131.09, 129.79, 127.29, 124.11, 121.38, 111.49, 107.99, 82.75, 54.18, 53.46, 21.23, 17.54, 11.41; LCMS C₂₄H₂₄F₃N₇OS method (B) Rₜ=5.882 min, ESI+ m/z=516.2 (M+H).

2-(2-((5-(6-Hydroxy-4-(trifluoromethyl)pyridin-3-yl)-2-methylphenyl)(propyl)amino)thiazol-4-yl)pyrimidine-4,6-diamine OR0655 (method A, 95%) as a light yellow powder. Rf=0.66 (DCM-MeOH—NH₄OH, 80:18:2); ¹H NMR (400 MHz, MeOD) δ 7.60 (s, 1H), 7.50 (s, 1H), 7.49 (d, J=7.9 Hz, 1H), 7.34 (dd, J=7.9, 2.0 Hz, 1H), 7.29 (d, J=2.0 Hz, 1H), 6.91 (s, 1H), 5.64 (s, 1H), 4.01 (brs, 2H), 2.30 (s, 3H), 1.70 (sext, J=7.4 Hz, 2H), 1.00 (t, J=7.4 Hz, 3H); ¹³C NMR (100 MHz, MeOD) δ 172.71, 163.90, 154.10, 145.52, 143.34, 142.64, 138.86, 138.58, 135.95, 133.42, 132.26, 131.93, 124.99, 122.25, 119.06, 118.28, 115.05, 80.92, 54.68, 22.08, 17.47, 11.70; LCMS C₂₃H₂₂F₃N₇OS method (B) Rₜ=4.323 min, ESI+ m/z=502.2 (M+H).

2-(2-((2-Methyl-5-(6-((4-methylpiperazin-1-yl)sulfonyl)-4-(trifluoromethyl)pyridin-3-yl)phenyl)(propyl)amino)thiazol-4-yl)pyrimidine-4,6-diamine OR0656 (method B, 59%) as a light yellow powder. Rf=0.23 (DCM-MeOH, 90:10); ¹H NMR (400 MHz, MeOD) δ 8.84 (s, 1H), 8.22 (s, 1H), 7.58 (d, J=8.2 Hz, 1H), 7.44 (dd, J=8.2, 1.9 Hz, 1H), 7.42 (d, J=1.9 Hz, 1H), 7.33 (s, 1H), 5.55 (s, 1H), 4.00 (brs, 2H), 3.42-3.36 (m, 4H), 2.56-2.50 (m, 4H), 2.35 (s, 3H), 2.31 (s, 3H), 1.71 (sext, J=7.5 Hz, 2H), 0.98 (t, J=7.4 Hz, 3H); ¹³C NMR (100 MHz, CDCl₃) δ 171.98, 164.96, 159.93, 157.81, 154.42, 150.90, 144.11, 140.02, 138.84, 135.45, 133.60, 131.68, 130.57, 125.14, 122.41, 120.14, 111.89, 82.60, 55.42, 54.75, 47.34, 45.89, 22.18, 17.66, 11.59; LCMS C₂₈H₃₂F₃N₉O₂S₂ method (B) Rₜ=4.511 min, ESI+ m/z=648.2 (M+H).

2-(2-((2-Methyl-5-(6-((4-methylpiperazin-1-yl)sulfonyl)-2-(trifluoromethyl)pyridin-3-yl)phenyl)(propyl)amino)thiazol-4-yl)pyrimidine-4,6-diamine OR0657 (method B, 55%) as a light yellow powder. Rf=0.23 (DCM-MeOH, 90:10); ¹H NMR (400 MHz, CDCl₃) δ 8.13 (d, J=8.0 Hz, 1H), 7.98 (d, J=8.0 Hz, 1H), 7.45 (d, J=7.9 Hz, 1H), 7.37 (s, 1H), 7.26 (dd, J=7.9, 1.9 Hz, 1H), 7.23 (d, J=1.9 Hz, 1H), 5.54 (s, 1H), 5.28 (brs, 4H), 3.99 (brs, 2H), 3.50-3.44 (m, 4H), 2.54-2.48 (m, 4H), 2.31 (s, 3H), 2.30 (s, 3H), 1.62 (sext, J=7.4 Hz, 2H), 0.93 (t, J=7.4 Hz, 3H); ¹³C NMR (100 MHz, CDCl₃) δ 170.83, 162.95, 155.47, 145.45, 145.11, 142.77, 142.57, 139.06, 138.86, 135.16, 132.47, 130.07, 128.75, 124.96, 122.57, 119.82, 111.78, 82.65, 54.56, 53.73, 46.79, 46.03, 21.25, 17.68, 11.42; LCMS C₂₈H₃₂F₃N₉O₂S₂ method (B) Rₜ=4.601 min, ESI+ m/z=648.2 (M+H).

2-(2-((2-Methyl-5-(6-(piperazin-1-ylsulfonyl)-2-(trifluoromethyl)pyridin-3-yl)phenyl)(propyl)amino)thiazol-4-yl)pyrimidine-4,6-diamine OR0658 (method C, 65%) as a light yellow powder. Rf=0.18 (DCM-MeOH, 90:10); ¹H NMR (400 MHz, CDCl₃) δ 8.12 (d, J=8.1 Hz, 1H), 7.98 (d, J=8.1 Hz, 1H), 7.45 (d, J=7.9 Hz, 1H), 7.36 (s, 1H), 7.26 (dd, J=7.9, 1.9 Hz, 1H), 7.26 (d, J=1.9 Hz, 1H), 5.45 (s, 1H), 5.00 (brs, 4H), 3.97 (brs, 2H), 3.46-3.40 (m, 4H), 3.00-2.94 (m, 4H), 2.31 (s, 3H), 1.62 (sext, J=7.5 Hz, 2H), 0.92 (t, J=7.4 Hz, 3H); ¹³C NMR (100 MHz, CDCl₃) δ 170.81, 163.41, 159.60, 155.57, 150.36, 145.40, 142.82, 142.61, 139.07, 138.90, 135.14, 132.45, 130.08, 128.70, 124.98, 121.22, 111.48, 82.82, 53.74, 47.70, 45.70, 21.21, 17.69, 11.38; LCMS C₂₇H₃₀F₃N₉O₂S₂ method (B) Rₜ=4.308 min, ESI+m/z=634.2 (M+H).

General Procedure for the Synthesis of 2-(2-((5-halogeno-2-methylphenyl)(alkyl)amino)thiazol-4-yl)pyrimidine-4,6-diamine. As previously described for synthesis of 2-[2-(biaryl-3-ylamino)-thiazol-4-yl]-pyrimidine-4,6-diamine derivative, method (A).

2-(2-((5-Iodo-2-methylphenyl)(propyl)amino)thiazol-4-yl)pyrimidine-4,6-diamine OR0607-1 (40%) as a light yellow solid. Rf=0.30 (DCM-MeOH—NH₄OH, 95:5:0.5); ¹H NMR (400 MHz, CDCl₃) δ 7.60 (dd, J=8.1, 1.7 Hz, 1H), 7.55 (d, J=1.7 Hz, 1H), 7.36 (s, 1H), 7.06 (d, J=8.1 Hz, 1H), 5.50 (s, 1H), 5.08 (brs, 4H), 3.97-3.89 (m, 2H), 2.17 (s, 3H), 1.60 (sext, J=7.4 Hz, 2H), 0.91 (t, J=7.4 Hz, 3H); ¹³C NMR (100 MHz, CDCl₃) δ 170.59, 163.34, 159.47, 150.29, 144.20, 138.28, 137.65, 137.58, 133.74, 111.53, 90.93, 82.85, 53.91, 21.22, 17.54, 11.37; LCMS C₁₇H₁₉N₆S method (B) Rₜ=5.226 min, ESI+ m/z=467.0 (M+H).

2-(2-((5-Bromo-2-methylphenyl)amino)thiazol-4-yl)pyrimidine-4,6-diamine OR0614-1 (23%) as a light brown solid. Rf=0.15 (DCM-MeOH—NH₄OH, 95:5:0.5); ¹H NMR (400 MHz, MeOD) δ 7.87 (d, J=1.8 Hz, 1H), 7.46 (s, 1H), 7.19 (dd, J=8.1, 1.8 Hz, 1H), 7.15 (d, J=8.1 Hz, 1H), 5.54 (s, 1H), 2.27 (s, 3H); LCMS C₁₄H₁₃BrN₆S method (B) Rₜ=4.752 min, ESI+ m/z=337.0 (M+H).

2-(2-((5-Bromo-2-methylphenyl)(propyl)amino)thiazol-4-yl)pyrimidine-4,6-diamine OR0652-1 (36%) as a light yellow solid. Rf=0.35 (DCM-MeOH—NH₄OH, 95:5:0.5); ¹H NMR (400 MHz, CDCl₃) δ 7.41 (dd, J=8.2, 2.0 Hz, 1H), 7.37 (d, J=2.0 Hz, 1H), 7.35 (s, 1H), 7.20 (d, J=8.2 Hz, 1H), 5.46 (s, 1H), 5.01 (brs, 4H), 3.95-3.87 (m, 2H), 2.16 (s, 3H), 1.60 (sext, J=7.4 Hz, 2H), 0.90 (t, J=7.4 Hz, 3H); LCMS C₁₇H₁₉BrN₆S method (B) Rₜ=5.340 min, ESI+m/z=419.0 (M+H). 2-(2-((5-Bromo-2-methylphenyl)(methyl)amino)thiazol-4-yl)pyrimidine-4,6-diamine OR0631-1 (50%) as a light brown solid. Rf=0.60 (DCM-MeOH—NH₄OH, 90:10:1); ¹H NMR (400 MHz, MeOD) δ 7.51 (d, J=2.0 Hz, 1H), 7.49 (dd, J=8.3, 2.0 Hz, 1H), 7.32 (d, J=8.3 Hz, 1H), 7.30 (s, 1H), 5.53 (s, 1H), 3.51 (s, 3H), 2.22 (s, 3H); LCMS C₁₅H₁₅BrN₆S method (B) Rₜ=5.129 min, ESI+ m/z=391.0 (M+H).

2-(2-((5-Bromo-2-methylphenyl)(ethyl)amino)thiazol-4-yl)pyrimidine-4,6-diamine OR0632-1 (40%) as a light brown solid. Rf=0.45 (DCM-MeOH—NH₄OH, 90:9:1); ¹H NMR (400 MHz, MeOD) δ 7.49 (dd, J=8.1, 2.0 Hz, 1H), 7.46 (d, J=2.0 Hz, 1H), 7.31 (d, J=8.1 Hz, 1H), 7.29 (s, 1H), 5.53 (s, 1H), 4.04 (q, J=7.1 Hz, 2H), 2.20 (s, 3H), 1.22 (t, J=7.1 Hz, 3H); ¹³C NMR (100 MHz, MeOD) δ 171.20, 165.46, 160.93, 151.84, 145.16, 138.02, 134.70, 133.57, 132.92, 120.94, 111.38, 82.94, 47.83, 17.37, 13.40; LCMS C₁₆H₁₇BrN₆S method (B) R=5.205 min, ESI+ m/z=405.0 (M+H).

2-(2-((5-Bromo-2-methylphenyl)(isopropyl)amino)thiazol-4-yl)pyrimidine-4,6-diamine OR0636-1 (35%) as a light yellow solid. Rf=0.45 (DCM-MeOH—NH₄OH, 90:9:1); ¹H NMR (400 MHz, MeOD) δ 7.53 (dd, J=8.3, 2.1 Hz, 1H), 7.41 (d, J=2.1 Hz, 1H), 7.35 (d, J=8.3 Hz, 1H), 7.28 (s, 1H), 5.54 (s, 1H), 5.13 (sept, J=6.7 Hz, 1H), 2.21 (s, 3H), 1.24 (d, J=6.7 Hz, 6H); ¹³C NMR (100 MHz, MeOD) δ 171.34, 165.39, 160.85, 151.69, 142.68, 139.55, 134.95, 134.74, 133.26, 120.67, 111.20, 82.85, 53.00, 21.21, 17.96; LCMS C₁₇H₁₉BrN₆S method (B) Rₜ=5.252 min, ESI+ m/z=419.0 (M+H).

2-(2-((5-Bromo-2-methylphenyl)(isobutyl)amino)thiazol-4-yl)pyrimidine-4,6-diamine OR0637-1 (40%) as a light brown solid. Rf=0.45 (DCM-MeOH—NH₄OH, 90:9:1); ¹H NMR (400 MHz, MeOD) δ 7.51-7.47 (m, 2H), 7.32 (d, J=8.8 Hz, 1H), 7.28 (s, 1H), 5.53 (s, 1H), 3.84 (brs, 2H), 2.21 (s, 3H), 2.02-1.89 (m, 1H), 1.01 (d, J=6.7 Hz, 6H); ¹³C NMR (100 MHz, MeOD) δ 172.24, 165.59, 161.19, 152.04, 146.07, 137.69, 135.00, 133.30, 132.76, 120.84, 111.40, 82.98, 60.56, 28.64, 20.71, 17.59; LCMS C₁₈H₂₁BrN₆S method (B) Rₜ=5.382 min, ESI+ m/z=433.0 (M+H).

General Procedure for the Synthesis of ethyl 2-([1,1′-biaryl]-3-ylamino)thiazole-4-carboxylate derivatives. Method (A): as previously described for dCKi-2-1, to a stirred solution of appropriate 1-([1,1′-biaryl]-3-yl)thiourea (10.0 mmol) in ethanol (100 mL) was added ethyl bromopyruvate (1.5 mL, 12.0 mmol). The resulting mixture was heated at reflux for 3 hrs, then allowed to cool to room temperature and concentrated under reduced pressure. The residue was purified by flash chromatography to afford expected ethyl 2-([1,1′-biaryl]-3-ylamino)thiazole-4-carboxylate derivative. Method (B): under argon, a suspension of appropriate aryl halide (1 mmol), ethyl 2-((5-(4,4,5,5-tetramethyl-1,3,2-dioxaborolan-2-yl)phenyl)amino) thiazole-4-carboxylate or analogue (1.1 mmol), PdCl₂(dppf) (36 mg, 0.05 mmol) and Na₂CO₃ (212 mg, 2 mmol) in a degassed mixture of 1,4-dioxane-water (5:1, 12 mL) was refluxed for 2 hrs, upon complete consumption of starting material. The solvent was distillated off under reduced pressure and the residue purified by flash chromatography to afford corresponding ethyl 2-([1,1′-biaryl]-3-ylamino)thiazole-4-carboxylate derivative or analogue. Method (C): under argon, to a solution of 4-halogenoarylsulfonyl chloride (1.0 mmol) in 1,4-dioxane (12 mL) were added successively appropriate amine (1.15 mmol) and K₂CO₃ (3.0 mmol). The reaction mixture was stirred at room temperature until complete consumption of starting material monitored by LCMS. To the resulting suspension, were added successively ethyl 2-((2-methyl-5-(4,4,5,5-tetramethyl-1,3,2-dioxaborolan-2-yl)phenyl)(propyl)amino)thiazole-4-carboxylate OR0625-2 (1.10 mmol), PdCl₂(dppf) (0.15 mmol), and water (2 mL). The reaction mixture was thoroughly degassed several times under argon fillings, and then heated at 80° C. for 2 hours. The solvent was distillated off and the residue was triturated with DCM (3×50 mL). The combined organic layers were dried over Na₂SO₄. The solvent was distillated off under reduced pressure, and the residue was purified by flash chromatography to afford expected ethyl 2-([1,1′-biaryl]-3-ylamino)thiazole-4-carboxylate.

Ethyl 2-((4-methyl-4′-(2-(4-methylpiperazin-1-yl)ethyl)-[1,1′-biphenyl]-3-yl)(propyl)amino)thiazole-4-carboxylate OR0600-1 (method A, 86%) as a yellow oil. ¹H NMR (400 MHz, CDCl₃) δ 7.53 (dd, J=8.0, 1.9 Hz, 1H), 7.49 (d, J=8.2 Hz, 2H), 7.43 (d, J=1.9 Hz, 1H), 7.40 (d, J=8.0 Hz, 1H), 7.29 (s, 1H), 7.28 (d, J=8.2 Hz, 2H), 4.36 (q, J=7.1 Hz, 2H), 3.85 (brs, 2H), 2.88-2.80 (m, 2H), 2.67-2.59 (m, 2H), 2.50 (brs, 8H), 2.31 (s, 3H), 2.25 (s, 3H), 1.69 (sext, J=7.4 Hz, 2H), 1.38 (t, J=7.1 Hz, 3H), 0.94 (t, J=7.4 Hz, 3H); LCMS C₂₉H₃₈N₄O₂S method (B) R_{c}=5.102 min. ESI+ m/z=507.3 (M+H).

2-((4-Methyl-4′-(3-(4-methylpiperazin-1-yl)propyl)-[1,1′-biphenyl]-3-yl)(propyl)amino)thiazole-4-carboxylate OR0601-1 (method A, 93%) as a beige foam. Rf=0.35 (DCM-MeOH—NH₄OH, 90:9:1); ¹H NMR (400 MHz, CDCl₃) δ 7.53 (dd, J=7.9, 1.9 Hz, 1H), 7.48 (d, J=8.1 Hz, 2H), 7.42 (d, J=1.9 Hz, 1H), 7.39 (d, J=7.9 Hz, 1H), 7.28 (s, 1H), 7.24 (d, J=8.1 Hz, 2H), 4.35 (q, J=7.1 Hz, 2H), 3.93 (brs, 2H), 2.73 (brs, 8H), 2.72-2.64 (m, 2H), 2.54-2.46 (m, 2H), 2.44 (s, 3H), 2.25 (s, 3H), 1.90 (quint, J=7.6 Hz, 2H), 1.69 (sext, J=7.4 Hz, 2H), 1.37 (t, J=7.1 Hz, 3H), 0.94 (t, J=7.4 Hz, 3H). ¹³C NMR (100 MHz, CDCl₃) δ 170.89, 162.09, 143.97, 143.18, 141.20, 140.96, 137.65, 135.87, 132.68, 129.06, 127.83, 127.24, 126.97, 116.75, 61.11, 57.47, 54.30, 53.68, 51.98, 45.31, 33.16, 28.02, 21.20, 17.37, 14.48, 11.43; LCMS C₃₀H₄₀N₄O₂S method (B) Rₜ=5.158 min, ESI+ m/z=521.4 (M+H).

Ethyl 2-((2-methyl-5-(6-(2-(4-methylpiperazin-1-yl)ethyl)pyridin-3-yl)phenyl)(propyl)amino)thiazole-4-carboxylate OR0602-1 (method A, 47%) as a light brown oil. LCMS C₂₈H₃₇N₅O₂S method (B) Rₜ=4.730 min, ESI+ m/z=508.2 (M+H).

Ethyl 2-(isobutyl(4-methyl-4′-(2-(4-methylpiperazin-1-yl)ethyl)-[1,1′-biphenyl]-3-yl)amino)thiazole-4-carboxylate OR0603-1 (method A, 57%) as a light brown solid. LCMS C₃₀H₄₀N₄O₂S method (B) Rₜ=5.363 min, ESI+ m/z=521.2 (M+H).

Ethyl 2-((cyclopropylmethyl)(4-methyl-4′-(2-(4-methylpiperazin-1-yl)ethyl)-[1,1′-biphenyl]-3-yl)amino)thiazole-4-carboxylate OR0604-1 (method A, 100%) as a yellow solid. LCMS C₃₀H₃₈N₄O₂S method (B) Rₜ=5.138 min, ESI+ m/z=519.3 (M+H).

Ethyl 2-(iso-pentyl(4-methyl-4′-(2-(4-methylpiperazin-1-yl)ethyl)-[1,1′-biphenyl]-3-yl)amino)thiazole-4-carboxylate OR0605-1 (method A, 31%) as a light yellow solid. Rf=0.45 (DCM-MeOH—NH₄OH, 90:9:1); LCMS C₃₁H₄₂N₄O₂S method (B) Rₜ=5.460 min, ESI+m/z=535.3 (M+H).

Ethyl 2-(butyl(4-methyl-4′-(2-(4-methylpiperazin-1-yl)ethyl)-[1,1′-biphenyl]-3-yl)amino)thiazole-4-carboxylate OR0606-1 (method A, 89%), a light yellow solid, engaged in the next step without further purification. Rf=0.45 (DCM-MeOH, 90:10); ¹H NMR (400 MHz, CDCl₃) δ 7.53 (dd, J=8.0, 1.9 Hz, 1H), 7.49 (d, J=8.2 Hz, 2H), 7.42 (d, J=1.9 Hz, 1H), 7.40 (d, J=8.0 Hz, 1H), 7.28 (s, 1H), 7.28 (d, J=8.2 Hz, 2H), 4.35 (q, J=7.1 Hz, 2H), 3.96 (brs, 2H), 2.90-2.82 (m, 2H), 2.72-2.64 (m, 2H), 2.58 (brs, 8H), 2.37 (s, 3H), 2.25 (s, 3H), 1.63 (quint, J=7.8 Hz, 2H), 1.42-1.34 (m, 2H), 1.38 (t, J=7.1 Hz, 2H), 0.91 (t, J=7.4 Hz, 3H). ¹³C NMR (100 MHz, CDCl₃) δ 170.89, 162.11, 143.97, 143.20, 140.95, 139.72, 137.86, 135.94, 132.70, 129.40, 127.87, 127.26, 127.01, 116.75, 61.12, 60.21, 54.93, 52.71, 51.88, 45.79, 33.18, 30.06, 20.26, 17.40, 14.49, 14.04; LCMS C₃₀H₄₀N₄O₂S method (B) Rₜ=5.117 min, ESI+ m/z=521.3 (M+H).

Ethyl 2-(isobutyl(2-methyl-5-(6-(2-(4-methylpiperazin-1-yl)ethoxy)pyridin-3-yl)phenyl)amino)thiazole-4-carboxylate OR0608-1 (method A, 100%) as a light brown solid. LCMS C₂₉H₃₉N₅O₃S method (B) Rₜ=5.274 min, ESI+ m/z=538.2 (M+H).

Ethyl 2-(isobutyl(2-methyl-5-(4-(methylsulfonamidomethyl)-1H-1,2,3-triazol-1-yl)phenyl)amino)thiazole-4-carboxylate OR0609-1 (method A, 38%) as a yellow solid. ¹H NMR (400 MHz, CDCl₃) δ 8.05 (s, 1H), 7.68 (d, J=2.0 Hz, 1H), 7.64 (dd, J=8.3, 2.0 Hz, 1H), 7.49 (d, J=8.3 Hz, 1H), 7.34 (s, 1H), 5.22 (brs, 1H), 4.52 (s, 2H), 4.34 (q, J=7.1 Hz, 2H), 3.81 (brs, 2H), 3.01 (s, 3H), 2.29 (s, 3H), 2.06-2.00 (m, 1H), 1.36 (t, J=7.1 Hz, 3H), 0.99 (d, J=6.8 Hz, 6H); LCMS C₂₁H₂₈N₆O₄S₂ method (B)Rₜ=6.189 min, ESI+ m/z=493.2 (M+H).

Ethyl 2-((2-((tert-butoxycarbonyl)amino)ethyl)(4-methyl-4′-(2-(4-methylpiperazin-1-yl)ethyl)-[1,1′-biphenyl]-3-yl)amino)thiazole-4-carboxylate OR0610-2 (method A, 399 mg, 74%) as a light yellow solid. Rf=0.47 (DCM-MeOH, 90:10); ¹H NMR (400 MHz, CDCl₃) δ 7.53 (dd, J=7.9, 1.8 Hz, 1H), 7.49 (d, J=8.2 Hz, 2H), 7.48 (d, J=1.8 Hz, 1H), 7.38 (d, J=7.9 Hz, 1H), 7.30 (s, 1H), 7.25 (d, J=8.2 Hz, 2H), 5.56 (brs, 1H), 4.34 (q, J=7.1 Hz, 2H), 3.55-3.47 (m, 2H), 2.88-2.80 (m, 2H), 2.69-2.61 (m, 2H), 2.58 (brs, 8H), 2.35 (s, 3H), 2.23 (s, 3H), 1.37 (t, J=7.1 Hz, 3H), 1.35 (s, 9H); LCMS C₃₃H₄₅N₅O₄S method (B) Rₜ=5.147 min, ESI+ m/z=608.3 (M+H).

Ethyl 2-((3-((tert-butoxycarbonyl)amino)propyl)(4-methyl-4′-(2-(4-methylpiperazin-1-yl)ethyl)-[1,1′-biphenyl]-3-yl)amino)thiazole-4-carboxylate OR0611-2 (method A, 67%) as a pale yellow solid. Rf=0.55 (DCM-MeOH, 90:10); ¹H NMR (400 MHz, CDCl₃) δ 7.54 (dd, J=8.0, 1.8 Hz, 1H), 7.47 (d, J=8.2 Hz, 2H), 7.40 (d, J=8.0 Hz, 1H), 7.36 (d, J=1.8 Hz, 1H), 7.29 (s, 1H), 7.27 (d, J=8.2 Hz, 2H), 6.25 (brs, 1H), 4.39 (q, J=6.9 Hz, 2H), 4.29 (brs, 1H), 3.70 (brs, 1H), 3.42-3.34 (m, 2H), 3.28-3.20 (m, 2H), 2.89-2.81 (m, 2H), 2.71-2.63 (m, 2H), 2.62 (brs, 8H), 2.39 (s, 3H), 2.24 (s, 3H), 1.83-1.75 (m, 2H), 1.46 (s, 9H), 1.39 (t, J=6.9 Hz, 3H); ¹³C NMR (100 MHz, CDCl₃) δ 171.27, 161.95, 156.50, 143.61, 143.10, 141.30, 139.72, 137.70, 135.42, 132.93, 129.39, 127.90, 127.58, 127.05, 116.80, 78.90, 61.14, 60.08, 54.79, 52.51, 49.56, 45.64, 37.64, 33.10, 28.95, 28.65, 17.31, 14.61; LCMS C₃₄H₄₇N₅O₄S method (B) Rₜ=5.401 min, ESI+ m/z=622.4 (M+H).

Ethyl 2-((2-methyl-5-(6-(4-methylpiperazine-1-carbonyl)pyridin-3-yl)phenyl)(propyl)amino)thiazole-4-carboxylate OR0612-1 (method A, 62%) as a light yellow oil. ¹H NMR (400 MHz, CDCl₃) δ 8.78 (d, J=2.0 Hz, 1H), 7.97 (dd, J=8.2, 2.0 Hz, 1H), 7.76 (d, J=8.2 Hz, 1H), 7.56 (dd, J=8.0, 1.9 Hz, 1H), 7.49 (d, J=8.0 Hz, 1H), 7.46 (d, J=1.9 Hz, 1H), 7.30 (s, 1H), 4.36 (q, J=7.1 Hz, 2H), 3.94 (brs, 4H), 3.81 (brs, 2H), 2.68 (brs, 2H), 2.60 (brs, 2H), 2.44 (s, 3H), 2.29 (s, 3H), 1.70 (sext, J=7.4 Hz, 2H), 1.38 (t, J=7.1 Hz, 3H), 0.96 (t, J=7.4 Hz, 3H); LCMS C₂₇H₃₃N₅O₃S method (B) Rₜ=5.007 min, ESI+ m/z=508.2 (M+H).

Ethyl 2-((2-methyl-5-(6-(4-methylpiperazine-1-carboxamido)pyridin-3-yl)phenyl)(propyl)amino)thiazole-4-carboxylate OR0616-1 (method A, 45%) as a yellow oil. ¹H NMR (400 MHz, CDCl₃) δ 8.39 (d, J=2.3 Hz, 1H), 8.07 (d, J=8.7 Hz, 1H), 7.85 (dd, J=8.7, 2.3 Hz, 1H), 7.50 (dd, J=8.0, 1.9 Hz, 1H), 7.43 (d, J=8.0 Hz, 1H), 7.40 (d, J=1.9 Hz, 1H), 7.30 (s, 1H), 4.35 (q, J=7.1 Hz, 2H), 3.91 (brs, 2H), 3.75 (brs, 4H), 2.73 (brs, 4H), 2.51 (s, 3H), 2.26 (s, 3H), 1.69 (sext, J=7.4 Hz, 2H), 1.37 (t, J=7.1 Hz, 3H), 0.95 (t, J=7.4 Hz, 3H); LCMS C₂₇H₃₄N₆O₃S method (B) Rₜ=5.089 min, ESI+ m/z=523.3 (M+H).

Ethyl 2-((2-methyl-5-(6-((4-methylpiperazine)-1-sulfonamido)pyridin-3-yl)phenyl)(propyl)amino)thiazole-4-carboxylate OR0625-1 (method B, 41%) as a white solid. ¹H NMR (400 MHz, CDCl₃) δ 8.34 (d, J=2.2 Hz, 1H), 7.88 (dd, J=9.1, 2.2 Hz, 1H), 7.54 (d, J=9.1 Hz, 1H), 7.47 (dd, J=8.0, 1.7 Hz, 1H), 7.44 (d, J=8.0 Hz, 1H), 7.36 (d, J=1.7 Hz, 1H), 7.31 (s, 1H), 4.35 (q, J=7.1 Hz, 2H), 3.91 (brs, 2H), 3.53 (brs, 4H), 2.91 (brs, 4H), 2.56 (s, 3H), 2.27 (s, 3H), 1.69 (sext, J=7.4 Hz, 2H), 1.38 (t, J=7.1 Hz, 3H), 0.95 (t, J=7.4 Hz, 3H); LCMS C₂₆H₃₄N₆O₄S₂ method (B) Rₜ=5.084 min, ESI+ m/z=559.3 (M+H).

Ethyl 2-((2-methyl-5-(6-((4-methylpiperazin-1-yl)sulfonyl)pyridin-3-yl)phenyl)(propyl)amino)thiazole-4-carboxylate OR0627-1 (method B, 68%) as a light brown solid. Rf=0.50 (DCM-MeOH, 95:5); ¹H NMR (400 MHz, CDCl₃) δ 8.88 (d, J=2.2 Hz, 1H), 8.04 (dd, J=8.1, 2.2 Hz, 1H), 7.97 (d, J=8.1 Hz, 1H), 7.56 (dd, J=7.9, 1.9 Hz, 1H), 7.51 (d, J=7.9 Hz, 1H), 7.46 (d, J=1.9 Hz, 1H), 7.31 (s, 1H), 4.36 (q, J=7.1 Hz, 2H), 3.94 (brs, 2H), 3.46 (brs, 4H), 2.63 (brs, 4H), 2.39 (s, 3H), 2.30 (s, 3H), 1.70 (sext, J=7.4 Hz, 2H), 1.38 (t, J=7.1 Hz, 3H), 0.96 (t, J=7.4 Hz, 3H); ¹³C NMR (100 MHz, CDCl₃) δ 170.37, 161.98, 154.68, 148.45, 144.14, 143.85, 138.78, 138.61, 136.00, 135.83, 133.53, 128.51, 127.59, 123.26, 116.90, 61.21, 54.34, 53.90, 46.13, 24.99, 21.27, 17.63, 14.49, 11.43; LCMS C₂₆H₃₃N₅O₄S₂ method (B) Rₜ=5.281 min, ESI+ m/z=544.2 (M+H).

Ethyl 2-((2-methyl-5-(6-(2-(4-methylpiperazin-1-yl)ethoxy)pyridin-3-yl)phenyl)(propyl)amino)thiazole-4-carboxylate OR0629-1 (method B, 84%) as a yellow oil. ¹H NMR (400 MHz, CDCl₃) δ 8.33 (d, J=2.3 Hz, 1H), 7.75 (dd, J=8.7, 2.3 Hz, 1H), 7.47 (dd, J=7.9, 1.9 Hz, 1H), 7.41 (d, J=7.9 Hz, 1H), 7.37 (d, J=1.9 Hz, 1H), 7.29 (s, 1H), 6.82 (d, J=8.7 Hz, 1H), 4.48 (t, J=5.8 Hz, 2H), 4.35 (q, J=7.1 Hz, 2H), 3.89 (brs, 2H), 2.84 (t, J=5.8 Hz, 2H), 2.71 (brs, 4H), 2.62 (brs, 4H), 2.37 (s, 3H), 2.25 (s, 3H), 1.69 (sext, J=7.4 Hz, 2H), 1.37 (t, J=7.1 Hz, 3H), 0.94 (t, J=7.4 Hz, 3H); LCMS C₂₈H₃₇N₅O₃S method (B) Rₜ=5.247 min, ESI+ m/z=524.3 (M+H).

Ethyl 2-((2-methyl-5-(6-(2-(4-methylpiperazin-1-yl)-2-oxoethoxy)pyridin-3-yl)phenyl)(propyl)amino)thiazole-4-carboxylate OR0630-1 (method B, 71%) as a light brown oil. ¹H NMR (400 MHz, CDCl₃) δ 8.29 (d, J=2.4 Hz, 1H), 7.79 (dd, J=8.6, 2.4 Hz, 1H), 7.47 (dd, J=8.0, 1.8 Hz, 1H), 7.41 (d, J=8.0 Hz, 1H), 7.36 (d, J=1.8 Hz, 1H), 7.29 (s, 1H), 6.96 (d, J=8.6 Hz, 1H), 5.05 (s, 2H), 4.35 (q, J=7.1 Hz, 2H), 3.91 (brs, 2H), 3.74 (brs, 2H), 3.63 (brs, 2H), 2.62-2.50 (m, 4H), 2.40 (s, 3H), 2.25 (s, 3H), 1.69 (sext, J=7.4 Hz, 2H), 1.37 (t, J=7.1 Hz, 3H), 0.94 (t, J=7.4 Hz, 3H); LCMS C₂₈H₃₅N₅O₄S method (B) Rₜ=5.234 min, ESI+ m/z=538.3 (M+H).

Ethyl 2-((5-fluoro-4-methyl-4′-((4-methylpiperazin-1-yl)sulfonyl)-[1,1′-biphenyl]-3-yl)(propyl)amino)thiazole-4-carboxylate OR0633-1 (method A, 86%) as a yellow oil. LCMS C₂₇H₃₃FN₄O₄S₂ method (B) Rₜ=5.584 min, ESI+ m/z=561.2 (M+H).

Ethyl 2-((3′-methoxy-4-methyl-4′-((4-methylpiperazin-1-yl)sulfonyl)-[1,1′-biphenyl]-3-yl)(propyl)amino)thiazole-4-carboxylate OR0635-1 (method B, 82%) as a light brown solid. Rf=0.50 (DCM-MeOH, 95:5); ¹H NMR (400 MHz, CDCl₃) δ 7.91 (d, J=8.0 Hz, 1H), 7.55 (dd, J=8.0, 1.9 Hz, 1H), 7.45 (d, J=8.2 Hz, 1H), 7.44 (d, J=1.9 Hz, 1H), 7.31 (s, 1H), 7.20 (dd, J=8.2, 1.4 Hz, 1H), 7.12 (d, J=1.4 Hz, 1H), 4.36 (q, J=7.1 Hz, 2H), 3.99 (s, 3H), 3.97 (brs, 2H), 3.44 (brs, 4H), 2.70 (brs, 4H), 2.46 (s, 3H), 2.29 (s, 3H), 1.69 (sext, J=7.4 Hz, 2H), 1.38 (t, J=7.1 Hz, 3H), 0.96 (t, J=7.4 Hz, 3H); LCMS C₂₈H₃₆FN₄O₅S₂ method (B) R=5.258 min, ESI+ m/z=574.3 (M+H).

Ethyl 2-((4′-((4-ethylpiperazin-1-yl)sulfonyl)-4-methyl-[1,1′-biphenyl]-3-yl)(propyl)amino)thiazole-4-carboxylate OR0638-1 (method B, 82%) as a colorless oil. LCMS C₂₈H₃₆N₄O₄S₂ method (B) Rₜ=5.183 min, ESI+ m/z=557.4 (M+H).

Ethyl 2-((4′-((4-(tert-butoxycarbonyl)piperazin-1-yl)sulfonyl)-4-methyl-[1,1′-biphenyl]-3-yl)(propyl)amino)thiazole-4-carboxylate OR0639-2 (method B, 56%) as a white solid. LCMS C₃₁H₄₀N₄O₆S₂ method (B) Rₜ=7.658 min.

Ethyl 2-((4-methyl-4′-((4-methylpiperazin-1-yl)sulfonyl)-[1,1′-biphenyl]-3-yl)(propyl)amino)oxazole-4-carboxylate OR0640-1 (method B, 78%) as a light brown solid. ¹H NMR (400 MHz, CDCl₃) δ 7.80 (d, J=8.5 Hz, 2H), 7.71 (d, J=8.5 Hz, 2H), 7.69 (s, 1H), 7.50 (dd, J=7.8, 1.8 Hz, 1H), 7.41 (d, J=7.8 Hz, 1H), 7.40 (d, J=1.8 Hz, 1H), 4.35 (q, J=7.1 Hz, 2H), 3.87-3.79 (m, 2H), 3.18 (brs, 4H), 2.66 (brs, 4H), 2.40 (s, 3H), 2.24 (s, 3H), 1.70 (sext, J=7.4 Hz, 2H), 1.36 (t, J=7.1 Hz, 3H), 0.94 (t, J=7.4 Hz, 3H); ¹³C NMR (100 MHz, CDCl₃) δ 182.08, 162.14, 160.86, 151.89, 145.03, 140.97, 139.05, 138.45, 138.17, 137.34, 134.13, 133.42, 132.38, 128.48, 127.77, 127.61, 126.81, 61.07, 53.95, 53.70, 45.40, 25.00, 21.20, 17.68, 14.49, 11.30; LCMS C₂₇H₃₄N₄O₅S method (B) Rₜ=5.157 min, ESI+ m/z=527.3 (M+H).

Ethyl 2-((2′-chloro-4-methyl-4′-((4-methylpiperazin-1-yl)sulfonyl)-[1,1′-biphenyl]-3-yl)(propyl)amino)thiazole-4-carboxylate OR0641-1 (method B, 73%) as a light brown solid. Rf=0.50 (DCM-MeOH, 95:5); ¹H NMR (400 MHz, CDCl₃) δ 7.85 (d, J=1.8 Hz, 1H), 7.67 (dd, J=8.0, 1.8 Hz, 1H), 7.50 (d, J=8.0 Hz, 1H), 7.46 (d, J=7.9 Hz, 1H), 7.37 (dd, J=7.9, 1.8 Hz, 1H), 7.33 (d, J=1.8 Hz, 1H), 7.30 (s, 1H), 4.35 (q, J=7.1 Hz, 2H), 3.93 (brs, 2H), 3.13-3.11 (m, 4H), 2.55-2.54 (m, 4H), 2.31 (s, 3H), 2.29 (s, 3H), 1.68 (sext, J=7.4 Hz, 2H), 1.37 (t, J=7.1 Hz, 3H), 0.95 (t, J=7.4 Hz, 3H); ¹³C NMR (100 MHz, CDCl₃) δ 170.64, 162.03, 144.03, 143.81, 142.63, 137.88, 137.41, 135.82, 133.75, 132.47, 131.86, 130.68, 129.53, 129.39, 126.31, 116.90, 61.15, 54.02, 53.66, 45.93, 45.67, 21.18, 17.56, 14.48, 11.43; LCMS C₂₇H₃₃ClN₄O₄S₂ method (B) Rₜ=5.248 min, ESI+ m/z=577.2 (M+H).

Ethyl 2-((4-methyl-4′-((4-methylpiperazin-1-yl)sulfonyl)-2′-(trifluoromethyl)-[1,1′-biphenyl]-3-yl)(propyl)amino) thiazole-4-carboxylate OR0642-1 (method B, 75%) as a light brown solid. Rf=0.65 (DCM-MeOH, 95:5); ¹H NMR (400 MHz, CDCl₃) δ 8.10 (d, J=1.6 Hz, 1H), 7.93 (dd, J=8.0, 1.6 Hz, 1H), 7.54 (d, J=8.0 Hz, 1H), 7.44 (d, J=7.9 Hz, 1H), 7.31 (s, 1H), 7.28 (dd, J=7.9, 1.4 Hz, 1H), 7.20 (d, J=1.4 Hz, 1H), 4.35 (q, J=7.1 Hz, 2H), 3.93 (brs, 2H), 3.16-3.13 (m, 4H), 2.59-2.56 (m, 4H), 2.34 (s, 3H), 2.29 (s, 3H), 1.66 (sext, J=7.4 Hz, 2H), 1.37 (t, J=7.1 Hz, 3H), 0.94 (t, J=7.4 Hz, 3H); ¹³C NMR (100 MHz, CDCl₃) δ 170.66, 162.04, 144.82, 144.01, 142.40, 137.96, 137.71, 135.41, 133.15, 132.28, 130.68, 129.99, 129.67, 129.01, 125.89, 116.98, 61.16, 53.97, 53.61, 45.82, 45.62, 21.13, 17.54, 14.48, 11.42; LCMS C₂₈H₃₃F₃N₄O₄S₂ method (B) Rₜ=5.454 min, ESI+ m/z=611.3 (M+H).

Ethyl 2-((2′-fluoro-4-methyl-4′-((4-methylpiperazin-1-yl)sulfonyl)-[1,1′-biphenyl]-3-yl)(propyl)amino)thiazole-4-carboxylate OR0643-1 (method B, 62%) as a light brown solid. Rf=0.50 (DCM-MeOH, 95:5); ¹H NMR (400 MHz, CDCl₃) δ 7.61-7.60 (m, 2H), 7.53-7.51 (m, 2H), 7.47 (d, J=8.0 Hz, 1H), 7.44 (s, 1H), 7.31 (s, 1H), 4.36 (q, J=7.1 Hz, 2H), 3.93 (brs, 2H), 3.23-3.20 (m, 4H), 2.69-2.67 (m, 4H), 2.42 (s, 3H), 2.29 (s, 3H), 1.70 (sext, J=7.4 Hz, 2H), 1.38 (t, J=7.1 Hz, 3H), 0.96 (t, J=7.4 Hz, 3H); ¹³C NMR (100 MHz, CDCl₃) δ 170.61, 162.05, 148.86, 144.08, 143.16, 138.30, 136.18, 133.69, 132.82, 131.46, 130.32, 129.31, 123.89, 116.92, 116.25, 115.99, 61.18, 53.90, 53.78, 53.57, 45.35, 21.20, 17.62, 14.50, 11.44; LCMS C₂₇H₃₃FN₄O₄S₂ method (B) Rₜ=5.220 min, ESI+ m/z=561.2 (M+H).

Ethyl 2-((2′,6′-difluoro-4-methyl-4′-((4-methylpiperazin-1-yl)sulfonyl)-[1,1′-biphenyl]-3-yl)(propyl)amino)thiazole-4-carboxylate OR0644-1 (method B, 64%) as a light yellow solid. Rf=0.28 (DCM-MeOH, 98:2); ¹H NMR (400 MHz, CDCl₃) δ 7.48 (d, J=8.0 Hz, 1H), 7.43 (d, J=8.0 Hz, 1H), 7.41-7.32 (m, 3H), 7.31 (s, 1H), 4.35 (q, J=7.1 Hz, 2H), 3.91 (brs, 2H), 3.16-3.13 (m, 4H), 2.56-2.54 (m, 4H), 2.32 (s, 3H), 2.30 (s, 3H), 1.68 (sext, J=7.4 Hz, 2H), 1.37 (t, J=7.1 Hz, 3H), 0.94 (t, J=7.4 Hz, 3H); ¹³C NMR (100 MHz, CDCl₃) δ 170.61, 162.04, 161.10, 158.57, 144.01, 142.90, 138.76, 136.73, 132.58, 131.60, 130.51, 127.08, 117.00, 111.60, 61.16, 54.00, 53.67, 45.68, 44.61, 21.12, 17.64, 14.47, 11.40; LCMS C₂₇H₃₂F₂N₄O₄S₂ method (B) Rₜ=5.283 min, ESI+ m/z=579.2 (M+H).

Ethyl 2-((2′-methoxy-4-methyl-4′-((4-methylpiperazin-1-yl)sulfonyl)-[1,1′-biphenyl]-3-yl)(propyl)amino)thiazole-4-carboxylate OR0645-1 (method B, 55%) as a light brown solid. Rf=0.35 (DCM-MeOH, 95:5); LCMS C₂₈H₃₆N₄O₅S₂ method (B) Rₜ=5.291 min, ESI+ m/z=573.2 (M+H).

Ethyl 2-((4-methyl-4′-(piperidin-4-ylsulfonyl)-[1,1′-biphenyl]-3-yl)(propyl)amino)thiazole-4-carboxylate OR0646-1 (method B, 70%) as a light brown solid. Rf=0.25 (DCM-MeOH, 95:5); ¹H NMR (400 MHz, CDCl₃) δ 7.92 (d, J=8.5 Hz, 2H), 7.74 (d, J=8.5 Hz, 2H), 7.58 (dd, J=8.0, 1.9 Hz, 1H), 7.48 (d, J=1.9 Hz, 1H), 7.47 (d, J=8.0 Hz, 1H), 7.30 (s, 1H), 4.35 (q, J=7.1 Hz, 2H), 3.95 (brs, 2H), 3.30-3.24 (m, 2H), 3.12-3.02 (m, 1H), 2.70-2.62 (m, 2H), 2.29 (s, 3H), 2.13-2.05 (m, 2H), 1.76-1.65 (m, 4H), 1.38 (t, J=7.1 Hz, 3H), 0.96 (t, J=7.4 Hz, 3H). ¹³C NMR (100 MHz, CDCl₃) δ 170.56, 162.03, 145.57, 144.10, 143.58, 139.02, 138.09, 135.40, 133.19, 129.96, 128.48, 127.74, 127.67, 116.84, 61.34, 61.21, 53.88, 44.70, 25.33, 21.25, 17.57, 14.49, 11.45; LCMS C₂₇H₃₃N₃O₄S₂ method (B) Rₜ=5.135 min, ESI+ m/z=528.2 (M+H).

Ethyl 2-((4′-((4-((tert-butoxycarbonyl)amino)piperidin-1-yl)sulfonyl)-4-methyl-[1,1′-biphenyl]-3-yl)(propyl)amino) thiazole-4-carboxylate OR0648-2 (method B, 88%) as a colorless oil. Rf=0.50 (DCM-MeOH, 98:2); ¹H NMR (400 MHz, CDCl₃) δ 7.81 (d, J=8.3 Hz, 2H), 7.71 (d, J=8.3 Hz, 2H), 7.58 (dd, J=8.0, 1.5 Hz, 1H), 7.48 (d, J=1.5 Hz, 1H), 7.46 (d, J=8.0 Hz, 1H), 7.30 (s, 1H), 4.41 (brs, 1H), 4.36 (q, J=7.1 Hz, 2H), 3.95 (brs, 2H), 3.73 (brs, 1H), 3.39 (brs, 1H), 2.52-2.43 (m, 2H), 2.29 (s, 3H), 2.02-1.95 (m, 2H), 1.71 (sext, J=7.4 Hz, 2H), 1.53-1.47 (m, 2H), 1.40 (s, 9H), 1.37 (t, J=7.1 Hz, 3H), 0.96 (t, J=7.4 Hz, 3H); LCMS C₃₂H₄₂N₄O₆S₂ method (B) Rₜ=7.522 min, ESI+ m/z=643.2 (M+H).

2-(4-((3′-((4-(Ethoxycarbonyl)thiazol-2-yl)(propyl)amino)-4′-methyl-[1,1′-biphenyl]-4-yl)sulfonyl)piperazin-1-yl)acetic acid OR0649-1 (method B, 42%) as a white solid. Rf=0.11 (DCM-MeOH, 95:5); ¹H NMR (400 MHz, MeOD) δ 7.91 (d, J=8.8 Hz, 2H), 7.87 (d, J=8.8 Hz, 2H), 7.73 (dd, J=8.0, 1.9 Hz, 1H), 7.64 (d, J=1.9 Hz, 1H), 7.56 (d, J=8.0 Hz, 1H), 7.45 (s, 1H), 4.33 (q, J=7.1 Hz, 2H), 3.91 (brs, 2H), 3.36 (s, 2H), 3.21 (brs, 4H), 3.03 (brs, 4H), 2.29 (s, 3H), 1.74 (sext, J=7.4 Hz, 2H), 1.36 (t, J=7.1 Hz, 3H), 0.98 (t, J=7.4 Hz, 3H); ¹³C NMR (100 MHz, MeOD) δ 172.17, 163.30, 145.93, 144.85, 144.66, 140.42, 138.85, 135.45, 134.29, 129.78, 129.26, 128.78, 128.72, 118.30, 62.13, 59.86, 55.21, 53.01, 45.86, 22.14, 17.52, 14.60, 11.55; LCMS C₂₈H₃₄N₄O₆S₂ method (B) Rₜ=5.694 min, ESI+ m/z=588.3 (M+H).

Ethyl 2-((4-methyl-4′-((4-methylpiperazin-1-yl)sulfonyl)-3′-(trifluoromethoxy)-[1,1′-biphenyl]-3-yl)(propyl)amino) thiazole-4-carboxylate OR0650-1 (method B, 82%) as a white solid. Rf=0.36 (DCM-MeOH, 95:5); ¹H NMR (400 MHz, CDCl₃) δ 8.00 (d, J=8.2 Hz, 1H), 7.56 (dd, J=8.2, 1.6 Hz, 1H), 7.54-7.52 (m, 1H), 7.53 (dd, J=8.0, 2.0 Hz, 1H), 7.48 (d, J=8.0 Hz, 1H), 7.43 (d, J=2.0 Hz, 1H), 7.31 (s, 1H), 4.35 (q, J=7.1 Hz, 2H), 3.94 (brs, 2H), 3.28 (brs, 4H), 2.50 (brs, 4H), 1.68 (sext, J=7.4 Hz, 2H), 1.37 (t, J=7.1 Hz, 3H), 0.95 (t, J=7.4 Hz, 3H); ¹³C NMR (100 MHz, CDCl₃) δ 170.41, 161.96, 146.72, 146.54, 144.11, 143.67, 138.66, 137.98, 133.32, 132.53, 128.87, 128.33, 127.50, 124.95, 121.64, 119.17, 116.87, 61.17, 54.49, 53.89, 45.86, 45.67, 21.23, 17.58, 14.46, 11.40; LCMS C₂₈H₃₃F₃N₄O₅S₂ method (B) Rₜ=5.512 min, ESI+ m/z=627.2 (M+H).

Ethyl 2-((4′—(N-(2-((tert-butoxycarbonyl)amino)ethyl)sulfamoyl)-4-methyl-[1,1′-biphenyl]-3-yl)(propyl)amino)thiazole-4-carboxylate OR0651-2 (method B, 66%) as a colorless oil. Rf=0.33 (DCM-MeOH, 95:5); ¹H NMR (400 MHz, CDCl₃) δ 7.92 (d, J=8.4 Hz, 2H), 7.68 (d, J=8.4 Hz, 2H), 7.57 (dd, J=7.8, 1.5 Hz, 1H), 7.47 (d, J=1.5 Hz, 1H), 7.46 (d, J=7.8 Hz, 1H), 7.30 (s, 1H), 5.40 (brs, 1H), 4.91 (brs, 1H), 4.35 (q, J=7.1 Hz, 2H), 3.95 (brs, 2H), 3.28-3.20 (m, 2H), 3.14-3.06 (m, 2H), 2.28 (s, 3H), 1.70 (sext, J=7.4 Hz, 2H), 1.41 (s, 9H), 1.37 (t, J=7.1 Hz, 3H), 0.95 (t, J=7.4 Hz, 3H); ¹³C NMR (100 MHz, CDCl₃) δ 170.61, 161.99, 156.74, 144.22, 143.98, 143.48, 139.28, 138.92, 137.72, 133.12, 128.32, 127.84, 127.65, 127.62, 116.82, 80.11, 61.22, 53.91, 43.97, 40.45, 28.46, 21.25, 17.53, 14.49, 11.44; LCMS C₂₉H₃₈N₄O₆S₂ method (B) Rₜ=7.124 min, ESI+ m/z=603.3 (M+H).

General Procedure for the Synthesis of 1-([1,1′-biaryl]-3-yl)thiourea Derivatives. As previously described above for 1-(3-nitroaryl)thiourea derivatives.

1-iso-Butyl-1-(4-methyl-4′-(2-(4-methylpiperazin-1-yl)ethyl)-[1,1′-biphenyl]-3-yl)thiourea OR0603-2 (95%) as a white solid. LCMS C₂₅H₃₆N₄S method (B) Rₜ=4.591 min, ESI+ m/z=425.3 (M+H).

1-(Cyclopropylmethyl)-1-(4-methyl-4′-(2-(4-methylpiperazin-1-yl)ethyl)-[1,1′-biphenyl]-3-yl)thiourea OR0604-2 (70%) as a white solid. ¹H NMR (400 MHz, CDCl₃) δ 7.53 (dd, J=8.0, 1.9 Hz, 1H), 7.47 (d, J=8.1 Hz, 2H), 7.46 (d, J=1.9 Hz, 1H), 7.37 (d, J=8.0 Hz, 1H), 7.29 (d, J=8.1 Hz, 2H), 5.52 (brs, 2H), 4.38-4.32 (m, 1H), 3.82-3.74 (m, 1H), 2.86-2.82 (m, 2H), 2.67-2.61 (m, 2H), 2.50 (brs, 8H), 2.31 (s, 3H), 2.29 (s, 3H), 1.20-1.14 (m, 1H), 0.49-0.43 (m, 2H), 0.22-0.16 (m, 2H); LCMS C₂₅H₃₄N₄S method (B) Rₜ=4.413 min, ESI+ m/z=423.2 (M+H).

1-iso-Pentyl-1-(4-methyl-4′-(2-(4-methylpiperazin-1-yl)ethyl)-[1,1′-biphenyl]-3-yl)thiourea OR0605-2 (92%) as a light yellow solid, used in the next step without further purification. Rf=0.18 (DCM-MeOH—NH₄OH, 90:9:1); LCMS C₂₆H₃₈N₄S method (B) Rₜ=4.820 min, ESI+ m/z=439.3 (M+H).

1-Butyl-1-(4-methyl-4′-(2-(4-methylpiperazin-1-yl)ethyl)-[1,1′-biphenyl]-3-yl)thiourea OR0606-2 (63%) as a light yellow solid. Rf=0.12 (DCM-MeOH, 95:5); ¹H NMR (400 MHz, CDCl₃) δ 7.53 (d, J=7.9 Hz, 1H), 7.47 (d, J=7.9 Hz, 2H), 7.38 (d, J=7.9 Hz, 1H), 7.33 (s, 1H), 7.29 (d, J=7.9 Hz, 2H), 5.51 (brs, 2H), 4.50-4.47 (m, 1H), 3.69-3.67 (m, 1H), 2.87-2.85 (m, 2H), 2.69 (brs, 8H), 2.66-2.64 (m, 2H), 2.38 (s, 3H), 2.26 (s, 3H), 1.73-1.71 (m, 1H), 1.60-1.58 (m, 1H), 1.36-1.35 (m, 2H), 0.91 (t, J=7.3 Hz, 3H); ¹³C NMR (100 MHz, CDCl₃) δ 181.86, 140.93, 140.19, 140.04, 137.31, 134.50, 132.64, 129.51, 127.59, 126.98, 126.25, 60.11, 54.85, 54.82, 52.63, 45.72, 33.13, 29.66, 20.14, 17.15, 13.97; LCMS C₂₅H₃₆N₄S method (B) Rₜ=4.593 min, ESI+ m/z=425.3 (M+H).

1-iso-Butyl-1-(2-methyl-5-(6-(2-(4-methylpiperazin-1-yl)ethoxy)pyridin-3-yl)phenyl)thiourea OR0608-2 (90%) as a white solid. ¹H NMR (400 MHz, CDCl₃) δ 8.31 (d, J=2.5 Hz, 1H), 7.73 (dd, J=8.6, 2.5 Hz, 1H), 7.47 (dd, J=8.0, 1.9 Hz, 1H), 7.41 (d, J=8.0 Hz, 1H), 7.31 (d, J=1.9 Hz, 1H), 6.84 (d, J=8.6 Hz, 1H), 5.49 (brs, 2H), 4.50 (t, J=5.7 Hz, 2H), 3.39-3.31 (m, 2H), 2.92-2.84 (m, 2H), 2.81 (brs, 8H), 2.48 (s, 3H), 2.27 (s, 3H), 1.99-1.98 (m, 1H), 0.97 (d, J=6.8 Hz, 6H); LCMS C₂₄H₃₅N₅OS method (B) Rₜ=4.530 min, ESI+ m/z=442.3 (M+H).

N-((1-(3-(1-iso-Butylthioureido)-4-methylphenyl)-1H-1,2,3-triazol-4-yl)methyl)methanesulfonamide OR0609-2 (95%) as a white solid. ¹H NMR (400 MHz, DMSO) δ 8.75 (s, 1H), 7.82-7.52 (m, 4H), 4.31 (s, 2H), 3.60 (brs, 2H), 2.95 (s, 3H), 2.18 (s, 3H), 1.90-1.78 (m, 1H), 0.96-0.86 (m, 6H); LCMS C₁₆H₂₄N₆O₂S₂ method (B) Rₜ=5.127 min, ESI+ m/z=397.2 (M+H).

tert-Butyl (2-(1-(4-methyl-4′-(2-(4-methylpiperazin-1-yl)ethyl)-[1,1′-biphenyl]-3-yl)thioureido)ethyl)carbamate OR0610-3 (98%) as a pale yellow solid, engaged in the next step without further purification. ¹H NMR (400 MHz, CDCl₃) δ 7.53 (dd, J=7.9, 1.4 Hz, 1H), 7.49 (d, J=8.1 Hz, 2H), 7.40-7.36 (m, 2H), 7.26 (d, J=8.1 Hz, 2H), 5.46 (brs, 1H), 4.80-4.72 (m, 1H), 3.86-3.78 (m, 1H), 3.54-3.38 (m, 2H), 2.89-2.81 (m, 2H), 2.72-2.64 (m, 2H), 2.63 (brs, 8H), 2.38 (s, 3H), 2.25 (s, 3H), 1.39 (s, 9H); LCMS C₂₈H₄₁N₅O₂S method (B) Rₜ=4.513 min, ESI+ m/z=512.3 (M+H).

tert-Butyl (3-(1-(4-methyl-4′-(2-(4-methylpiperazin-1-yl)ethyl)-[1,1′-biphenyl]-3-yl)thioureido)propyl)carbamate OR0611-3 (90%) a pale yellow solid, engaged in the next step without further purification. ¹H NMR (400 MHz, CDCl₃) δ 7.53 (dd, J=7.9, 1.8 Hz, 1H), 7.46 (d, J=8.1 Hz, 2H), 7.39 (d, J=7.9 Hz, 1H), 7.31 (d, J=1.8 Hz, 1H), 7.28 (d, J=8.2 Hz, 1H), 5.56 (brs, 2H), 5.38 (brs, 1H), 4.74-4.66 (m, 1H), 3.72-3.63 (m, 1H), 3.35-3.13 (m, 2H), 2.88-2.80 (m, 2H), 2.67-2.59 (m, 2H), 2.52 (brs, 8H), 2.31 (s, 3H), 2.25 (s, 3H), 1.94-1.71 (m, 2H), 1.42 (s, 9H); LCMS C₂₉H₄₃N₅O₂S method (B) Rₜ=4.607 min, ESI+ m/z=526.3 (M+H).

1-(2-Methyl-5-(6-(4-methylpiperazine-1-carbonyl)pyridin-3-yl)phenyl)-1-propylthiourea OR0612-2 (82%) as a yellow solid. LCMS C₂₂H₂₉N₅OS method (B) Rₜ=4.215 min, ESI+ m/z=412.2 (M+H).

4-Methyl-N-(5-(4-methyl-3-(1-propylthioureido)phenyl)pyridin-2-yl)piperazine-1-carboxamide OR0616-2 (88%) as a yellow solid. LCMS C₂₂H₃₀N₆OS method (B) Rₜ=4.309 min, ESI+ m/z=427.3 (M+H).

1-(5-Fluoro-4-methyl-4′-((4-methylpiperazin-1-yl)sulfonyl)-[1,1′-biphenyl]-3-yl)-1-propylthiourea OR0633-2 (97%) as a yellow solid. LCMS C₂₂H₂₉FN₄O₂S₂ method (B) Rₜ=4.870 min, ESI+ m/z=465.2 (M+H).

General Procedure for the Synthesis of N-([1,1′-biaryl]-3-ylcarbamothioyl)benzamide Derivatives. As previously described above.

N-((4-Methyl-4′-(2-(4-methylpiperazin-1-yl)ethyl)-[1,1′-biphenyl]-3-yl)(propyl)carbamothioyl)benzamide OR0600-3 (quantitative), as a pale yellow solid, was used in the next step without further purification. LCMS C₃₁H₃₈N₄OS method (B) Rₜ=4.868 min, ESI+ m/z=515.3 (M+H).

N-(iso-Butyl(4-methyl-4′-(2-(4-methylpiperazin-1-yl)ethyl)-[1,1′-biphenyl]-3-yl)carbamothioyl)benzamide OR0603-3 (quantitative) as a white solid. LCMS C₃₂H₄₀N₄OS method (B) Rₜ=5.080 min, ESI+ m/z=529.6 (M+H).

N-((Cyclopropylmethyl)(4-methyl-4′-(2-(4-methylpiperazin-1-yl)ethyl)-[1,1′-biphenyl]-3-yl)carbamothioyl)benzamide OR0604-3 (quantitative) as a white solid. LCMS C₃₂H₃SN₄OS method (B) Rₜ=4.915 min, ESI+ m/z=527.3 (M+H).

N-(iso-Pentyl(4-methyl-4′-(2-(4-methylpiperazin-1-yl)ethyl)-[1,1′-biphenyl]-3-yl)carbamothioyl)benzamide OR0605-3 (96%), as a light yellow solid, used in the next step without further purification. Rf=0.40 (DCM-MeOH—NH₄OH, 90:9:1); LCMS C₃₃H₄₂N₄OS method (B) Rₜ=5.154 min, ESI+ m/z=543.3 (M+H).

N-(Butyl(4-methyl-4′-(2-(4-methylpiperazin-1-yl)ethyl)-[1,1′-biphenyl]-3-yl)carbamothioyl)benzamide OR0606-3 (96%) as a light yellow solid, used in the next step without further purification. Rf=0.40 (DCM-MeOH—NH₄OH, 90:9:1); LCMS C₃₂H₄₀N₄OS method (B) Rₜ=5.059 min, ESI+ m/z=529.3 (M+H).

N-(iso-Butyl(2-methyl-5-(6-(2-(4-methylpiperazin-1-yl)ethoxy)pyridin-3-yl)phenyl)carbamothioyl)benzamide OR0608-3 (quantitative) as a white solid. LCMS C₃₁H₃₉N₅O₂S method (B) Rₜ=5.074 min, ESI+ m/z=546.3 (M+H).

N-(iso-Butyl(2-methyl-5-(4-(methylsulfonamidomethyl)-1H-1,2,3-triazol-1-yl)phenyl)carbamothioyl)benzamide OR0609-3 (quantitative) as a white solid. LCMS C₂₃H₂₈N₆O₃S₂ method (B) Rₜ=5.882 min, ESI+ m/z=501.2 (M+H).

tert-Butyl (2-(3-benzoyl-1-(4-methyl-4′-(2-(4-methylpiperazin-1-yl)ethyl)-[1,1′-biphenyl]-3-yl)thioureido)ethyl) carbamate OR0610-4 (96%) as a pale yellow solid, used in the next step without further purification. LCMS C₃₅H₄₅N₅O₃S method (B) Rₜ=4.938 min, ESI+ m/z=616.3 (M+H).

tert-Butyl (3-(3-benzoyl-1-(4-methyl-4′-(2-(4-methylpiperazin-1-yl)ethyl)-[1,1′-biphenyl]-3-yl)thioureido)propyl) carbamate OR0611-4 (98%) as a light yellow solid, used in the next step without further purification. LCMS C₃₆H₄₇N₅O₃S method (B) Rₜ=4.936 min, ESI+ m/z=630.3 (M+H).

N-((2-Methyl-5-(6-(4-methylpiperazine-1-carbonyl)pyridin-3-yl)phenyl)(propyl)carbamothioyl)benzamide OR0612-3 (quantitative) as a beige solid. LCMS C₂₉H₃₃N₅O₂S method (B) Rₜ=4.773 min, ESI+ m/z=516.3 (M+H).

N-(5-(3-(3-Benzoyl-1-propylthioureido)-4-methylphenyl)pyridin-2-yl)-4-methylpiperazine-1-carboxamide OR0616-3 (quantitative) as a yellow solid. LCMS C₂₉H₃₄N₆O₂S method (B) Rₜ=4.875 min, ESI+ m/z=531.3 (M+H).

N-((5-Fluoro-4-methyl-4′-((4-methylpiperazin-1-yl)sulfonyl)-[1,1′-biphenyl]-3-yl)(propyl)carbamothioyl)benzamide OR0633-3 (quantitative) as a yellow solid. LCMS C₂₉H₃₃FN₄₀₃S₂ method (B) Rₜ=5.434 min, ESI+ m/z=569.2 (M+H).

General Procedure for the Synthesis of [1,1′-biaryl]-3-N-alkylamine derivatives. As previously described above.

N-iso-Butyl-4-methyl-4′-(2-(4-methylpiperazin-1-yl)ethyl)-[1,1′-biphenyl]-3-amine OR0603-4 (75%) as a white solid. ¹H NMR (400 MHz, CDCl₃) δ 7.50 (d, J=8.2 Hz, 2H), 7.25 (d, J=8.2 Hz, 2H), 7.10 (d, J=7.7 Hz, 1H), 6.84 (dd, J=7.7, 1.6 Hz, 1H), 6.77 (d, J=1.6 Hz, 1H), 3.60 (brs, 1H), 3.04 (d, J=6.7 Hz, 2H), 2.88-2.80 (m, 2H), 2.69-2.61 (m, 2H), 2.53 (brs, 8H), 2.32 (s, 3H), 2.17 (s, 3H), 2.02-1.92 (m, 1H), 1.02 (d, J=6.7 Hz, 6H); LCMS C₂₄H₃₅N₃ method (B) Rₜ=4.324 min, ESI+ m/z=366.4 (M+H).

N-(Cyclopropylmethyl)-4-methyl-4′-(2-(4-methylpiperazin-1-yl)ethyl)-[1,1′-biphenyl]-3-amine OR0604-4 (71%) as a colorless oil. ¹H NMR (400 MHz, CDCl₃) δ 7.50 (d, J=8.1 Hz, 2H), 7.25 (d, J=8.1 Hz, 2H), 7.10 (d, J=7.8 Hz, 1H), 6.86 (dd, J=7.8, 1.6 Hz, 1H), 6.77 (d, J=1.6 Hz, 1H), 3.06 (d, J=6.9 Hz, 2H), 2.87-2.81 (m, 2H), 2.68-2.60 (m, 2H), 2.51 (brs, 8H), 2.31 (s, 3H), 2.20 (s, 3H), 1.20-1.14 (m, 1H), 0.61-0.55 (m, 2H), 0.30-0.24 (m, 2H); LCMS C₂₄H₃₃N₃ method (B) Rₜ=4.392 min, ESI+ m/z=364.3 (M+H).

N-iso-Pentyl-4-methyl-4′-(2-(4-methylpiperazin-1-yl)ethyl)-[1,1′-biphenyl]-3-amine OR0605-4 (68%) as a light yellow oil. Rf=0.45 (DCM-MeOH—NH₄OH, 90:9:1); LCMS C₂₅H₃₇N₃ method (B) Rₜ=4.966 min, ESI+ m/z=380.6 (M+H).

N-Butyl-4-methyl-4′-(2-(4-methylpiperazin-1-yl)ethyl)-[1,1′-biphenyl]-3-amine OR0606-4 (63%) as a colorless oil, used in the next step without further purification. Rf=0.45 (DCM-MeOH—NH₄OH, 90:9:1); LCMS C₂₄H₃₅N₃ method (B) Rₜ=4.590 min, ESI+ m/z=366.3 (M+H).

N-iso-Butyl-2-methyl-5-(6-(2-(4-methylpiperazin-1-yl)ethoxy)pyridin-3-yl)aniline OR0608-4 (44%) as a colorless oil. LCMS C₂₃H₃₄N₄O method (B) Rₜ=4.859 min, ESI+ m/z=383.3 (M+H).

N-((1-(3-(iso-Butylamino)-4-methylphenyl)-1H-1,2,3-triazol-4-yl)methyl)methanesulfonamide OR0609-4 (68%) as a white solid. ¹H NMR (400 MHz, CDCl₃) δ 8.02 (s, 1H), 7.32-7.28 (m, 1H), 7.20 (d, J=8.0 Hz, 1H), 7.05-7.00 (m, 1H), 5.44 (brs, 1H), 4.55 (d, J=5.4 Hz, 2H), 3.08 (d, J=6.8 Hz, 2H), 3.03 (s, 3H), 2.31 (s, 3H), 2.14-2.04 (m, 1H), 1.09 (d, J=6.8 Hz, 6H); LCMS C₁₅H₂₃N₅O₂S method (B) Rₜ=5.794 min, ESI+m/z=338.2 (M+H).

tert-Butyl (2-((4-methyl-4′-(2-(4-methylpiperazin-1-yl)ethyl)-[1,1′-biphenyl]-3-yl)amino)ethyl)carbamate OR0610-5 (35%) as a colorless oil. Rf=0.65 (DCM-MeOH—NH₄OH, 90:9:1); ¹H NMR (400 MHz, CDCl₃) δ 7.49 (d, J=8.1 Hz, 2H), 7.24 (d, J=8.1 Hz, 2H), 7.10 (d, J=7.6 Hz, 1H), 6.85 (dd, J=7.6, 1.2 Hz, 1H), 6.75 d, J=1.2 Hz, 1H), 4.88 (brs, 1H), 4.18 (brs, 1H), 3.51-3.43 (m, 2H), 3.37-3.29 (m, 2H), 2.89-2.81 (m, 2H), 2.70-2.62 (m, 2H), 2.57 (brs, 8H), 2.35 (s, 3H), 2.17 (s, 3H), 1.45 (s, 9H); ¹³C NMR (100 MHz, CDCl₃) δ 156.90, 146.39, 140.17, 139.99, 138.80, 130.42, 128.94, 127.16, 121.22, 115.69, 107.98, 79.68, 60.35, 54.96, 52.87, 45.86, 45.20, 40.01, 33.14, 28.39, 17.20; LCMS C₂₇H₄₀N₄O₂ method (B) R=4.851 min, ESI+ m/z=453.4 (M+H).

tert-Butyl (3-((4-methyl-4′-(2-(4-methylpiperazin-1-yl)ethyl)-[1,1′-biphenyl]-3-yl)amino)propyl)carbamate OR0611-5 (46%) as a colorless oil. Rf=0.65 (DCM-MeOH—NH₄OH, 90:9:1); ¹H NMR (400 MHz, CDCl₃) δ 7.49 (d, J=8.2 Hz, 2H), 7.24 (d, J=8.2 Hz, 2H), 7.10 (d, J=7.8 Hz, 1H), 6.85 (dd, J=7.8, 1.6 Hz, 1H), 6.78 (d, J=1.6 Hz, 1H), 4.70 (brs, 1H), 3.32-3.24 (m, 4H), 2.89-2.81 (m, 2H), 2.71-2.63 (m, 2H), 2.61 (brs, 8H), 2.36 (s, 3H), 2.18 (s, 3H), 1.84 (quint, J=6.6 Hz, 2H), 1.44 (s, 9H); LCMS C₂₈H₄₂N₄O₂ method (B) R=4.818 min, ESI+ m/z=467.4 (M+H).

(5-(4-Methyl-3-(propylamino)phenyl)pyridin-2-yl)(4-methylpiperazin-1-yl)methanone OR0612-4 (95%) as a beige oil. ¹H NMR (400 MHz, CDCl₃) δ 8.79 (d, J=2.1 Hz, 1H), 7.97 (dd, J=8.1, 2.1 Hz, 1H), 7.71 (d, J=8.1 Hz, 1H), 7.15 (d, J=7.8 Hz, 1H), 6.85 (dd, J=7.5, 1.8 Hz, 1H), 6.77 (d, J=1.8 Hz, 1H), 3.89 (brs, 2H), 3.76 (brs, 2H), 3.20 (t, J=7.1 Hz, 2H), 2.59 (brs, 2H), 2.51 (brs, 2H), 2.37 (s, 3H), 2.19 (s, 3H), 1.73 (sext, J=7.4 Hz, 2H), 1.05 (t, J=7.4 Hz, 3H); LCMS C₂₁H₂₈N₄O method (B) Rₜ=4.358 min, ESI+ m/z=353.3 (M+H).

4-Methyl-N-(5-(4-methyl-3-(propylamino)phenyl)pyridin-2-yl)piperazine-1-carboxamide OR0616-4 (90%) as a beige oil. ¹H NMR (400 MHz, CDCl₃) δ 8.40 (d, J=2.2 Hz, 1H), 8.05 (d, J=8.7 Hz, 1H), 7.87 (dd, J=8.7, 2.2 Hz, 1H), 7.38 (brs, 1H), 7.11 (d, J=7.7 Hz, 1H), 6.81 (dd, J=7.7, 1.7 Hz, 1H), 6.73 (d, J=1.7 Hz, 1H), 3.65-3.59 (m, 4H), 3.19 (t, J=7.1 Hz, 2H), 2.57-2.53 (m, 4H), 2.39 (s, 3H), 2.17 (s, 3H), 1.72 (sext, J=7.4 Hz, 2H), 1.04 (t, J=7.4 Hz, 3H); LCMS C₂₁H₂₉N₅O method (B) Rₜ=4.217 min, ESI+ m/z=368.3 (M+H).

5-Fluoro-4-methyl-4′-((4-methylpiperazin-1-yl)sulfonyl)-N-propyl-[1,1′-biphenyl]-3-amine OR0633-4 (55%) as a yellow oil. ¹H NMR (400 MHz, CDCl₃) δ 7.78 (d, J=8.5 Hz, 2H), 7.69 (d, J=8.5 Hz, 2H), 6.65 (dd, J=10.2, 1.6 Hz, 1H), 6.55 (d, J=1.6 Hz, 1H), 3.20 (t, J=7.4 Hz, 2H), 3.19 (brs, 4H), 2.65 (brs, 4H), 2.38 (s, 3H), 2.08 (s, 3H), 1.72 (sext, J=7.4 Hz, 2H), 1.05 (t, J=7.4 Hz, 3H); LCMS C₂₁H₂₈FN₃O₂S method (B) Rₜ=5.345 min, ESI+ m/z=406.2 (M+H).

General Procedure for the Synthesis of [1,1′-biaryl]-3-amine Derivatives. Method (A): a suspension of appropriate 3-nitro-1,1′-biaryl (1 mmol), iron powder (280 mg, 5 mmol) and ammonium chloride (539 mg, 10 mmol) in a mixture of water-ethanol (1:1, 20 mL) was refluxed until the reaction was complete as indicated by TLC monitoring. The reaction mixture was filtrated through a short pad of celite and rinsed with EtOH. The solvent was distillated off under reduced pressure and the residue was purified by flash chromatography to afford the corresponding [1,1′-biaryl]-3-amine. Method (B): as already described for 3-nitro-1,1′-biaryl derivatives, starting from commercially available 3-amino-4-methylphenylboronic acid and appropriate aryl halide.

2-Methyl-5-(6-(2-(4-methylpiperazin-1-yl)ethoxy)pyridin-3-yl)aniline OR0608-5 (method B, 85%) as a colorless oil. ¹H NMR (400 MHz, CDCl₃) δ 8.31 (d, J=2.5 Hz, 1H), 7.74 (dd, J=8.6, 2.5 Hz, 1H), 7.11 (d, J=7.7 Hz, 1H), 6.85 (dd, J=7.7, 1.8 Hz, 1H), 6.82 (d, J=1.8 Hz, 1H), 6.79 (d, J=8.6 Hz, 1H), 4.48 (t, J=5.8 Hz, 2H), 3.70 (brs, 2H), 2.84 (t, J=5.8 Hz, 2H), 2.69 (brs, 4H), 2.58 (brs, 4H), 2.35 (s, 3H), 2.20 (s, 3H); LCMS C₁₉H₂₆N₄O method (B) Rₜ=3.514 min, ESI+ m/z=327.2 (M+H).

N-((1-(3-Amino-4-methylphenyl)-1H-1,2,3-triazol-4-yl)methyl)methanesulfonamide OR0609-5 (method A, 97%) as a pale yellow solid. LCMS C₁₉H₁₅N₅O₂S method (B) R=4.179 min, ESI+ m/z=282.2 (M+H).

(5-(3-Amino-4-methylphenyl)pyridin-2-yl)(4-methylpiperazin-1-yl)methanone OR0612-5 (method B, 90%) as a beige solid. ¹H NMR (400 MHz, CDCl₃) δ 8.75 (s, 1H), 7.92 (dd, J=8.1, 1.9 Hz, 1H), 7.69 (d, J=8.1 Hz, 1H), 7.15 (d, J=7.7 Hz, 1H), 6.91 (d, J=7.7 Hz, 1H), 6.88 (s, 1H), 3.87 (brs, 2H), 3.76 (brs, 2H), 3.72 (brs, 2H), 2.56 (brs, 2H), 2.47 (brs, 2H), 2.35 (s, 3H), 2.21 (s, 3H); LCMS C₁₅H₂₂N₄O method (B) Rₜ=2.725 min, ESI+ m/z=311.2 (M+H).

N-(5-(3-Amino-4-methylphenyl)pyridin-2-yl)-4-methylpiperazine-1-carboxamide OR0616-5 (method B, 70%) as an orange solid. ¹H NMR (400 MHz, CDCl₃) δ 8.37 (d, J=2.2 Hz, 1H), 8.04 (d, J=8.7 Hz, 1H), 7.83 (dd, J=8.7, 2.2 Hz, 1H), 7.43 (brs, 1H), 7.11 (d, J=7.7 Hz, 1H), 6.88 (dd, J=7.7, 1.7 Hz, 1H), 6.85 (d, J=1.7 Hz, 1H), 3.62-3.57 (m, 4H), 2.53-2.47 (m, 4H), 2.36 (s, 3H), 2.20 (s, 3H); LCMS C₁₅H₂₃N₅O method (B) Rₜ=2.298 min, ESI+m/z=326.1 (M+H).

5-Fluoro-4-methyl-4′-((4-methylpiperazin-1-yl)sulfonyl)-[1,1′-biphenyl]-3-amine OR0633-5 (method A, quantitative) as a yellow oil. LCMS C₁₈H₂₂FN₃O₂S method (B) Rₜ=4.657 min, ESI+ m/z=364.1 (M+H).

1-(2-((5-Bromopyridin-2-yl)oxy)ethyl)-4-methylpiperazine OR0608-6′ From commercially available 5-bromo-2-fluoropyridine and 2-(4-methylpiperazin-1-yl)ethan-1-ol, (62%) as a colorless oil. ¹H NMR (400 MHz, CDCl₃) δ 8.16 (d, J=2.5 Hz, 1H), 7.62 (dd, J=8.8, 2.5 Hz, 1H), 6.66 (d, J=8.8 Hz, 1H), 4.40 (t, J=5.8 Hz, 2H), 2.78 (t, J=5.8 Hz, 2H), 2.63 (brs, 4H), 2.52 (brs, 4H), 2.31 (s, 3H); LCMS C₁₂H₁₈BrN₃O method (B) Rₜ=3.571 min, ESI+ m/z=300.0 (M+H).

N-((1-(4-Methyl-3-nitrophenyl)-1H-1,2,3-triazol-4-yl)methyl)methanesulfonamide OR0609-6

To a mixture of N-(prop-2-yn-1-yl)methanesulfonamide (620 mg, 4.66 mmol), CuSO₄ pentahydrate (30 mg, 0.12 mmol), sodium ascorbate (118 mg, 0.60 mmol) in a mixture of tert-butanol-H₂O (1:1, 30 mL) was added 4-azido-1-methyl-2-nitrobenzeneⁱⁱ (817 mg, 4.59 mmol).

The suspension was stirred for 48 hrs at room temperature, until consumption of starting material monitored by LCMS. The solvent was distillated off under reduced pressure and the residue was taken up into 10% aqueous NH₄OH solution (20 mL) and extracted with EtOAc (2×50 mL). The combined organic layers were dried over Na₂SO₄ and concentrated under reduced pressure. The residue was purified by flash chromatography, gradient DCM-EtOAc (100:0 to 65:35) to afford N-((1-(4-Methyl-3-nitrophenyl)-1H-1,2,3-triazol-4-yl)methyl)methanesulfonamide OR0609-6 (1.24 g, 87%) as a beige solid. ¹H NMR (400 MHz, DMSO) δ 8.87 (s, 1H), 8.53 (d, J=2.3 Hz, 1H), 8.20 (dd, J=8.3, 2.3 Hz, 1H), 7.74 (d, J=8.3 Hz, 1H), 7.64 (s, 1H), 4.33 (s, 2H), 2.96 (s, 3H), 2.57 (s, 3H); LCMS C₁₁H₁₃N₅O₄S method (B) R=5.027 min, ESI+ m/z=312.1 (M+H).

N-(5-Bromopyridin-2-yl)-4-methylpiperazine-1-carboxamide OR0616-6

From 4-nitrophenyl (5-bromopyridin-2-yl)carbamate OR0616-7, (79%) as a yellow oil. ¹H NMR (400 MHz, CDCl₃) δ 8.23 (d, J=2.4 Hz, 1H), 7.96 (d, J=8.9 Hz, 1H), 7.73 (dd, J=8.9, 2.4 Hz, 1H), 3.61-3.54 (m, 4H), 2.54-2.47 (m, 4H), 2.36 (s, 3H).

4-Nitrophenyl (5-bromopyridin-2-yl)carbamate OR0616-7ⁱⁱⁱ

From commercially available 5-bromopyridin-2-amine and 4-nitrophenyl chloroformate, (43%) as a white powder. LCMS C₁₂H₈BrN₃O₄ method (B) Rₜ=6.490 min, ESI+ m/z=338.0 (M+H).

1-((3′-Fluoro-4′-methyl-5′-nitro-[1,1′-biphenyl]-4-yl)sulfonyl)-4-methylpiperazine OR0633-6

As previously described for 3-nitro-1,1′-biaryl derivatives, from commercially available 5-bromo-1-fluoro-2-methyl-3-nitrobenzene and 1-methyl-4-((4-(4,4,5,5-tetramethyl-1,3,2-dioxaborolan-2-yl)phenyl)sulfonyl)piperazine, (quantitative) as a yellow oil. LCMS C₁₈H₂₀FN₃O₄S method (B) Rₜ=5.007 min, ESI+ m/z=394.2 (M+H).

General Procedure for the Synthesis of 4,4,5,5-tetramethyl-2-aryl-1,3,2-dioxaborolane. Method (A); under argon, a suspension of appropriate aryl halide (5.6 mmol), bis(pinacolato)diboron (3.63 g, 14.3 mmol), potassium acetate (2.80 g, 28.6 mmol), PdCl₂(dppf) (697 mg, 0.9 mmol), in degassed 1,4-dioxane (50 mL) was refluxed for 2 hrs upon complete consumption of starting material. The resulting mixture was successively allowed to cool to room temperature, filtered through a short pad of celite and washed with EtOAc. The solvent was distillated off under reduced pressure and the residue was purified by flash chromatography, to afford corresponding 4,4,5,5-tetramethyl-2-aryl-1,3,2-dioxaborolane. Method (B); under argon, at −78° C., to a solution of appropriate aryl halide (1.0 mmol) in tetrahydrofuran (5 mL) were added successively 2-isopropoxy-4,4,5,5-tetramethyl-1,3,2-dioxaborolane (0.37 mL, 1.8 mmol) and then n-BuLi (2.5 M in hexane, 0.47 mL, 1.18 mmol) over 5 minutes. After addition, the resulting mixture was stirred at −78° C. for 30 min and then quenched with water (5 mL). The aqueous layer was successively washed with DCM (3×5 mL), acidified with 2M aqueous HCl solution until pH=7, and extracted with EtOAc (3×5 mL). The combined organic layers were dried on Na₂SO₄ and the solvent was distillated off under reduced pressure affording expected 4,4,5,5-tetramethyl-2-aryl-1,3,2-dioxaborolane.

Ethyl 2-((2-methyl-5-(4,4,5,5-tetramethyl-1,3,2-dioxaborolan-2-yl)phenyl)(propyl)amino)thiazole-4-carboxylate OR0625-2 (method A, quantitative) as a white solid. ¹H NMR (400 MHz, CDCl₃) δ 7.73 (dd, J=7.5, 1.2 Hz, 1H), 7.62 (d, J=1.2 Hz, 1H), 7.35 (d, J=7.5 Hz, 1H), 7.26 (s, 1H), 4.35 (q, J=7.1 Hz, 2H), 4.02-3.75 (m, 2H), 2.24 (s, 3H), 1.67 (sext, J=7.4 Hz, 2H), 1.37 (t, J=7.1 Hz, 3H), 1.34 (s, 12H), 0.92 (t, J=7.4 Hz, 3H); LCMS C₂₂H₃₁BN₂O₄S method (B) Rₜ=6.109 min, ESI+ m/z=349.2 (ArB(OH)₂+H).

1-Methyl-4-((5-(4,4,5,5-tetramethyl-1,3,2-dioxaborolan-2-yl)pyridin-2-yl)sulfonyl)piperazine OR0627-2 (method B, 43%) as a white solid. ¹H NMR (400 MHz, CDCl₃) δ 9.00 (d, J=1.7 Hz, 1H), 8.26 (dd, J=7.7, 1.7 Hz, 1H), 7.88 (d, J=7.7 Hz, 1H), 3.50 (brs, 4H), 2.71 (brs, 4H), 2.45 (s, 3H), 1.36 (s, 12H); LCMS C₁₆H₂₆BN₃O₄S method (B) Rₜ=1.149 min. ESI+ m/z=286.1 (ArB(OH)₂+H).

1-Methyl-4-((4-(4,4,5,5-tetramethyl-1,3,2-dioxaborolan-2-yl)-3-(trifluoromethyl)phenyl)sulfonyl)piperazine OR0642-2 (method A, 83%) as a white solid. ¹H NMR (400 MHz, CDCl₃) δ 8.01 (s, 1H), 7.92 (d, J=7.8 Hz, 1H), 7.87 (dd, J=7.8, 1.4 Hz, 1H), 3.17 (brs, 4H), 2.63 (brs, 4H), 2.38 (s, 3H), 1.38 (s, 12H); LCMS C₁₈H₂₆BF₃N₂O₄S method (B) Rₜ=4.951 min, ESI+ m/z=435.2 (M+H).

(4-Methyl-6-((4-methylpiperazin-1-yl)sulfonyl)pyridin-3-yl)boronic acid OR0652-2 (method B), aqueous layer was acidified until pH=3. The aqueous layer was washed with DCM (3×50 mL) and then concentrated under reduced pressure to afford (4-methyl-6-((4-methylpiperazin-1-yl)sulfonyl)pyridin-3-yl)boronic acid OR0652-2 (40%) as a white solid. LCMS C₁₁H₁₈BN₃O₄S method (B) Rₜ=1.542 min, ESI+ m/z=300.2 (M+H).

General Procedure for the Synthesis of 1-(4-halogeno-benzenesulfonyl)-4-methyl-piperazine Derivative. Under argon, at 0° C., to a solution of appropriate 4-halogenobenzenesulfonyl chloride (1.0 mmol) in dichloromethane (6 mL) was added 1-methyl-piperazine (125 μL, 1.12 mmol). The reaction mixture was stirred at 0° C. for 30 min. and allowed to warm to room temperature. After stirring for 2 hours, the mixture was diluted with DCM (15 mL), then saturated Na₂CO₃ aqueous solution was added (5 mL) and the aqueous layer was extracted with DCM (2×15 mL). The combined organic layers were dried over Na₂SO₄ and the solvent was distillated off under reduced pressure to afford corresponding 1-(4-halogeno-benzenesulfonyl)-4-methyl-piperazine used in the next step without further purification.

1-((5-Bromopyridin-2-yl)sulfonyl)-4-methylpiperazine OR0627-3 (quantitative). ¹H NMR (400 MHz, CDCl₃) δ 8.73 (d, J=2.0 Hz, 1H), 8.03 (dd, J=8.3, 2.0 Hz, 1H), 7.81 (d, J=8.3 Hz, 1H), 3.38-3.30 (m, 4H), 2.52-2.44 (m, 4H), 2.29 (s, 3H); LCMS C₁₀H₁₄BrN₃O₂S method (B) Rₜ=3.703 min, ESI+ m/z=320.0 (M+H).

1-((4-Bromo-3-(trifluoromethyl)phenyl)sulfonyl)-4-methylpiperazine OR0642-3 (99%). ¹H NMR (400 MHz, CDCl₃) δ 8.02 (d, J=1.9 Hz, 1H), 7.90 (d, J=8.3 Hz, 1H), 7.74 (dd, J=8.3, 2.0 Hz, 1H), 3.12-3.04 (m, 4H), 2.56-2.48 (m, 4H), 2.29 (s, 3H); LCMS C₁₀H₁₄BrN₃O₂S method (B) Rₜ=4.419 min. ESI+ m/z=387.0 (M+H).

1-((5-Bromo-4-methylpyridin-2-yl)sulfonyl)-4-methylpiperazine OR0652-3 (63%) as a white solid. ¹H NMR (400 MHz, CDCl₃) δ 8.69 (s, 1H), 7.78 (s, 1H), 3.41-3.32 (m, 4H), 2.56-2.50 (m, 4H), 2.49 (s, 3H), 2.32 (s, 3H); LCMS C₁₁H₁₆BrN₃O₂S method (B) Rₜ=4.075 min, ESI+ m/z=334.0 (M+H).

N-(5-Bromopyridin-2-yl)-4-methylpiperazine-1-sulfonamide OR0625-4

Using a modified procedure described by Borghese et al.,^{iv} a mixture of N-(5-bromopyridin-2-yl)-2-oxooxazolidine-3-sulfonamide OR0625-5 (1.73 g, 5.37 mmol), 1-methylpiperazine (600 μL, 5.37 mmol) and triethylamine (2.25 mL, 16.13 mmol) in acetonitrile (27 mL) was refluxed for 4 days. Upon complete consumption of the starting material monitored by LCMS, the solvent was distillated off under reduced pressure, then saturated aqueous NaHCO₃ solution was added until pH=8, then the aqueous layer was extracted with DCM (3×40 mL). The combined organic layers were dried over Na₂SO₄, and the solvent was distillated off under reduced pressure. The residue was purified by flash chromatography, gradient DCM-MeOH—NH₄OH (100:0:0 to 90:9:1) to afford N-(5-bromopyridin-2-yl)-4-methylpiperazine-1-sulfonamide OR0625-4 (311 mg, 17%) as a white solid. ¹H NMR (400 MHz, CDCl₃) δ 8.44 (d, J=2.4 Hz, 1H), 7.78 (dd, J=8.9, 2.4 Hz, 1H), 7.28 (d, J=8.9 Hz, 1H), 3.39-3.32 (m, 4H), 2.55-2.48 (m, 4H), 2.33 (s, 3H); LCMS C₁₀H₁₅BrN₄O₂S method (B) Rₜ=3.567 min, ESI+ m/z=335.0 (M+H).

N-(5-Bromopyridin-2-yl)-2-oxooxazolidine-3-sulfonamide OR0625-5

Using a modified procedure described by Borghese et al.,^{iv} under argon, at 0° C., to a solution of chlorosulfonyl isocyanate (503 μL, 5.78 mmol) in dry dichloromethane (20 mL) was added dropwise 2-chloroethan-1-ol (390 μL, 5.78 mmol). After stirring at 0° C. for 1 hour, triethylamine (2.5 mL, 17.30 mmol) was added, followed by a solution of 5-bromopyridin-2-amine (1.0 g, 5.78 mmol) in dry dichloromethane (15 mL) dropwise. The resulting mixture was stirred for an additional hour at 0° C., then allowed to warm to room temperature and stirred overnight. The reaction was quenched by addition of 0.25N HCl aqueous solution, extracted with DCM (2×40 mL), and the combined organic layers were dried over anhydrous Na₂SO₄. The solvent was distillated off under reduced pressure and the crude residue was triturated with cyclohexane affording N-(5-bromopyridin-2-yl)-2-oxooxazolidine-3-sulfonamide OR0625-5 (1.73 g, 93%) as a white solid. LCMS C₈H₈BrN₃O₄S method (B) Rₜ=4.761 min, ESI+ m/z=322.0 (M+H).

2-((5-Bromopyridin-2-yl)oxy)-1-(4-methylpiperazin-1-yl)ethan-1-one OR0630-2 By peptide coupling from commercially available 2-((5-bromopyridin-2-yl)oxy)acetic acid, using same procedure as described for di-tert-butyl (2-(2-((3-benzamidoaryl)amino)thiazol-4-yl)pyrimidin-4-yl)carbamate derivatives. ¹H NMR (400 MHz, CDCl₃) δ 8.13 (d, J=2.4 Hz, 1H), 7.67 (dd, J=8.8, 2.4 Hz, 1H), 6.80 (d, J=8.8 Hz, 1H), 4.97 (s, 2H), 3.67 (brs, 2H), 3.53 (brs, 2H), 2.48 (brs, 2H), 2.45 (brs, 2H), 2.34 (s, 3H); LCMS C₁₂H₁₆BrN₃O₂ method (B) Rₜ=3.649 min, ESI+ m/z=314.0 (M+H).

General Procedure for the Synthesis of ethyl 2-((5-halogeno-2-methylphenyl)amino)thiazole-4-carboxylate Derivative and analogues. Method (A): as previously described for synthesis of ethyl 2-([1,1′-biaryl]-3-ylamino)thiazole-4-carboxylate derivatives, method (A). Method (B): To a suspension of ethyl 2-((5-halogeno-2-methylphenyl)amino)thiazole-4-carboxylate or analogue (0.16 mol) and cesium carbonate (73 g, 0.22 mol) in dimethylformamide (1 L) was added appropriate alkyl halide (20 mL, 0.22 mol). The reaction mixture was stirred at r.t for 72 hrs until complete consumption of starting material. The solvent was distillated off under reduced pressure and the residue was poured onto water (300 mL) and extracted with Et₂O (3×300 mL). The combined organic layers were dried over Na₂SO₄ and concentrated under vacuum. The residue was purified by flash chromatography, to afford corresponding ethyl 2-((5-halogeno-2-methylphenyl)(alkyl)amino)thiazole-4-carboxylate or analogue.

Ethyl 2-((5-iodo-2-methylphenyl)(propyl)amino)thiazole-4-carboxylate OR0607-2 (method A, 73%) as a pale yellow solid. Rf=0.20 (DCM-PE, 80:20); ¹H NMR (400 MHz, CDCl₃) δ 7.62 (dd, J=8.1, 1.8 Hz, 1H), 7.54 (d, J=1.8 Hz, 1H), 7.30 (s, 1H), 7.08 (d, J=8.1 Hz, 1H), 4.35 (q, J=7.1 Hz, 2H), 3.88 (brs, 2H), 2.17 (s, 3H), 1.64 (sext, J=7.4 Hz, 2H), 1.37 (t, J=7.1 Hz, 3H), 0.93 (t, J=7.4 Hz, 3H); ¹³C NMR (100 MHz, CDCl₃) δ 170.27, 161.88, 144.08, 143.88, 138.22, 137.96, 137.26, 133.88, 116.96, 91.05, 61.20, 53.96, 21.11, 17.42, 14.47, 11.37; LCMS C₁₆H₁₉IN₂O₂S method (B) Rₜ=8.676 min, ESI+ m/z=431.0 (M+H).

Ethyl 2-((5-bromo-2-methylphenyl)(propyl)amino)thiazole-4-carboxylate OR0625-3 (method B, 54%) as a light brown solid. Rf=0.31 (DCM); ¹H NMR (400 MHz, CDCl₃) δ 7.44 (dd, J=8.2, 2.0 Hz, 1H), 7.36 (d, J=2.0 Hz, 1H), 7.31 (s, 1H), 7.22 (d, J=8.2 Hz, 1H), 4.35 (q, J=7.1 Hz, 2H), 3.89 (brs, 2H), 2.17 (s, 3H), 1.64 (sext, J=7.4 Hz, 2H), 1.37 (t, J=7.1 Hz, 3H), 0.94 (t, J=7.4 Hz, 3H); ¹³C NMR (100 MHz, CDCl₃) δ 170.27, 161.90, 143.95, 143.90, 136.50, 133.63, 132.39, 132.06, 120.22, 117.00, 61.22, 53.95, 21.13, 17.34, 14.47, 11.37; LCMS C₁₆H₁₉BrN₂O₂S method (B) Rₜ=7.376 min, ESI+ m/z=383.0 (M+H).

Ethyl 2-((5-bromo-2-methylphenyl)(methyl)amino)thiazole-4-carboxylate OR0631-2 (method B, 37%) as a light brown solid. Rf=0.29 (DCM); ¹H NMR (400 MHz, CDCl₃) δ 7.43 (dd, J=8.2, 2.0 Hz, 1H), 7.39 (d, J=2.0 Hz, 1H), 7.35 (s, 1H), 7.22 (d, J=8.2 Hz, 1H), 4.36 (q, J=7.1 Hz, 2H), 3.50 (s, 3H), 2.18 (s, 3H), 1.38 (t, J=7.1 Hz, 3H); ¹³C NMR (100 MHz, CDCl₃) δ 170.17, 161.80, 145.38, 143.87, 135.77, 133.61, 132.13, 131.39, 120.39, 117.47, 61.34, 40.01, 27.04, 17.17, 14.48; LCMS C₁₄H₁₅BrN₂O₂S method (B) Rₜ=7.212 min, ESI+m/z=355.0 (M+H).

Ethyl 2-((5-bromo-2-methylphenyl)(ethyl)amino)thiazole-4-carboxylate OR0632-2 (method B, 41%) as a light brown solid. Rf=0.29 (DCM); ¹H NMR (400 MHz, CDCl₃) δ 7.44 (dd, J=8.2, 2.0 Hz, 1H), 7.36 (d, J=2.0 Hz, 1H), 7.31 (s, 1H), 7.23 (d, J=8.2 Hz, 1H), 4.35 (q, J=7.1 Hz, 2H), 4.01 (brs, 2H), 2.18 (s, 3H), 1.37 (t, J=7.1 Hz, 3H), 1.21 (t, J=7.2 Hz, 3H); ¹³C NMR (100 MHz, CDCl₃) δ 170.00, 161.96, 144.01, 143.57, 136.65, 133.57, 132.52, 132.09, 120.23, 117.05, 61.21, 46.91, 17.33, 14.49, 13.12; LCMS C₁₅H₁₇BrN₂O₂S method (B) R=7.570 min, ESI+ m/z=369.0 (M+H).

Ethyl 2-((5-bromo-2-methylphenyl)(isopropyl)amino)thiazole-4-carboxylate OR0636-2 (method B, 67%) as a light yellow solid. Rf=0.34 (DCM); ¹H NMR (400 MHz, CDCl₃) δ 7.47 (dd, J=8.2, 2.1 Hz, 1H), 7.33 (d, J=2.1 Hz, 1H), 7.28 (s, 1H), 7.24 (d, J=8.2 Hz, 1H), 5.07 (sept, J=6.7 Hz, 1H), 4.35 (q, J=7.1 Hz, 2H), 2.18 (s, 3H), 1.37 (t, J=7.1 Hz, 3H), 1.25-1.23 (m, 6H); ¹³C NMR (100 MHz, CDCl₃) δ 169.99, 161.99, 143.95, 141.33, 138.00, 133.75, 133.56, 132.32, 119.97, 116.64, 61.14, 52.25, 20.76, 17.83, 14.49; LCMS C₁₆H₁₉BrN₂O₂S method (B) Rₜ=7.818 min, ESI+ m/z=383.0 (M+H).

Ethyl 2-((5-bromo-2-methylphenyl)(isobutyl)amino)thiazole-4-carboxylate OR637-2 (method B, 60%) as a light yellow solid. Rf=0.34 (DCM); ¹H NMR (400 MHz, CDCl₃) δ 7.43 (dd, J=8.2, 2.0 Hz, 1H), 7.39 (d, J=2.0 Hz, 1H), 7.30 (s, 1H), 7.22 (d, J=8.2 Hz, 1H), 4.34 (q, J=7.1 Hz, 2H), 3.73 (brs, 2H), 2.17 (s, 3H), 2.05-1.92 (m, 1H), 1.36 (t, J=7.1 Hz, 3H), 0.98 (d, J=6.7 Hz, 6H); ¹³C NMR (100 MHz, CDCl₃) δ 170.81, 161.85, 144.52, 143.86, 136.21, 133.84, 132.28, 131.96, 120.14, 117.00, 61.16, 59.87, 27.50, 20.51, 17.50, 14.46; LCMS C₁₇H₂₁BrN₂O₂S method (B) Rₜ=7.806 min, ESI+ m/z=397.0 (M+H).

Ethyl 2-((5-bromo-2-methylphenyl)(propyl)amino)oxazole-4-carboxylate OR0640-2. (method B, 60%) as a yellow oil. ¹H NMR (400 MHz, CDCl₃) δ 7.67 (s, 1H), 7.38 (dd, J=8.2, 2.0 Hz, 1H), 7.33 (d, J=2.0 Hz, 1H), 7.16 (d, J=8.2 Hz, 1H), 4.35 (q, J=7.1 Hz, 2H), 3.81-3.73 (m, 2H), 2.12 (s, 3H), 1.66 (sext, J=7.4 Hz, 2H), 1.35 (t, J=7.1 Hz, 3H), 0.92 (t, J=7.4 Hz, 3H); LCMS C₁₆H₁₉BrN₂O₃ method (B) Rₜ=7.253 min, ESI+ m/z=367.0 (M+H).

Ethyl 2-((5-bromo-2-methylphenyl)amino)thiazole-4-carboxylate OR0614-2 (method A, quantitative) as a yellow solid, used in the next step without further purification. Rf=0.50 (PE-EtOAc, 70:30); ¹H NMR (400 MHz, CDCl₃) δ 7.66 (d, J=2.0 Hz, 1H), 7.49 (s, 1H), 7.23 (dd, J=8.1, 2.0 Hz, 1H), 7.11 (d, J=8.1 Hz, 1H), 4.29 (q, J=7.1 Hz, 2H), 2.23 (s, 3H), 1.33 (t, J=7.1 Hz, 3H); ¹³C NMR (100 MHz, CDCl₃) δ 166.65, 161.45, 143.18, 139.75, 132.67, 129.64, 128.43, 124.36, 120.19, 117.02, 61.46, 17.59, 14.45; LCMS C₁₃H₁₃BrN₂O₂S method (B) Rₜ=6.664 min, ESI+ m/z=341.0 (M+H).

Ethyl 2-((5-bromo-2-methylphenyl)amino)oxazole-4-carboxylate OR0640-3 (method A, 21%) as a white solid. LCMS C₁₃H₁₃BrN₂O₃ method (B) Rₜ=6.396 min, ESI+ m/z=325.0 (M+H).

General Procedure for the Synthesis of 1-(5-halogeno-2-methylphenyl)thiourea Derivative. As previously described for synthesis of 1-(3-nitroaryl)thiourea.

1-(5-Iodo-2-methylphenyl)-1-propylthiourea OR0607-3 (98%) as a yellow solid, engaged in the next step without further purification. ¹H NMR (400 MHz, CDCl₃) δ 7.62 (dd, J=8.1, 1.7 Hz, 1H), 7.48 (d, J=1.8 Hz, 1H), 7.07 (d, J=8.1 Hz, 1H), 5.51 (brs, 2H), 4.38-0.30 (m, 1H), 3.64-3.56 (m, 1H), 2.18 (s, 3H), 1.80-1.55 (m, 2H), 0.90 (t, J=7.4 Hz, 3H); LCMS C₁₁H₁₅IN₂S method (B) Rₜ=6.324 min, ESI+ m/z=335.0 (M+H).

1-(5-Bromo-2-methylphenyl)thiourea OR0614-3 (71%) as a yellow solid. Rf=0.18 (PE-EtOAc, 70:30); ¹H NMR (400 MHz, MeOD) δ 7.42 (d, J=1.9 Hz, 1H), 7.38 (dd, J=8.2, 2.0 Hz, 1H), 7.21 (d, J=8.2 Hz, 1H), 2.23 (s, 3H); LCMS C₈H₉BrN₂S method (B) Rₜ=4.773 min, ESI+ m/z=245.0 (M+H).

1-(5-Bromo-2-methylphenyl)urea OR0640-4

To a solution of 5-bromo-2-methylaniline (2.0 g, 10.75 mmol) in glacial acetic acid (22 mL) was added portionwise potassium cyanate (1.75 g, 21.5 mmol). The reaction mixture was stirred at r.t for 2 hours upon complete consumption of the starting material monitored by LCMS. The resulting suspension was diluted with water (200 mL) and the precipitate was collected by filtration affording 1-(5-bromo-2-methylphenyl)urea OR0640-4 (2.46 g, quantitative), as a beige solid, used in the next step without further purification. LCMS C₈H₉BrN₂O method (B) Rₜ=4.833 min, ESI+ m/z=229.0 (M+H).

General Procedure for the Synthesis of N-((5-halogeno-2-methylphenyl)carbamothioyl)benzamide Derivative. As previously described for synthesis of N-([1,1′-biaryl]-3-ylcarbamothioyl)benzamide.

N-((5-Iodo-2-methylphenyl)(propyl)carbamothioyl)benzamide OR0607-4 (quantitative), as a yellow solid, used in the next step without further purification. LCMS C₁₅H₁₉IN₂OS method (B) Rₜ=7.119 min, ESI− m/z=437.0 (M−H).

N-((5-Bromo-2-methylphenyl)carbamothioyl)benzamide OR0614-4 (quantitative) as a yellow solid, used in the next step without further purification. LCMS C₁₅H₁₃BrN₂OS method (B) Rₜ=7.166 min, ESI+ m/z=349.0 (M+H).

5-Iodo-2-methyl-N-propylaniline OR0607-5

Using the same procedure as previously described for synthesis of [1,1′-biaryl]-3-N-alkylamine, (90%) as a light yellow solid, used in the next step without further purification. Rf=0.58 (EP-DCM, 70:30); ¹H NMR (400 MHz, CDCl₃) δ 6.95 (dd, J=7.7, 1.7 Hz, 1H), 6.89 (d, J=1.7 Hz, 1H), 6.75 (d, J=7.7 Hz, 1H), 3.51 (brs, 1H), 3.09 (t, J=7.3 Hz, 2H), 2.07 (s, 3H), 1.69 (sext, J=7.3 Hz, 2H), 1.03 (t, J=7.3 Hz, 3H); ¹³C NMR (100 MHz, CDCl₃) δ 147.72, 131.56, 125.50, 121.30, 118.31, 92.36, 45.68, 22.70, 17.23, 11.77; LCMS C₁₀H₁₄IN method (B) Rₜ=7.538 min, ESI+ m/z=276.1 (M+H).

### CHEMISTRY REFERENCES

ⁱM., Tabart. Patent WO2014053568A1.
ⁱⁱK., Ankali. Synthesis and Molecular Docking of novel 1,3-Thiazole Derived 1,2,3-Triazoles and In vivo Biological Evaluation for their Anti anxiety and Anti inflammatory Activity. J Mol Struc, 2021, 1236, 130357.
ⁱⁱⁱG.-L., Lu; A. S. T., Tong; D., Conole; H. S., Sutherland; P. J., Choi; S. G., Franzblau; A. M., Upton; M. U., Lotlikar; C. B, Cooper; W. A. Denny; B. D. Palmer. Synthesis and structure-activity relationships for tetrahydroisoquinoline-based inhibitors of Mycobacterium tuberculosis. Bioorg Med Chem, 2020, 28, 115784.
^{iv}a) A., Borghese; L., Antoine; J. P., Van Hoeck; Mockel; A., Merschaert. Org. Process Res. Dev. 2006, 10, 770-775; b) M., Veguillas; G. M., Rosair; M. W. P., Bebbington; A.-L., Lee. ACS Catal. 2019, 9, 2552-2557.

### Example B—Biology

### Material & Methods

### Protein Expression and Purification

dCK protein contained three solvent exposed cysteines mutated to serines in order to generate better quality crystals (C9S, C45S, C59S) and a serine mutated to glutamic acid (S74E). The S74E mutation mimics the phosphorylated state of this serine, which favors the open conformation of the enzyme, making it competent for nucleoside binding to evaluate the binding affinity with compounds. BL21 pRIL Escherichia Coli cells were transformed with dCK-plasmid. Cells were grown in LB media containing 100 μg/ml ampicillin and 34 μg/ml chloramphenicol at 18° C. for 20 h after induction with 1 mM IPTG. Cells were re-suspended in 50 mM TRIS, pH 8, 500 mM NaCl, 30 mM imidazole and 10% glycerol buffer containing one EDTA free anti-protease tablet (Roche). After sonication and centrifugation (30,000×g), the supernatant was loaded on a 5 ml HisTrap FF column (GE healthcare) pre-equilibrated with resuspension buffer. Protein was then eluted by re-suspension buffer containing 500 mM imidazole. Protein eluted fractions were mixed and concentrated. Protein was further purified by size exclusion chromatography (superdex 200 16/600, GE healthcare) in 20 mM HEPES, pH 7.5, and 200 mM NaCl before concentration to 30 mg/ml and storage at −80° C.

### Affinity Assay

Thermal Shift Assay (TSA) experiments were performed in triplicate in 384-well PCR white plates (Thermo Scientific, AB-3384) in the assay buffer containing 20 mM HEPES, pH 7.5 and 200 mM NaCl. Final concentrations were adjusted to 5 sM of protein, 50 sM of compound (final 2% DMSO) and dye diluted to 1:1000, as recommended by manufacturer (Protein Thermal Shift Dye, ThermoFisher Scientific). Controls with compounds alone or compounds with dye were performed to verify that compounds do not present autofluorescence. Plates were centrifuged at 1000 rpm for 2 min at 4° C. Thermal melting experiments were carried using a CFX-384 Connect RT-PCR (Bio-Rad). Plates were first equilibrated at 25° C.; then, the plates were heated from 25 to 95° C. by 0.5° C./min steps. Raw fluorescence data were treated, and the melting temperatures (Tₘ) were calculated using CFX Manager 3.1 software (Bio-Rad). ΔTₘ represents the difference in Tₘ between the protein in the presence of compound and the protein alone (both at 2% DMSO). Each experiment was performed at least twice (with technical triplicates) and data are presented as the mean f SD in Table 1.

### Cellular Assay

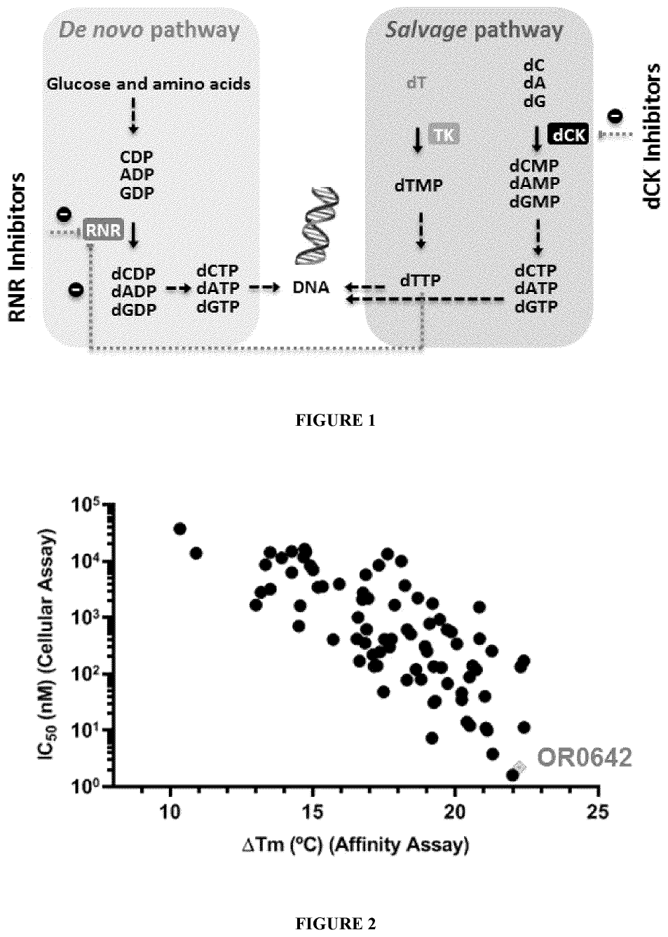

The cellular assay was set up to assess the ability to inhibit dCK activity in a cellular model. The model was developed with a T-cell acute lymphoblastic leukemia cell line (CCRF-CEM), because of its high dependence on the salvage pathway, thus on dCK activity. Cellular assays were performed to assess the inhibition of cell proliferation under two conditions: i) in the presence of the molecule alone to determine the non-specific effect on cells, independent of dCK (non-specific toxicity) and ii) in the presence of an inhibitor of the De Novo pathway to determine the ability of molecules to inhibit dCK and decrease tumor proliferation (activity on dCK). The inhibitor of the De Novo pathway chosen for the experiments was thymidine (dT), a physiological inhibitor of ribonucleotide reductase which arrests cell proliferation in phase S. The dTTP produced via thymidine kinase from dT acts as a RNR inhibitor by an allosteric regulation of the R1 subunit (FIG. 1). The addition of the nucleoside deoxycytidine (dC) in the medium allows the rescue of cell proliferation by passing only through the salvage pathway, mediated by dCK. Therefore, cell proliferation under these conditions (+dT+dC) is dCK dependent and it is possible to determine the IC₅₀ of dCK inhibitors on the inhibition of cell proliferation. Results are expressed as % cell proliferation relative to the control with DMSO in Table 1.

CCFR-CEM cells were grown at 37° C. in RPMI 1640 with L-glutamine, supplemented with 100 units/ml penicillin, 100 mg/ml streptomycin, and 10% heat-inactivated foetal calf serum. The experiments were carried out on 96-well plates under the following conditions: 14,000 cells per well, RPMI 1640 culture medium supplemented with 10% heat-inactivated foetal calf serum, 200 sM of dT, 1 sM of dC and a range of dilutions of each molecule ranging from 40 μM to 10 μM, depending on the activity of the molecules (final 0.2% DMSO). Cells were incubated for 72 hours under these different conditions and finally, incubated with 10 μl/well of CellTiter-Blue reagent (Promega) for 4 hours at 37° C. The amount of reduced dye formed by metabolically active cells was quantified by its fluorescence measured at 560 nm (excitation) and 590 nm (emission) in a microplate reader (CLARIOstar, BMG Labtech). A blank well without cells was used as a background control. Each experiment was performed at least three times (with technical triplicates) and data are presented as the mean f SD.

### Animal Model

All experiments were performed using standard methods in agreement with the French Guidelines for animal handling and approved by the local ethics committee APAFIS #6743. NOD-SCID-IL2R common-7-chain-knockout mice (NSG) were purchased from Charles River Laboratories and maintained under pathogen-free conditions on a 12-h light and 12-h dark cycle. Healthy 6 to 9 weeks-old male mice received 0.1×10⁶ luciferase transduced-CCRF-CEM cells in the tail vein on Day 0 and were randomly assigned in four groups to receive intraperitoneal injections from Day 3 (Vehicle (n=8), dT (n=8), OR0642 (n=9) and dT+OR0642 (n=9)). Treatments were administered during 19 days at cycles of 5 days (BID) and 2 days (QD). Doses administered were: dT at 1.5 g/Kg, OR0642 at 40 mg/Kg and dT+OR0642 at 1.5 g/Kg and 40 mg/Kg, respectively. Vehicle was saline solution (0.9% NaCl) supplemented with 10% Kolliphor EL. OR0642 was administered in its hydrochloride salt form. Drug administration protocol is detailed in FIG. 4. Bioluminescence analysis was performed once/week using PhotonIMAGER Optima (Biospace Lab) following addition of endotoxin-free luciferin (30 mg/kg, Promega). Peripheral blood was obtained at Day 21 to determine the fraction of human blasts using flow cytometry. Mononuclear cells were labelled with Pacific blue-conjugated anti-hCD45 (Biolegend, 304029), APC eFluor780-conjugated anti-mCD45 (ThermoFisher Scientific, 47-0451-82) and live/dead Fixable Far Red Dead Cell Stain kit (ThermoFisher Scientific, L10120) to determine the fraction of human blasts (Live Dead-/hCD45+/mCD45− cells) using flow cytometry. Analyses were performed on a Life Science Research Fortessa flow cytometer with DIVA software (BD Biosciences). The number of ALL cells/L peripheral blood was determined by using CountBright beads (Invitrogen, C36950) using described protocol. Daily monitoring of mice for symptoms of disease (ruffled coat, hunched back, weakness and reduced motility) determined the time of killing for injected animals with signs of distress. Kaplan-Meier curves, bioluminescence quantifications and statistical analyses were generated using Prism (Graphpad Software). Bioluminescence data were presented as the mean f SEM. Comparison of bioluminescence data at Day 21 was performed using One way ANOVA Kruskal-Wallis Test and Dunn's Multiple Comparison Test. Comparison of hCD45 populations was performed using One way ANOVA Kruskal-Wallis Test and Dunn's Multiple Comparison Test. Median survival times were compared using Log-Rank (Mantel-Cox) Test.

### Results

### Affinity Assay and Cellular (In Vitro) Assay

Table 1 below shows results obtained with some compounds according to the invention for Affinity Assay (i.e., binding assay) via Thermal Shift Assay expressed in +ΔTₘ (°) and Cellular Assay (i.e., in vitro experiment on T-cell acute lymphoblastic leukemia cell line CCRF-CEM in presence of 200 μM dT and 1 μM dC) expressed in IC₅₀ (nM).

TABLE 1
affinity assay and cellular assay

| | | AFFINITY | CELLULAR |
|---|---|---|---|
| | | ASSAY | ASSAY |
| COMPOUND | STRUCTURE | +ΔTm (°) | IC₅₀ (nM) |
| dCKi1 | 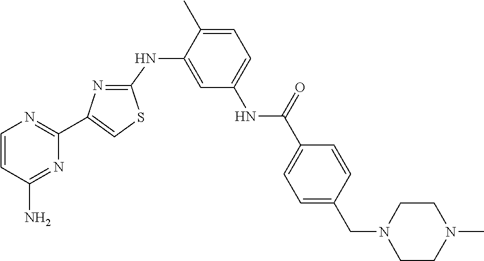 [SMILES: Cc1ccc(NC(=O)c2ccc(CN3CCN(C)CC3)cc2)cc1Nc1nc(-c2nccc(N)n2)cs1] | 14.3 ± 0.4 | 6954 ± 2532 |
| dCKi2 | 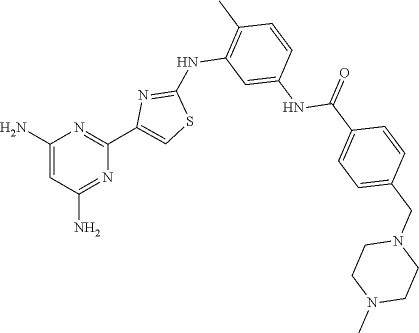 [SMILES: Cc1ccc(NC(=O)c2ccc(CN3CCN(C)CC3)cc2)cc1Nc1nc(-c2nc(N)cc(N)n2)cs1] | 17.3 ± 0.2 | 8906 ± 3339 |
| OR0105 | 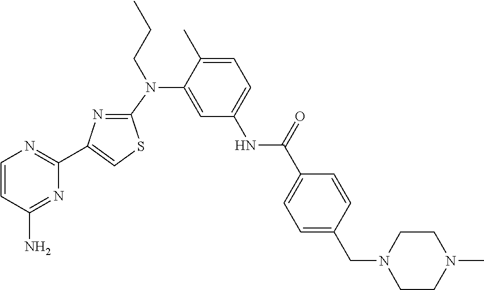 [SMILES: CCCN(c1nc(-c2nccc(N)n2)cs1)c1cc(NC(=O)c2ccc(CN3CCN(C)CC3)cc2)ccc1C] | 14.5 ± 0.2 | 1620 ± 254 |
| OR0125 | 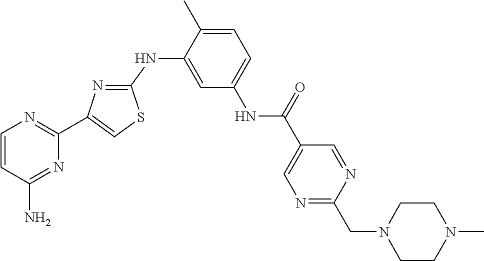 [SMILES: Cc1ccc(NC(=O)c2cnc(CN3CCN(C)CC3)nc2)cc1Nc1nc(-c2nccc(N)n2)cs1] | 10.3 ± 0.0 | 37669 ± 4383 |
| OR0143 | 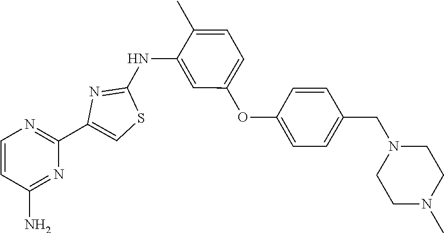 [SMILES: Cc1ccc(Oc2ccc(CN3CCN(C)CC3)cc2)cc1Nc1nc(-c2nccc(N)n2)cs1] | 13.2 ± 0.2 | 2794 ± 1736 |
| OR0146 | 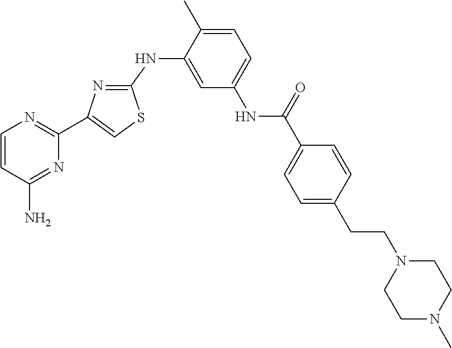 [SMILES: Cc1ccc(NC(=O)c2ccc(CCN3CCN(C)CC3)cc2)cc1Nc1nc(-c2nccc(N)n2)cs1] | 14.8 ± 0.4 | 14622 ± 1191 |
| OR0153 | 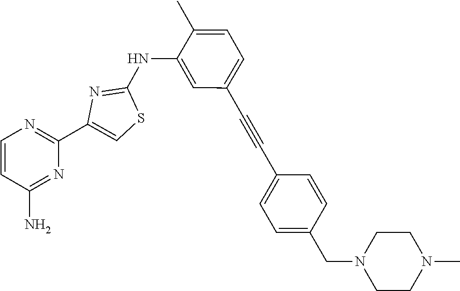 [SMILES: Cc1ccc(C#Cc2ccc(CN3CCN(C)CC3)cc2)cc1Nc1nc(-c2nccc(N)n2)cs1] | 16.9 ± 0.4 | 2175 ± 388 |
| OR0155 | 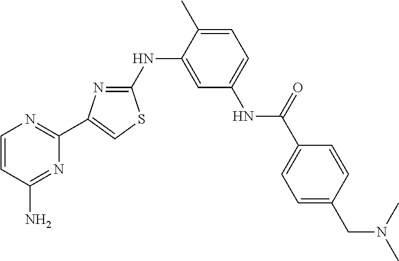 [SMILES: Cc1ccc(NC(=O)c2ccc(CN(C)C)cc2)cc1Nc1nc(-c2nccc(N)n2)cs1] | 10.9 ± 0.4 | 13809 ± 394 |
| OR0156 | 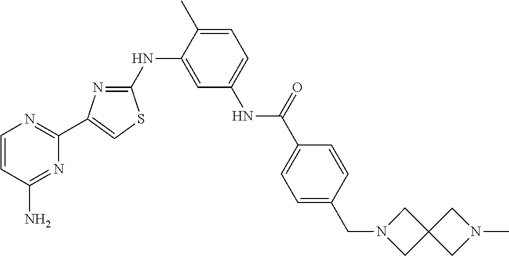 [SMILES: Cc1ccc(NC(=O)c2ccc(CN3CC4(CN(C)C4)C3)cc2)cc1Nc1nc(-c2nccc(N)n2)cs1] | 12.6 ± 0.1 | >40000 |
| OR0232 | 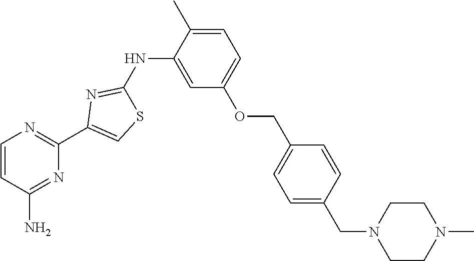 [SMILES: Cc1ccc(OCc2ccc(CN3CCN(C)CC3)cc2)cc1Nc1nc(-c2nccc(N)n2)cs1] | 15.2 ± 0.0 | 3456 ± 638 |
| OR0237 | 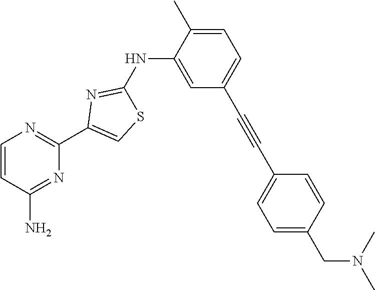 [SMILES: Cc1ccc(C#Cc2ccc(CN(C)C)cc2)cc1Nc1nc(-c2nccc(N)n2)cs1] | 15.3 ± 0.5 | 3345 ± 1453 |
| OR0239 | 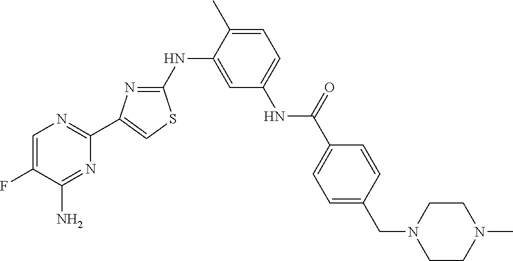 [SMILES: Cc1ccc(NC(=O)c2ccc(CN3CCN(C)CC3)cc2)cc1Nc1nc(-c2ncc(F)c(N)n2)cs1] | 14.3 ± 0.4 | 14845 ± 1578 |
| OR0241 | 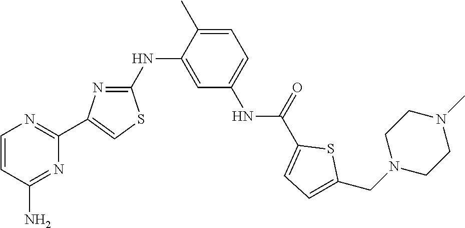 [SMILES: Cc1ccc(NC(=O)c2ccc(CN3CCN(C)CC3)s2)cc1Nc1nc(-c2nccc(N)n2)cs1] | 13.5 ± 0.0 | 14250 ± 1164 |
| OR0274 | 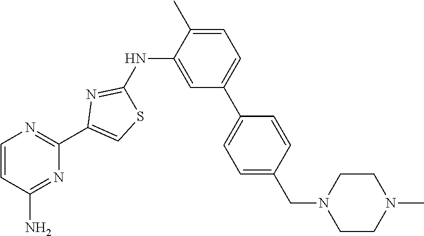 [SMILES: Cc1ccc(-c2ccc(CN3CCN(C)CC3)cc2)cc1Nc1nc(-c2nccc(N)n2)cs1] | 16.7 ± 0.3 | 2096 ± 554 |
| OR0289 | 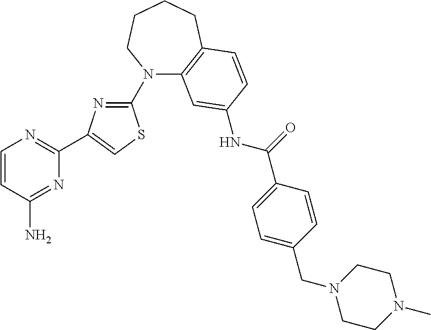 [SMILES: CN1CCN(Cc2ccc(C(=O)Nc3ccc4c(c3)N(c3nc(-c5nccc(N)n5)cs3)CCCC4)cc2)CC1] | 15.9 ± 0.5 | 3919 ± 461 |
| OR0320 | 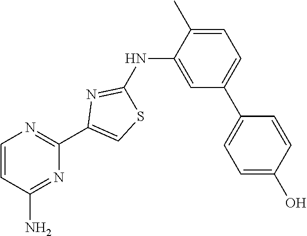 [SMILES: Cc1ccc(-c2ccc(O)cc2)cc1Nc1nc(-c2nccc(N)n2)cs1] | 15.0 ± 0.2 | 6772 ± 2812 |
| OR0321 | 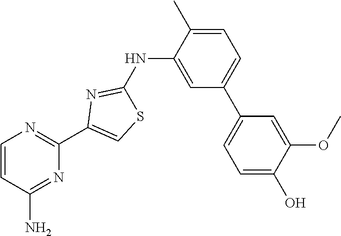 [SMILES: COc1cc(-c2ccc(C)c(Nc3nc(-c4nccc(N)n4)cs3)c2)ccc1O] | 13.3 ± 0.2 | 8289 ± 2714 |
| OR0325 | 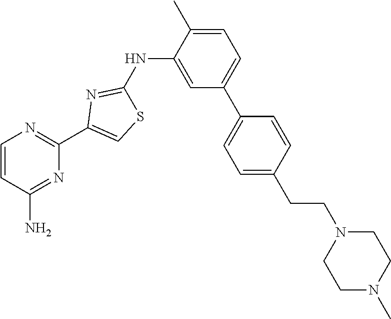 [SMILES: Cc1ccc(-c2ccc(CCN3CCN(C)CC3)cc2)cc1Nc1nc(-c2nccc(N)n2)cs1] | 17.9 ± 0.3 | 1676 ± 242 |
| OR0331 | 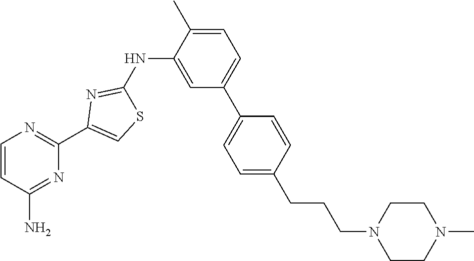 [SMILES: Cc1ccc(-c2ccc(CCCN3CCN(C)CC3)cc2)cc1Nc1nc(-c2nccc(N)n2)cs1] | 19.4 ± 0.4 | 918 ± 107 |
| OR0345 | 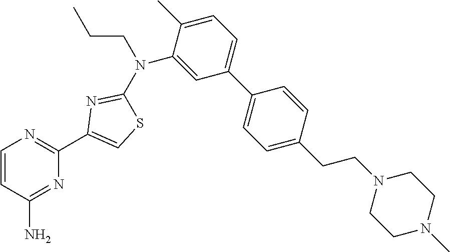 [SMILES: CCCN(c1nc(-c2nccc(N)n2)cs1)c1cc(-c2ccc(CCN3CCN(C)CC3)cc2)ccc1C] | 18.4 ± 0.2 | 505 ± 149 |
| OR0402 | 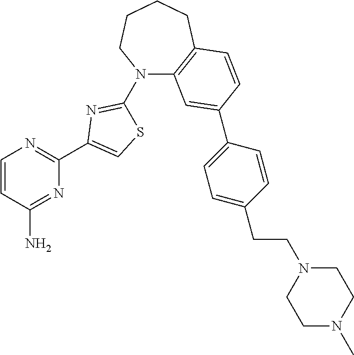 [SMILES: CN1CCN(CCc2ccc(-c3ccc4c(c3)N(c3nc(-c5nccc(N)n5)cs3)CCCC4)cc2)CC1] | 19.2 ± 0.3 | 1765 ± 160 |
| OR0596 | 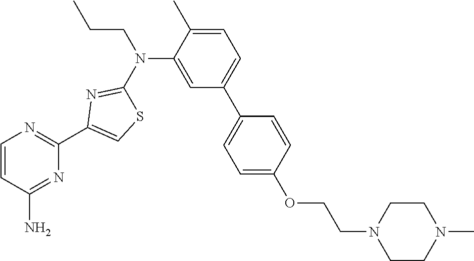 [SMILES: CCCN(c1nc(-c2nccc(N)n2)cs1)c1cc(-c2ccc(OCCN3CCN(C)CC3)cc2)ccc1C] | 18.9 ± 0.1 | 307 ± 50 |
| OR0597 | 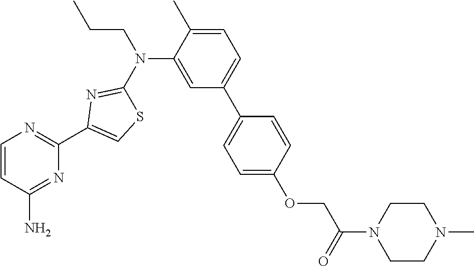 [SMILES: CCCN(c1nc(-c2nccc(N)n2)cs1)c1cc(-c2ccc(OCC(=O)N3CCN(C)CC3)cc2)ccc1C] | 17.1 ± 0.1 | 220 ± 127 |
| OR0598 | 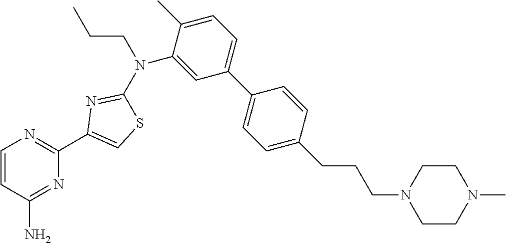 [SMILES: CCCN(c1nc(-c2nccc(N)n2)cs1)c1cc(-c2ccc(CCCN3CCN(C)CC3)cc2)ccc1C] | 19.1 ± 0.2 | 776 ± 206 |
| OR0599 | 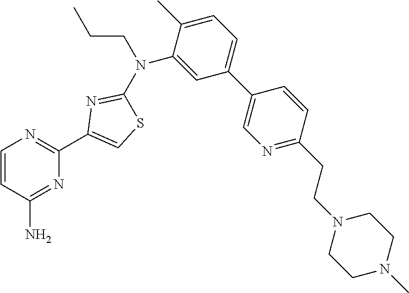 [SMILES: CCCN(c1nc(-c2nccc(N)n2)cs1)c1cc(-c2ccc(CCN3CCN(C)CC3)nc2)ccc1C] | 17.1 ± 0.2 | 136 ± 53 |
| OR0600 | 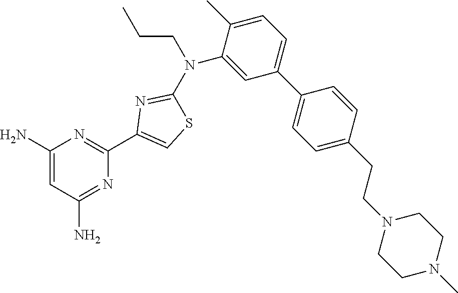 [SMILES: CCCN(c1nc(-c2nc(N)cc(N)n2)cs1)c1cc(-c2ccc(CCN3CCN(C)CC3)cc2)ccc1C] | 20.0 ± 0.6 | 341 ± 133 |
| OR0601 | 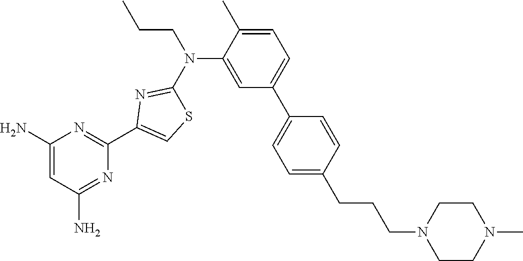 [SMILES: CCCN(c1nc(-c2nc(N)cc(N)n2)cs1)c1cc(-c2ccc(CCCN3CCN(C)CC3)cc2)ccc1C] | 20.8 ± 0.7 | 420 ± 83 |
| OR0602 | 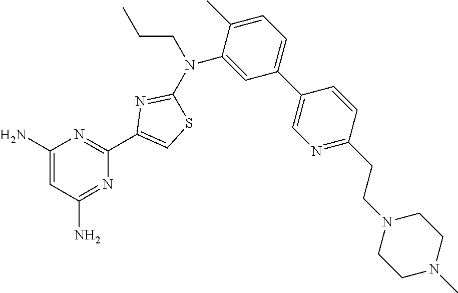 [SMILES: CCCN(c1nc(-c2nc(N)cc(N)n2)cs1)c1cc(-c2ccc(CCN3CCN(C)CC3)nc2)ccc1C] | 19.5 ± 0.5 | 130 ± 57 |
| OR0603 | 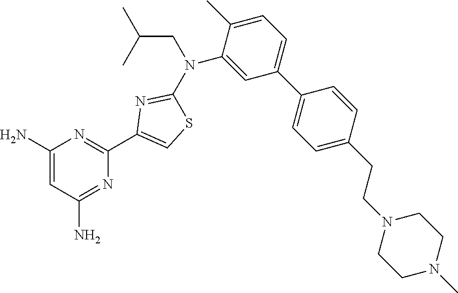 [SMILES: Cc1ccc(-c2ccc(CCN3CCN(C)CC3)cc2)cc1N(CC(C)C)c1nc(-c2nc(N)cc(N)n2)cs1] | 21.3 ± 0.8 | 255 ± 70 |
| OR0604 | 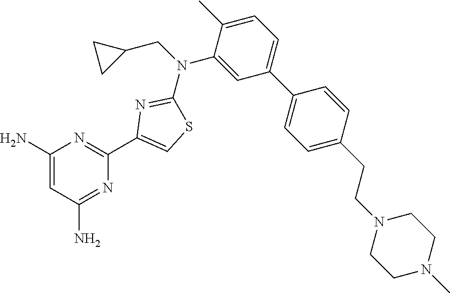 [SMILES: Cc1ccc(-c2ccc(CCN3CCN(C)CC3)cc2)cc1N(CC1CC1)c1nc(-c2nc(N)cc(N)n2)cs1] | 19.7 ± 1.0 | 609 ± 8 |
| OR0605 | 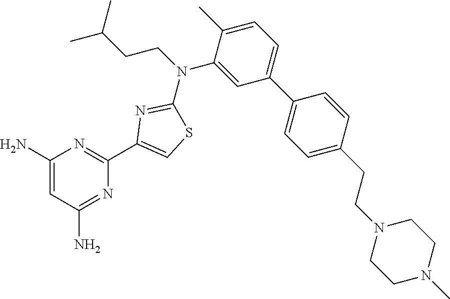 [SMILES: Cc1ccc(-c2ccc(CCN3CCN(C)CC3)cc2)cc1N(CCC(C)C)c1nc(-c2nc(N)cc(N)n2)cs1] | 18.7 ± 0.6 | 2222 ± 304 |
| OR0606 | 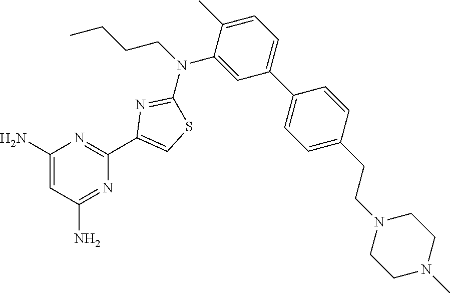 [SMILES: CCCCN(c1nc(-c2nc(N)cc(N)n2)cs1)c1cc(-c2ccc(CCN3CCN(C)CC3)cc2)ccc1C] | 19.9 ± 0.5 | 552 ± 102 |
| OR0607 | 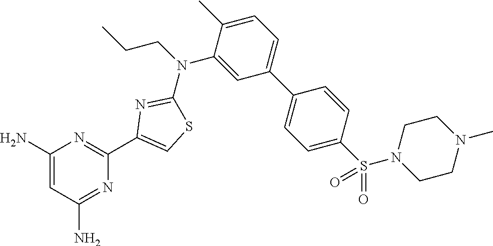 [SMILES: CCCN(c1nc(-c2nc(N)cc(N)n2)cs1)c1cc(-c2ccc(S(=O)(=O)N3CCN(C)CC3)cc2)ccc1C] | 19.2 ± 0.6 | 31 ± 27 |
| OR0608 | 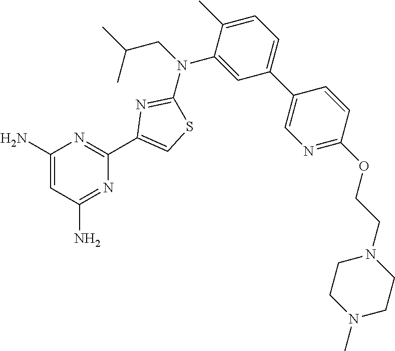 [SMILES: Cc1ccc(-c2ccc(OCCN3CCN(C)CC3)nc2)cc1N(CC(C)C)c1nc(-c2nc(N)cc(N)n2)cs1] | 21.0 ± 0.5 | 40 ± 13 |
| OR0609 | 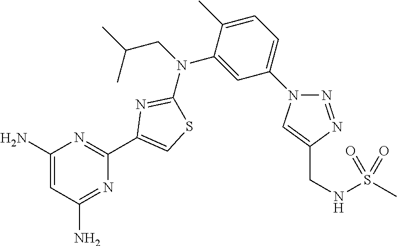 [SMILES: Cc1ccc(-n2cc(CNS(C)(=O)=O)nn2)cc1N(CC(C)C)c1nc(-c2nc(N)cc(N)n2)cs1] | 13.5 ± 0.7 | 3177 ± 611 |
| OR0610 | 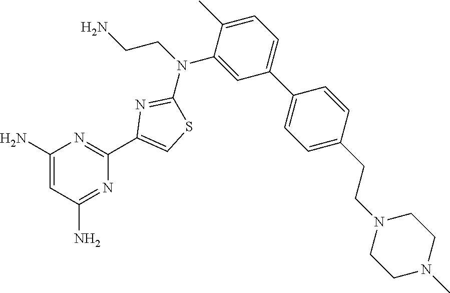 [SMILES: Cc1ccc(-c2ccc(CCN3CCN(C)CC3)cc2)cc1N(CCN)c1nc(-c2nc(N)cc(N)n2)cs1] | 14.7 ± 0.2 | 16411 ± 4375 |
| OR0611 | 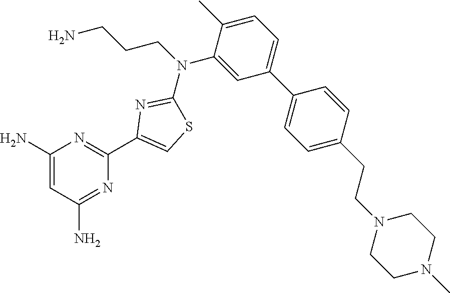 [SMILES: Cc1ccc(-c2ccc(CCN3CCN(C)CC3)cc2)cc1N(CCCN)c1nc(-c2nc(N)cc(N)n2)cs1] | 14.7 ± 0.5 | 11804 ± 100 |
| OR0612 | 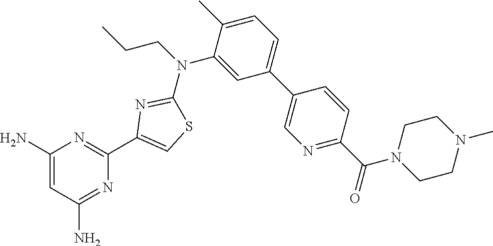 [SMILES: CCCN(c1nc(-c2nc(N)cc(N)n2)cs1)c1cc(-c2ccc(C(=O)N3CCN(C)CC3)nc2)ccc1C] | 16.6 ± 0.8 | 1005 ± 300 |
| OR0613 | 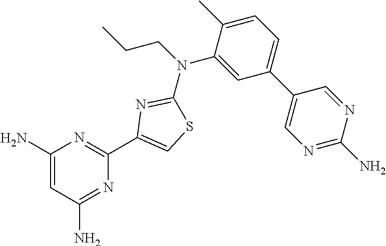 [SMILES: CCCN(c1nc(-c2nc(N)cc(N)n2)cs1)c1cc(-c2cnc(N)nc2)ccc1C] | 14.5 ± 0.7 | 703 ± 54 |
| OR0614 | 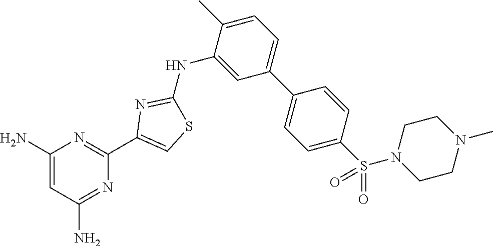 [SMILES: Cc1ccc(-c2ccc(S(=O)(=O)N3CCN(C)CC3)cc2)cc1Nc1nc(-c2nc(N)cc(N)n2)cs1] | 18.1 ± 0.8 | 10028 ± 55 |
| OR0615 | 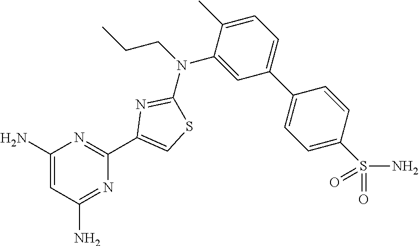 [SMILES: CCCN(c1nc(-c2nc(N)cc(N)n2)cs1)c1cc(-c2ccc(S(N)(=O)=O)cc2)ccc1C] | 17.5 ± 0.7 | 411 ± 151 |
| OR0616 | 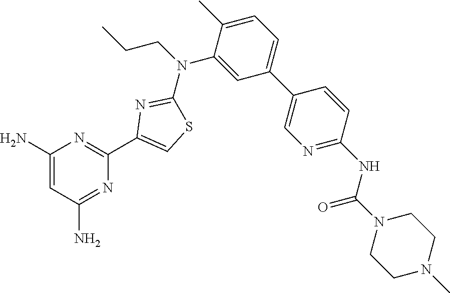 [SMILES: CCCN(c1nc(-c2nc(N)cc(N)n2)cs1)c1cc(-c2ccc(NC(=O)N3CCN(C)CC3)nc2)ccc1C] | 19.2 ± 0.8 | 135 ± 51 |
| OR0617 | 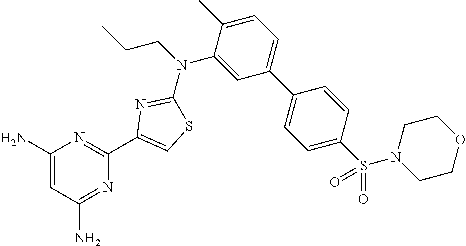 [SMILES: CCCN(c1nc(-c2nc(N)cc(N)n2)cs1)c1cc(-c2ccc(S(=O)(=O)N3CCOCC3)cc2)ccc1C] | 18.3 ± 1.1 | 78 ± 45 |
| OR0618 | 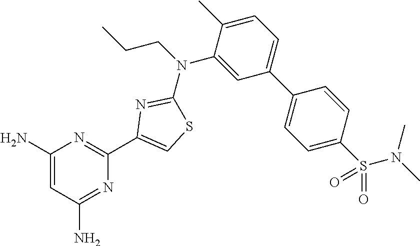 [SMILES: CCCN(c1nc(-c2nc(N)cc(N)n2)cs1)c1cc(-c2ccc(S(=O)(=O)N(C)C)cc2)ccc1C] | 18.8 ± 0.6 | 80 ± 37 |
| OR0619 | 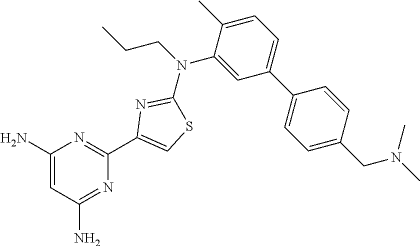 [SMILES: CCCN(c1nc(-c2nc(N)cc(N)n2)cs1)c1cc(-c2ccc(CN(C)C)cc2)ccc1C] | 16.9 ± 0.5 | 614 ± 93 |
| OR0620 | 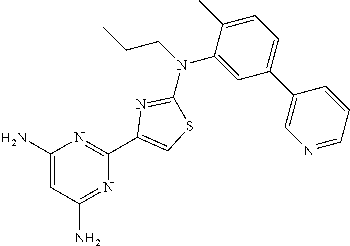 [SMILES: CCCN(c1nc(-c2nc(N)cc(N)n2)cs1)c1cc(-c2cccnc2)ccc1C] | 15.7 ± 0.5 | 407 ± 25 |
| OR0621 | 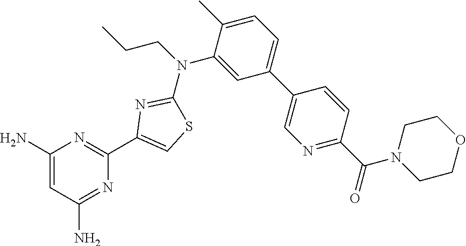 [SMILES: CCCN(c1nc(-c2nc(N)cc(N)n2)cs1)c1cc(-c2ccc(C(=O)N3CCOCC3)nc2)ccc1C] | 16.6 ± 0.9 | 169 ± 48 |
| OR0622 | 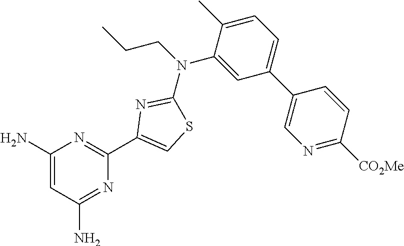 [SMILES: CCCN(c1nc(-c2nc(N)cc(N)n2)cs1)c1cc(-c2ccc(C(=O)OC)nc2)ccc1C] | 16.5 ± 0.1 | 416 ± 194 |
| OR0625 | 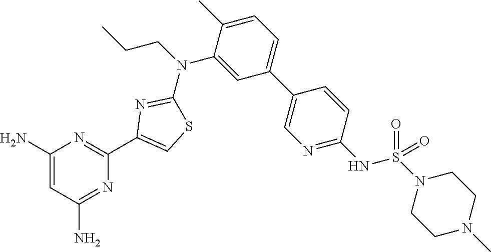 [SMILES: CCCN(c1nc(-c2nc(N)cc(N)n2)cs1)c1cc(-c2ccc(NS(=O)(=O)N3CCN(C)CC3)nc2)ccc1C] | 16.8 ± 0.7 | 353 ± 121 |
| OR0626 | 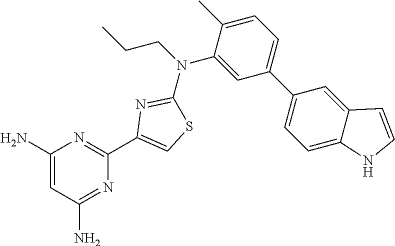 [SMILES: CCCN(c1nc(-c2nc(N)cc(N)n2)cs1)c1cc(-c2ccc3[nH]ccc3c2)ccc1C] | 16.9 ± 0.5 | 5750 ± 1276 |
| OR0627 | 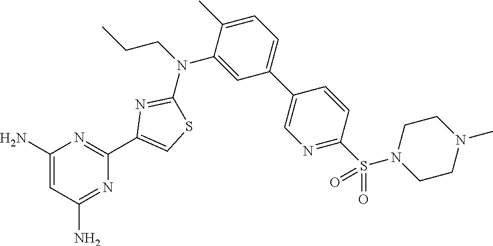 [SMILES: CCCN(c1nc(-c2nc(N)cc(N)n2)cs1)c1cc(-c2ccc(S(=O)(=O)N3CCN(C)CC3)nc2)ccc1C] | 17.5 ± 0.4 | 48 ± 17 |
| OR0629 | 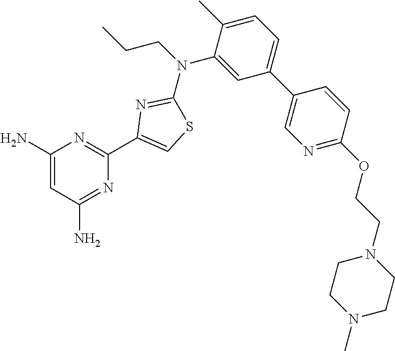 [SMILES: CCCN(c1nc(-c2nc(N)cc(N)n2)cs1)c1cc(-c2ccc(OCCN3CCN(C)CC3)nc2)ccc1C] | 20.6 ± 0.1 | 139 ± 77 |
| OR0630 | 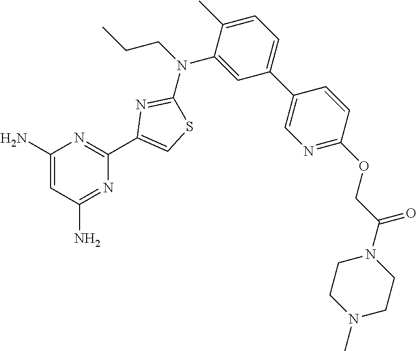 [SMILES: CCCN(c1nc(-c2nc(N)cc(N)n2)cs1)c1cc(-c2ccc(OCC(=O)N3CCN(C)CC3)nc2)ccc1C] | 17.4 ± 0.2 | 245 ± 171 |
| OR0631 | 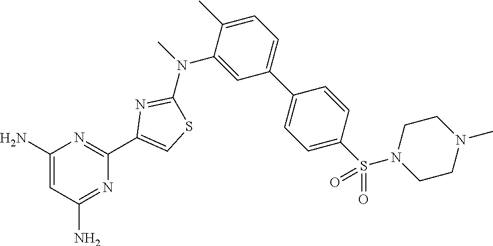 [SMILES: Cc1ccc(-c2ccc(S(=O)(=O)N3CCN(C)CC3)cc2)cc1N(C)c1nc(-c2nc(N)cc(N)n2)cs1] | 16.8 ± 0.1 | 2740 ± 1088 |
| OR0632 | 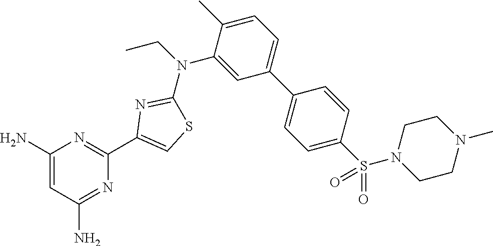 [SMILES: CCN(c1nc(-c2nc(N)cc(N)n2)cs1)c1cc(-c2ccc(S(=O)(=O)N3CCN(C)CC3)cc2)ccc1C] | 17.8 ± 0.1 | 413 ± 131 |
| OR0633 | 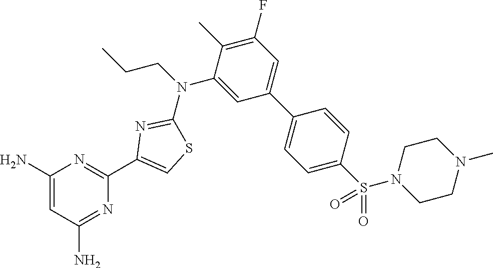 [SMILES: CCCN(c1nc(-c2nc(N)cc(N)n2)cs1)c1cc(-c2ccc(S(=O)(=O)N3CCN(C)CC3)cc2)cc(F)c1C] | 17.3 ± 0.1 | 140 ± 57 |
| OR0635 | 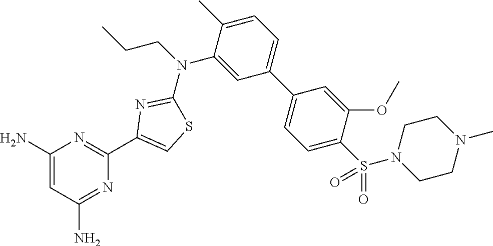 [SMILES: CCCN(c1nc(-c2nc(N)cc(N)n2)cs1)c1cc(-c2ccc(S(=O)(=O)N3CCN(C)CC3)c(OC)c2)ccc1C] | 20.40 ± 0.9 | 15.4 ± 10 |
| OR0636 | 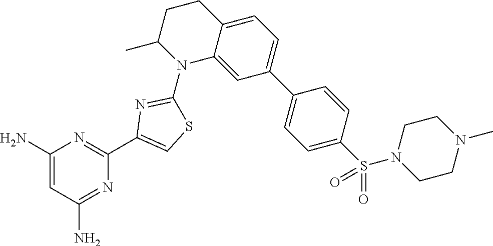 [SMILES: CC1CCc2ccc(-c3ccc(S(=O)(=O)N4CCN(C)CC4)cc3)cc2N1c1nc(-c2nc(N)cc(N)n2)cs1] | 17.7 ± 0.9 | 301 ± 116 |
| OR0637 | 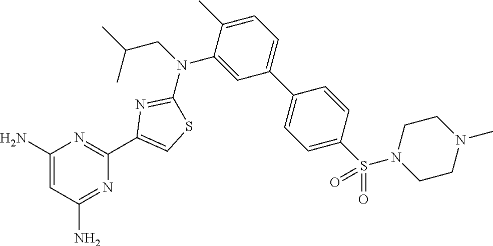 [SMILES: Cc1ccc(-c2ccc(S(=O)(=O)N3CCN(C)CC3)cc2)cc1N(CC(C)C)c1nc(-c2nc(N)cc(N)n2)cs1] | 20.5 ± 0.9 | 84 ± 49 |
| OR0637-1 | 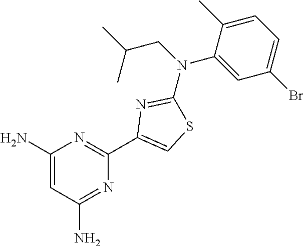 [SMILES: Cc1ccc(Br)cc1N(CC(C)C)c1nc(-c2nc(N)cc(N)n2)cs1] | 14.9 ± 0.2 | 8309 ± 1869 |
| OR0638 | 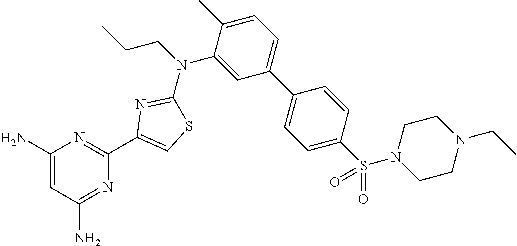 [SMILES: CCCN(c1nc(-c2nc(N)cc(N)n2)cs1)c1cc(-c2ccc(S(=O)(=O)N3CCN(CC)CC3)cc2)ccc1C] | 20.2 ± 0.9 | 35 ± 17 |
| OR0639 | 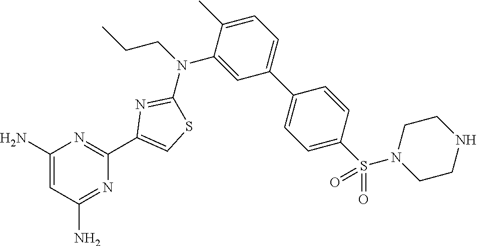 [SMILES: CCCN(c1nc(-c2nc(N)cc(N)n2)cs1)c1cc(-c2ccc(S(=O)(=O)N3CCNCC3)cc2)ccc1C] | 20.7 ± 0.9 | 119 ± 36 |
| OR0640 | 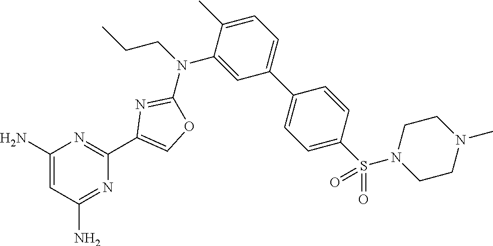 [SMILES: CCCN(c1nc(-c2nc(N)cc(N)n2)co1)c1cc(-c2ccc(S(=O)(=O)N3CCN(C)CC3)cc2)ccc1C] | 18.2 ± 0.9 | 3670 ± 2151 |
| OR0641 | 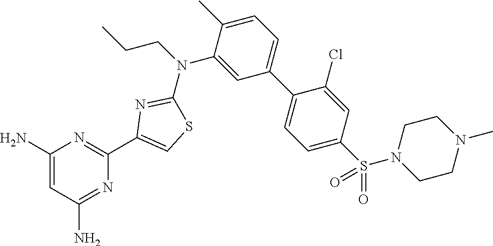 [SMILES: CCCN(c1nc(-c2nc(N)cc(N)n2)cs1)c1cc(-c2ccc(S(=O)(=O)N3CCN(C)CC3)cc2Cl)ccc1C] | 21.1 ± 1.1 | 10.5 ± 5.0 |
| OR0642 | 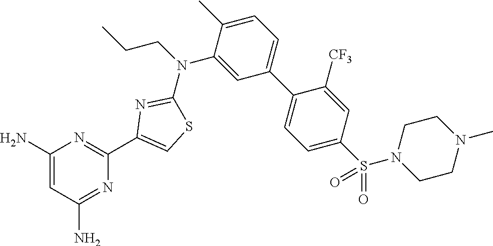 [SMILES: CCCN(c1nc(-c2nc(N)cc(N)n2)cs1)c1cc(-c2ccc(S(=O)(=O)N3CCN(C)CC3)cc2C(F)(F)F)ccc1C] | 22.2 ± 0.9 | 2.2 ± 2.0 |
| OR0643 | 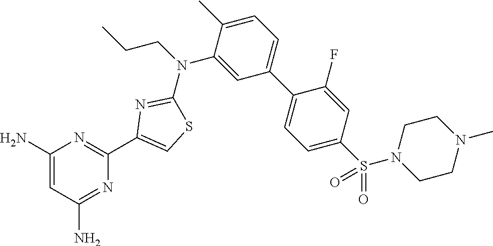 [SMILES: CCCN(c1nc(-c2nc(N)cc(N)n2)cs1)c1cc(-c2ccc(S(=O)(=O)N3CCN(C)CC3)cc2F)ccc1C] | 20.2 ± 0.9 | 46 ± 18 |
| OR0644 | 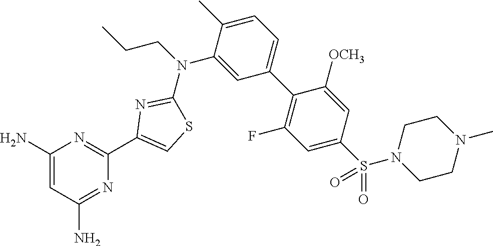 [SMILES: CCCN(c1nc(-c2nc(N)cc(N)n2)cs1)c1cc(-c2c(F)cc(S(=O)(=O)N3CCN(C)CC3)cc2OC)ccc1C] | 20.5 ± 1.2 | 12.1 ± 7.7 |
| OR0645 | 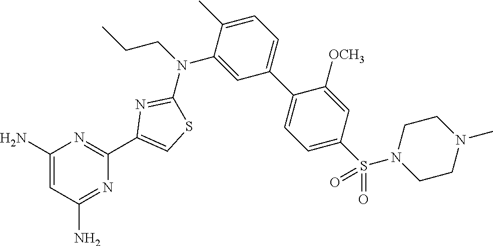 [SMILES: CCCN(c1nc(-c2nc(N)cc(N)n2)cs1)c1cc(-c2ccc(S(=O)(=O)N3CCN(C)CC3)cc2OC)ccc1C] | 19.7 ± 0.9 | 67 ± 22 |
| OR0646 | 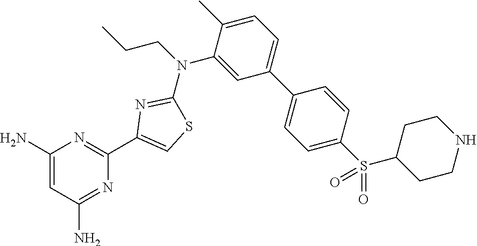 [SMILES: CCCN(c1nc(-c2nc(N)cc(N)n2)cs1)c1cc(-c2ccc(S(=O)(=O)C3CCNCC3)cc2)ccc1C] | 22.4 ± 1.1 | 171 ± 48 |
| OR0647 | 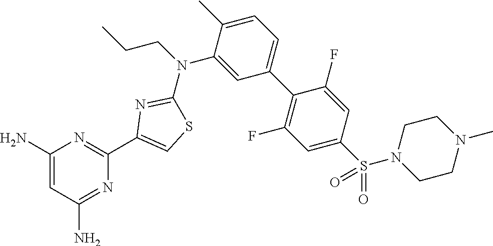 [SMILES: CCCN(c1nc(-c2nc(N)cc(N)n2)cs1)c1cc(-c2c(F)cc(S(=O)(=O)N3CCN(C)CC3)cc2F)ccc1C] | 21.1 ± 1.6 | 10.9 ± 4.1 |
| OR0648 | 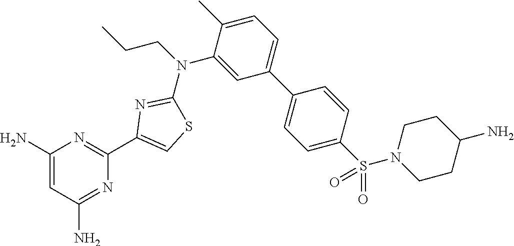 [SMILES: CCCN(c1nc(-c2nc(N)cc(N)n2)cs1)c1cc(-c2ccc(S(=O)(=O)N3CCC(N)CC3)cc2)ccc1C] | 22.3 ± 1.1 | 134 ± 38 |
| OR0649 | 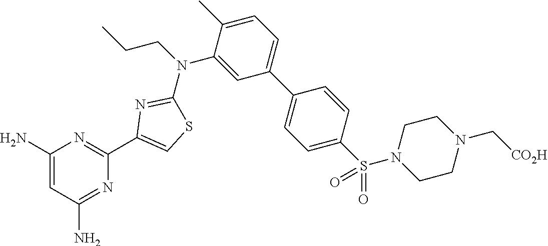 [SMILES: CCCN(c1nc(-c2nc(N)cc(N)n2)cs1)c1cc(-c2ccc(S(=O)(=O)N3CCN(CC(=O)O)CC3)cc2)ccc1C] | 17.6 ± 1.3 | 13417 ± 546 |
| OR0650 | 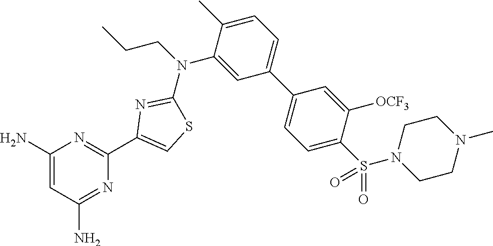 [SMILES: CCCN(c1nc(-c2nc(N)cc(N)n2)cs1)c1cc(-c2ccc(S(=O)(=O)N3CCN(C)CC3)c(OC(F)(F)F)c2)ccc1C] | 18.6 ± 1.5 | 120 ± 25 |
| OR0651 | 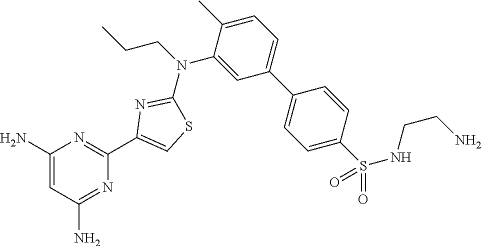 [SMILES: CCCN(c1nc(-c2nc(N)cc(N)n2)cs1)c1cc(-c2ccc(S(=O)(=O)NCCN)cc2)ccc1C] | 20.8 ± 1.2 | 1525 ± 178 |
| OR0652 | 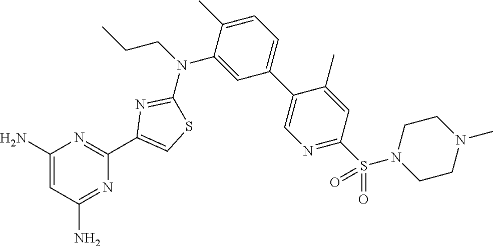 [SMILES: CCCN(c1nc(-c2nc(N)cc(N)n2)cs1)c1cc(-c2cnc(S(=O)(=O)N3CCN(C)CC3)cc2C)ccc1C] | 19.2 ± 0.1 | 7.3 ± 2.5 |
| OR0652-1 | 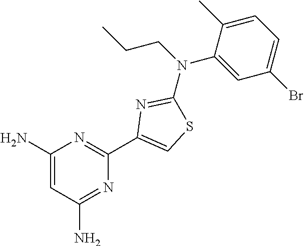 [SMILES: CCCN(c1nc(-c2nc(N)cc(N)n2)cs1)c1cc(Br)ccc1C] | 13.9 ± 0.2 | 11347 ± 1369 |
| OR0653 | 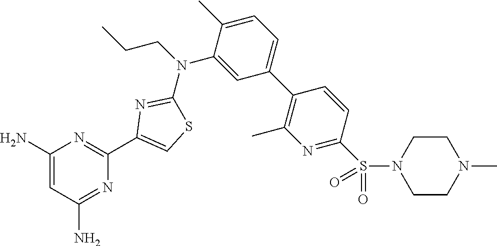 [SMILES: CCCN(c1nc(-c2nc(N)cc(N)n2)cs1)c1cc(-c2ccc(S(=O)(=O)N3CCN(C)CC3)nc2C)ccc1C] | 21.6 ± 0.4 | 2.8 ± 1.3 |
| OR0654 | 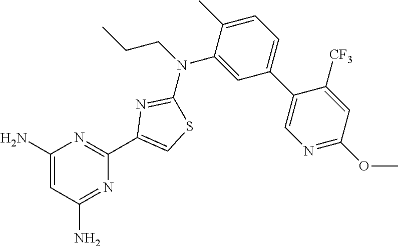 [SMILES: CCCN(c1nc(-c2nc(N)cc(N)n2)cs1)c1cc(-c2cnc(OC)cc2C(F)(F)F)ccc1C] | 18.3 ± 0.4 | 610 ± 153 |
| OR0655 | 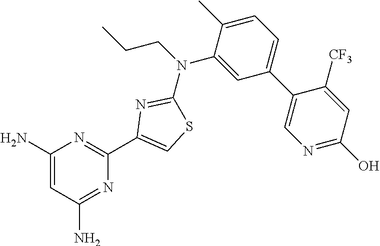 [SMILES: CCCN(c1nc(-c2nc(N)cc(N)n2)cs1)c1cc(-c2cnc(O)cc2C(F)(F)F)ccc1C] | 19.0 ± 0.0 | 251 ± 36 |
| OR0656 | 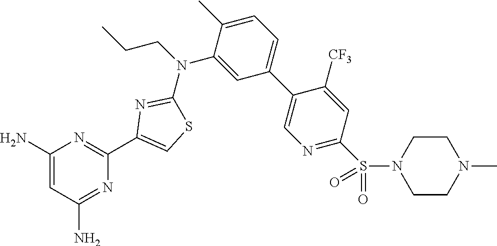 [SMILES: CCCN(c1nc(-c2nc(N)cc(N)n2)cs1)c1cc(-c2cnc(S(=O)(=O)N3CCN(C)CC3)cc2C(F)(F)F)ccc1C] | 21.3 ± 0.8 | 3.8 ± 2.0 |
| OR0657 | 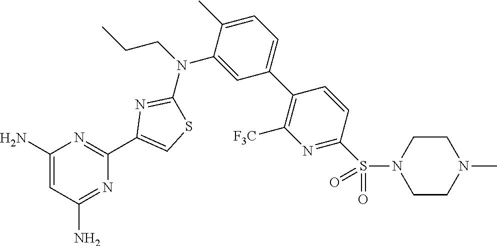 [SMILES: CCCN(c1nc(-c2nc(N)cc(N)n2)cs1)c1cc(-c2ccc(S(=O)(=O)N3CCN(C)CC3)nc2C(F)(F)F)ccc1C] | 22.0 ± 0.4 | 1.6 ± 0.9 |
| OR0658 | 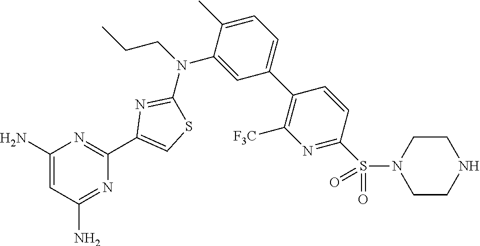 [SMILES: CCCN(c1nc(-c2nc(N)cc(N)n2)cs1)c1cc(-c2ccc(S(=O)(=O)N3CCNCC3)nc2C(F)(F)F)ccc1C] | 22.4 ± 0.1 | 11.3 ± 2.1 |

The experimental evaluations by biochemical affinity assay and cellular assay obtained for 82 compounds are summarized in Table 1 and FIG. 2. The binding of the compound of the invention to dCK was assessed using Thermal Shift Assay. The dCK thermal stabilization produced by the binding to these compounds ranged from +4.1 to +22.4° C. (ΔTₘ) (Table 1).

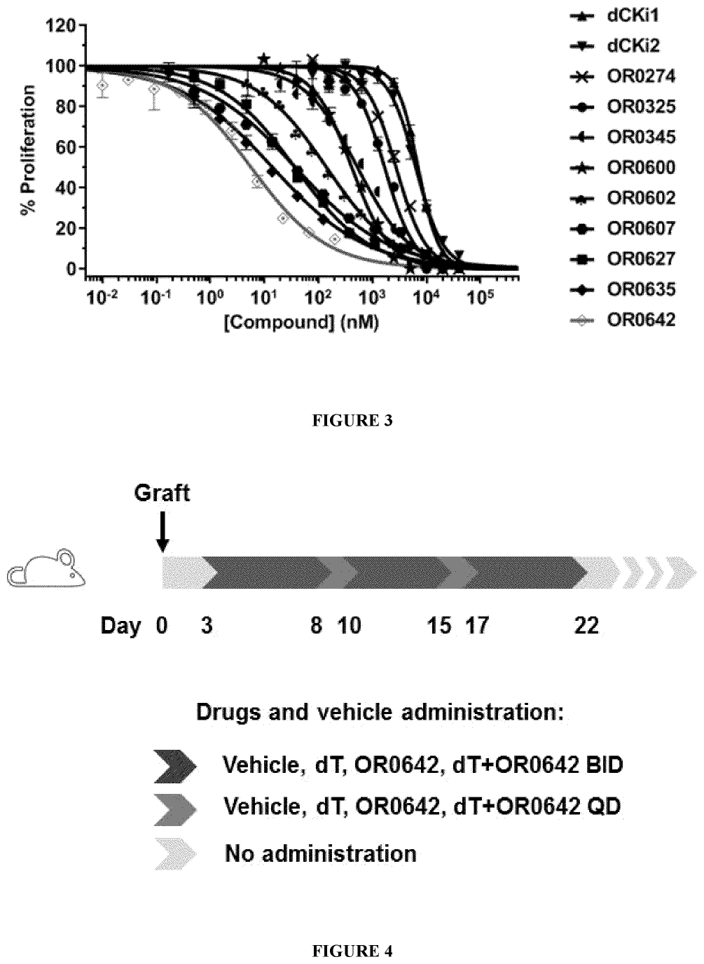

Inventors developed a cellular assay to validate whether a combinatory inhibition of the De Novo and Salvage pathways by the combination of a dCK inhibitor with a Ribonucleotide Reductase (RNR) inhibitor could lead to the inhibition of cellular proliferation. Results on the human leukemia cell line CCRF-CEM (T-ALL) revealed that new compounds were able to inhibit cancer cells proliferation in combination with the physiologic RNR inhibitor dT in presence of dC. IC₅₀ cell proliferation values were obtained between 37 μM and around 2 nM, more precisely between 37 μM and 1.6 nM (Table 1, FIG. 2, and FIG. 3).

### In Vivo Assay

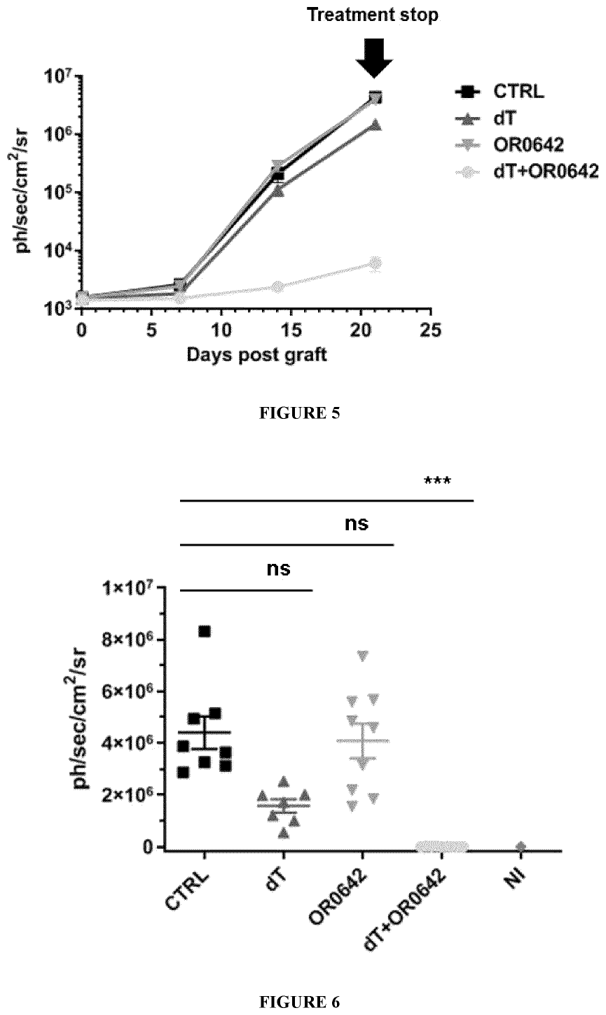

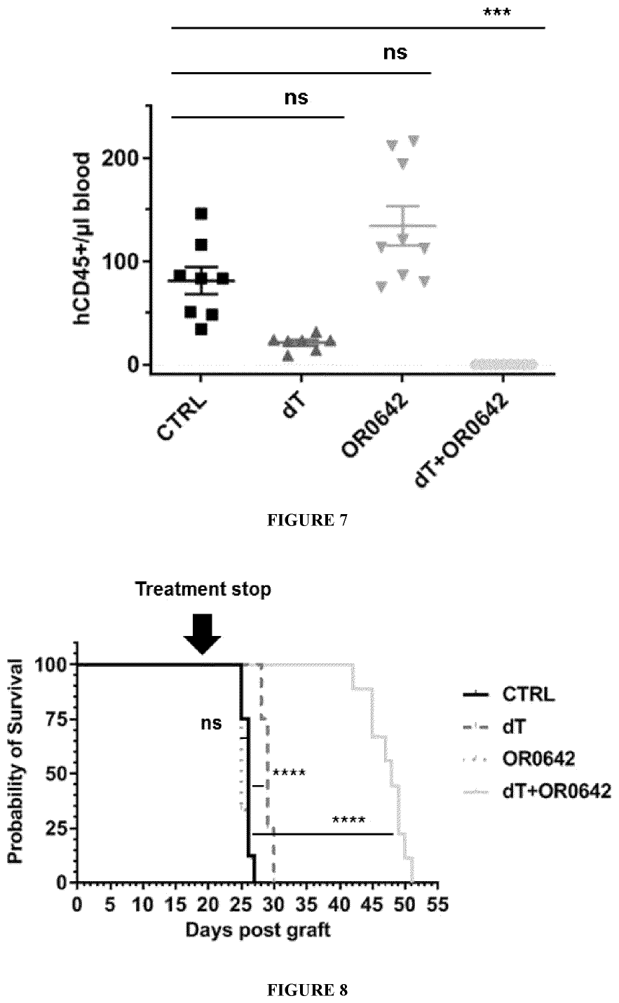

To investigate whether the efficacy observed in vitro could be correlated in vivo, compound OR0642 (hydrochlorhydre salt) was challenged in a systemic leukemia in vivo mice model. Inventors studied the in vivo efficacy and tolerability of co-targeting the alternative nucleotide biosynthetic pathways De Novo Pathway (DNP) and Salvage Pathway (SP) in a NOD-SCID-IL2Rγc null (NSG) mice model inoculated intravenously with luciferase expressing the human CCRF-CEM T-ALL cell line. The DNP was inhibited with dT, a physiologic inhibitor of RNR, and the SP with the newly developed dCK inhibitor OR0642. Treatment was initiated on day 3 after graft and was maintained during 19 days with cycles of 5 days (BID, twice/day) and 2 days (QD, once/day) (FIG. 4). Inventors assessed the efficacy of the combination therapy and also the two components when administered separately. Leukemic progression was monitored by bioluminescent imaging once a week (FIG. 5). On Day 21, mice in the combination treatment (dT+OR0642) had more than 100-fold lower bioluminescent signal than control mice and individual therapies, indicating significant decrease in systemic leukemic burden and promising therapeutic efficacy (FIG. 6). Moreover, hCD45 populations in blood samples were determined by flux cytometry on day 21 and combination therapy decreased significantly disease progression (FIG. 7). As expected, the two components separately were poorly efficacious in vivo; OR0642 did not show any anti-proliferative effect and dT scarcely reduced the disease burden (FIG. 5). As a consequence, most of the mice died around day 26 (FIG. 8). Importantly, only the combination therapy (dT+OR0642) dramatically reduced disease progression (FIG. 5) and prolonged survival in these mice (48 days) even after a treatment arrest at day 21 (FIG. 8), indicating strong synergy between these two therapeutic agents and significant survival advantage. Therefore, pharmacological co-targeting of both the DNP and SP biosynthetic pathways thanks to OR0642 is highly effective against T-ALL leukemic cells in vivo.

1-18. (canceled)

19. A compound having the following formula (I): 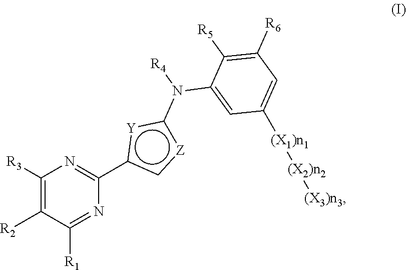
wherein:
Y and Z represent independently NH, N, O, or S;
R₁, R₂, and R₃ represent independently a radical selected from the group consisting of a hydrogen, a —NR₇R₈ group with R₇ and R₈ being independently a hydrogen or a (C₁-C₆)alkyl group, and a halogen;
R₄ and R₅ represent independently a hydrogen, a (C₁-C₆)alkyl group optionally substituted by a radical selected from the group consisting of a cycloalkyl, and a —NR₉R₁₀ group with R₉ and R₁₀ being independently a hydrogen or a (C₁-C₆)alkyl group, or R₄ and R₅ form together an azepanyl;
R₆ represents a hydrogen or a halogen;
X₁ represents a —NHCO— group, an oxygen atom, a halogen, a —C≡C— group, or a —O—CH₂— group;
X₂ represents a 5-12 membered ring optionally substituted by at least one radical selected from the group consisting of a (C₁-C₆)alkyl group optionally substituted by at least one halogen, a (C₁-C₆)alkoxy group optionally substituted by at least one halogen, a halogen, and a hydroxy;
X₃ represents a radical selected from the group consisting of:
a (C₁-C₆)alkyl substituted by at least one radical A,
a (C₁-C₆)alkoxy substituted by at least one radical A,
a —C(O)— substituted by at least one radical A,
a —SO₂— substituted by at least one radical A,
a —NH—SO₂— substituted by at least one radical A,
a —NHCO— substituted by at least one radical A,
in which said at least one radical A is selected from the group consisting of:
a piperazinyl optionally substituted by a (C₁-C₆)alkyl or by a (C₁-C₆)alkyl substituted by a —COOH,
a —CO-piperazinyl substituted by a (C₁-C₆)alkyl,
a morpholinyl,
a piperidinyl optionally substituted by a —NR₇R₈ group with R₇ and R₈ being independently a hydrogen or a (C₁-C₆)alkyl group, or
a 2,6-diazaspiro[3.3]heptanyl substituted by a (C₁-C₆)alkyl,
a —NR₁₁R₁₂ group with R₁₁ and R₁₂ being independently a hydrogen, a (C₁-C₆)alkyl group, or a —SO₂—CH₃ group,
a —NH—(C₁-C₆)alkyl-NR₁₃R₁₄ group with R₁₃ and R₁₄ being independently a hydrogen, or a (C₁-C₆)alkyl group,
a (C₁-C₆)alkoxy, and
a hydroxy,
a hydroxy, and
a —NR₁₅R₁₆ group with R₁₅ and R₁₆ being independently a hydrogen or a (C₁-C₆)alkyl group; and
n₁, n₂, and n₃ are independently 0 or 1;
and the pharmaceutically acceptable salts, the tautomers, and the solvates thereof.

20. The compound according to claim 19, wherein:
Y and Z represent independently NH, N, O, or S;
R₁, R₂, and R₃ represent independently a radical selected from the group consisting of a hydrogen, a —NR₇R₈ group with R₇ and R₈ being independently a hydrogen or a (C₁-C₆)alkyl group, and a halogen;
R₄ and R₅ represent independently a hydrogen, a (C₁-C₆)alkyl group optionally substituted by a radical selected from the group consisting of a cycloalkyl, and a —NR₉R₁₀ group with R₉ and R₁₀ being independently a hydrogen or a (C₁-C₆)alkyl group, or R₄ and R₅ form together an azepanyl;
R₆ represents a hydrogen or a halogen;
X₁ represents a —NHCO— group, an oxygen atom, a —C≡C— group, or a —O—CH₂— group;
X₂ represents a 5-12 membered ring optionally substituted by at least one radical selected from the group consisting of a (C₁-C₆)alkyl group optionally substituted by at least one halogen, a (C₁-C₆)alkoxy group optionally substituted by at least one halogen, and a halogen;
X₃ represents a radical selected from the group consisting of:
a (C₁-C₆)alkyl substituted by at least one radical A,
a (C₁-C₆)alkoxy substituted by at least one radical A,
a —C(O)— substituted by at least one radical A,
a —SO₂— substituted by at least one radical A,
a —NH—SO₂— substituted by at least one radical A,
a —NHCO— substituted by at least one radical A,
in which said at least one radical A is selected from the group consisting of:
a piperazinyl optionally substituted by a (C₁-C₆)alkyl or by a (C₁-C₆)alkyl substituted by a —COOH,
a —CO-piperazinyl substituted by a (C₁-C₆)alkyl,
a morpholinyl,
a piperidinyl optionally substituted by a —NR₇R₈ group with R₇ and R₈ being independently a hydrogen or a (C₁-C₆)alkyl group, or
a 2,6-diazaspiro[3.3]heptanyl substituted by a (C₁-C₆)alkyl,
a —NR₁₁R₁₂ group with R₁₁ and R₁₂ being independently a hydrogen, a (C₁-C₆)alkyl group, or a —SO₂—CH₃ group,
a —NH—(C₁-C₆)alkyl-NR₁₃R₁₄ group with R₁₃ and R₁₄ being independently a hydrogen, or a (C₁-C₆)alkyl group,
a (C₁-C₆)alkoxy, and
a hydroxy,
a hydroxy, and
a —NR₁₅R₁₆ group with R₁₅ and R₁₆ being independently a hydrogen or a (C₁-C₆)alkyl group; and
n₁, n₂, and n₃ are independently 0 or 1.

21. The compound according to claim 19, wherein said compound has the following formula (I′): 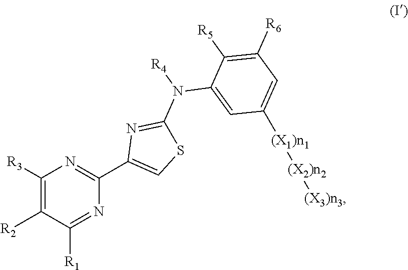
wherein:
R₁, R₂, and R₃ represent independently a radical selected from the group consisting of a hydrogen, a —NR₇R₈ group with R₇ and R₈ being independently a hydrogen or a (C₁-C₆)alkyl group, and a halogen;
R₄ and R₅ represent independently a hydrogen, a (C₁-C₆)alkyl group optionally substituted by a radical selected from the group consisting of a cycloalkyl, and a —NR₉R₁₀ group with R₉ and R₁₀ being independently a hydrogen or a (C₁-C₆)alkyl group, or R₄ and R₅ form together an azepanyl;
R₆ represents a hydrogen or a halogen;
X₁ represents a —NHCO— group, an oxygen atom, a halogen, a —C≡C— group, or a —O—CH₂— group;
X₂ represents a 5-12 membered ring optionally substituted by at least one radical selected from the group consisting of a (C₁-C₆)alkyl group optionally substituted by at least one halogen, a (C₁-C₆)alkoxy group optionally substituted by at least one halogen, a halogen, and a hydroxy;
X₃ represents a radical selected from the group consisting of:
a (C₁-C₆)alkyl substituted by at least one radical A,
a (C₁-C₆)alkoxy substituted by at least one radical A,
a —C(O) substituted by at least one radical A,
a —SO₂ substituted by at least one radical A,
a —NH—SO₂ substituted by at least one radical A,
a —NHCO substituted by at least one radical A,
in which said at least one radical A is selected from the group consisting of:
a piperazinyl optionally substituted by a (C₁-C₆)alkyl or by a (C₁-C₆)alkyl substituted by a —COOH,
a —CO-piperazinyl substituted by a (C₁-C₆)alkyl,
a morpholinyl,
a piperidinyl optionally substituted by a —NR₇R₈ group with R₇ and R₈ being independently a hydrogen or a (C₁-C₆)alkyl group, or
a 2,6-diazaspiro[3.3]heptanyl substituted by a (C₁-C₆)alkyl,
a —NR₁₁R₁₂ group with R₁₁ and R₁₂ being independently a hydrogen, a (C₁-C₆)alkyl group, or a —SO₂—CH₃ group,
a —NH—(C₁-C₆)alkyl-NR₁₃R₁₄ group with R₁₃ and R₁₄ being independently a hydrogen, or a (C₁-C₆)alkyl group,
a (C₁-C₆)alkoxy, and
a hydroxy,
a hydroxy, and
a —NR₁₅R₁₆ group with R₁₅ and R₁₆ being independently a hydrogen or a (C₁-C₆)alkyl group; and
n₁, n₂, and n₃ are independently 0 or 1.

22. The compound according to claim 19, wherein at least one group chosen among R₁, R₂, and R₃ is not a hydrogen.

23. The compound according to claim 19, wherein:
R₁ represents a —NR₇R₈ group with R₇ and R₈ being a hydrogen;
R₂ represents a hydrogen or a halogen; and
R₃ represents a hydrogen or a —NR₇R₈ group with R₇ and R₈ being a hydrogen.

24. The compound according to claim 19, wherein:
R₄ represents a hydrogen or a (C₁-C₆)alkyl group optionally substituted by a cycloalkyl or by a —NR₉R₁₀ group with R₉ and R₁₀ being a hydrogen; and
R₅ represents a (C₁-C₆)alkyl group.

25. The compound according to claim 19, wherein n₁+n₂+n₃ is equal or greater than 1.

26. The compound according to claim 19, wherein n₁ is 0.

27. The compound according to claim 19, wherein n₁ is 1 and X₁ represents a —NHCO— group.

28. The compound according to claim 19, wherein n₂ is 1 and X₂ represents a 5-12 membered ring selected from the group consisting of a phenyl, a pyrimidinyl, a thiophenyl, a pyridinyl, a triazolyl, and an indolyl, said 5-12 membered ring being optionally substituted by at least one radical selected from the group consisting of a methoxy, a trifluoromethoxy, a halogen, a hydroxy, a methyl, and a trifluromethyl.

29. The compound according to claim 19, wherein n₃ is 1 and X₃ represents a radical selected from the group consisting of a (C₁-C₆)alkyl, a (C₁-C₆)alkoxy, a —C(O)—, a —SO₂—, a —NH—SO₂—, and a —NHCO—, said radical being substituted by a piperazinyl optionally substituted by a (C₁-C₆)alkyl.

30. The compound according to claim 19, wherein n₃ is 1 and X₃ represents a (C₁-C₆)alkyl substituted by at least one radical A selected from the group consisting of a piperazinyl substituted by a methyl, a —N(CH₃)₂, a 2,6-diazaspiro[3.3]heptanyl substituted by a methyl, and a —NHSO₂CH₃.

31. The compound according to claim 19, wherein n₃ is 1 and X₃ represents a —SO₂— substituted by at least one radical A selected from the group consisting of a piperazinyl substituted by a methyl, a —NR₁₁R₁₂ group with R₁₁ and R₁₂ being independently a hydrogen or a methyl group, and a morpholinyl.

32. The compound according to claim 19, wherein:
R₁, R₂, and R₃ represent independently a hydrogen, or a —NR₇R₈ group with R₇ and R₈ being independently a hydrogen;
R₄ and R₅ represent independently a (C₁-C₆)alkyl group;
R₆ represents a hydrogen;
X₂ represents a 5-6 membered ring selected from the group consisting of a phenyl, and a pyridinyl, said 5-6 membered ring being optionally substituted by at least one radical selected from the group consisting of a trifluoromethyl, a trifluoromethoxy, a halogen, a hydroxy, a methoxy, and a methyl;
X₃ represents a —SO₂— substituted by at least one radical selected from the group consisting of a piperazinyl optionally substituted by a (C₁-C₆)alkyl or by a (C₁-C₆)alkyl substituted by a —COOH, a piperidinyl optionally substituted by a —NH₂, and a —NH—(C₁-C₆)alkyl-NH₂;
n₁ is 0; and
nm and n₃ are 1.

33. The compound according to claim 19, wherein said compound is selected from the group consisting of:
N-(3-((4-(4-Aminopyrimidin-2-yl)thiazol-2-yl)amino)-4-methylphenyl)-4-((4-methylpiperazin-1-yl)methyl)benzamide (dCKi-1);
N-(3-((4-(4,6-Diaminopyrimidin-2-yl)thiazol-2-yl)amino)-4-methylphenyl)-4-((4-methylpiperazin-1-yl)methyl)benzamide (dCKi-2);
N-(3-((4-(4-Aminopyrimidin-2-yl)thiazol-2-yl)(propyl)amino)-4-methylphenyl)-4-((4-methylpiperazin-1-yl)methyl)benzamide (OR0105);
N-(3-((4-(4-Aminopyrimidin-2-yl)thiazol-2-yl)(propyl)amino)-4-methylphenyl)-2-((4-methylpiperazin-1-yl)methyl)pyrimidine-5-carboxamide (OR0125);
4-(4-Aminopyrimidin-2-yl)-N-(2-methyl-5-(4-((4-methylpiperazin-1-yl)methyl)phenoxy)phenyl)thiazol-2-amine (OR0143);
N-(3-((4-(4-Aminopyrimidin-2-yl)thiazol-2-yl)amino)-4-methylphenyl)-4-(2-(4-methylpiperazin-1-yl)ethyl)benzamide (OR0146);
4-(4-Aminopyrimidin-2-yl)-N-(2-methyl-5-((4-((4-methylpiperazin-1-yl)methyl)phenyl)ethynyl)phenyl)thiazol-2-amine (OR0153);
N-(3-((4-(4-Aminopyrimidin-2-yl)thiazol-2-yl)amino)-4-methylphenyl)-4-((dimethylamino)methyl)benzamide (OR0155);
N-(3-((4-(4-Aminopyrimidin-2-yl)thiazol-2-yl)amino)-4-methylphenyl)-4-((6-methyl-2,6-diazaspiro[3.3]heptan-2-yl)methyl)benzamide (OR0156);
4-(4-Aminopyrimidin-2-yl)-N-(2-methyl-5-(4-((4-methylpiperazin-1-yl)methyl)benzyloxy)phenyl)thiazol-2-amine (OR0232);
4-(4-Aminopyrimidin-2-yl)-N-(5-((4-((dimethylamino)methyl)phenyl)ethynyl)-2-methylphenyl)thiazol-2-amine (OR0237);
N-(3-((4-(4-Amino-5-fluoropyrimidin-2-yl)thiazol-2-yl)amino)-4-methylphenyl)-4-((4-methylpiperazin-1-yl)methyl)benzamide (OR0239);
N-(3-((4-(4-Aminopyrimidin-2-yl)thiazol-2-yl)amino)-4-methylphenyl)-5-((4-methylpiperazin-1-yl)methyl)thiophene-2-carboxamide (OR0241);
4-(4-Aminopyrimidin-2-yl)-N-(4-methyl-4′-((4-methylpiperazin-1-yl)methyl)-[1,1′-biphenyl]-3-yl)thiazol-2-amine (OR0274);
N-(1-(4-(4-Aminopyrimidin-2-yl)thiazol-2-yl)-2,3,4,5-tetrahydro-1H-benzo[b]azepin-8-yl)-4-((4-methylpiperazin-1-yl)methyl)benzamide (OR0289);
3′-((4-(4-Aminopyrimidin-2-yl)thiazol-2-yl)amino)-4′-methyl-[1,1′-biphenyl]-4-ol (OR0320);
3′-((4-(4-Aminopyrimidin-2-yl)thiazol-2-yl)amino)-4′-methyl-[1,1′-biphenyl]-4-ol (OR0321);
4-(4-Aminopyrimidin-2-yl)-N-(4-methyl-4′-(2-(4-methylpiperazin-1-yl)ethyl)-[1,1′-biphenyl]-3-yl)thiazol-2-amine (OR0325);
4-(4-Aminopyrimidin-2-yl)-N-(4-methyl-4′-(3-(4-methylpiperazin-1-yl)propyl)-[1,1′-biphenyl]-3-yl)thiazol-2-amine (OR0331);
4-(4-Aminopyrimidin-2-yl)-N-(4-methyl-4′-(3-(4-methylpiperazin-1-yl)ethyl)-[1,1′-biphenyl]-3-yl)-N-propylthiazol-2-amine (OR0345);
2-(2-(8-(4-(2-(4-Methylpiperazin-1-yl)ethyl)phenyl)-2,3,4,5-tetrahydro-1H-benzo[b]azepin-1-yl)thiazol-4-yl)pyrimidin-4-amine (OR0402);
4-(4-Aminopyrimidin-2-yl)-N-(4-methyl-4′-(2-(4-methylpiperazin-1-yl)ethoxy)-[1,1′-biphenyl]-3-yl)-N-propylthiazol-2-amine (OR0596);
4-(4-Aminopyrimidin-2-yl)-N-(4-methyl-4′-(2-(4-methylpiperazin-1-yl)-2-oxoethoxy)-[1,1′-biphenyl]-3-yl)-N-propylthiazol-2-amine (OR0597);
4-(4-Aminopyrimidin-2-yl)-N-(4-methyl-4′-(3-(4-methylpiperazin-1-yl)propyl)-[1,1′-biphenyl]-3-yl)-N-propylthiazol-2-amine (OR0598);
4-(4-Aminopyrimidin-2-yl)-N-(2-methyl-5-(6-(2-(4-methylpiperazin-1-yl)ethyl)pyridin-3-yl)phenyl)-N-propylthiazol-2-amine (OR0599);
2-(2-((4-Methyl-4′-(2-(4-methylpiperazin-1-yl)ethyl)-[1,1′-biphenyl]-3-yl)(propyl)amino)thiazol-4-yl)pyrimidine-4,6-diamine (OR0600);
2-(2-((4-Methyl-4′-(3-(4-methylpiperazin-1-yl)propyl)-[1,1′-biphenyl]-3-yl)(propyl)amino)thiazol-4-yl)pyrimidine-4,6-diamine (OR0601);
2-(2-((2-Methyl-5-(6-(2-(4-methylpiperazin-1-yl)ethyl)pyridin-3-yl)phenyl)(propyl)amino)thiazol-4-yl)pyrimidine-4,6-diamine (OR0602);
2-(2-(iso-Butyl(4-methyl-4′-(2-(4-methylpiperazin-1-yl)ethyl)-[1,1′-biphenyl]-3-yl)amino)thiazol-4-yl)pyrimidine-4,6-diamine (OR0603);
2-(2-((Cyclopropylmethyl)(4-methyl-4′-(2-(4-methylpiperazin-1-yl)ethyl)-[1,1′-biphenyl]-3-yl)amino)thiazol-4-yl)pyrimidine-4,6-diamine (OR0604);
2-(2-(iso-Pentyl(4-methyl-4′-(2-(4-methylpiperazin-1-yl)ethyl)-[1,1′-biphenyl]-3-yl)amino)thiazol-4-yl)pyrimidine-4,6-diamine (OR0605);
2-(2-(Butyl(4-methyl-4′-(2-(4-methylpiperazin-1-yl)ethyl)-[1,1′-biphenyl]-3-yl)amino)thiazol-4-yl)pyrimidine-4,6-diamine (OR0606);
2-(2-((4-Methyl-4′-((4-methylpiperazin-1-yl)sulfonyl)-[1,1′-biphenyl]-3-yl)(propyl)amino)thiazol-4-yl)pyrimidine-4,6-diamine (OR0607);
2-(2-(iso-Butyl(2-methyl-5-(6-(2-(4-methylpiperazin-1-yl)ethoxy)pyridin-3-yl)phenyl)amino)thiazol-4-yl)pyrimidine-4,6-diamine (OR0608);
N-((1-(3-((4-(4,6-Diaminopyrimidin-2-yl)thiazol-2-yl)(isobutyl)amino)-4-methylphenyl)-1H-1,2,3-triazol-4-yl)methyl) methanesulfonamide (OR0609);
2-(2-((2-Aminoethyl)(4-methyl-4′-(2-(4-methylpiperazin-1-yl)ethyl)-[1,1′-biphenyl]-3-yl)amino)thiazol-4-yl)pyrimidine-4,6-diamine (OR0610);
2-(2-((3-Aminopropyl)(4-methyl-4′-(2-(4-methylpiperazin-1-yl)ethyl)-[1,1′-biphenyl]-3-yl)amino)thiazol-4-yl)pyrimidine-4,6-diamine (OR0611);
2-[2-({2-Methyl-5-[6-(4-methyl-piperazin-1-ylcarbonyl)-pyridin-3-yl]-phenyl}-propyl-amino)-thiazol-4-yl]-pyrimidine-4,6-diamine (OR0612);
2-(2-((5-(2-Aminopyrimidin-5-yl)-2-methylphenyl)(propyl)amino)thiazol-4-yl)pyrimidine-4,6-diamine (OR0613);
2-(2-((4-Methyl-4′-((4-methylpiperazin-1-yl)sulfonyl)-[1,1′-biphenyl]-3-yl)amino)thiazol-4-yl)pyrimidine-4,6-diamine (OR0614);
3′-((4-(4,6-Diaminopyrimidin-2-yl)thiazol-2-yl)(propyl)amino)-4′-methyl-[1,1′-biphenyl]-4-sulfonamide (OR0615);
N-(5-(3-((4-(4,6-Diaminopyrimidin-2-yl)thiazol-2-yl)(propyl)amino)-4-methylphenyl)pyridin-2-yl)-4-methylpiperazine-1-carboxamide (OR0616);
2-(2-((4-Methyl-4′-(morpholinosulfonyl)-[1,1′-biphenyl]-3-yl)(propyl)amino)thiazol-4-yl)pyrimidine-4,6-diamine (OR0617);
3′-((4-(4,6-Diaminopyrimidin-2-yl)thiazol-2-yl)(propyl)amino)-N,N,4′-trimethyl-[1,1′-biphenyl]-4-sulfonamide (OR0618);
2-(2-((4′-((Dimethylamino)methyl)-4-methyl-[1,1′-biphenyl]-3-yl)(propyl)amino)thiazol-4-yl)pyrimidine-4,6-diamine (OR0619);
2-(2-((2-Methyl-5-(pyridin-3-yl)phenyl)(propyl)amino)thiazol-4-yl)pyrimidine-4,6-diamine (OR0620);
(5-(3-((4-(4,6-Diaminopyrimidin-2-yl)thiazol-2-yl)(propyl)amino)-4-methylphenyl)pyridin-2-yl)(morpholino) methanone (OR0621);
Methyl 5-(3-((4-(4,6-diaminopyrimidin-2-yl)thiazol-2-yl)(propyl)amino)-4-methylphenyl)picolinate (OR0622);
N-(5-(3-((4-(4,6-Diaminopyrimidin-2-yl)thiazol-2-yl)(propyl)amino)-4-methylphenyl)pyridin-2-yl)-4-methylpiperazine-1-sulfonamide (OR0625);
2-(2-((5-(1H-Indol-5-yl)-2-methylphenyl)(propyl)amino)thiazol-4-yl)pyrimidine-4,6-diamine (OR0626);
2-(2-((2-Methyl-5-(6-((4-methylpiperazin-1-yl)sulfonyl)pyridin-3-yl)phenyl)(propyl)amino)thiazol-4-yl)pyrimidine-4,6-diamine (OR0627);
2-(2-((2-Methyl-5-(6-(2-(4-methylpiperazin-1-yl)ethoxy)pyridin-3-yl)phenyl)(propyl)amino)thiazol-4-yl)pyrimidine-4,6-diamine (OR0629);
2-((5-(3-((4-(4,6-Diaminopyrimidin-2-yl)thiazol-2-yl)(propyl)amino)-4-methylphenyl)pyridin-2-yl)oxy)-1-(4-methylpiperazin-1-yl)ethan-1-one (OR0630);
2-(2-(Methyl(4-methyl-4′-((4-methylpiperazin-1-yl)sulfonyl)-[1,1′-biphenyl]-3-yl)amino)thiazol-4-yl)pyrimidine-4,6-diamine (OR0631);
2-(2-(Ethyl(4-methyl-4′-((4-methylpiperazin-1-yl)sulfonyl)-[1,1′-biphenyl]-3-yl)amino)thiazol-4-yl)pyrimidine-4,6-di-amine (OR0632);
2-(2-((5-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine (OR0633);
2-(2-((3′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine (OR0635);
2-(2-(iso-Propyl(4-methyl-4′-((4-methylpiperazin-1-yl)sulfonyl)-[1,1′-biphenyl]-3-yl)amino)thiazol-4-yl)pyrimidine-4,6-diamine (OR0636);
2-(2-(iso-Butyl (4-methyl-4′-((4-methylpiperazin-1-yl)sulfonyl)-[1,1′-biphenyl]-3-yl)amino)thiazol-4-yl)pyrimidine-4,6-diamine (OR0637);
2-(2-((4′-((4-Ethylpiperazin-1-yl)sulfonyl)-4-methyl-[1,1′-biphenyl]-3-yl)(propyl)amino)thiazol-4-yl)pyrimidine-4,6-diamine (OR0638);
2-(2-((4-Methyl-4′-(piperazin-1-ylsulfonyl)-[1,1′-biphenyl]-3-yl)(propyl)amino)thiazol-4-yl)pyrimidine-4,6-diamine (OR0639);
2-(2-((4-Methyl-4′-((4-methylpiperazin-1-yl)sulfonyl)-[1,1′-biphenyl]-3-yl)(propyl)amino)oxazol-4-yl)pyrimidine-4,6-diamine (OR0640);
2-(2-((2′-Chloro-4-methyl-4′-((4-methylpiperazin-1-yl)sulfonyl)-[1,1′-biphenyl]-3-yl)(propyl)amino)thiazol-4-yl)pyrimidine-4,6-diamine (OR0641);
2-(2-((4-Methyl-4′-((4-methylpiperazin-1-yl)sulfonyl)-2′-(trifluoromethyl)-[1,1′-biphenyl]-3-yl)(propyl)amino) thiazol-4-yl)pyrimidine-4,6-diamine (OR0642);
2-(2-((2′-Fluoro-4-methyl-4′-((4-methylpiperazin-1-yl)sulfonyl)-[1,1′-biphenyl]-3-yl)(propyl)amino)thiazol-4-yl)pyrimidine-4,6-diamine (OR0643);
2-(2-((2′-Fluoro-6′-methoxy-4-methyl-4′-((4-methylpiperazin-1-yl)sulfonyl)-[1,1′-biphenyl]-3-yl)(propyl)amino) thiazol-4-yl)pyrimidine-4,6-diamine (OR0644);
2-(2-((2′-Methoxy-4-methyl-4′-((4-methylpiperazin-1-yl)sulfonyl)-[1,1′-biphenyl]-3-yl)(propyl)amino)thiazol-4-yl)pyrimidine-4,6-diamine (OR0645);
2-(2-((4-Methyl-4′-(piperidin-4-ylsulfonyl)-[1,1′-biphenyl]-3-yl)(propyl)amino)thiazol-4-yl)pyrimidine-4,6-diamine (OR0646);
2-(2-((2′,6′-Difluoro-4-methyl-4′-((4-methylpiperazin-1-yl)sulfonyl)-[1,1′-biphenyl]-3-yl)(propyl)amino)thiazol-4-yl)pyrimidine-4,6-diamine (OR0647);
2-(2-((4′-((4-Aminopiperidin-1-yl)sulfonyl)-4-methyl-[1,1′-biphenyl]-3-yl)(propyl)amino)thiazol-4-yl)pyrimidine-4,6-diamine (OR0648);
2-(4-((3′-((4-(4,6-Diaminopyrimidin-2-yl)thiazol-2-yl)(propyl)amino)-4′-methyl-[1,1′-biphenyl]-4-yl)sulfonyl) piperazin-1-yl)acetic acid (OR0649);
2-(2-((4-Methyl-4′-((4-methylpiperazin-1-yl)sulfonyl)-3′-(trifluoromethoxy)-[1,1′-biphenyl]-3-yl)(propyl)amino) thiazol-4-yl)pyrimidine-4,6-diamine (OR0650);
N-(2-Aminoethyl)-3′-((4-(4,6-diaminopyrimidin-2-yl)thiazol-2-yl)(propyl)amino)-4′-methyl-[1,1′-biphenyl]-4-sulfonamide (OR0651);
2-(2-((2-Methyl-5-(4-methyl-6-((4-methylpiperazin-1-yl)sulfonyl)pyridin-3-yl)phenyl)(propyl)amino)thiazol-4-yl)pyrimidine-4,6-diamine (OR0652);
2-(2-((5-Bromo-2-methylphenyl)(isobutyl)amino)thiazol-4-yl)pyrimidine-4,6-diamine (OR0637-1);
2-(2-((5-Bromo-2-methylphenyl)(propyl)amino)thiazol-4-yl)pyrimidine-4,6-diamine (OR0652-1);
2-(2-((2-Methyl-5-(2-methyl-6-((4-methylpiperazin-1-yl)sulfonyl)pyridin-3-yl)phenyl)(propyl)amino)thiazol-4-yl)pyrimidine-4,6-diamine (OR0653);
2-(2-((5-(6-Methoxy-4-(trifluoromethyl)pyridin-3-yl)-2-methylphenyl)(propyl)amino)thiazol-4-yl)pyrimidine-4,6-diamine (OR0654);
2-(2-((5-(6-Hydroxy-4-(trifluoromethyl)pyridin-3-yl)-2-methylphenyl)(propyl)amino)thiazol-4-yl)pyrimidine-4,6-diamine (OR0655);
2-(2-((2-Methyl-5-(6-((4-methylpiperazin-1-yl)sulfonyl)-4-(trifluoromethyl)pyridin-3-yl)phenyl)(propyl)amino)thiazol-4-yl)pyrimidine-4,6-diamine (OR0656);
2-(2-((2-Methyl-5-(6-((4-methylpiperazin-1-yl)sulfonyl)-2-(trifluoromethyl)pyridin-3-yl)phenyl)(propyl)amino)thiazol-4-yl)pyrimidine-4,6-diamine (OR0657); and
2-(2-((2-Methyl-5-(6-(piperazin-1-ylsulfonyl)-2-(trifluoromethyl)pyridin-3-yl)phenyl)(propyl)amino)thiazol-4-yl)pyrimidine-4,6-diamine (OR0658).

34. A pharmaceutical composition comprising a compound according to claim 19 and a pharmaceutically acceptable excipient.

35. The pharmaceutical composition according to claim 34, further comprising an inhibitor of the de novo nucleotide biosynthesis pathway.

36. A method of treating a cancer comprising administering a compound according to claim 19, or a pharmaceutical composition thereof, to a subject having cancer.

37. The method according to claim 36, wherein the cancer is acute lymphoblastic leukemia or T-cell acute lymphoblastic leukemia.
